SEQUENCE LISTING (69 sequences)

SEQ ID NO: 1 - DNA, 43 bp, Artificial Sequence
  Encodes Kex2 linker
ctcgaggagtcctcttatgacaccattaggacctgcttcctcc

SEQ ID NO: 2 - DNA, 36 bp, Artificial Sequence
  Primer MAM227
ctcgaggagtcctcttacaccattaggacctgcttc

SEQ ID NO: 3 - DNA, 30 bp, Artificial Sequence
  Primer MAM228
gagctcggccggccttattatggttgagcc

SEQ ID NO: 4 - DNA, 37 bp, Artificial Sequence
  Primer MAM304
aaaaaagaattccgaaaaatgagcaccctgacattgc

SEQ ID NO: 5 - DNA, 149 bp, Artificial Sequence
  Primer MAM305
aaaaaaaggcctcttaaccaaagaacctccaccttcgtccgtacgagcacagccggtgatagaagtgggtttcatgtcctccggaaatcacttctatcaccggctgtgctcgtacggacgaaggtggaggttctttggttaagaggatg

SEQ ID NO: 6 - protein, 174 aa, Artificial Sequence
  GCSF, GenBank NP_757373, precursor molecule
TPLGPASSLPQSFLLKCLEQVRKIQGDGAALQEKLCATYKLCHPEELVLLGHSLGIPWAPLSSCPSQALQLAGCLSQLHSGLFLYQGLLQALEGISPELGPTLDTLQLDVADFATTIWQQMEELGMAPALQPTQGAMPAFASAFQRRAGGVLVASHLQSFLEVSYRVLRHLAQP

SEQ ID NO: 7 - DNA, 536 bp, Artificial Sequence
  Encodes GCSF
acaccattaggacctgcttcctccttgccccaatcattccttctgaagtgtttggaacaagtgcgaaagatacaaggtgatggagctgcccttcaagaaaaactatgtgcaacctacaagctgtgtcatcctgaggaattggtactgctgggacattcattaggtattccatgggccccattgtcttcttgtccaagtcaagctttacaactagccggttgtttgtcacagttacattctggtttgttcctataccaaggattactgcaagcactggaaggaatttcacctgaattgggtcctacattagatactttacaattggatgttgctgatttcgctactactatttggcaacaaatggaagagctaggtatggctccagcacttcaacctacgcaaggagcaatgccagcttttgcctctgcctttcagcgtcgagctggcggggtgttagttgcatctcacttacagtctttcctggaagttagttaccgtgtcctaagacatttggctcaaccataataaggccggcc

SEQ ID NO: 8 - protein, 174 aa, Artificial Sequence; identical to SEQ ID NO: 6
  Mature GCSF

SEQ ID NO: 9 - DNA, 1845 bp, Pichia pastoris
atgagcaccctgacattgctggctgtgctgttgtcgcttcaaaattcagctcttgctgctcaagctgaaactgcatccctatatcaccaatgtggtggtgcaaactgggagggagcaacccagtgtatttctggtgcctactgtcaatcgcagaacccatactactatcaatgtgttgctacttcttggggttactacactaacacctcaatctcttcgacggccacccttccttcttcttctactactgtctctccaaccagcagtgtggtgcccactggcttggtgtccccattgtatgggcaatgtgggggacagaattggaatggagccacatcttgtgctcagggaagctactgcaagtatatgaacaattattacttccaatgtgttcctgaagctgatggaaaccctgcagaaattagcactttttccgagaatggagagattatcgttactgcaatcgaagctcctacatgggctcaatgtggtggtcatggctactacggcccaactaaatgtcaagtgggaacatcatgccgtgaattaaacgcttggtattatcagtgtatcccagacgatcacaccgatgcctctactaccactttggatcctacttccagttttgtgagtacgacatcattatcgactcttccagcttcttcagaaacgacaattgtaactcctacctcaattgctgctgagcaagtacctctttggggacaatgtggaggaattggttacactggctctacgatttgtgagcagggatcgtgtgtttacttgaacgattggtactatcagtgtctaataagtgatcaaggtacagcatcaactgccagtgcaacgactagtataacttccttcaatgtttcatcgtcgtcagaaacgacggtaatagcccctacctcaatttctactgaggatgtcccactttggggccaatgtggaggaattggatataccggttcgaccacttgtagccagggatcatgcatttacttaaatgactggtattttcaatgtttaccagaggaggaaacgacttcatcaacttcgtcatcttcctcatcttcctcatcttccacatcttccgcatcttccacatcttccacatcatccacatcctccacatcctccacatcttcctcaacaagtagctcatccattccgacttctacaagctcatcgggagactttgagacaatccccaacggtttctcgggaactggaagaaccacgagatattgggattgttgtaagccaagctgctcatggcctgggaaatccaacagcgtaacaggaccagtgagatcttgtggtgtctctggcaacgtcctggacgccaacgcccaaagtggatgtattggtggtgaagctttcacttgtgatgagcaacaaccttggtccatcaacgacgacctagcctatggttttgccgcagcaagcctagctggtggatctgaggattcctcttgctgcacctgtatgaagctgacattcacctcatcttccattgctggaaagacaatgatcgttcaactgaccaatactggagctgatcttggatcgaatcactttgacattgctcttcctggtggagggcttggaatcttcaccgaaggatgctctagtcaatttggaagcggttaccaatggggtaaccagtatggtggtatctcttcgcttgctgagtgtgatggcctaccatcagaactgcagccaggctgtcagtttagatttggctggtttgagaacgctgataacccttcagtggagtttgaacaggtttcatgtcctccggaaatcacttctatcaccggctgtgctcgtacggacgaataa

SEQ ID NO: 10 - protein, 614 aa, Pichia pastoris
MSTLTLLAVLLSLQNSALAAQAETASLYHQCGGANWEGATQCISGAYCQSQNPYYYQCVATSWGYYTNTSISSTATLPSSSTTVSPTSSVVPTGLVSPLYGQCGGQNWNGATSCAQGSYCKYMNNYYFQCVPEADGNPAEISTFSENGEIIVTAIEAPTWAQCGGHGYYGPTKCQVGTSCRELNAWYYQCIPDDHTDASTTTLDPTSSFVSTTSLSTLPASSETTIVTPTSIAAEQVPLWGQCGGIGYTGSTICEQGSCVYLNDWYYQCLISDQGTASTASATTSITSFNVSSSSETTVIAPTSISTEDVPLWGQCGGIGYTGSTTCSQGSCIYLNDWYFQCLPEEETTSSTSSSSSSSSSSTSSASSTSSTSSTSSTSSTSSSTSSSSIPTSTSSSGDFETIPNGFSGTGRTTRYWDCCKPSCSWPGKSNSVTGPVRSCGVSGNVLDANAQSGCIGGEAFTCDEQQPWSINDDLAYGFAAASLAGGSEDSSCCTCMKLTFTSSSIAGKTMIVQLTNTGADLGSNHFDIALPGGGLGIFTEGCSSQFGSGYQWGNQYGGISSLAECDGLPSELQPGCQFRFGWFENADNPSVEFEQVSCPPEITSITGCARTDE

SEQ ID NO: 11 - DNA, 2397 bp, Artificial Sequence
  Encodes CLP1-rHuMetGCSF gene fusion
atgagcaccctgacattgctggctgtgctgttgtcgcttcaaaattcagctcttgctgctcaagctgaaactgcatccctatatcaccaatgtggtggtgcaaactgggagggagcaacccagtgtatttctggtgcctactgtcaatcgcagaacccatactactatcaatgtgttgctacttcttggggttactacactaacacctcaatctcttcgacggccacccttccttcttcttctactactgtctctccaaccagcagtgtggtgcccactggcttggtgtccccattgtatgggcaatgtgggggacagaattggaatggagccacatcttgtgctcagggaagctactgcaagtatatgaacaattattacttccaatgtgttcctgaagctgatggaaaccctgcagaaattagcactttttccgagaatggagagattatcgttactgcaatcgaagctcctacatgggctcaatgtggtggtcatggctactacggcccaactaaatgtcaagtgggaacatcatgccgtgaattaaacgcttggtattatcagtgtatcccagacgatcacaccgatgcctctactaccactttggatcctacttccagttttgtgagtacgacatcattatcgactcttccagcttcttcagaaacgacaattgtaactcctacctcaattgctgctgagcaagtacctctttggggacaatgtggaggaattggttacactggctctacgatttgtgagcagggatcgtgtgtttacttgaacgattggtactatcagtgtctaataagtgatcaaggtacagcatcaactgccagtgcaacgactagtataacttccttcaatgtttcatcgtcgtcagaaacgacggtaatagcccctacctcaatttctactgaggatgtcccactttggggccaatgtggaggaattggatataccggttcgaccacttgtagccagggatcatgcatttacttaaatgactggtattttcaatgtttaccagaggaggaaacgacttcatcaacttcgtcatcttcctcatcttcctcatcttccacatcttccgcatcttccacatcttccacatcatccacatcctccacatcctccacatcttcctcaacaagtagctcatccattccgacttctacaagctcatcgggagactttgagacaatccccaacggtttctcgggaactggaagaaccacgagatattgggattgttgtaagccaagctgctcatggcctgggaaatccaacagcgtaacaggaccagtgagatcttgtggtgtctctggcaacgtcctggacgccaacgcccaaagtggatgtattggtggtgaagctttcacttgtgatgagcaacaaccttggtccatcaacgacgacctagcctatggttttgccgcagcaagcctagctggtggatctgaggattcctcttgctgcacctgtatgaagctgacattcacctcatcttccattgctggaaagacaatgatcgttcaactgaccaatactggagctgatcttggatcgaatcactttgacattgctcttcctggtggagggcttggaatcttcaccgaaggatgctctagtcaatttggaagcggttaccaatggggtaaccagtatggtggtatctcttcgcttgctgagtgtgatggcctaccatcagaactgcagccaggctgtcagtttagatttggctggtttgagaacgctgataacccttcagtggagtttgaacaggtttcatgtcctccggaaatcacttctatcaccggctgtgctcgtacggacgaaggtggaggttctttggttaagaggatgacaccattaggacctgcttcctccttgccccaatcattccttctgaagtgtttggaacaagtgcgaaagatacaaggtgatggagctgcccttcaagaaaaactatgtgcaacctacaagctgtgtcatcctgaggaattggtactgctgggacattcattaggtattccatgggccccattgtcttcttgtccaagtcaagctttacaactagccggttgtttgtcacagttacattctggtttgttcctataccaaggattactgcaagcactggaaggaatttcacctgaattgggtcctacattagatactttacaattggatgttgctgatttcgctactactatttggcaacaaatggaagagctaggtatggctccagcacttcaacctacgcaaggagcaatgccagcttttgcctctgcctttcagcgtcgagctggcggggtgttagttgcatctcacttacagtctttcctggaagttagttaccgtgtcctaagacatttggctcaaccataataa

SEQ ID NO: 12 - protein, 789 aa, Artificial Sequence
  CLP1-rHuMetGCSF gene fusion
MSTLTLLAVLLSLQNSALAAQAETASLYHQCGGANWEGATQCISGAYCQSQNPYYYQCVATSWGYYTNTSISSTATLPSSSTTVSPTSSVVPTGLVSPLYGQCGGQNWNGATSCAQGSYCKYMNNYYFQCVPEADGNPAEISTFSENGEIIVTAIEAPTWAQCGGHGYYGPTKCQVGTSCRELNAWYYQCIPDDHTDASTTTLDPTSSFVSTTSLSTLPASSETTIVTPTSIAAEQVPLWGQCGGIGYTGSTICEQGSCVYLNDWYYQCLISDQGTASTASATTSITSFNVSSSSETTVIAPTSISTEDVPLWGQCGGIGYTGSTTCSQGSCIYLNDWYFQCLPEEETTSSTSSSSSSSSSSTSSASSTSSTSSTSSTSSTSSSTSSSSIPTSTSSSGDFETIPNGFSGTGRTTRYWDCCKPSCSWPGKSNSVTGPVRSCGVSGNVLDANAQSGCIGGEAFTCDEQQPWSINDDLAYGFAAASLAGGSEDSSCCTCMKLTFTSSSIAGKTMIVQLTNTGADLGSNHFDIALPGGGLGIFTEGCSSQFGSGYQWGNQYGGISSLAECDGLPSELQPGCQFRFGWFENADNPSVEFEQVSCPPEITSITGCARTDEMTPLGPASSLPQSFLLKCLEQVRKIQGDGAALQEKLCATYKLCHPEELVLLGHSLGIPWAPLSSCPSQALQLAGCLSQLHSGLFLYQGLLQALEGISPELGPTLDTLQLDVADFATTIWQQMEELGMAPALQPTQGAMPAFASAFQRRAGGVLVASHLQSFLEVSYRVLRHLAQP

SEQ ID NO: 13 - protein, 603 aa, Artificial Sequence
  secreted CLP1p fusion protein
AQAETASLYHQCGGANWEGATQCISGAYCQSQNPYYYQCVATSWGYYTNTSISSTATLPSSSTTVSPTSSVVPTGLVSPLYGQCGGQNWNGATSCAQGSYCKYMNNYYFQCVPEADGNPAEISTFSENGEIIVTAIEAPTWAQCGGHGYYGPTKCQVGTSCRELNAWYYQCIPDDHTDASTTTLDPTSSFVSTTSLSTLPASSETTIVTPTSIAAEQVPLWGQCGGIGYTGSTICEQGSCVYLNDWYYQCLISDQGTASTASATTSITSFNVSSSSETTVIAPTSISTEDVPLWGQCGGIGYTGSTTCSQGSCIYLNDWYFQCLPEEETTSSTSSSSSSSSSSTSSASSTSSTSSTSSTSSTSSSTSSSSIPTSTSSSGDFETIPNGFSGTGRTTRYWDCCKPSCSWPGKSNSVTGPVRSCGVSGNVLDANAQSGCIGGEAFTCDEQQPWSINDDLAYGFAAASLAGGSEDSSCCTCMKLTFTSSSIAGKTMIVQLTNTGADLGSNHFDIALPGGGLGIFTEGCSSQFGSGYQWGNQYGGISSLAECDGLPSELQPGCQFRFGWFENADNPSVEFEQVSCPPEITSITGCARTDEGGGSLVKR

SEQ ID NO: 14 - protein, 175 aa, Artificial Sequence
  Secreted rHuMetGCSF protein
MTPLGPASSLPQSFLLKCLEQVRKIQGDGAALQEKLCATYKLCHPEELVLLGHSLGIPWAPLSSCPSQALQLAGCLSQLHSGLFLYQGLLQALEGISPELGPTLDTLQLDVADFATTIWQQMEELGMAPALQPTQGAMPAFASAFQRRAGGVLVASHLQSFLEVSYRVLRHLAQP

SEQ ID NO: 15 - protein, 8 aa, Artificial Sequence
  Kex2 linker
GGGSLVKR

SEQ ID NO: 16 - DNA, 24 bp, Artificial Sequence
  Encodes Kex2 linker
ggtggaggttctttggttaagagg

SEQ ID NO: 17 - DNA, 7430 bp, Artificial Sequence
  VPS10-1 region
aaactaagtgggccagattatataaatatggatcaacatgaagccttgaaagatttcaaggacaggcttaggaattacgaaaaagtttacgagactattgacgaccaggaggaagaggagaacgaacggtacaatattcagtatctgaagataatcaacgcaggaaagaagatagtcagttataacataaatgggtatttatcgtcccacaccgttttttatctcctgaatttcaatcttgcagaacgtcaaatatggttgacgacgaatggagagacagagtataaccttcaaaataggattggaggtgattccaaattaagcaatgagggatggaaatttgccaaagcattgcccaagtttatagcacagaaaagaaaagagtttcaacttagacagttgaccaaacactatatcgagactcaaacgcccattgaagacgtaccgttggaggagcacaccaagccagtcaaatattctgatctgcatttccatgtttggtcatcggctttaaagagatctactcaatcaacaacattttttccatcggaaaattactctctgaagcaattcagaacgttgaatgatctctgttgcggatcactggatggtttgactgaacaagagttcaaaagtaaatacaaagaagaataccagaattctcagactgataaactgagtttcagtttccctggtatcggtggggagtcttatttggacgtgatcaaccgtttgagaccactaatagttgaactagaaaggttgccagaacatgtcctggtcattacccaccgggtcatagtaaggattttactaggatatttcatgaatttggatagaaatctgttgacagatttggaaattttgcatgggtatgtttattgtattgagccgaaaccttatggtttagacttaaagatctggcagtatgatgaggcggacaacgagtttaatgaagttgataagctggaattcatgaaaagaagaagaaaatcgatcaacgtcaacacgacagatttcagaatgcagttaaacaaagagttgcaacaggacgctctcaataatagtcctggtaataatagtccgggcgtatcatctctatcttcatactcgtcgtcctcttccctttccgctgacgggagcgagggagaaacattaataccacaagtatcccaggcggagagctacaactttgaatttaactctctttcatcatcagtttcatcgttgaaaaggacgacatcttcttcccaacatttgagctccaatcctagttgtctgagcatgcataatgcctcattggacgagaatgacgacgaacatttaatagacccggcttctacagacgacaagctaaacatggtattacaggacaaaacgctaattaaaaagctcaaaagtttactacttgacgaggccgaaggctagacaatccacagttaattttgatactgtactttataacgagtaacatacatatcttatgtaatcatctatgtcacgtcacgtgcgcgcgacattattccgagaacttgcgccctgctagctccactgtcagagtgataacttccccaaaataggatccaactgtttccaattgcttttggaaatgtggattgaaagaaacctcatagcgtctatattactattttcaacttcagcttatgcggcattcaaacccaggatagttaaaaaggaatttgatgaccttttgaatccaatatactttaacgattcatcgacagtactaggtctagtagatcagacgctgttaatttccaacgatgatggaaaatcatggactaacttgcaggaggttattacacctggggaaattgatccgctgacaattgtaaacattgaattcaatccatccgcatctaaggcttttgtattcactgctagtaagcactaccttactttagacaaaggatccacctggaaagaatttcaaattcctcttgaaaaatatggtaacagaatagcctacgacgttgagtttaattttgttaacgaagaacatgcaatcataagaacaaggtcttgcaaacgtcgttttgattgtaaggatgagtatttttattcgttagatgacttgcaaagcgttgacaagatcaccatttctgacgaaattgtcaattgccagttttcacaatcttccactagctcagattcccgcaaaaacgatgccatcacttgcgtaacgcgtaaactggattccaaccgacacttcttggagtcgaacgttctgacaaccttgaactttttcaaggatgttactagcttgcccgccagtgatccattaactaagatgcttatcaaggatatacgtgttgttcaaaattacattgtattgtttgtcagttcggatagatacaacaaatattcacccactcttcttttcatttccaaagatggaaatacgtttaaggaagccagtttaccagattctgaaggtacatcaccgtcggtgcactttttgaaaagtcctaatcccaatttgataagagcaattcggctagggaaaaagaactcactagatggtggtggcttttattcagaagttctacaatctgactctacagggttacactttcacgttcttctggaccacttagaagcaaatttgctttcgtactatcaaatagagaacttagcgaaccttgaaggaatctggattgccaaccaaatcgacacttccagcaagtttggctcaaaatccgttataacatttgatgcaggtttaacgtggtctcctgtgacagtagatgaagacgaagataaaagtttgcacatcattgcgtttgctggtgaaaatagcctttatgagtccaagtttccggtttcgactccaggaattgccttgaggatagggcttattggcgatagtagtgatgcacttgatattggcagctataggacatttttaaccagagatgcagggctaacatggtctcaagtttttgataatgtctctgtttgcggctttggaaactatggaaacatcatattatgctgttcgtatgatccactacttcgatctgagcctttgaaatttcgttattctttggatcaaggtcttaactgggaaagtattgatttaggcttcaacggagtcgctgttggcgttttgaacaatatagacaatagcagtcctcaattccttgtgatgacgattgccacggatggtaagtcttcaaaggctcagcatttcttgtattcagttgatttttctgatgcgtatgagaagaaaatatgtgatgttacaaaagacgaattatttgaagaatggacgggaagaatagatccggtgacgaagctgcctatttgtgttaacggtcacaaggaaaaattcagaagacggaaggctgacgctgaatgcttctctggtgaactttttcaagacctaactccaattgaagagccatgtgattgtgatccggatattgattacgaatgttcgcttggatttgagttcgatgcagagtctaaccgatgtgagccaaatttgtcaatcctgtccagtcactattgtgttgggaaaaacttaaagagaaaagtgaaagtagatagaaagtcgaaagttgcaggcacaaaatgtaaaaaggatgtcaaacttaaggataattctttcactttagactgttccaaaacatctgaaccagatctcagcgagcaaagaattgttagtaccaccataagctttgaaggttctccagtacaatacatttatttgaaacaggggaccaacacaacccttcttgacgaaacagtcattttaagaacatcactacgaactgtgtacgtgtctcataacgggggaacaacttttgatagagttagtatcgaagatgatgtgtcatttattgacatctatacaaaccattactttccagataatgtttatttgatcactgatacagatgagctgtacgtttcggataatagagctatctctttccagaaagttgacatgccttcaagagctggtttggagcttggagttcgagctctaacctttcataagagtgaccctaacaagtttatttggttcggtgagaaagattgtaactctatttttgacagaagttgtcaaacacaagcttatattacggaagacaacggcttatctttcaagcctcttttggaaaatgttagatcatgttactttgttggaacaacttttgattccaagctgtatgattttgacccgaacttaatcttttgcgagcagagagttccaaatcaacgtttcttgaaacttgtagccagtaaggactatttctatgatgacaaagaagagctgtatcctaagattattggaattgctactaccatgagctttgttatcgtagcgactatcaacgaagacaatagatcattgaaggcgtttataaccgcggatgggtctacttttgcggagcaattgtttcctgcagatctggattttggaagagaagtagcgtacacagttattgacaattgggaatcaaaaacacccaatttctttttccatttgacaacttctgaagataaagatttggaatttggagctttactgaaatcaaactacaatggaacaacctatacgcttgctgccaacaatgtcaatagaaacgatagaggttacgttgactatgaaatcgttctaaacttaaacggcattgctctcatcaatacagttattaactcgaaggaacttgaatccgagcagtcccttgaaactgctaaaaaactgaaaactcaaataacgtacaacgacgggtctgaatgggtgtatctgaaaccgccaaccattgattcagaaaagaacaagttttcgtgcgtcaaagataagttgagcttggaaaaatgctcattgaacctcaagggtgccactgatcggccagacagcagagactccatttcttctggttctgctgttggtctactttttggagtaggtaacgttggggaatacctgaaccaagattcatcaggtctagcattgtatttttcgaaggatgcgggcatctcttggaaggagattgccaaaggagattatatgtgggaatttggagatcaaggaacaatcctcgtaattgttgagttcaagaagaaggttgacactttgaaatactcattggatgaaggagaaacgtggttcgactacaagtttgcaaatgaaaaaacatatgttttggacctagcaactgtgccttcagatacttcacggaagttcatcatcctcgccaacagaggcgaggagggagatcatgaaactgttgttcacacaatagacttcagtaaggttcaccagcgtcaatgtttattgaatttacaagatagtaacgctggtgatgatttcgaatattggagtccgaagaacccaagcgctgttgacgggtgtatgctagggcatgaagagtcttacctaaaaaggattgcatcccactcggattgttttattgggaacgcacccctatcagagaaatacaaagtgattaagaactgcgcttgcacaaggagagattacgaatgtgattacaattttgctcttgccaatgatggaacttgtaaattggtggaaggagagtctcctttggattactctgaagtttgtagaagggatccaacttccattgaatattttttgcctactgggtacagaaaggtgggattgagtacttgtgaaggcggactagaactggataattggaatcccgttccatgtccaggaaaaaccagagaattcaatagaaaatacggcaccggcgccaccggatacaagattgtggtcatagtagcagtgcctttattggttctcttgagcgccacttggttcctatatgagaaaggaataaaaaggaatggaggttttgccagatttggagttattcgattaggcgaagatgacgacgatgacttgcaaatgattgaggagaataatactgacaaagtagtcaatgttgtagtgaaaggcctcattcatgcattcagagcagtttttgtgagctatttatttttccgcaaacgtgcggccaagatgtttggtggatcgtccttttcacacagacacatattgcctcaagatgaggatgctcaagcctttttagccagcgacttggagtcagagagtggagagcttttccgatatgcaagcgacgatgacgatgcccgagagattgacagcgtgatcgagggaggaattgatgtcgaagacgacgacgaggagaatatcaattttgattcccggtagatagctcacccacggtcacacacacaaacacacatacacattaacacacagagttattagttaacagagaaaactctaacaaagtatttattttcgttacgtaatccgacttttctttttaccgttttctattgctcctctcatttgcccctaaaagttgctcctcattactaaaatcaccacaccatgctcgaatatgatgttactaaatgcaaattgtagtcgtgcctcttgtggtaatactatagggaatatctctcgattactcgattctggttaattttttctttttttataggggaagtttttttttcttcccctttctctccagtttatttatttactaagaaaatccaacagataccaaccacccaaaaagatcctaaacagcctgtttttgaggagtttttcagcagctaagcttcatcagttttttaatacttaatttattgcccttcactttgtttcttgtggcttttaaggctctccggaacagcggtttcaaaatcaaatctcagttatttgtttgctccgctttgtcagttcaaagatcatggtttccgaaaacaagaatcaatcttcgattttgatggacaactccaagaagctctctccgaagcccattttgaataacaagaatgaaccgtttggcatcggcgtcgatggacttcaacatcctcaaccgactttatgccgcacagaatcggaactcttgttcaacttgagccaagtcaataaatcccaaataactttggacggtgcagttactccacctgctgatggtaatgggaatgaagcaaaaagagcaaatctcatctcttttgatgttccatcgtctcaagtgaaacatagagggtctattagtgcaaggccctcggcagtgaatgtgtcccaaattaccggggccctttctcaatccggatcttctagaaatccctacgatcaaacacagtcacctccacctagcacttacgcctccaggcagaactccacccatggaaataatatcgatagcttgcaatatttggcaacaagagatcttagtgctttaaggctggaaagagatgcttccgcacgagaagctacctcttctgcagtgtccactcctgttcagttcgatgtacccaaacaacatcatctccttcatttagaacaagacccgacaaggcccatccctattgccgacaaaaag

SEQ ID NO: 18 - DNA, 2957 bp, Artificial Sequence
  PEP4 region
atttgagtcacctgctttagggctggaagatatttggttactagattttagtacaaactcttgctttgtcaatgacattaaaataggcaagaatcgcaaaactcaaatatttcatggagatgagatatgcttgttcaaagatgcccagaaaaaagagcaactcgtttatagggttcatattgatgatggaacaggccttttccagggaggtgaaagaacccaagccaattctgatgacattctggatattgatgaggttgatgaaaagttaagagaactattgacaagagcctcaaggaaacggcatatcacccctgcattggaaactcctgataaacgtgtaaaaagagcttatttgaacagtattactgataactcttgatggaccttaaagatgtataatagtagacagaattcataatggtgagattaggtaatcgtccggaataggaatagtggtttggggcgattaatcgcacctgccttatatggtaagtaccttgaccgataaggtggcaactatttagaacaaagcaagccacctttctttatctgtaactctgtcgaagcaagcatctttactagagaacatctaaaccattttacattctagagttccatttctcaattactgataatcaatttaaagatgatatttgacggtactacgatgtcaattgccattggtttgctctctactctaggtattggtgctgaagccaaagttcattctgctaagatacacaagcatccagtctcagaaactttaaaagaggccaattttgggcagtatgtctctgctctggaacataaatatgtttctctgttcaacgaacaaaatgctttgtccaagtcgaattttatgtctcagcaagatggttttgccgttgaagcttcgcatgatgctccacttacaaactatcttaacgctcagtattttactgaggtatcattaggtacccctccacaatcgttcaaggtgattcttgacacaggatcctccaatttatgggttcctagcaaagattgtggatcattagcttgcttcttgcatgctaagtatgaccatgatgagtcttctacttataagaagaatggtagtagctttgaaattaggtatggatccggttccatggaagggtatgtttctcaggatgtgttgcaaattggggatttgaccattcccaaagttgattttgctgaggccacatcggagccggggttggccttcgcttttggcaaatttgacggaattttggggcttgcttatgattcaatatcagtaaataagattgttcctccaatttacaaggctttggaattagatctccttgacgaaccaaaatttgccttctacttgggggatacggacaaagatgaatccgatggcggtttggccacatttggtggtgtggacaaatctaagtatgaaggaaagatcacctggttgcctgtcagaagaaaggcttactgggaggtctcttttgatggtgtaggtttgggatccgaatatgctgaattgcaaaaaactggtgcagccatcgacactggaacctcattgattgctttgcccagtggcctagctgaaattctcaatgcagaaattggtgctaccaagggttggtctggtcaatacgctgtggactgtgacactagagactctttgccagacttaactttaaccttcgccggttacaactttaccattactccatatgactatactttggaggtttctgggtcatgtattagtgctttcacccccatggactttcctgaaccaataggtcctttggcaatcattggtgactcgttcttgagaaaatattactcagtttatgacctaggcaaagatgcagtaggtttagccaagtctatttaggcaagaataaaagttgctcagctgaacttatttggttacttatcaggtagtgaagatgtagagaatatatgtttaggtatttttttttagtttttctcctataactcatcttcagtacgtgattgcttgtcagctaccttgacaggggcgcataagtgatatcgtgtactgctcaatcaagatttgcctgctccattgataagggtataagagacccacctgctcctctttaaaattctctcttaactgttgtgaaaatcatcttcgaagcaaattcgagtttaaatctatgcggttggtaactaaaggtatgtcatggtggtatatagtttttcattttaccttttactaatcagttttacagaagaggaacgtctttctcaagatcgaaataggactaaatactggagacgatggggtccttatttgggtgaaaggcagtgggctacagtaagggaagactattccgatgatggagatgcttggtctgcttttccttttgagcaatctcatttgagaacttatcgctggggagaggatggactagctggagtctcagacaatcatcaactaatttgtttctcaatggcactgtggaatgagaatgatgatattttgaaggagcgattatttggggtcactggagaggctgcaaatcatggagaggatgttaaggagctttattattatcttgataatacaccttctcactcttatatgaaatacctttacaaatatccacaatcgaaatttccttacgaagaattgatttcagagaaccgtaaacgttccagattagaaagagagtacgagattactgactctgaagtactgaaggataacagatattttgatgtgatctttgaaatggcaaaggacgatgaagatgagaatgaactttactttagaattaccgcttacaaccgaggtcccacccctgcccctttacatgtcgctccacaggtaacctttagaaatacctggtcctggggtatagatgaggaaaaggatcacgacaaacctatagcttgcaaggaataccaagacaacaactattctattcggttagatagtt

SEQ ID NO: 19 - DNA, 5040 bp, Artificial Sequence
  PRB1 region
ctaaacgtgaatgaagatgcgaggaagggtgtggcagaatgaaggaagaattggtggcaatactgacctggctaaaacctattcaaactgggctaaatacaggattcatgagtttcctgatctcaatatttttcagtcctccttgcccttgcaacgttttcttattcaatgcccaaactctcccatcgacgtcgcctcgaaactttctgaaaatcatgaccgtctgtttaatctcccgagactcttcttctctatgaacattcactcgttagcttccctaaatgagtcaattagaaatcttttttaaaaagattcattctacgattcggcttcccgaaaaagaggcaagtgaattgctcaagaaacaattgactatgaacccaaaatctcctcatctcccaaaacttcaagtggatctacagaatcaatctgaacaaaccataagcaaattcgtgcaagatcaacagttctttggtggcgactgggctcggttcgaaagccttattgtcagctatttaaaatttgttagaaactttgacccctggtcgatattgaaatccattgatctaatgattaacgttgttgacgagttggcaagttctctcaacaaacaacagcattacaagtacctgtttgggactcttgttgattatgtcattcttttgcatcctcttgtcaaattggttgataaaaaattgctaattatcaaaaagaggaacagctattatccaaggcttacgcagatgtctaccattttgcagaaagctttcaacaatattagaaatcaaagagatccaaccggccagatatcaagggaccaacaactggtcttattcttgcttggtataaagacttgctacatctactttaacatcaatcatctcttgagatgcaatgatatcttctccaacatgaacgtgttgaacttggacgccaaaattatccctaagtcccagctaattcagtatagatttttgttgggaaagtttaacttcatacagaataacttcatgactgcatttgttcaattgaactggtgtttgaacaacgcctacatcaataataccaatcatcggacgaaaaatatggaattaatactaaaatatcttatcccctccagtcttatagttggtaagataccaaatttgaacatcctgaaccagctgctgtcatctcaagaggcacaccctctgattgagctttatcgaccactgatttcaaccctcaaaaagggtaatgttttcgaattccacaaatacctgtttgataatgagtcatactttttaaagatgaacgttctcctgccgctacttcaacggttgcgtattttgctgttcagaaatctggtccgaaagctggcccttatagagccaccagtcaacaactctctgagattttcatccatcaaaacagcccttttcgtttccatttcacccaatcaaaacgcatactttcagaacaattattcatacctgattgttaccaacgagtcccagatagacgactcctttgtggagaacctcatgatcagtctaatcgatcaaaacctaattaagggtaaactcgtcaacgataaccaccgaataattgtctccaaggccgatacattcccggagatccctacgatttattcgactaagtttgccgtagactcgtcattcgattggctggaccaatagacgtcctttttttttttttttttatcgtgtctgccgtttaatgtcacgcctcatgtttcaagttacgataacttatcatgcagatactaaatagtcacatgacgaatgacgattttttgcgggttgctcagaggaatatgcctctgataagcgaggtaaatgtcgagcataagccacttactgtataaatacccctttatcgccactttatcttttctccttgtccgttatctacaacaccccagtaaaacattacaaacactctagtgttgttttactgtcccttttaactctcttcaaacaaatctccatattatttaaactatgcaattgcgtcattccgttggattggctatcttatctgccatagcagtccaaggattgctaattcctaacattgagtcattacccagccagtttggtgctaatggtgacagtgaacaaggtgtattagcccaccatggtaaacatcctaaagttgatatggctcaccatggaaagcatcctaaaatcgctaaggattccaagggacaccctaagctttgccctgaagctttgaagaagatgaaagaaggccacccttcggctccagtcattactacccattccgcttctaaaaacttaatcccttactcttatattatagtcttcaagaagggtgtcacttcagaggatatcgacttccaccgtgaccttatctccactcttcatgaagagtctgtgagcaaattaagagagtcagatccaaatcactcatttttcgtttctaatgagaatggcgaaacaggttacaccggtgacttctccgttggtgacttgctcaagggttacaccggatacttcacggatgacactttagagcttatcagtaagcatccagcagttgctttcattgaaagggattcgagagtatttgccaccgattttgaaactcaaaacggtgctccttggggtttggccagagtctctcacagaaagcctctttccctaggcagcttcaacaagtacttatatgatggagctggtggtgaaggtgttacttcctatgttatcgatacaggtatccacgtcactcacaaagaattccagggtagagcatcttggggtaagaccattccagctggagacgttgatgacgatggaaacggtcacggaactcactgtgctggtaccattgcttctgaaagctacggtgttgccaagaaggctaatgttgttgccatcaaggtcttgagatctaatggttctggttcgatgtcagatgttctgaagggtgttgagtatgccacccaatcccacttggatgctgttaaaaagggcaacaagaaatttaagggctctaccgctaacatgtcactgggtggtggtaaatctcctgctttggaccttgcagtcaatgctgctgttaagaatggtattcactttgccgttgcagcaggtaacgaaaaccaagatgcttgtaacacctcgccagcagctgctgagaatgccatcaccgtcggtgcatcaaccttatcagacgctagagcttacttttctaactacggtaaatgtgttgacattttcgctccaggtttaaacattctttctacctacactggttcggatgacgcaactgctaccttgtctggtacttcaatggcctctcctcacattgctggtctgttgacttacttcctatcattgcagcctgctgctggatctctgtactctaacggaggatctgagggtgtcacacctgctcaattgaaaaagaacctcctcaagtatgcatctgtcggagtattagaggatgttccagaagacactccaaacctcttggtttacaatggtggtggacaaaacctttcttctttctggggaaaggagacagaagacaatgttgcttcctccgacgatactggtgagtttcactcttttgtgaacaagcttgaatcagctgttgaaaacttggcccaagagtttgcacattcagtgaaggagctggcttctgaacttatttagattggagaaaaggaatacacaaggagttaaaaaaagtgtggtagaaagtgcatttgtcataattttccatatgttgctgtcactgtaatcttttatattttgttttgttttatgtagtatttcaaaaggttcttatcatcttactggcataaacttgatgtacgcagagatagcaaccgttgcttaggtaagcatagtaaaaatggctggttttctgtcttattttaaggccactgttgggacaaaacacaataactagattttatcggattgaacagtgtaaaggcttcactggcttatatcttgtatgagtacgatacattatccagttccatcaaggcctgtggaaatattacagccaggacatgaacctgaaagggagtttagtgggatcactgtagataataggaacagacttaatgaagaaaagtattatcagacgaaaatagacgaagcgttgaaaaggggcacagaaagacgttacgttgatgatcatagcagaggtcatgagtctccaagttcagatttggaggacactccggatcaattcttggaatttcacattcatgataacggagataggaagatttcaaggccagacactgcttcgtcattgattagtgaaaacgacatggactacgatgatttgtttgttgacagaaagcaaccaaaacatgctacttctcatgtaaagcagtttattaggaagaatgtgttccaaaagaagactcatctaccaaacattggggctagagaactggaattacagaaacggcttgctttattagagggcccaatagatgacgatgagattattagtgctatgcccatggtagcgtgtccctctgactataacgatcaacctgctgattcaaattcaagtaaagcgttacagagttcaaccgcctctaatccctccagttcattgcctaaaaaagaagaggaggcaattaaagctgtacgggaagatgagcaggatactgcaccagacggagatgcctatggcattggaagcttggtggcagacgctgcttttaagtttctcaactacattttgccttcggattctagctccaaccccagttcgacagctatctccacagtagataaggcattgccgccagctccaacatttatgtcgtcaggtccctgtttagatggtgctagacccagttcaacttctccctgtacgagaaccacgccgctttattcgtacatggctccaaaagattcaagcagaaatcaaacggtaattttgaaagctttcaaacgcccattttcaaagaaatcaagttcaagcgtctctcctaagcgggaaaatcacactgaattaattcctagtactggccccttgtgg

SEQ ID NO: 20 - DNA, 2610 bp, Pichia pastoris
atgacatctcggacagctgagaacccgttcgatatagagcttcaagagaatctaagtccacgttcttccaattcgtccatattggaaaacattaatgagtatgctagaagacatcgcaatgattcgctttcccaagaatgtgataatgaagatgagaacgaaaatctcaattatactgataacttggccaagttttcaaagtctggagtatcaagaaagagctgtatgctaatatttggtatttgctttgttatctggctgtttctctttgccttgtatgcgagggacaatcgattttccaatttgaacgagtacgttccagattcaaacagccacggaactgcttctgccaccacgtctatcgttgaaccaaaacagactgaattacctgaaagcaaagattctaacactgattatcaaaaaggagctaaattgagccttagcggctggagatcaggtctgtacaatgtctatccaaaactgatctctcgtggtgaagatgacatatactatgaacacagttttcatcgtatagatgaaaagaggattacagactctcaacacggtcgaactgtatttaactatgagaaaattgaagtaaatggaatcacgtatacagtgtcatttgtcaccatttctccttacgattctgccaaattcttagtcgcatgcgactatgaaaaacactggagacattctacgtttgcaaaatatttcatatatgataaggaaagcgaccaagaggatagctttgtacctgtctacgatgacaaggcattgagcttcgttgaatggtcgccctcaggtgatcatgtagtattcgtttttgaaaacaatgtatacctcaaacaactctcaactttagaggttaagcaggtaacttttgatggtgatgagagtatttacaatggtaagcctgactggatctatgaagaggaagttttaagtagcgacagagccatatggtggaatgacgatggatcgtactttacgttcttgagacttgatgacagcaatgtcccaaccttcaacttgcagcatttttttgaagaaacaggctctgtgtcgaaatatccggtcattgatcgattgaaatatccaaaaccaggatttgacaaccccctggtttctttgtttagttacaacgttgccaagcaaaagttagaaaagctaaatattggagcagcagtttctttgggagaagacttcgtgctttacagtttaaaatggatagacaattcttttttcttgtcgaagttcacagaccgcacttcgaaaaaaatggaagttactctagtggacattgaagccaattctgcttcggtggtgagaaaacatgatgcaactgagtataacggctggttcactggagaattttctgtttatcctgtcgttggagataccattggttacattgatgtaatctattatgaggactacgatcacttggcttattatccagactgcacatccgataagtatattgtgcttacagatggttcatggaatgttgttggacctggagttttagaagtgcttgaagatagagtctactttatcggcaccaaagaatcatcaatggaacatcacttgtattatacatcattaacgggacccaaggttaaggctgttatggatatcaaagaacctgggtactttgatgtaaacattaagggaaaatatgctttactatcttacagaggccccaaactcccataccagaaatttattgatctttctgaccctagtacaacaagtcttgatgacattttatcgtctaatagaggaattgtcgaggttagtttagcaactcacagcgttcctgtttctacctatactaatgtaacacttgaggacggcgtcacactgaacatgattgaagtgttgcctgccaattttaatcctagcaagaagtacccactgttggtcaacatttatggtggaccgggctcccagaagttagatgtgcagttcaacattgggtttgagcatattatttcttcgtcactggatgcaatagtgctttacatagatccgagaggtactggaggtaaaagctgggcttttaaatcttacgctacagagaaaataggctactgggaaccacgagacatcactgcagtagtttccaagtggatttcagatcactcatttgtgaatcctgacaaaactgcgatatgggggtggtcttacggtgggttcactacgcttaagacattggaatatgattctggagaggttttcaaatatggtatggctgttgctccagtaactaattggcttttgtatgactccatctacactgaaagatacatgaaccttccaaaggacaatgttgaaggctacagtgaacacagcgtcattaagaaggtttccaattttaagaatgtaaaccgattcttggtttgtcacgggactactgatgataacgtgcattttcagaacacactaaccttactggaccagttcaatattaatggtgttgtgaattacgatcttcaggtgtatcccgacagtgaacatagcattgcccatcacaacgcaaataaagtgatctacgagaggttattcaagtggttagagcgggcatttaacgatagatttttgtaa

SEQ ID NO: 21 - DNA, 2448 bp, Pichia pastoris
atgtatcccgaacacaagtatcgggagtatcaacggagggtgcccttatggcagtactccctgttggtgattgtactgctatacgggtctcatttgcttatcagcaccatcaacttgatacactataaccacaaaaattatcatgcacacccagtcaatagtggtatcgttcttaatgagtttgctgatgacgattcattctctttgaatggcactctgaacttggagaactggagaaatggtaccttttcccctaaatttcattccattcagtggaccgaaataggtcaggaagatgaccagggatattacattctctcttccaattcctcttacatagtaaagtctttatccgacccagactttgaatctgttctattcaacgagtctacaatcacttacaacggtgaagaacatcatgtggaagacgtcatagtgtccaataatcttcaatatgcattggtagttacggataagagacataattggcgccattctttttttgcgaattactggctgtataaagtcaacaatcctgaacaggttcagcctttgtttgatacagatctatcgttgaatggtcttattagccttgtccattggtctccggattcttcccaagttgcatttgtgttggaaaataacatatatttgaagcatcttaacaacttttctgattcaaggattgatcaactaacttatgatggaggcgaaaacatattttatggcaaaccagattgggtttatgaagaagaagtgtttgaaagcaactctgctatgtggtggtctccaaatggaaagtttttatcaatattgcgaactaatgacacccaagtgcctgtctatcctattccatattttgttcagtctgatgctgaaacagctatcgatgaataccctcttctgaaacacataaaatacccaaaggcaggatttcccaatccagttgttgatgtgattgtatacgatgttcaacgccagcacatatctaggttacctgctggtgatcctttctacaacgatgagaacattaccaatgaggacagacttatcactgagatcatctgggttggtgattcacggttcctgaccaagattacgaacagggaaagtgacttgttagcattttatctggtagacgctgaggctaacaatagtaagctggtaagattccaagatgctaagagcaccaagtcttggtttgaaattgaacacaacacattgtatattcctaaggatacttcagtgggaagggcacaagatggctacatcgacaccatagatgttaacggctacaaccatttagcctatttctcaccaccagacaacccagaccccaaggtcattcttacgcgtggtgattgggaagtcgttgacagtccatctgcatttgacttcaaaagaaatttggtttactttacagcaaccaagaaatcctcaatagaaagacatgtttattgtgttgggatagacgggaaacaattcaacaatgtaactgatgtttcatcagatggatactacagtacaagcttttcccctggagcaagatatgtattgctatcacaccaaggtccccgtgtaccttatcaaaagatgatagatcttgtcaaaggcaccgaagaaataatcgaatctaacgaagatttgaaagactccgttgctttatttgatttacctgatgtcaagtacggcgaaatcgagcttgaaaaaggtgtcaagtcaaactacgttgagatcaggcctaagaacttcgatgaaagcaaaaagtatccggttttattttttgtgtatggggggccaggttcccaattggtaacaaagacattttctaagagtttccagcatgttgtatcctctgagcttgacgtcattgttgtcacggtggatggaagagggactggatttaaaggtagaaaatatagatccatagtgcgggacaacttgggtcattatgaatccctggaccaaatcacggcaggaaaaatttgggcagcaaagccttacgttgatgagaatagactggccatttggggttggtcttatggaggttacatgacgctaaaggttttagaacaggataaaggtgaaacattcaaatatggaatgtctgttgcccctgtgacgaattggaaattctatgattctatctacacagaaagatacatgcacactcctcaggacaatccaaactattataattcgtcaatccatgagattgataatttgaagggagtgaagaggttcttgctaatgcacggaactggtgacgacaatgttcacttccaaaatacactcaaagttctagatttatttgatttacatggtcttgaaaactatgatatccacgtgttccctgatagtgatcacagtattagatatcacaacggtaatgttatagtgtatgataagctattccattggattaggcgtgcattcaaggctggcaaa

SEQ ID NO: 22 - DNA, 60 bp, Artificial Sequence
  Encodes Alpha amylase signal peptide (from Aspergillus niger  alpha-amylase)
atggttgcttggtggtccttgttcttgtacggattgcaagttgctgctccagctttggct

SEQ ID NO: 23 - protein, 20 aa, Artificial Sequence
  Alpha amylase signal peptide (from Aspergillus niger  alpha-amylase)
MVAWWSLFLYGLQVAAPALA

SEQ ID NO: 24 - DNA, 57 bp, Artificial Sequence
  Encodes Saccharomyces cerevisiae mating factor pre-signal peptide DNA
atgagattcccatccatcttcactgctgttttgttcgctgcttcttctgctttggct

SEQ ID NO: 25 - protein, 19 aa, Artificial Sequence
  Saccharomyces cerevisiae mating factor pre-signal peptide DNA
MRFPSIFTAVLFAASSALA

SEQ ID NO: 26 - DNA, 141 bp, Artificial Sequence
  Encodes Saccharomyces cerevisiae mating factor pre-pro signal peptide (MFIL-1beta prepro) DNA
atgcgatttccttccatttttactgctgttttgtttgccgcctcctcagctttggcctcactgaactgtacactgcgtgattcacagcagaaaagtctggtcatgtccggaccatacgaacttaaagccttagttaaaaga

SEQ ID NO: 27 - protein, 47 aa, Artificial Sequence
  Saccharomyces cerevisiae mating factor pre-pro signal peptide (MFIL-1beta prepro) DNA
MRFPSIFTAVLFAASSALASLNCTLRDSQQKSLVMSGPYELKALVKR

SEQ ID NO: 28 - DNA, 54 bp, Artificial Sequence
  Encodes HSA signal peptide
atgaagtgggttacctttatctctttgttgtttcttttctcttctgcttactct

SEQ ID NO: 29 - protein, 18 aa, Artificial Sequence
  HSA signal peptide
MKWVTFISLLFLFSSAYS

SEQ ID NO: 30 - DNA, 1143 bp, Pichia pastoris
atggctatattcgccgtttctgtcatttgcgttttgtacggaccctcacaacaattatcatctccaaaaatagactatgatccattgacgctccgatcacttgatttgaagactttggaagctccttcacagttgagtccaggcaccgtagaagataatcttcgaagacaattggagtttcattttccttaccgcagttacgaaccttttccccaacatatttggcaaacgtggaaagtttctccctctgatagttcctttccgaaaaacttcaaagacttaggtgaaagttggctgcaaaggtccccaaattatgatcattttgtgatacccgatgatgcagcatgggaacttattcaccatgaatacgaacgtgtaccagaagtcttggaagctttccacctgctaccagagcccattctaaaggccgattttttcaggtatttgattctttttgcccgtggaggactgtatgctgacatggacactatgttattaaaaccaatagaatcgtggctgactttcaatgaaactattggtggagtaaaaaacaatgctgggttggtcattggtattgaggctgatcctgatagacctgattggcacgactggtatgctagaaggatacaattttgccaatgggcaattcagtccaaacgaggacacccagcactgcgtgaactgattgtaagagttgtcagcacgactttacggaaagagaaaagcggttacttgaacatggtggaaggaaaggatcgtggaagtgatgtgatggactggacgggtccaggaatatttacagacactctatttgattatatgactaatgtcaatacaacaggccactcaggccaaggaattggagctggctcagcgtattacaatgccttatcgttggaagaacgtgatgccctctctgcccgcccgaacggagagatgttaaaagagaaagtcccaggtaaatatgcacagcaggttgttttatgggaacaatttaccaacctgcgctcccccaaattaatcgacgatattcttattcttccgatcaccagcttcagtccagggattggccacagtggagctggagatttgaaccatcaccttgcatatattaggcatacatttgaaggaagttggaaggac

SEQ ID NO: 31 - protein, 381 aa, Pichia pastoris
MAIFAVSVICVLYGPSQQLSSPKIDYDPLTLRSLDLKTLEAPSQLSPGTVEDNLRRQLEFHFPYRSYEPFPQHIWQTWKVSPSDSSFPKNFKDLGESWLQRSPNYDHFVIPDDAAWELIHHEYERVPEVLEAFHLLPEPILKADFFRYLILFARGGLYADMDTMLLKPIESWLTFNETIGGVKNNAGLVIGIEADPDRPDWHDWYARRIQFCQWAIQSKRGHPALRELIVRVVSTTLRKEKSGYLNMVEGKDRGSDVMDWTGPGIFTDTLFDYMTNVNTTGHSGQGIGAGSAYYNALSLEERDALSARPNGEMLKEKVPGKYAQQVVLWEQFTNLRSPKLIDDILILPITSFSPGIGHSGAGDLNHHLAYIRHTFEGSWKD

SEQ ID NO: 32 - protein, 4 aa, Artificial Sequence
  CYP sorting signal
QRPL

SEQ ID NO: 33 - protein, 4 aa, Artificial Sequence
  Cryptic CYP sorting signal
QSFL

SEQ ID NO: 34 - DNA, 1494 bp, Artificial Sequence
  Tricoderma reesei alpha-1,2-mannosidase catalytic domain
cgcgccggatctcccaaccctacgagggcggcagcagtcaaggccgcattccagacgtcgtggaacgcttaccaccattttgcctttccccatgacgacctccacccggtcagcaacagctttgatgatgagagaaacggctggggctcgtcggcaatcgatggcttggacacggctatcctcatgggggatgccgacattgtgaacacgatccttcagtatgtaccgcagatcaacttcaccacgactgcggttgccaaccaaggcatctccgtgttcgagaccaacattcggtacctcggtggcctgctttctgcctatgacctgttgcgaggtcctttcagctccttggcgacaaaccagaccctggtaaacagccttctgaggcaggctcaaacactggccaacggcctcaaggttgcgttcaccactcccagcggtgtcccggaccctaccgtcttcttcaaccctactgtccggagaagtggtgcatctagcaacaacgtcgctgaaattggaagcctggtgctcgagtggacacggttgagcgacctgacgggaaacccgcagtatgcccagcttgcgcagaagggcgagtcgtatctcctgaatccaaagggaagcccggaggcatggcctggcctgattggaacgtttgtcagcacgagcaacggtacctttcaggatagcagcggcagctggtccggcctcatggacagcttctacgagtacctgatcaagatgtacctgtacgacccggttgcgtttgcacactacaaggatcgctgggtccttgctgccgactcgaccattgcgcatctcgcctctcacccgtcgacgcgcaaggacttgacctttttgtcttcgtacaacggacagtctacgtcgccaaactcaggacatttggccagttttgccggtggcaacttcatcttgggaggcattctcctgaacgagcaaaagtacattgactttggaatcaagcttgccagctcgtactttgccacgtacaaccagacggcttctggaatcggccccgaaggcttcgcgtgggtggacagcgtgacgggcgccggcggctcgccgccctcgtcccagtccgggttctactcgtcggcaggattctgggtgacggcaccgtattacatcctgcggccggagacgctggagagcttgtactacgcataccgcgtcacgggcgactccaagtggcaggacctggcgtgggaagcgttcagtgccattgaggacgcatgccgcgccggcagcgcgtactcgtccatcaacgacgtgacgcaggccaacggcgggggtgcctctgacgatatggagagcttctggtttgccgaggcgctcaagtatgcgtacctgatctttgcggaggagtcggatgtgcaggtgcaggccaacggcgggaacaaatttgtctttaacacggaggcgcacccctttagcatccgttcatcatcacgacggggcggccaccttgcttaa

SEQ ID NO: 35 - DNA, 898 bp, Artificial Sequence
  5′-Region of PpURA5
atcggcctttgttgatgcaagttttacgtggatcatggactaaggagttttatttggaccaagttcatcgtcctagacattacggaaagggttctgctcctctttttggaaactttttggaacctctgagtatgacagcttggtggattgtacccatggtatggcttcctgtgaatttctattttttctacattggattcaccaatcaaaacaaattagtcgccatggctttttggcttttgggtctatttgtttggaccttcttggaatatgctttgcatagatttttgttccacttggactactatcttccagagaatcaaattgcatttaccattcatttcttattgcatgggatacaccactatttaccaatggataaatacagattggtgatgccacctacacttttcattgtactttgctacccaatcaagacgctcgtcttttctgttctaccatattacatggcttgttctggatttgcaggtggattcctgggctatatcatgtatgatgtcactcattacgttctgcatcactccaagctgcctcgttatttccaagagttgaagaaatatcatttggaacatcactacaagaattacgagttaggctttggtgtcacttccaaattctgggacaaagtctttgggacttatctgggtccagacgatgtgtatcaaaagacaaattagagtatttataaagttatgtaagcaaataggggctaatagggaaagaaaaattttggttctttatcagagctggctcgcgcgcagtgtttttcgtgctcctttgtaatagtcatttttgactactgttcagattgaaatcacattgaagatgtcactcgaggggtaccaaaaaaggtttttggatgctgcagtggcttcgc

SEQ ID NO: 36 - DNA, 1060 bp, Artificial Sequence
  3′-Region of PpURA5
ggtcttttcaacaaagctccattagtgagtcagctggctgaatcttatgcacaggccatcattaacagcaacctggagatagacgttgtatttggaccagcttataaaggtattcctttggctgctattaccgtgttgaagttgtacgagctcggcggcaaaaaatacgaaaatgtcggatatgcgttcaatagaaaagaaaagaaagaccacggagaaggtggaagcatcgttggagaaagtctaaagaataaaagagtactgattatcgatgatgtgatgactgcaggtactgctatcaacgaagcatttgctataattggagctgaaggtgggagagttgaaggtagtattattgccctagatagaatggagactacaggagatgactcaaataccagtgctacccaggctgttagtcagagatatggtacccctgtcttgagtatagtgacattggaccatattgtggcccatttgggcgaaactttcacagcagacgagaaatctcaaatggaaacgtatagaaaaaagtatttgcccaaataagtatgaatctgcttcgaatgaatgaattaatccaattatcttctcaccattattttcttctgtttcggagctttgggcacggcggcgggtggtgcgggctcaggttccctttcataaacagatttagtacttggatgcttaatagtgaatggcgaatgcaaaggaacaatttcgttcatctttaaccctttcactcggggtacacgttctggaatgtacccgccctgttgcaactcaggtggaccgggcaattcttgaactttctgtaacgttgttggatgttcaaccagaaattgtcctaccaactgtattagtttccttttggtcttatattgttcatcgagatacttcccactctccttgatagccactctcactcttcctggattaccaaaatcttgaggatgagtcttttcaggctccaggatgcaaggtatatccaagtacctgcaagcatctaatattgtctttgccagggggttctccacaccatactccttttggcgcatgc

SEQ ID NO: 37 - DNA, 957 bp, Artificial Sequence
  Encodes URA5 auxotrophic marker
tctagagggacttatctgggtccagacgatgtgtatcaaaagacaaattagagtatttataaagttatgtaagcaaataggggctaatagggaaagaaaaattttggttctttatcagagctggctcgcgcgcagtgtttttcgtgctcctttgtaatagtcatttttgactactgttcagattgaaatcacattgaagatgtcactggaggggtaccaaaaaaggtttttggatgctgcagtggcttcgcaggccttgaagtttggaactttcaccttgaaaagtggaagacagtctccatacttctttaacatgggtcttttcaacaaagctccattagtgagtcagctggctgaatcttatgctcaggccatcattaacagcaacctggagatagacgttgtatttggaccagcttataaaggtattcctttggctgctattaccgtgttgaagttgtacgagctgggcggcaaaaaatacgaaaatgtcggatatgcgttcaatagaaaagaaaagaaagaccacggagaaggtggaagcatcgttggagaaagtctaaagaataaaagagtactgattatcgatgatgtgatgactgcaggtactgctatcaacgaagcatttgctataattggagctgaaggtgggagagttgaaggttgtattattgccctagatagaatggagactacaggagatgactcaaataccagtgctacccaggctgttagtcagagatatggtacccctgtcttgagtatagtgacattggaccatattgtggcccatttgggcgaaactttcacagcagacgagaaatctcaaatggaaacgtatagaaaaaagtatttgcccaaataagtatgaatctgcttcgaatgaatgaattaatccaattatcttctcaccattattttcttctgtttcggagctttgggcacggcggcggatcc

SEQ ID NO: 38 - DNA, 709 bp, Artificial Sequence
  Encodes part of E. coli LacZ
cctgcactggatggtggcgctggatggtaagccgctggcaagcggtgaagtgcctctggatgtcgctccacaaggtaaacagttgattgaactgcctgaactaccgcagccggagagcgccgggcaactctggctcacagtacgcgtagtgcaaccgaacgcgaccgcatggtcagaagccgggcacatcagcgcctggcagcagtggcgtctggcggaaaacctcagtgtgacgctccccgccgcgtcccacgccatcccgcatctgaccaccagcgaaatggatttttgcatcgagctgggtaataagcgttggcaatttaaccgccagtcaggctttctttcacagatgtggattggcgataaaaaacaactgctgacgccgctgcgcgatcagttcacccgtgcaccgctggataacgacattggcgtaagtgaagcgacccgcattgaccctaacgcctgggtcgaacgctggaaggcggcgggccattaccaggccgaagcagcgttgttgcagtgcacggcagatacacttgctgatgcggtgctgattacgaccgctcacgcgtggcagcatcaggggaaaaccttatttatcagccggaaaacctaccggattgatggtagtggtcaaatggcgattaccgttgatgttgaagtggcgagcgatacaccgcatccggcgcggattggcctgaactgccag

SEQ ID NO: 39 - DNA, 2875 bp, Artificial Sequence
  5′ region of PpOCH1
aaaaccttttttcctattcaaacacaaggcattgcttcaacacgtgtgcgtatccttaacacagatactccatacttctaataatgtgatagacgaatacaaagatgttcactctgtgttgtgtctacaagcatttcttattctgattggggatattctagttacagcactaaacaactggcgatacaaacttaaattaaataatccgaatctagaaaatgaacttttggatggtccgcctgttggttggataaatcaataccgattaaatggattctattccaatgagagagtaatccaagacactctgatgtcaataatcatttgcttgcaacaacaaacccgtcatctaatcaaagggtttgatgaggcttaccttcaattgcagataaactcattgctgtccactgctgtattatgtgagaatatgggtgatgaatctggtcttctccactcagctaacatggctgtttgggcaaaggtggtacaattatacggagatcaggcaatagtgaaattgttgaatatggctactggacgatgcttcaaggatgtacgtctagtaggagccgtgggaagattgctggcagaaccagttggcacgtcgcaacaatccccaagaaatgaaataagtgaaaacgtaacgtcaaagacagcaatggagtcaatattgataacaccactggcagagcggttcgtacgtcgttttggagccgatatgaggctcagcgtgctaacagcacgattgacaagaagactctcgagtgacagtaggttgagtaaagtattcgcttagattcccaaccttcgttttattctttcgtagacaaagaagctgcatgcgaacatagggacaacttttataaatccaattgtcaaaccaacgtaaaaccctctggcaccattttcaacatatatttgtgaagcagtacgcaatatcgataaatactcaccgttgtttgtaacagccccaacttgcatacgccttctaatgacctcaaatggataagccgcagcttgtgctaacataccagcagcaccgcccgcggtcagctgcgcccacacatataaaggcaatctacgatcatgggaggaattagttttgaccgtcaggtcttcaagagttttgaactcttcttcttgaactgtgtaaccttttaaatgacgggatctaaatacgtcatggatgagatcatgtgtgtaaaaactgactccagcatatggaatcattccaaagattgtaggagcgaacccacgataaaagtttcccaaccttgccaaagtgtctaatgctgtgacttgaaatctgggttcctcgttgaagaccctgcgtactatgcccaaaaactttcctccacgagccctattaacttctctatgagtttcaaatgccaaacggacacggattaggtccaatgggtaagtgaaaaacacagagcaaaccccagctaatgagccggccagtaaccgtcttggagctgtttcataagagtcattagggatcaataacgttctaatctgttcataacatacaaattttatggctgcatagggaaaaattctcaacagggtagccgaatgaccctgatatagacctgcgacaccatcatacccatagatctgcctgacagccttaaagagcccgctaaaagacccggaaaaccgagagaactctggattagcagtctgaaaaagaatcttcactctgtctagtggagcaattaatgtcttagcggcacttcctgctactccgccagctactcctgaatagatcacatactgcaaagactgcttgtcgatgaccttggggttatttagcttcaagggcaatttttgggacattttggacacaggagactcagaaacagacacagagcgttctgagtcctggtgctcctgacgtaggcctagaacaggaattattggctttatttgtttgtccatttcataggcttggggtaatagatagatgacagagaaatagagaagacctaatattttttgttcatggcaaatcgcgggttcgcggtcgggtcacacacggagaagtaatgagaagagctggtaatctggggtaaaagggttcaaaagaaggtcgcctggtagggatgcaatacaaggttgtcttggagtttacattgaccagatgatttggctttttctctgttcaattcacatttttcagcgagaatcggattgacggagaaatggcggggtgtggggtggatagatggcagaaatgctcgcaatcaccgcgaaagaaagactttatggaatagaactactgggtggtgtaaggattacatagctagtccaatggagtccgttggaaaggtaagaagaagctaaaaccggctaagtaactagggaagaatgatcagactttgatttgatgaggtctgaaaatactctgctgctttttcagttgctttttccctgcaacctatcattttccttttcataagcctgccttttctgttttcacttatatgagttccgccgagacttccccaaattctctcctggaacattctctatcgctctccttccaagttgcgccccctggcactgcctagtaatattaccacgcgacttatattcagttccacaatttccagtgttcgtagcaaatatcatcagccatggcgaaggcagatggcagtttgctctactataatcctcacaatccacccagaaggtattacttctacatggctatattcgccgtttctgtcatttgcgttttgtacggaccctcacaacaattatcatctccaaaaatagactatgatccattgacgctccgatcacttgatttgaagactttggaagctccttcacagttgagtccaggcaccgtagaagataatcttcg

SEQ ID NO: 40 - DNA, 997 bp, Artificial Sequence
  3′ region of PpOCH1
aaagctagagtaaaatagatatagcgagattagagaatgaataccttcttctaagcgatcgtccgtcatcatagaatatcatggactgtatagttttttttttgtacatataatgattaaacggtcatccaacatctcgttgacagatctctcagtacgcgaaatccctgactatcaaagcaagaaccgatgaagaaaaaaacaacagtaacccaaacaccacaacaaacactttatcttctcccccccaacaccaatcatcaaagagatgtcggaaccaaacaccaagaagcaaaaactaaccccatataaaaacatcctggtagataatgctggtaacccgctctccttccatattctgggctacttcacgaagtctgaccggtctcagttgatcaacatgatcctcgaaatgggtggcaagatcgttccagacctgcctcctctggtagatggagtgttgtttttgacaggggattacaagtctattgatgaagataccctaaagcaactgggggacgttccaatatacagagactccttcatctaccagtgttttgtgcacaagacatctcttcccattgacactttccgaattgacaagaacgtcgacttggctcaagatttgatcaatagggcccttcaagagtctgtggatcatgtcacttctgccagcacagctgcagctgctgctgttgttgtcgctaccaacggcctgtcttctaaaccagacgctcgtactagcaaaatacagttcactcccgaagaagatcgttttattcttgactttgttaggagaaatcctaaacgaagaaacacacatcaactgtacactgagctcgctcagcacatgaaaaaccatacgaatcattctatccgccacagatttcgtcgtaatctttccgctcaacttgattgggtttatgatatcgatccattgaccaaccaacctcgaaaagatgaaaacgggaactacatcaaggtacaaggccttcca

SEQ ID NO: 41 - DNA, 870 bp, Artificial Sequence
  5′ region of PpBMT2
ggccgagcgggcctagattttcactacaaatttcaaaactacgcggatttattgtctcagagagcaatttggcatttctgagcgtagcaggaggcttcataagattgtataggaccgtaccaacaaattgccgaggcacaacacggtatgctgtgcacttatgtggctacttccctacaacggaatgaaaccttcctctttccgcttaaacgagaaagtgtgtcgcaattgaatgcaggtgcctgtgcgccttggtgtattgtttttgagggcccaatttatcaggcgccttttttcttggttgttttcccttagcctcaagcaaggttggtctatttcatctccgcttctataccgtgcctgatactgttggatgagaacacgactcaacttcctgctgctctgtattgccagtgttttgtctgtgatttggatcggagtcctccttacttggaatgataataatcttggcggaatctccctaaacggaggcaaggattctgcctatgatgatctgctatcattgggaagcttcaacgacatggaggtcgactcctatgtcaccaacatctacgacaatgctccagtgctaggatgtacggatttgtcttatcatggattgttgaaagtcaccccaaagcatgacttagcttgcgatttggagttcataagagctcagattttggacattgacgtttactccgccataaaagacttagaagataaagccttgactgtaaaacaaaaggttgaaaaacactggtttacgttttatggtagttcagtctttctgcccgaacacgatgtgcattacctggttagacgagtcatcttttcggctgaaggaaaggcgaactctccagtaacatc

SEQ ID NO: 42 - DNA, 1733 bp, Artificial Sequence
  3′ region of PpBMT2
ccatatgatgggtgtttgctcactcgtatggatcaaaattccatggtttcttctgtacaacttgtacacttatttggacttttctaacggtttttctggtgatttgagaagtccttattttggtgttcgcagcttatccgtgattgaaccatcagaaatactgcagctcgttatctagtttcagaatgtgttgtagaatacaatcaattctgagtctagtttgggtgggtcttggcgacgggaccgttatatgcatctatgcagtgttaaggtacatagaatgaaaatgtaggggttaatcgaaagcatcgttaatttcagtagaacgtagttctattccctacccaaataatttgccaagaatgcttcgtatccacatacgcagtggacgtagcaaatttcactttggactgtgacctcaagtcgttatcttctacttggacattgatggtcattacgtaatccacaaagaattggatagcctctcgttttatctagtgcacagcctaatagcacttaagtaagagcaatggacaaatttgcatagacattgagctagatacgtaactcagatcttgttcactcatggtgtactcgaagtactgctggaaccgttacctcttatcatttcgctactggctcgtgaaactactggatgaaaaaaaaaaaagagctgaaagcgagatcatcccattttgtcatcatacaaattcacgcttgcagttttgcttcgttaacaagacaagatgtctttatcaaagacccgttttttcttcttgaagaatacttccctgttgagcacatgcaaaccatatttatctcagatttcactcaacttgggtgcttccaagagaagtaaaattcttcccactgcatcaacttccaagaaacccgtagaccagtttctcttcagccaaaagaagttgctcgccgatcaccgcggtaacagaggagtcagaaggtttcacacccttccatcccgatttcaaagtcaaagtgctgcgttgaaccaaggttttcaggttgccaaagcccagtctgcaaaaactagttccaaatggcctattaattcccataaaagtgttggctacgtatgtatcggtacctccattctggtatttgctattgttgtcgttggtgggttgactagactgaccgaatccggtctttccataacggagtggaaacctatcactggttcggttcccccactgactgaggaagactggaagttggaatttgaaaaatacaaacaaagccctgagtttcaggaactaaattctcacataacattggaagagttcaagtttatattttccatggaatggggacatagattgttgggaagggtcatcggcctgtcgtttgttcttcccacgttttacttcattgcccgtcgaaagtgttccaaagatgttgcattgaaactgcttgcaatatgctctatgataggattccaaggtttcatcggctggtggatggtgtattccggattggacaaacagcaattggctgaacgtaactccaaaccaactgtgtctccatatcgcttaactacccatcttggaactgcatttgttatttactgttacatgatttacacagggcttcaagttttgaagaactataagatcatgaaacagcctgaagcgtatgttcaaattttcaagcaaattgcgtctccaaaattgaaaactttcaagagactctcttcagttctattaggcctggtg

SEQ ID NO: 43 - DNA, 411 bp, Artificial Sequence
  5′ region of PpBMT1
catatggtgagagccgttctgcacaactagatgttttcgagcttcgcattgtttcctgcagctcgactattgaattaagatttccggatatctccaatctcacaaaaacttatgttgaccacgtgctttcctgaggcgaggtgttttatatgcaagctgccaaaaatggaaaacgaatggccatttttcgcccaggcaaattattcgattactgctgtcataaagacagtgttgcaaggctcacatttttttttaggatccgagataaagtgaatacaggacagcttatctctatatcttgtaccattcgtgaatcttaagagttcggttagggggactctagttgagggttggcactcacgtatggctgggcgcagaaataaaattcaggcgcagcagcacttatcgatg

SEQ ID NO: 44 - DNA, 692 bp, Artificial Sequence
  3′ region of PpBMT1
gaattcacagttataaataaaaacaaaaactcaaaaagtttgggctccacaaaataacttaatttaaatttttgtctaataaatgaatgtaattccaagattatgtgatgcaagcacagtatgcttcagccctatgcagctactaatgtcaatctcgcctgcgagcgggcctagattttcactacaaatttcaaaactacgcggatttattgtctcagagagcaatttggcatttctgagcgtagcaggaggcttcataagattgtataggaccgtaccaacaaattgccgaggcacaacacggtatgctgtgcacttatgtggctacttccctacaacggaatgaaaccttcctctttccgcttaaacgagaaagtgtgtcgcaattgaatgcaggtgcctgtgcgccttggtgtattgtttttgagggcccaatttatcaggcgccttttttcttggttgttttcccttagcctcaagcaaggttggtctatttcatctccgcttctataccgtgcctgatactgttggatgagaacacgactcaacttcctgctgctctgtattgccagtgttttgtctgtgatttggatcggagtcctccttacttggaatgataataatcttggcggaatctccctaaacggaggcaaggattctgcctatgatgatctgctatcattgggaagctt

SEQ ID NO: 45 - DNA, 546 bp, Artificial Sequence
  5′ region of PpBMT3
gatatctccctggggacaatatgtgttgcaactgttcgttgttggtgccccagtcccccaaccggtactaatcggtctatgttcccgtaactcatattcggttagaactagaacaataagtgcatcattgttcaacattgtggttcaattgtcgaacattgctggtgcttatatctacagggaagacgataagcctttgtacaagagaggtaacagacagttaattggtatttctttgggagtcgttgccctctacgttgtctccaagacatactacattctgagaaacagatggaagactcaaaaatgggagaagcttagtgaagaagagaaagttgcctacttggacagagctgagaaggagaacctgggttctaagaggctggactttttgttcgagagttaaactgcataattttttctaagtaaatttcatagttatgaaatttctgcagcttagtgtttactgcatcgtttactgcatcaccctgtaaataatgtgagcttttttccttccattgcttggtatcttccttgctgctgttt

SEQ ID NO: 46 - DNA, 378 bp, Artificial Sequence
  3′ region of PpBMT3
acaaaacagtcatgtacagaactaacgcctttaagatgcagaccactgaaaagaattgggtcccatttttcttgaaagacgaccaggaatctgtccattttgtttactcgttcaatcctctgagagtactcaactgcagtcttgataacggtgcatgtgatgttctatttgagttaccacatgattttggcatgtcttccgagctacgtggtgccactcctatgctcaatcttcctcaggcaatcccgatggcagacgacaaagaaatttgggtttcattcccaagaacgagaatatcagattgcgggtgttctgaaacaatgtacaggccaatgttaatgctttttgttagagaaggaacaaacttttttgctgagc

SEQ ID NO: 47 - DNA, 1043 bp, Artificial Sequence
  5′ region of PpBMT4
aagcttgttcaccgttgggacttttccgtggacaatgttgactactccaggagggattccagctttctctactagctcagcaataatcaatgcagccccaggcgcccgttctgatggcttgatgaccgttgtattgcctgtcactatagccaggggtagggtccataaaggaatcatagcagggaaattaaaagggcatattgatgcaatcactcccaatggctctcttgccattgaagtctccatatcagcactaacttccaagaaggaccccttcaagtctgacgtgatagagcacgcttgctctgccacctgtagtcctctcaaaacgtcaccttgtgcatcagcaaagactttaccttgctccaatactatgacggaggcaattctgtcaaaattctctctcagcaattcaaccaacttgaaagcaaattgctgtctcttgatgatggagacttttttccaagattgaaatgcaatgtgggacgactcaattgcttcttccagctcctcttcggttgattgaggaacttttgaaaccacaaaattggtcgttgggtcatgtacatcaaaccattctgtagatttagattcgacgaaagcgttgttgatgaaggaaaaggttggatacggtttgtcggtctctttggtatggccggtggggtatgcaattgcagtagaagataattggacagccattgttgaaggtagagaaaaggtcagggaacttgggggttatttataccattttaccccacaaataacaactgaaaagtacccattccatagtgagaggtaaccgacggaaaaagacgggcccatgttctgggaccaatagaactgtgtaatccattgggactaatcaacagacgattggcaatataatgaaatagttcgttgaaaagccacgtcagctgtcttttcattaactttggtcggacacaacattttctactgttgtatctgtcctactttgcttatcatctgccacagggcaagtggatttccttctcgcgcggctgggtgaaaacggttaacgtgaa

SEQ ID NO: 48 - DNA, 695 bp, Artificial Sequence
  3′ region of PpBMT4
gccttgggggacttcaagtctttgctagaaactagatgaggtcaggccctcttatggttgtgtcccaattgggcaatttcactcacctaaaaagcatgacaattatttagcgaaataggtagtatattttccctcatctcccaagcagtttcgtttttgcatccatatctctcaaatgagcagctacgactcattagaaccagagtcaagtaggggtgagctcagtcatcagccttcgtttctaaaacgattgagttcttttgttgctacaggaagcgccctagggaactttcgcactttggaaatagattttgatgaccaagagcgggagttgatattagagaggctgtccaaagtacatgggatcaggccggccaaattgattggtgtgactaaaccattgtgtacttggacactctattacaaaagcgaagatgatttgaagtattacaagtcccgaagtgttagaggattctatcgagcccagaatgaaatcatcaaccgttatcagcagattgataaactcttggaaagcggtatcccattttcattattgaagaactacgataatgaagatgtgagagacggcgaccctctgaacgtagacgaagaaacaaatctacttttggggtacaatagagaaagtgaatcaagggaggtatttgtggccataatactcaactctatcattaatg

SEQ ID NO: 49 - DNA, 937 bp, Artificial Sequence
  5′-Region of PpPNO1 and PpMNN4
tcattctatatgttcaagaaaagggtagtgaaaggaaagaaaaggcatataggcgagggagagttagctagcatacaagataatgaaggatcaatagcggtagttaaagtgcacaagaaaagagcacctgttgaggctgatgataaagctccaattacattgccacagagaaacacagtaacagaaataggaggggatgcaccacgagaagagcattcagtgaacaactttgccaaattcataaccccaagcgctaataagccaatgtcaaagtcggctactaacattaatagtacaacaactatcgattttcaaccagatgtttgcaaggactacaaacagacaggttactgcggatatggtgacacttgtaagtttttgcacctgagggatgatttcaaacagggatggaaattagatagggagtgggaaaatgtccaaaagaagaagcataatactctcaaaggggttaaggagatccaaatgtttaatgaagatgagctcaaagatatcccgtttaaatgcattatatgcaaaggagattacaaatcacccgtgaaaacttcttgcaatcattatttttgcgaacaatgtttcctgcaacggtcaagaagaaaaccaaattgtattatatgtggcagagacactttaggagttgctttaccagcaaagaagttgtcccaatttctggctaagatacataataatgaaagtaataaagtttagtaattgcattgcgttgactattgattgcattgatgtcgtgtgatactttcaccgaaaaaaaacacgaagcgcaataggagcggttgcatattagtccccaaagctatttaattgtgcctgaaactgttttttaagctcatcaagcataattgtatgcattgcgacgtaaccaacgtttaggcgcagtttaatcatagcccactgctaagcc

SEQ ID NO: 50 - DNA, 1906 bp, Artificial Sequence
  3′-Region of PpPNO1 and PpMNN4
cggaggaatgcaaataataatctccttaattacccactgataagctcaagagacgcggtttgaaaacgatataatgaatcatttggattttataataaaccctgacagtttttccactgtattgttttaacactcattggaagctgtattgattctaagaagctagaaatcaatacggccatacaaaagatgacattgaataagcaccggcttttttgattagcatataccttaaagcatgcattcatggctacatagttgttaaagggcttcttccattatcagtataatgaattacataatcatgcacttatatttgcccatctctgttctctcactcttgcctgggtatattctatgaaattgcgtatagcgtgtctccagttgaaccccaagcttggcgagtttgaagagaatgctaaccttgcgtattccttgcttcaggaaacattcaaggagaaacaggtcaagaagccaaacattttgatccttcccgagttagcattgactggctacaattttcaaagccagcagcggatagagccttttttggaggaaacaaccaagggagctagtacccaatgggctcaaaaagtatccaagacgtgggattgctttactttaataggatacccagaaaaaagtttagagagccctccccgtatttacaacagtgcggtacttgtatcgcctcagggaaaagtaatgaacaactacagaaagtccttcttgtatgaagctgatgaacattggggatgttcggaatcttctgatgggtttcaaacagtagatttattaattgaaggaaagactgtaaagacatcatttggaatttgcatggatttgaatccttataaatttgaagctccattcacagacttcgagttcagtggccattgcttgaaaaccggtacaagactcattttgtgcccaatggcctggttgtcccctctatcgccttccattaaaaaggatcttagtgatatagagaaaagcagacttcaaaagttctaccttgaaaaaatagataccccggaatttgacgttaattacgaattgaaaaaagatgaagtattgcccacccgtatgaatgaaacgttggaaacaattgactttgagccttcaaaaccggactactctaatataaattattggatactaaggttttttccctttctgactcatgtctataaacgagatgtgctcaaagagaatgcagttgcagtcttatgcaaccgagttggcattgagagtgatgtcttgtacggaggatcaaccacgattctaaacttcaatggtaagttagcatcgacacaagaggagctggagttgtacgggcagactaatagtctcaaccccagtgtggaagtattgggggcccttggcatgggtcaacagggaattctagtacgagacattgaattaacataatatacaatatacaataaacacaaataaagaatacaagcctgacaaaaattcacaaattattgcctagacttgtcgttatcagcagcgacctttttccaatgctcaatttcacgatatgccttttctagctctgctttaagcttctcattggaattggctaactcgttgactgcttggtcagtgatgagtttctccaaggtccatttctcgatgttgttgttttcgttttcctttaatctcttgatataatcaacagccttctttaatatctgagccttgttcgagtcccctgttggcaacagagcggccagttcctttattccgtggtttatattttctcttctacgcctttctacttctttgtgattctctttacgcatcttatgccattcttcagaaccagtggctggcttaaccgaatagccagagcctgaagaagccgcactagaagaagcagtggcattgttgactatgg

SEQ ID NO: 51 - DNA, 1128 bp, Artificial Sequence
  5′-Region of PpMNN4L1
gatctggccattgtgaaacttgacactaaagacaaaactcttagagtttccaatcacttaggagacgatgtttcctacaacgagtacgatccctcattgatcatgagcaatttgtatgtgaaaaaagtcatcgaccttgacaccttggataaaagggctggaggaggtggaaccacctgtgcaggcggtctgaaagtgttcaagtacggatctactaccaaatatacatctggtaacctgaacggcgtcaggttagtatactggaacgaaggaaagttgcaaagctccaaatttgtggttcgatcctctaattactctcaaaagcttggaggaaacagcaacgccgaatcaattgacaacaatggtgtgggttttgcctcagctggagactcaggcgcatggattctttccaagctacaagatgttagggagtaccagtcattcactgaaaagctaggtgaagctacgatgagcattttcgatttccacggtcttaaacaggagacttctactacagggcttggggtagttggtatgattcattcttacgacggtgagttcaaacagtttggtttgttcactccaatgacatctattctacaaagacttcaacgagtgaccaatgtagaatggtgtgtagcgggttgcgaagatggggatgtggacactgaaggagaacacgaattgagtgatttggaacaactgcatatgcatagtgattccgactagtcaggcaagagagagccctcaaatttacctctctgcccctcctcactccttttggtacgcataattgcagtataaagaacttgctgccagccagtaatcttatttcatacgcagttctatatagcacataatcttgcttgtatgtatgaaatttaccgcgttttagttgaaattgtttatgttgtgtgccttgcatgaaatctctcgttagccctatccttacatttaactggtctcaaaacctctaccaattccattgctgtacaacaatatgaggcggcattactgtagggttggaaaaaaattgtcattccagctagagatcacacgacttcatcacgcttattgctcctcattgctaaatcatttactcttgacttcgacccagaaaagttcgcc

SEQ ID NO: 52 - DNA, 1231 bp, Artificial Sequence
  3′-Region of PpMNN4L1
gcatgtcaaacttgaacacaacgactagatagttgttttttctatataaaacgaaacgttatcatctttaataatcattgaggtttacccttatagttccgtattttcgtttccaaacttagtaatcttttggaaatatcatcaaagctggtgccaatcttcttgtttgaagtttcaaactgctccaccaagctacttagagactgttctaggtctgaagcaacttcgaacacagagacagctgccgccgattgttcttttttgtgtttttcttctggaagaggggcatcatcttgtatgtccaatgcccgtatcctttctgagttgtccgacacattgtccttcgaagagtttcctgacattgggcttcttctatccgtgtattaattttgggttaagttcctcgtttgcatagcagtggatacctcgatttttttggctcctatttacctgacataatattctactataatccaacttggacgcgtcatctatgataactaggctctcctttgttcaaaggggacgtcttcataatccactggcacgaagtaagtctgcaacgaggcggcttttgcaacagaacgatagtgtcgtttcgtacttggactatgctaaacaaaaggatctgtcaaacatttcaaccgtgtttcaaggcactctttacgaattatcgaccaagaccttcctagacgaacatttcaacatatccaggctactgcttcaaggtggtgcaaatgataaaggtatagatattagatgtgtttgggacctaaaacagttcttgcctgaagattcccttgagcaacaggcttcaatagccaagttagagaagcagtaccaaatcggtaacaaaagggggaagcatataaaacctttactattgcgacaaaatccatccttgaaagtaaagctgtttgttcaatgtaaagcatacgaaacgaaggaggtagatcctaagatggttagagaacttaacgggacatactccagctgcatcccatattacgatcgctggaagacttttttcatgtacgtatcgcccaccaacctttcaaagcaagctaggtatgattttgacagttctcacaatccattggttttcatgcaacttgaaaaaacccaactcaaacttcatggggatccatacaatgtaaatcattacgagagggcgaggttgaaaagtttccattgcaatcacgtcgcatcatggctactgaaaggccttaac

SEQ ID NO: 53 - DNA, 1815 bp, Artificial Sequence
  PpTRP2 gene integration locus
taatggccaaacggtttctcaattactatatactactaaccatttacctgtagcgtatttcttttccctcttcgcgaaagctcaagggcatcttcttgactcatgaaaaatatctggatttcttctgacagatcatcacccttgagcccaactctctagcctatgagtgtaagtgatagtcatcttgcaacagattattttggaacgcaactaacaaagcagatacacccttcagcagaatcctttctggatattgtgaagaatgatcgccaaagtcacagtcctgagacagttcctaatctttaccccatttacaagttcatccaatcagacttcttaacgcctcatctggcttatatcaagcttaccaacagttcagaaactcccagtccaagtttcttgcttgaaagtgcgaagaatggtgacaccgttgacaggtacacctttatgggacattcccccagaaaaataatcaagactgggcctttagagggtgctgaagttgaccccttggtgcttctggaaaaagaactgaagggcaccagacaagcgcaacttcctggtattcctcgtctaagtggtggtgccataggatacatctcgtacgattgtattaagtactttgaaccaaaaactgaaagaaaactgaaagatgttttgcaacttccggaagcagctttgatgttgttcgacacgatcgtggcttttgacaatgtttatcaaagattccaggtaattggaaacgtttctctatccgttgatgactcggacgaagctattcttgagaaatattataagacaagagaagaagtggaaaagatcagtaaagtggtatttgacaataaaactgttccctactatgaacagaaagatattattcaaggccaaacgttcacctctaatattggtcaggaagggtatgaaaaccatgttcgcaagctgaaagaacatattctgaaaggagacatcttccaagctgttccctctcaaagggtagccaggccgacctcattgcaccctttcaacatctatcgtcatttgagaactgtcaatccttctccatacatgttctatattgactatctagacttccaagttgttggtgcttcacctgaattactagttaaatccgacaacaacaacaaaatcatcacacatcctattgctggaactcttcccagaggtaaaactatcgaagaggacgacaattatgctaagcaattgaagtcgtctttgaaagacagggccgagcacgtcatgctggtagatttggccagaaatgatattaaccgtgtgtgtgagcccaccagtaccacggttgatcgtttattgactgtggagagattttctcatgtgatgcatcttgtgtcagaagtcagtggaacattgagaccaaacaagactcgcttcgatgctttcagatccattttcccagcaggaaccgtctccggtgctccgaaggtaagagcaatgcaactcataggagaattggaaggagaaaagagaggtgtttatgcgggggccgtaggacactggtcgtacgatggaaaatcgatggacacatgtattgccttaagaacaatggtcgtcaaggacggtgtcgcttaccttcaagccggaggtggaattgtctacgattctgacccctatgacgagtacatcgaaaccatgaacaaaatgagatccaacaataacaccatcttggaggctgagaaaatctggaccgataggttggccagagacgagaatcaaagtgaatccgaagaaaacgatcaatgaacggaggacgtaagtaggaatttatggtttggccat

SEQ ID NO: 54 - DNA, 486 bp, Artificial Sequence
  PpGAPDH promoter
tttttgtagaaatgtcttggtgtcctcgtccaatcaggtagccatctctgaaatatctggctccgttgcaactccgaacgacctgctggcaacgtaaaattctccggggtaaaacttaaatgtggagtaatggaaccagaaacgtctcttcccttctctctccttccaccgcccgttaccgtccctaggaaattttactctgctggagagcttcttctacggcccccttgcagcaatgctcttcccagcattacgttgcgggtaaaacggaggtcgtgtacccgacctagcagcccagggatggaaaagtcccggccgtcgctggcaataatagcgggcggacgcatgtcatgagattattggaaaccaccagaatcgaatataaaaggcgaacacctttcccaattttggtttctcctgacccaaagactttaaatttaatttatttgtccctatttcaatcaattgaacaactatcaaaacaca

SEQ ID NO: 55 - DNA, 376 bp, Artificial Sequence
  PpALG3 terminator
atttacaattagtaatattaaggtggtaaaaacattcgtagaattgaaatgaattaatatagtatgacaatggttcatgtctataaatctccggcttcggtaccttctccccaattgaatacattgtcaaaatgaatggttgaactattaggttcgccagtttcgttattaagaaaactgttaaaatcaaattccatatcatcggttccagtgggaggaccagttccatcgccaaaatcctgtaagaatccattgtcagaacctgtaaagtcagtttgagatgaaatttttccggtctttgttgacttggaagcttcgttaaggttaggtgaaacagtttgatcaaccagcggctcccgttttcgtcgcttagtag

SEQ ID NO: 56 - DNA, 934 bp, Artificial Sequence
  PpAOX1 promoter and integration locus
aacatccaaagacgaaaggttgaatgaaacctttttgccatccgacatccacaggtccattctcacacataagtgccaaacgcaacaggaggggatacactagcagcagaccgttgcaaacgcaggacctccactcctcttctcctcaacacccacttttgccatcgaaaaaccagcccagttattgggcttgattggagctcgctcattccaattccttctattaggctactaacaccatgactttattagcctgtctatcctggcccccctggcgaggttcatgtttgtttatttccgaatgcaacaagctccgcattacacccgaacatcactccagatgagggctttctgagtgtggggtcaaatagtttcatgttccccaaatggcccaaaactgacagtttaaacgctgtcttggaacctaatatgacaaaagcgtgatctcatccaagatgaactaagtttggttcgttgaaatgctaacggccagttggtcaaaaagaaacttccaaaagtcggcataccgtttgtcttgtttggtattgattgacgaatgctcaaaaataatctcattaatgcttagcgcagtctctctatcgcttctgaaccccggtgcacctgtgccgaaacgcaaatggggaaacacccgctttttggatgattatgcattgtctccacattgtatgcttccaagattctggtgggaatactgctgatagcctaacgttcatgatcaaaatttaactgttctaacccctacttgacagcaatatataaacagaaggaagctgccctgtcttaaacctttttttttatcatcattattagcttactttcataattgcgactggttccaattgacaagcttttgattttaacgacttttaacgacaacttgagaagatcaaaaaacaactaattattcgaaacg

SEQ ID NO: 57 - DNA, 293 bp, Artificial Sequence
  ScCYC1 terminator
acaggccccttttcctttgtcgatatcatgtaattagttatgtcacgcttacattcacgccctcctcccacatccgctctaaccgaaaaggaaggagttagacaacctgaagtctaggtccctatttattttttttaatagttatgttagtattaagaacgttatttatatttcaaatttttcttttttttctgtacaaacgcgtgtacgcatgtaacattatactgaaaaccttgcttgagaaggttttgggacgctcgaaggctttaatttgcaagctgccggctcttaag

SEQ ID NO: 58 - DNA, 427 bp, Artificial Sequence
  ScTEF1 promoter
gatcccccacacaccatagcttcaaaatgtttctactccttttttactcttccagattttctcggactccgcgcatcgccgtaccacttcaaaacacccaagcacagcatactaaatttcccctctttcttcctctagggtgtcgttaattacccgtactaaaggtttggaaaagaaaaaagagaccgcctcgtttctttttcttcgtcgaaaaaggcaataaaaatttttatcacgtttctttttcttgaaaatttttttttttgatttttttctctttcgatgacctcccattgatatttaagttaataaacggtcttcaatttctcaagtttcagtttcatttttcttgttctattacaactttttttacttcttgctcattagaaagaaagcatagcaatctaatctaagttttaattacaaa

SEQ ID NO: 59 - DNA, 375 bp, Artificial Sequence
  Encodes Sh ble ORF (Zeocin resistance marker)
atggccaagttgaccagtgccgttccggtgctcaccgcgcgcgacgtcgccggagcggtcgagttctggaccgaccggctcgggttctcccgggacttcgtggaggacgacttcgccggtgtggtccgggacgacgtgaccctgttcatcagcgcggtccaggaccaggtggtgccggacaacaccctggcctgggtgtgggtgcgcggcctggacgagctgtacgccgagtggtcggaggtcgtgtccacgaacttccgggacgcctccgggccggccatgaccgagatcggcgagcagccgtgggggcgggagttcgccctgcgcgacccggccggcaactgcgtgcacttcgtggccgaggagcaggactga

SEQ ID NO: 60 - DNA, 582 bp, Artificial Sequence
  Encodes NATR
atgggtaccactcttgacgacacggcttaccggtaccgcaccagtgtcccgggggacgccgaggccatcgaggcactggatgggtccttcaccaccgacaccgtcttccgcgtcaccgccaccggggacggcttcaccctgcgggaggtgccggtggacccgcccctgaccaaggtgttccccgacgacgaatcggacgacgaatcggacgacggggaggacggcgacccggactcccggacgttcgtcgcgtacggggacgacggcgacctggcgggcttcgtggtcgtctcgtactccggctggaaccgccggctgaccgtcgaggacatcgaggtcgccccggagcaccgggggcacggggtcgggcgcgcgttgatggggctcgcgacggagttcgcccgcgagcggggcgccgggcacctctggctggaggtcaccaacgtcaacgcaccggcgatccacgcgtaccggcggatggggttcaccctctgcggcctggacaccgccctgtacgacggcaccgcctcggacggcgagcaggcgctctacatgagcatgccctgcccctaatcagtactg

SEQ ID NO: 61 - DNA, 1231 bp, Artificial Sequence
  5′ region of PpPRO1 locus
gaagggccatcgaattgtcatcgtctcctcaggtgccatcgctgtgggcatgaagagagtcaacatgaagcggaaaccaaaaaagttacagcaagtgcaggcattggctgctataggacaaggccgtttgataggactttgggacgaccttttccgtcagttgaatcagcctattgcgcagattttactgactagaacggatttggtcgattacacccagtttaagaacgctgaaaatacattggaacagcttattaaaatgggtattattcctattgtcaatgagaatgacaccctatccattcaagaaatcaaatttggtgacaatgacaccttatccgccataacagctggtatgtgtcatgcagactacctgtttttggtgactgatgtggactgtctttacacggataaccctcgtacgaatccggacgctgagccaatcgtgttagttagaaatatgaggaatctaaacgtcaataccgaaagtggaggttccgccgtaggaacaggaggaatgacaactaaattgatcgcagctgatttgggtgtatctgcaggtgttacaacgattatttgcaaaagtgaacatcccgagcagattttggacattgtagagtacagtatccgtgctgatagagtcgaaaatgaggctaaatatctggtcatcaacgaagaggaaactgtggaacaatttcaagagatcaatcggtcagaactgagggagttgaacaagctggacattcctttgcatacacgtttcgttggccacagttttaatgctgttaataacaaagagttttggttactccatggactaaaggccaacggagccattatcattgatccaggttgttataaggctatcactagaaaaaacaaagctggtattcttccagctggaattatttccgtagagggtaatttccatgaatacgagtgtgttgatgttaaggtaggactaagagatccagatgacccacattcactagaccccaatgaagaactttacgtcgttggccgtgcccgttgtaattaccccagcaatcaaatcaacaaaattaagggtctacaaagctcgcagatcgagcaggttctaggttacgctgacggtgagtatgttgttcacagggacaacttggctttcccagtatttgccgatccagaactgttggatgttgttgagagtaccctgtctgaacaggagagagaatccaaaccaaataaatag

SEQ ID NO: 62 - DNA, 1425 bp, Artificial Sequence
  3′ region of PpPRO1 locus
aatttcacatatgctgcttgattatgtaattataccttgcgttcgatggcatcgatttcctcttctgtcaatcgcgcatcgcattaaaagtatacttttttttttttcctatagtactattcgccttattataaactttgctagtatgagttctacccccaagaaagagcctgatttgactcctaagaagagtcagcctccaaagaatagtctcggtgggggtaaaggctttagtgaggagggtttctcccaaggggacttcagcgctaagcatatactaaatcgtcgccctaacaccgaaggctcttctgtggcttcgaacgtcatcagttcgtcatcattgcaaaggttaccatcctctggatctggaagcgttgctgtgggaagtgtgttgggatcttcgccattaactctttctggagggttccacgggcttgatccaaccaagaataaaatagacgttccaaagtcgaaacagtcaaggagacaaagtgttctttctgacatgatttccacttctcatgcagctagaaatgatcactcagagcagcagttacaaactggacaacaatcagaacaaaaagaagaagatggtagtcgatcttctttttctgtttcttcccccgcaagagatatccggcacccagatgtactgaaaactgtcgagaaacatcttgccaatgacagcgagatcgactcatctttacaacttcaaggtggagatgtcactagaggcatttatcaatgggtaactggagaaagtagtcaaaaagataacccgcctttgaaacgagcaaatagttttaatgatttttcttctgtgcatggtgacgaggtaggcaaggcagatgctgaccacgatcgtgaaagcgtattcgacgaggatgatatctccattgatgatatcaaagttccgggagggatgcgtcgaagttttttattacaaaagcatagagaccaacaactttctggactgaataaaacggctcaccaaccaaaacaacttactaaacctaatttcttcacgaacaactttatagagtttttggcattgtatgggcattttgcaggtgaagatttggaggaagacgaagatgaagatttagacagtggttccgaatcagtcgcagtcagtgatagtgagggagaattcagtgaggctgacaacaatttgttgtatgatgaagagtctctcctattagcacctagtacctccaactatgcgagatcaagaataggaagtattcgtactcctacttatggatctttcagttcaaatgttggttcttcgtctattcatcagcagttaatgaaaagtcaaatcccgaagctgaagaaacgtggacagcacaagcataaaacacaatcaaaaatacgctcgaagaagcaaactaccaccgtaaaagcagtgttgctgctattaaa

SEQ ID NO: 63 - DNA, 1407 bp, Artificial Sequence
  Encodes Mm ManI catalytic doman (FB)
gagcccgctgacgccaccatccgtgagaagagggcaaagatcaaagagatgatgacccatgcttggaataattataaacgctatgcgtggggcttgaacgaactgaaacctatatcaaaagaaggccattcaagcagtttgtttggcaacatcaaaggagctacaatagtagatgccctggatacccttttcattatgggcatgaagactgaatttcaagaagctaaatcgtggattaaaaaatatttagattttaatgtgaatgctgaagtttctgtttttgaagtcaacatacgcttcgtcggtggactgctgtcagcctactatttgtccggagaggagatatttcgaaagaaagcagtggaacttggggtaaaattgctacctgcatttcatactccctctggaataccttgggcattgctgaatatgaaaagtgggatcgggcggaactggccctgggcctctggaggcagcagtatcctggccgaatttggaactctgcatttagagtttatgcacttgtcccacttatcaggagacccagtctttgccgaaaaggttatgaaaattcgaacagtgttgaacaaactggacaaaccagaaggcctttatcctaactatctgaaccccagtagtggacagtggggtcaacatcatgtgtcggttggaggacttggagacagcttttatgaatatttgcttaaggcgtggttaatgtctgacaagacagatctcgaagccaagaagatgtattttgatgctgttcaggccatcgagactcacttgatccgcaagtcaagtgggggactaacgtacatcgcagagtggaaggggggcctcctggaacacaagatgggccacctgacgtgctttgcaggaggcatgtttgcacttggggcagatggagctccggaagcccgggcccaacactaccttgaactcggagctgaaattgcccgcacttgtcatgaatcttataatcgtacatatgtgaagttgggaccggaagcgtttcgatttgatggcggtgtggaagctattgccacgaggcaaaatgaaaagtattacatcttacggcccgaggtcatcgagacatacatgtacatgtggcgactgactcacgaccccaagtacaggacctgggcctgggaagccgtggaggctctagaaagtcactgcagagtgaacggaggctactcaggcttacgggatgtttacattgcccgtgagagttatgacgatgtccagcaaagtttcttcctggcagagacactgaagtatttgtacttgatattttccgatgatgaccttcttccactagaacactggatcttcaacaccgaggctcatcctttccctatactccgtgaacagaagaaggaaattgatggcaaagagaaatga

SEQ ID NO: 64 - DNA, 108 bp, Artificial Sequence
  Encodes Mnn2 leader (53)
atgctgcttaccaaaaggttttcaaagctgttcaagctgacgttcatagttttgatattgtgcgggctgttcgtcattacaaacaaatacatggatgagaacacgtcg

SEQ ID NO: 65 - DNA, 3029 bp, Pichia pastoris
aggcctcgcaacaacctataattgagttaagtgcctttccaagctaaaaagtttgaggttataggggcttagcatccacacgtcacaatctcgggtatcgagtatagtatgtagaattacggcaggaggtttcccaatgaacaaaggacaggggcacggtgagctgtcgaaggtatccattttatcatgtttcgtttgtacaagcacgacatactaagacatttaccgtatgggagttgttgtcctagcgtagttctcgctcccccagcaaagctcaaaaaagtacgtcatttagaatagtttgtgagcaaattaccagtcggtatgctacgttagaaaggcccacagtattcttctaccaaaggcgtgcctttgttgaactcgatccattatgagggcttccattattccccgcatttttattactctgaacaggaataaaaagaaaaaacccagtttaggaaattatccgggggcgaagaaatacgcgtagcgttaatcgaccccacgtccagggtttttccatggaggtttctggaaaaactgacgaggaatgtgattataaatccctttatgtgatgtctaagacttttaaggtacgcccgatgtttgcctattaccatcatagagacgtttcttttcgaggaatgcttaaacgactttgtttgacaaaaatgttgcctaagggctctatagtaaaccatttggaagaaagatttgacgactttttttttttggatttcgatcctataatccttcctcctgaaaagaaacatataaatagatatgtattattcttcaaaacattctcttgttcttgtgctttttttttaccatatatcttacttttttttttctctcagagaaacaagcaaaacaaaaagcttttcttttcactaacgtatatgatgcttttgcaagctttccttttccttttggctggttttgcagccaaaatatctgcatcaatgacaaacgaaactagcgatagacctttggtccacttcacacccaacaagggctggatgaatgacccaaatgggttgtggtacgatgaaaaagatgccaaatggcatctgtactttcaatacaacccaaatgacaccgtatggggtacgccattgttttggggccatgctacttccgatgatttgactaattgggaagatcaacccattgctatcgctcccaagcgtaacgattcaggtgctttctctggctccatggtggttgattacaacaacacgagtgggtttttcaatgatactattgatccaagacaaagatgcgttgcgatttggacttataacactcctgaaagtgaagagcaatacattagctattctcttgatggtggttacacttttactgaataccaaaagaaccctgttttagctgccaactccactcaattcagagatccaaaggtgttctggtatgaaccttctcaaaaatggattatgacggctgccaaatcacaagactacaaaattgaaatttactcctctgatgacttgaagtcctggaagctagaatctgcatttgccaatgaaggtttcttaggctaccaatacgaatgtccaggtttgattgaagtcccaactgagcaagatccttccaaatcttattgggtcatgtttatttctatcaacccaggtgcacctgctggcggttccttcaaccaatattttgttggatccttcaatggtactcattttgaagcgtttgacaatcaatctagagtggtagattttggtaaggactactatgccttgcaaactttcttcaacactgacccaacctacggttcagcattaggtattgcctgggcttcaaactgggagtacagtgcctttgtcccaactaacccatggagatcatccatgtctttggtccgcaagttttctttgaacactgaatatcaagctaatccagagactgaattgatcaatttgaaagccgaaccaatattgaacattagtaatgctggtccctggtctcgttttgctactaacacaactctaactaaggccaattcttacaatgtcgatttgagcaactcgactggtaccctagagtttgagttggtttacgctgttaacaccacacaaaccatatccaaatccgtctttgccgacttatcactttggttcaagggtttagaagatcctgaagaatatttgagaatgggttttgaagtcagtgcttcttccttctttttggaccgtggtaactctaaggtcaagtttgtcaaggagaacccatatttcacaaacagaatgtctgtcaacaaccaaccattcaagtctgagaacgacctaagttactataaagtgtacggcctactggatcaaaacatcttggaattgtacttcaacgatggagatgtggtttctacaaatacctacttcatgaccaccggtaacgctctaggatctgtgaacatgaccactggtgtcgataatttgttctacattgacaagttccaagtaagggaagtaaaatagaggttataaaacttattgtcttttttatttttttcaaaagccattctaaagggctttagctaacgagtgacgaatgtaaaactttatgatttcaaagaatacctccaaaccattgaaaatgtatttttatttttattttctcccgaccccagttacctggaatttgttctttatgtactttatataagtataattctcttaaaaatttttactactttgcaatagacatcattttttcacgtaataaacccacaatcgtaatgtagttgccttacactactaggatggacctttttgcctttatctgttttgttactgacacaatgaaaccgggtaaagtattagttatgtgaaaatttaaaagcattaagtagaagtataccatattgtaaaaaaaaaaagcgttgtcttctacgtaaaagtgttctcaaaaagaagtagtgagggaaatggataccaagctatctgtaacaggagctaaaaaatctcagggaaaagcttctggtttgggaaacggtcgac

SEQ ID NO: 66 - DNA, 2159 bp, Artificial Sequence
  Encodes K. lactis UDP-GlcNAc transporter gene (KIMNN2-2)
aaacgtaacgcctggcactctattttctcaaacttctgggacggaagagctaaatattgtgttgcttgaacaaacccaaaaaaacaaaaaaatgaacaaactaaaactacacctaaataaaccgtgtgtaaaacgtagtaccatattactagaaaagatcacaagtgtatcacacatgtgcatctcatattacatcttttatccaatccattctctctatcccgtctgttcctgtcagattctttttccataaaaagaagaagaccccgaatctcaccggtacaatgcaaaactgctgaaaaaaaaagaaagttcactggatacgggaacagtgccagtaggcttcaccacatggacaaaacaattgacgataaaataagcaggtgagcttctttttcaagtcacgatccctttatgtctcagaaacaatatatacaagctaaacccttttgaaccagttctctcttcatagttatgttcacataaattgcgggaacaagactccgctggctgtcaggtacacgttgtaacgttttcgtccgcccaattattagcacaacattggcaaaaagaaaaactgctcgttttctctacaggtaaattacaatttttttcagtaattttcgctgaaaaatttaaagggcaggaaaaaaagacgatctcgactttgcatagatgcaagaactgtggtcaaaacttgaaatagtaattttgctgtgcgtgaactaataaatatatatatatatatatatatatatttgtgtattttgtatatgtaattgtgcacgtcttggctattggatataagattttcgcgggttgatgacatagagcgtgtactactgtaatagttgtatattcaaaagctgctgcgtggagaaagactaaaatagataaaaagcacacattttgacttcggtaccgtcaacttagtgggacagtcttttatatttggtgtaagctcatttctggtactattcgaaacagaacagtgttttctgtattaccgtccaatcgtttgtcatgagttttgtattgattttgtcgttagtgttcggaggatgttgttccaatgtgattagtttcgagcacatggtgcaaggcagcaatataaatttgggaaatattgttacattcactcaattcgtgtctgtgacgctaattcagttgcccaatgctttggacttctctcactttccgtttaggttgcgacctagacacattcctcttaagatccatatgttagctgtgtttttgttctttaccagttcagtcgccaataacagtgtgtttaaatttgacatttccgttccgattcatattatcattagattttcaggtaccactttgacgatgataataggttgggctgtttgtaataagaggtactccaaacttcaggtgcaatctgccatcattatgacgcttggtgcgattgtcgcatcattataccgtgacaaagaattttcaatggacagtttaaagttgaatacggattcagtgggtatgacccaaaaatctatgtttggtatctttgttgtgctagtggccactgccttgatgtcattgttgtcgttgctcaacgaatggacgtataacaagtacgggaaacattggaaagaaactttgttctattcgcatttcttggctctaccgttgtttatgttggggtacacaaggctcagagacgaattcagagacctcttaatttcctcagactcaatggatattcctattgttaaattaccaattgctacgaaacttttcatgctaatagcaaataacgtgacccagttcatttgtatcaaaggtgttaacatgctagctagtaacacggatgctttgacactttctgtcgtgcttctagtgcgtaaatttgttagtcttttactcagtgtctacatctacaagaacgtcctatccgtgactgcatacctagggaccatcaccgtgttcctgggagctggtttgtattcatatggttcggtcaaaactgcactgcctcgctgaaacaatccacgtctgtatgatactcgtttcagaatttttttgattttctgccggatatggtttctcatctttacaatcgcattcttaattataccagaacgtaattcaatgatcccagtgactcgtaactcttatatgtcaatttaagc

SEQ ID NO: 67 - DNA, 981 bp, Artificial Sequence
  Encodes MmSLC35A3 UDP-GlcNAc transporter
atgtctgccaacctaaaatatctttccttgggaattttggtgtttcagactaccagtctggttctaacgatgcggtattctaggactttaaaagaggaggggcctcgttatctgtcttctacagcagtggttgtggctgaatttttgaagataatggcctgcatctttttagtctacaaagacagtaagtgtagtgtgagagcactgaatagagtactgcatgatgaaattcttaataagcccatggaaaccctgaagctcgctatcccgtcagggatatatactcttcagaacaacttactctatgtggcactgtcaaacctagatgcagccacttaccaggttacatatcagttgaaaatacttacaacagcattattttctgtgtctatgcttggtaaaaaattaggtgtgtaccagtggctctccctagtaattctgatggcaggagttgcttttgtacagtggccttcagattctcaagagctgaactctaaggacctttcaacaggctcacagtttgtaggcctcatggcagttctcacagcctgtttttcaagtggctttgctggagtttattttgagaaaatcttaaaagaaacaaaacagtcagtatggataaggaacattcaacttggtttctttggaagtatatttggattaatgggtgtatacgtttatgatggagaattggtctcaaagaatggattttttcagggatataatcaactgacgtggatagttgttgctctgcaggcacttggaggccttgtaatagctgctgtcatcaaatatgcagataacattttaaaaggatttgcgacctccttatccataatattgtcaacaataatatcttatttttggttgcaagattttgtgccaaccagtgtctttttccttggagccatccttgtaatagcagctactttcttgtatggttacgatcccaaacctgcaggaaatcccactaaagcatag

SEQ ID NO: 68 - DNA, 685 bp, Artificial Sequence
  5′-region of PpTRP1 locus
ggccttggaggccgcggaaacggcagtaaacaatggagcttcattagtgggtgttattatggtccctggccgggaacgaacggtgaaacaagaggttgcgagggaaatttcgcagatggtgcgggaaaagagaatttcaaagggctcaaaatacttggattccagacaactgaggaaagagtgggacgactgtcctctggaagactggtttgagtacaacgtgaaagaaataaacagcagtggtccatttttagttggagtttttcgtaatcaaagtatagatgaaatccagcaagctatccacactcatggtttggatttcgtccaactacatgggtctgaggattttgattcgtatatacgcaatatcccagttcctgtgattaccagatacacagataatgccgtcgatggtcttaccggagaagacctcgctataaatagggccctggtgctactggacagcgagcaaggaggtgaaggaaaaaccatcgattgggctcgtgcacaaaaatttggagaacgtagaggaaaatatttactagccggaggtttgacacctgataatgttgctcatgctcgatctcatactggctgtattggtgttgacgtctctggtggggtagaaacaaatgcctcaaaagatatggacaagatcacacaatttatcagaaacgctacataa

SEQ ID NO: 69 - DNA, 847 bp, Artificial Sequence
  3′-region of PpTRP1 locus
aagtcaattaaatacacgcttgaaaggacattacatagctttcgatttaagcagaaccagaaatgtagaaccacttgtcaatagattggtcaatcttagcaggagcggctgggctagcagttggaacagcagaggttgctgaaggtgagaaggatggagtggattgcaaagtggtgttggttaagtcaatctcaccagggctggttttgccaaaaatcaacttctcccaggcttcacggcattcttgaatgacctcttctgcatacttcttgttcttgcattcaccagagaaagcaaactggttctcaggttttccatcagggatcttgtaaattctgaaccattcgttggtagctctcaacaagcccggcatgtgcttttcaacatcctcgatgtcattgagcttaggagccaatgggtcgttgatgtcgatgacgatgaccttccagtcagtctctccctcatccaacaaagccataacaccgaggaccttgacttgcttgacctgtccagtgtaacctacggcttcaccaatttcgcaaacgtccaatggatcattgtcacccttggccttggtctctggatgagtgacgttagggtcttcccatgtctgagggaaggcaccgtagttgtgaatgtatccgtggtgagggaaacagttacgaacgaaacgaagttttcccttctttgtgtcctgaagaattgggttcagtttctcctccttggaaatctccaacttggcgttggtccaacgggggacttcaacaaccatgttgagaaccttcttggattcgtcagcataaagtgggatgtcgtggaaaggagatacgacttggccgtcttggcc

(19) United States
(12) Patent Application Publication
(10) Pub. No.: US 2012/0213728 A1
(43) Pub. Date: Aug. 23, 2012

(54) GRANULOCYTE-COLONY STIMULATING FACTOR PRODUCED IN GLYCOENGINEERED PICHIA PASTORIS

(75) Inventors: Michael Meehl, Lebanon, NH (US); Sandra Rios, Lebanon, NH (US); Sujatha Gomathinayagam, Hanover, NH (US); Huijuan Li, Hanover, NH (US); Piotr Bobrowicz, Hanover, NH (US)

(73) Assignee: MERCK, Rahway, NJ (US)

(21) Appl. No.: 13/504,528

(22) PCT Filed: Oct. 25, 2010
(86) PCT No.: PCT/US10/53920

§ 371 Date: Apr. 27, 2012

Related U.S. Application Data
(60) Provisional application No. 61/256,379, filed on Oct. 30, 2009.

(51) Int. Cl. A61K 38/19; C12P 21/02; C12N 15/62; C12N 1/19

(52) U.S. Cl. 424/85.1; 435/254.23; 435/69.4; 536/23.4

(57) ABSTRACT

Compositions comprising granulocyte-colony stimulating factor (GCSF) produced in a strain of Pichia pastoris glycoengineered to produce a GCSF wherein greater than 18% of the molecules comprise an 0-glycan with one mannose per (0-glycan is described. In particular aspects, the GCSF is PEGylated at the JV-terminus.

### BACKGROUND OF THE INVENTION

(1) Field of the Invention

The present invention relates to a method for making recombinant human Granulocyte-Colony Stimulating Factor (rHuGCSF) produced in glycoengineered Pichia pastoris that has a clinical profile at least as efficacious as the clinical profile of rHuGCSF produced in mammalian or bacterial cells. The present invention further provides compositions of rHuGCSF wherein greater than 18% of the rHuGCSF in the composition have only one mannose residue P-linked to threonine 133. In further aspects, the rHuGCSF molecules in the compositions include a polyethylene glycol polymer at the N-terminus covalently linked to monomethoxypolyethylene glycol (mPEG).

(2) Description of Related Art

The process by which white blood cells grow, divide and differentiate in the bone marrow is called hematopoiesis (Dexter & Spooner, Ann. Rev. Cell. Biol. 3: 423 (1987)). Each of the blood cell types arises from pluripotent stem cells. There are generally three classes of blood cells produced in vivo: red blood cells (erythrocytes), platelets, and white blood cells (leukocytes), the majority of the latter being involved in host immune defense. Proliferation and differentiation of hematopoietic precursor cells are regulated by a family of cytokines, including colony-stimulating factors (CSF's) such as GCSF and interleukins (Arai et al., Ann. Rev. Biochem., 59:783-836 (1990)). The principal biological effect of GCSF in vivo is to stimulate the growth and development of certain white blood cells known as neutrophilic granulocytes or neutrophils (Welte et al., Proc. Natl. Acad. Sci. USA 82: 1526-1530 (1985); Souza et al., Science 232: 61-65 (1986)). When released into the blood stream, neutrophilic granulocytes function to fight bacterial infection.

The amino acid sequence of human GCSF (HuGCSF) was reported by Nagata et al. Nature 319: 415-418 (1986). The natural human GCSF exists in two forms, 174 and 177 amino acids long. The two polypeptides differ by 3 amino acids Val-Ser-Glu at position 36-38. Expression studies indicate that both have authentic GCSF activity. HuGCSF is a monomeric protein that dimerizes the GCSF receptor by formation of a 2:2 complex of two GCSF molecules and two receptors (Horan et al., Biochem. 35(15): 4886-96 (1996)). In its native form, HuGCSF does not undergo N-linked glycosylation, but is O-glycosylated at the Thr-133 position with N-acetylgalactosamine and extended with galactose and sialic acid (Kubota et al. 1990, J Biochem, 107, 486-492). The O-glycosylation of GCSF is not required for its bioactivity although studies comparing filgrastim with a recombinant glycosylated, non-PEGylated GCSF (Lenograstim) suggest that the absence of glycosylation may confer a slight decrease in in vitro potency. Oheda et al., J. Biol. Chem. 265: 11432-11435 (1990) provide evidence that suggests that the O-glycosylation of GCSF protects it against polymerization and denaturation, thus allowing it to retain its biological activity. Aritomi et al., Nature 401: 713-717 (1999) have described the X-ray structure of a complex between HuGCSF and the BN-BC domains of the GCSF receptor.

Expression of rHuGCSF in Escherichia coli, Saccharomyces cerevisiae (U.S. Pat. No. 6,391,585; Bae et al., Biotechnol. Bioeng. 57: 600-609 (1998); Bae et al., Appl. Microbial. & Biotechnol. 52(3): 338-44 (1999)), Pichia pastoris (Lasnik et al., Pfüger Arch—Eur. J. Physiol. 442 (Suppl. 1): R184-186 (2001); Lasnik et al., Biotechnol. Bioengineer. 81: 768-774 (2003); Zhang et al., Biotechnol. Prog. 22: 1090-1095 (2006); Bahraini et al., Iranina J. Biotechnol. 5: 162-169 (2007); Bahraini et al., Biotechnol. & Appl. Biochem. 52: 141-148, E.Pub. 14 May 2008; Saeedinia et al., Biotechnol. 7: 569-573 (2008); Apse-Deshpande et al., J. Biotechnol. 143: 44-50 (2009)), and mammalian cells (Souza et al., Science 232:61-65, (1986); Nagata et al., Nature 319: 415-418, (1986); Robinson & Wittrup, Biotechnol. Prog. 11: 171-177 (1985)) has been reported.

Recombinant human GCSF is generally used for treating various forms of leukopenia. Commercial preparations of recombinant human GCSF are available. These preparations include an N-terminal methionine recombinant human GCSF available under the name filgrastim (GRAN, NEUPOGEN, and a PEGylated form sold as NEULASTA, all trademarks of Amgen); a recombinant human GCSF available under the name lenograstim (GRANOCYTE, trademark of Sanofi-Aventis); and a recombinant human GCSF mutein available under the name nartograstim (NEU-UP, trademark of Kyowa Hakko Kogyo Co. Ltd.). Filgrastim, which has an additional N-terminal methionine residue, is produced in recombinant E. coli cells and as such, is not O-glycosylated. Lenograstim, which has an amino acid sequence identical to the amino acid sequence of native human GCSF, is produced in recombinant Chinese hamster ovary (CHO) cells and as such, is O-glycosylated (See for example, Oheda et al., J. Biochem. (Tokyo) 103: 544-546 (1988)). Nartograstim is a non-glycosylated GCSF mutein produced in recombinant E. coli cells in which five amino acids at the N-terminal region of intact human GCSF are replaced with alternate amino acids.

A few protein-engineered variants of HuGCSF have been reported (U.S. Pat. No. 5,581,476; U.S. Pat. No. 5,214,132, U.S. Pat. No. 5,362,853, U.S. Pat. No. 4,904,584, and Riedhaar-Olson et al. Biochemistry 35: 9034-9041 (1996). Modification of HuGCSF and other polypeptides so as to introduce at least one additional carbohydrate chain as compared to the native polypeptide has been suggested (U.S. Pat. No. 5,218,092). It is stated that the amino acid sequence of the polypeptide may be modified by amino acid substitution, amino acid deletion or amino acid insertion so as to effect addition of an additional carbohydrate chain. In addition, polymer modifications of native HuGCSF, including attachment of PEG groups, have been reported (Satake-Ishikawa et al., Cell Struct. Funct. 17: 157-160 (1992); U.S. Pat. No. 5,824,778, U.S. Pat. No. 5,824,784; WO 96/11953; WO 95/21629; WO 94/20069).

Bowen et al., Exper. Hematol. 27 425-432 (1999) disclose a study of the relationship between molecule mass and duration of activity of PEG-conjugated GCSF mutein. An apparent inverse correlation was suggested between molecular weight of the PEG moieties conjugated to the protein and in vitro activity, whereas in vivo activities increased with increasing molecular weight. It is speculated that a lower affinity of the conjugates act to increase the half-life because receptor-mediated endocytosis is an important mechanism regulating levels of hematopoietic growth factors.

A need therefore still exists for providing novel molecules exhibiting GCSF activity that are useful in the treatment of leukopenia. The present invention relates to such molecules.

### BRIEF SUMMARY OF THE INVENTION

The invention provides compositions of recombinant human granulocyte-colony stimulating factor (rHuGCSF) covalently linked to monomethoxypolyethylene glycol (mPEG) wherein greater than 18% of the rHuGCSF in the composition have only one mannose residue O-linked to threonine 133. The present invention provides Pichia pastoris strains that produce the GCSF in high yield.

In one aspect, the present invention provides a composition comprising recombinant human granulocyte-colony stimulating factor (rHuGCSF) in a pharmaceutically acceptable carrier wherein about at least 18% of the rHuGCSF molecules in the composition have a mannose O-glycan. In general, the rHuGCSF molecules do not contain any detectable mannotriose or mannotetrose O-glycans. In particular embodiments, about 40 to 50% of the rHuGCSF molecules in the composition have a mannose O-glycan, which in further embodiments, do not contain detectable mannobiose or larger O-glycans. In particular embodiments, the rHuGCSF molecules have an N-terminal methionine residue.

In the embodiments and aspects herein, the composition lacks detectable cross-reactivity with antibodies specific for host cell antigens. In particular embodiments, the rHuGCSF comprises at least one covalently attached hydrophilic polymer, which can be a hydrophilic polymer such as polyethylene glycol polymer. The polyethylene glycol polymer can have a molecular weight between about 20 and 40 kD. In particular aspects, the polyethylene glycol polymer has a molecular weight of about 20 kD, 30 kD, or 40 kD.

The present invention also provides a Pichia pastoris host cell that produces a recombinant human granulocyte-colony stimulating factor (rHuGCSF) in which about 40 to 50% of the rHuGCSF obtained from the host cell have mannose O-glycans comprising (a) a nucleic acid molecule encoding the rHuGCSF; and (b) one or more nucleic acid molecules, each encoding at least one secreted chimeric α-1,2-mannosidase I comprising at least the catalytic domain of an α-1,2-mannosidase 1 and a heterologous N-terminal signal sequence for directing extracellular secretion of the secreted chimeric α-1,2-mannosidase I, wherein when there is more than one secreted chimeric α-1,2-mannosidase 1, the secreted chimeric α-1,2-mannosidase I can be the same or different. In particular embodiments, the nucleic acid molecule in (a) encodes the rHuGCSF with an N-terminal methionine.

In further aspects of the host cell, the nucleic acid molecule in (a) encodes a rHuGCSF fusion protein having the structure A-B-C wherein A is a carrier protein having an N-terminal signal sequence for directing extracellular secretion of the fusion protein, B is a linker peptide that includes a protease cleavage site immediately preceding C, and C is the rHuGCSF.

In particular aspects of the host cell, A is human serum albumin, Pichia pastoris cellulase-like protein I (Clp1p), Aspergillus niger glucoamylase, or anti-CD20 light chain. In further still aspects, the protease cleavage site in B is a Kex2p or enterokinase cleavage site. In a particular embodiment, A is a Pichia pastoris cellulase-like protein 1 (Clp1p), the protease cleavage site in B is a Kex 2p cleavage site, and C is rHuGCSF with an N-terminal methionine residue.

In particular aspects, the α-1,2-mannosidase I is a fungal α-1,2-mannosidase I. Examples of fungal α-1,2-mannosidases include but are not limited to Trichoderma reesei α-1,2-mannosidase I, Saccharomyces sp. α-1,2-mannosidase I, Aspergillus sp. α-1,2-mannosidase I, Coccidiodes sp. α-1,2-mannosidase I, Coccidiodes posadasii α-1,2-mannosidase I, and Coccidiodes immitis α-1,2-mannosidase I.

In further aspects, the Pichia pastoris host cell further includes a deletion or disruption of its VPS10-1 gene. In further still aspects, In particular aspects, the host cell further includes a deletion or disruption one or more genes selected from the group consisting of BMT1, BMT2, BMT3, and BMT4. In further particular aspects, the host cell further includes a deletion or disruption the STE13 and/or DAP2 genes and in further still particular aspects, the host cell further includes a deletion or disruption PEP4 and/or PRB1 genes. In further still particular aspects, the host cell includes a deletion or disruption of the PN01, MNN4A, and MNN4B genes.

In further aspects, the Pichia pastoris host cell has been modified to produce glycoproteins that have human-like N-glycans, such N-glycans include hybrid N-glycans and/or complex N-glycans. In further aspects, the Pichia pastoris host cell includes a deletion or disruption of the OCH1 gene and includes one or more nucleic acid molecules encoding an α-1,2-mannosidase I catalytic domain fused to a heterologous cellular targeting signal peptide that targets the enzyme to the ER or Golgi apparatus of the host cell where the enzyme functions optimally. In further still aspects, the host cell further includes one or more nucleic acid molecules encoding one or more enzymes selected from the group consisting of sugar transporters, GlcNAc transferases, galactosyltransferases, and sialic acid transferases.

The present invention further provides a nucleic acid molecule encoding a fusion protein having the structure A-B-C wherein A is a carrier protein having an N-terminal signal sequence for directing extracellular secretion of the fusion protein, B is a linker peptide that includes a protease cleavage site immediately preceding C, and C is a rHuGCSF. In particular aspects of the nucleic acid, the nucleic acid encodes a rHuGCSF that includes an N-terminal methionine residue. In a particular embodiment, A is a Pichia pastoris cellulase-like protein 1 (Clp1p), the protease cleavage site in B is a Kex 2p cleavage site, and C is rHuGCSF with an N-terminal methionine residue.

The present invention further provides a method for making a composition of recombinant human granulocyte-colony stimulating factor (rHuGCSF) in which about 40 to 50% of the rHuGCSF in the composition have mannose O-glycans in Pichia pastoris comprising: (a) providing a recombinant Pichia pastoris host cell that includes (i) a nucleic acid molecule encoding the rHuGCSF; and (ii) one or more nucleic acid molecules, each encoding at least one secreted chimeric α-1,2-mannosidase I comprising at least the catalytic domain of an α-1,2-mannosidase I and a heterologous N-terminal signal sequence for directing extracellular secretion of the secreted chimeric α-1,2-mannosidase I, wherein when there is more than one secreted chimeric α-1,2-mannosidase I, the secreted chimeric α-1,2-mannosidase 1 can be the same or different; (b) growing the host cell in a medium under conditions that induce expression of the nucleic acid molecule encoding the rHuGCSF to produce the rHuGCSF, which secreted into the medium; and (c) recovering the rHuGCSF from the medium to produce the composition of recombinant human granulocyte-colony stimulating factor (rHuGCSF) in which about 40 to 50% of the rHuGCSF in the composition have mannose O-glycans. In particular embodiments, the nucleic acid molecule in (a) encodes the rHuGCSF with an N-terminal methionine.

In further aspects of the method, the nucleic acid molecule in (a) encodes a rHuGCSF fusion protein having the structure A-B-C wherein A is a carrier protein having an N-terminal signal sequence for directing extracellular secretion of the fusion protein, B is a linker peptide that includes a protease cleavage site immediately preceding C, and C is the rHuGCSF.

In particular aspects of the method, A is human serum albumin, Pichia pastoris cellulase-like protein I (Clp1p), Aspergillus niger glucoamylase, or anti-CD20 light chain. In further still aspects, the protease cleavage site in B is a Kex2p or enterokinase cleavage site. In a particular embodiment, A is a Pichia pastoris cellulase-like protein 1 (Clp1p), the protease cleavage site in B is a Kex 2p cleavage site, and C is rHuGCSF with an N-terminal methionine residue.

In particular aspects of the method, the α-1,2-mannosidase I is a fungal α-1,2-mannosidase I. Examples of fungal α-1,2-mannosidases include but are not limited to Trichoderma reesei α-1,2-mannosidase I, Saccharomyces sp. α-1,2-mannosidase 1, Aspergillus sp. α-1,2-mannosidase 1, Coccidiodes sp. α-1,2-mannosidase I, Coccidiodes posadasii α-1,2-mannosidase I, and Coccidiodes immitis α-1,2-mannosidase 1.

In further aspects of the method, the Pichia pastoris host cell further includes a deletion or disruption of its VPS10-1 gene. In further still aspects, In particular aspects, the host cell further includes a deletion or disruption one or more genes selected from the group consisting of BMT1, BMT2, BMT3, and BMT4. In further particular aspects, the host cell further includes a deletion or disruption the STE13 and/or DAP2 genes and in further still particular aspects, the host cell further includes a deletion or disruption PEP4 and/or PRB1 genes. In further still particular aspects, the host cell includes a deletion or disruption of the PNO1, MNN4A, and MNN4B genes.

In further aspects of the method, the rHuGCSF is conjugated to at least one hydrophilic polymer. The rHuGCSF produced can comprise at least one covalently attached hydrophilic polymer, which can be a hydrophilic polymer such as polyethylene glycol polymer. The polyethylene glycol polymer can have a molecular weight between 20 and 40kD. In particular aspects, the polyethylene glycol polymer has a molecular weight of about 20 kD, 30 kD, or 40 kD.

### BRIEF DESCRIPTION OF THE DRAWINGS

FIG. 1A-E shows the construction of the glycoengineered Pichia pastoris strain YGLY8538 expressing rHuGCSF.

FIG. 2 shows a map of plasmid pGLY6. Plasmid pGLY6 is an integration vector that targets the URA5 locus and contains a nucleic acid molecule comprising the S. cerevisiae invertase gene or transcription unit (ScSUC2) flanked on one side by a nucleic acid molecule comprising a nucleotide sequence from the 5′ region of the P. pastoris URA5 gene (PpURA5-5′) and on the other side by a nucleic acid molecule comprising the a nucleotide sequence from the 3′ region of the P. pastoris URA5 gene (PpURA5-3′).

FIG. 3 shows a map of plasmid pGLY40. Plasmid pGLY40 is an integration vector that targets the OCH1 locus and contains a nucleic acid molecule comprising the P. pastoris URA5 gene or transcription unit (PpURA5) flanked by nucleic acid molecules comprising lacZ repeats (lacZ repeat) which in turn is flanked on one side by a nucleic acid molecule comprising a nucleotide sequence from the 5′ region of the OCH1 gene (PpOCH1-5′) and on the other side by a nucleic acid molecule comprising a nucleotide sequence from the 3′ region of the OCH1 gene (PpOCH1-3′).

FIG. 4 shows a map of plasmid pGLY43a. Plasmid pGLY43a is an integration vector that targets the BMT2 locus and contains a nucleic acid molecule comprising the K. lactis UDP-N-acetylglucosamine (UDP-GlcNAc) transporter gene or transcription unit (KlGlcNAc Transp.) adjacent to a nucleic acid molecule comprising the P. pastoris URA5 gene or transcription unit (PpURA5) flanked by nucleic acid molecules comprising lacZ repeats (lacZ repeat). The adjacent genes are flanked on one side by a nucleic acid molecule comprising a nucleotide sequence from the 5′ region of the BMT2 gene (PpPBS2-5′) and on the other side by a nucleic acid molecule comprising a nucleotide sequence from the 3′ region of the BMT2 gene (PpPBS2-3′).

FIG. 5 shows a map of plasmid pGLY48. Plasmid pGLY48 is an integration vector that targets the MNN4L1 locus and contains an expression cassette comprising a nucleic acid molecule encoding the mouse homologue of the UDP-GlcNAc transporter (MmGlcNAc Transp.) open reading frame (ORF) operably linked at the 5′ end to a nucleic acid molecule comprising the P. pastoris GAPDH promoter (PpGAPDH Prom) and at the 3′ end to a nucleic acid molecule comprising the S. cerevisiae CYC termination sequence (ScCYC TT) adjacent to a nucleic acid molecule comprising the P. pastoris URA5 gene or transcription unit (PpURA5) flanked by lacZ repeats (lacZ repeat) and in which the expression cassettes together are flanked on one side by a nucleic acid molecule comprising a nucleotide sequence from the 5′ region of the P. Pastoris MNN4L1 gene (PpMNN4L1-5′) and on the other side by a nucleic acid molecule comprising a nucleotide sequence from the 3′ region of the MNN4L1 gene (PpMNN4L1-3′).

FIG. 6 shows as map of plasmid pGLY45. Plasmid pGLY45 is an integration vector that targets the PNO1/MNN4 loci contains a nucleic acid molecule comprising the P. pastoris URA5 gene or transcription unit (PpURA5) flanked by nucleic acid molecules comprising lacZ repeats (lacZ repeat) which in turn is flanked on one side by a nucleic acid molecule comprising a nucleotide sequence from the 5′ region of the PNO1 gene (PpPNO1-5′) and on the other side by a nucleic acid molecule comprising a nucleotide sequence from the 3′ region of the MNN4 gene (PpMNN4-3′).

FIG. 7 shows the construction of optimized rHuGCSF-expression strains derived from YGLY8538.

FIG. 8A-B shows the construction of plasmid vector pGLY5178 encoding rHuMetGCSF and targeting the Pichia pastoris AOX1 locus.

FIG. 9 shows the construction of plasmid vector pGLY5192 used to delete the VPS10-1 vacuolar receptor gene by homologous recombination.

FIG. 10A-B shows the construction of plasmid vector pGLY729 used to delete the PEP4 protease gene by homologous recombination.

FIG. 11A-B shows the construction of plasmid vector pGLY1614 used to delete the PRB1 protease gene by homologous recombination.

FIG. 12A shows the construction of plasmid vector pGLY1162 encoding the T. reesei α-1,2 mannosidase (TrMNS1) and targeting the Pichia pastoris PRO1 locus.

FIG. 12B shows the construction of plasmid vectors pGLY1896 and pGFI207t, both encoding the T. reesei α-1,2 mannosidase (TrMNS1) and the mouse α-1,2 mannosidase I catalytic domain fused to the S. cerevisiae MNN2 leader peptide and targeting the Pichia pastoris PRO1 locus.

FIG. 13 shows the construction of plasmid vector pGFI204t encoding the T. reesei α-1,2 mannosidase (TrMNS1) and targeting the Pichia pastoris TRP1 locus.

FIG. 14 shows the construction of the glycoengineered Pichia pastoris strain YGLY7553 expressing rHuGCSF.

FIG. 15 shows the construction of the glycoengineered Pichia pastoris strains YGLY8063 and YGLY8543 expressing rHuMetGCSF.

FIG. 16 shows a map of plasmid pGLY3419 (pSH1110). Plasmid pGLY3430 (pSH1115) is an integration vector that contains an expression cassette comprising the P. pastoris URA5 gene or transcription unit (PpURA5) flanked by lacZ repeats (lacZ repeat) flanked on one side with the 5′ nucleotide sequence of the P. pastoris BMT1 gene (PBS1 5′) and on the other side with the 3′ nucleotide sequence of the P. pastoris BMT1 gene (PBS1 3′)

FIG. 17 shows a map of plasmid pGLY3411 (pSH 1092). Plasmid pGLY3411 (pSH1092) is an integration vector that contains the expression cassette comprising the P. pastoris URA5 gene or transcription unit (PpURA5) flanked by lacZ repeats (lacZ repeat) flanked on one side with the 5′ nucleotide sequence of the P. pastoris BMT4 gene (PpPBS4 5′) and on the other side with the 3′ nucleotide sequence of the P. pastoris BMT4 gene (PpPBS4 3′).

FIG. 18 shows a map of plasmid pGLY3421 (pSH1106). Plasmid pGLY4472 (pSH1186) contains an expression cassette comprising the P. pastoris URA5 gene or transcription unit (PpURA5) flanked by lacZ repeats (lacZ repeat) flanked on one side with the 5′ nucleotide sequence of the P. pastoris BMT3 gene (PpPBS3 5′) and on the other side with the 3′ nucleotide sequence of the P. pastoris BMT3 gene (PpPBS3 3′).

FIG. 19 shows a map of plasmid pGLY4521 (pSH1234). Plasmid pGLY4521 (pSH1234) contains an expression cassette comprising the P. pastoris URA5 gene or transcription unit (PpURA5) flanked by lacZ repeats (lacZ repeat) flanked on one side with the 5′ nucleotide sequence of the P. pastoris DAP2 gene and on the other side with the 3′ nucleotide sequence of the P. pastoris DAP2 gene.

FIG. 20 shows a map of plasmid pGLY5018 (pSH1245). Plasmid pGLY5018 (pSH1245) is an integration vector that contains an expression cassette comprising a nucleic acid molecule encoding the Nourseothricin resistance ORF (NAT) operably linked to the P. pastoris TEF1 promoter (PTEF) and P. pastoris TEF1 termination sequence (TTEF) flanked one side with the 5′ nucleotide sequence of the P. pastoris STE13 gene and on the other side with the 3′ nucleotide sequence of the P. pastoris STE13 gene.

FIG. 21 shows the results of an electrospray mass spectroscopy analysis of the integrity of rHuGCSF produced in glycoengineered Pichia pastoris strain YGLY7553. The rHuGCSF was produced in the form that lacks an N-terminal methionine.

FIG. 22 shows the results of an electrospray mass spectroscopy analysis of the integrity of rHuGCSF produced in glycoengineered Pichia pastoris strain YGLY8063. The rHuGCSF was produced in the form that has an N-terminal methionine.

FIG. 23 shows the results of an electrospray mass spectroscopy analysis of the integrity of rHuGCSF produced in glycoengineered Pichia pastoris strain YGLY10556. The rHuGCSF was produced in the form that has an N-terminal methionine.

FIG. 24 shows the results of an electrospray mass spectroscopy analysis of the integrity of rHuGCSF produced in glycoengineered Pichia pastoris strain YGLY11090. The rHuGCSF was produced in the form that has an N-terminal methionine.

FIG. 25 shows a Western blot comparing the size of rHuGCSF produced in a strain with wild-type STE13 and DAP2 (lanes 27-30) compared to rHuGCSF produced in a strain in which the genes encoding ste13p and dap2p have been deleted (lanes 32-34), rHuMetGCSF with an N-terminal methionine residue produced in a strain with wild-type STE13 and DAP2 (lane 31); and rHuMetGCSF with an N-terminal methionine residue produced in a strain in which the genes encoding ste13p and dap2p have been deleted (lanes 35-36). The rHuGCSF was isolated from the medium of Sixfors fermentations, resolved on SDS gels, and transferred to membranes that were then probed with anti-GCSF antibodies.

FIG. 26 shows a chart comparing the yield of rHuGCSF produced in strain YGLY7553 (ScMF-1L1β-rHuGCSF fusion protein) to the yield of rHuGCSF produced in strain YGLY8538 (Clp1p-rHuMetGCSF fusion protein; Δste13/dap2). Also, shown is the yield of rHuMetGCSF produced in strain YGLY8063 (human serum albumin-rHuMetGCSF fusion protein) and strain YGLY8543 (human serum albumin-rHuGCSF fusion protein in strain that is OCH1⁺).

FIG. 27 shows a chart comparing the yield of rHuGCSF produced in strain YGLY7553 (ScMF-1L1β-rHuGCSF fusion protein) to the yield of rHuGCSF produced in strain YGLY8538 (Clp1p-rHuMetGCSF fusion protein; Δste13/dap2) to the yield produced in strain YGLY9933 (Clp1p-rHuMetGCSF fusion protein; Δste13/dap2/vps10-1).

FIG. 28 shows an SDS polyacrylamide gel stained with Coomassie blue showing the rHuMetGCSF species that were generated in a PEGylation reaction.

FIG. 29 shows a chromatogram of the purification of rHuMetGCSF from strain YGLY8538 PEGylated at the N-terminus. The first three small peaks in the chromatogram refer to di-PEG-rHuMetGCSF. The fourth single huge peak for mono-PEG-rHuMetGCSF. An aliquot of the fourth peak was electrophoresed on and SDS-PAGE Gel.

FIG. 30 shows an SDS polyacrylamide gel stained with Coomassie blue showing that the fourth peak contained mono-PEGylated rHuMetGCSF.

### DETAILED DESCRIPTION OF THE INVENTION

The present invention provides methods for producing a recombinant human granulocyte-colony stimulating factor in recombinant glycoengineered Pichia pastoris strains in high yield. The present invention further provides compositions comprising recombinant human GCSF wherein the recombinant human GCSF is O-glycosylated at threonine residue 133/134 with a single mannose residue at an occupancy of about 40 to 60% wherein the composition lacks mannobiose or larger O-glycans and wherein the composition lacks detectable cross-reactivity with antibodies specific for host cell antigens (HCA). In further embodiments, the recombinant human GCSF in the compositions is covalently linked to monomethoxypolyethylene glycol (mPEG), predominantly at the N-terminus. The present invention further provides recombinant Pichia pastoris strains that have been genetically engineered to produce the recombinant human GCSF.

The recombinant human GCSF that can be produced using the methods herein includes (1) recombinant human GCSF in which the amino acid sequence of the GCSF is identical to the amino acid sequence of native human GCSF (rHuGCSF), (2) recombinant human GCSF in which the GCSF includes an N-terminal methionine residue (rHuMetGCSF), and (3) recombinant human GCSF muteins (rHuGCSFm) in which one or more amino acid additions, substitutions, or deletions other than the presence or lack of an N-terminal methionine residue. As used herein, the term “rHuGCSF” will be understood to refer to all three classes of recombinant human GCSF unless specifically stated otherwise. It is further understood that when the recombinant GCSF has an amino acid sequence identical to human native GCSF, the O-glycosylated threonine residue is at position 133 and when the GCSF further includes an N-terminal methionine residue, the O-glycosylated threonine residue is at position 134.

Lasnik et al., Pfüger Arch Eur. J. Physiol. 442 (Suppl. 1): R184-186 (2001); Lasnik et al., Biotechnol. Bioengineer. 81: 768-774 (2003); Zhang et al., Biotechnol. Prog. 22: 1090-1095 (2006); Bahraini et al., Iranina 3. Biotechnol. 5: 162-169 (2007); Bahrami et al., Biotechnol. & Appl. Biochem. 52: 141-148, E.Pub. 14 May 2008; and Saeedinia et al., Biotechnol. 7: 569-573 (2008) have reported producing rHuGCSF in the GS115 strain of Pichia pastoris that possesses wild-type fungal glycosylation patterns. However, the present invention provides improvements to the current methods for producing rHuGCSF in Pichia pastoris. These improvements enable the production in Pichia pastoris of rHuGCSF that is of a quality wherein the rHuGCSF is essentially full-length and intact (e.g., nor N-terminal protease degradation) and is O-glycosylated with a single mannose residue with about 40 to 60% occupancy. Further improvements to producing rHuGCSF in Pichia pastoris, include genetically engineered mutations described herein that inhibit transport of the rHuGCSF to the vacuole where it is degraded. These mutations that inhibit transport of rHuGCSF to the vacuole substantially improved the yield of the rHuGCSF.

In addition, production of the rHuGCSF using the recombinant Pichia pastoris strains herein also provides rHuGCSF compositions that lack cross-reactivity with antibodies made against host cell antigens (HCAs). Antibodies against HCA are generally made by using a NORF strain (generally, a strain that is the same as the strain encoding GCSF but which lacks the GCSF ORF) to raise the anti-HCA polyclonal antibodies. HCA are residual host cell protein and cell wall contaminants that may carry over to recombinant protein compositions that can be immunogenic and which can alter therapeutic efficacy or safety of a therapeutic protein. In general, the test for whether a composition contains cross-reactivity with antibodies made against HCA is to test the composition with polyclonal antibodies that have made against the total proteins and cellular components of the host cell that does not make the therapeutic protein to see if the antibodies recognize any antigen within the composition. A composition that has cross-reactivity with antibodies made against HCA means that the composition contains some contaminating host cell material, usually N-glycans with phosphomannose residues or beta-mannose residues or mannobiose or larger O-glycans. Wild-type strains of Pichia pastoris will produce glycoproteins that have these N-glycan and O-glycan structures. Antibody preparations made against total host cell proteins would be expected to include antibodies against these structures. GCSF does not contain N-glycans but is O-glycosylated; rHuGCSF isolated from wild-type Pichia pastoris might include contaminating material (proteins or the like) that cross-react with antibodies made against the host cell. The strains described herein include genetically engineered mutations that enable rHuGCSF compositions to be made that lack cross-reactivity with antibodies against host cell antigens.

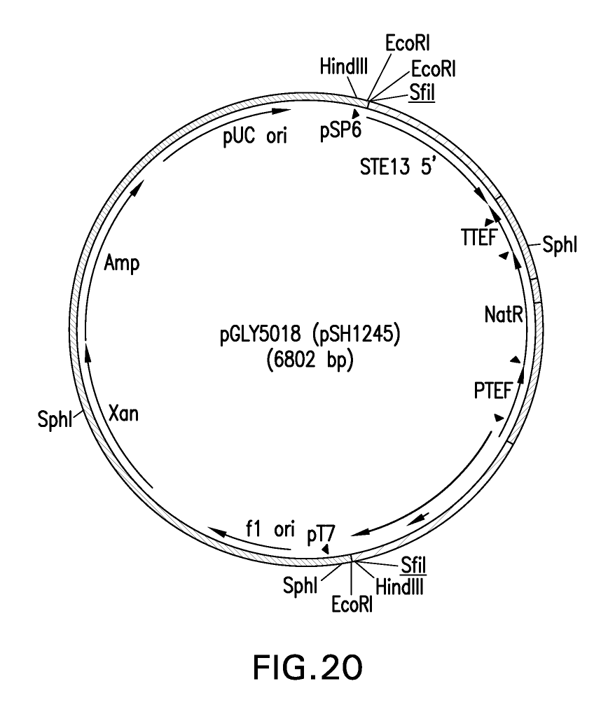

The inventors have discovered that producing rHuGCSF in Pichia pastoris glycoengineered to produce therapeutic proteins that lacked cross-reactivity with antibodies made against host cell antigens and lacked Pichia pastoris O-glycosylation patterns, e.g., O-glycans with one to four mannose residues (e.g., mannose, mannobiose, mannotriose, and mannotetrose O-glycan structures) would be suitable for use in compositions intended for treating humans, produced a mixture of full-length and truncated rHuGCSF molecules (See FIG. 20). The rHuGCSF also comprised a mixture of mannose and mannobiose O-glycans. Host cell diaminopeptidase activity resulted in the loss of amino acid residues at the N-terminus and host cell carboxypeptidase activity resulted in the loss of amino acid residues at the C-terminus. In addition, the yield of rHuGCSF produced in the glycoengineered Pichia pastoris was about 1 mg/L, too low for the host cells to be useful for manufacturing rHuGCSF.

To reduce or eliminate production of compositions of rHuGCSF that lack cross-reactivity to antibodies against HCA, the glycoengineered Pichia pastoris strain has been constructed to delete or disrupt the genes involved in producing yeast N-glycans, e.g., deletion or disruption of the genes encoding initiating α-1,6-mannosyltransferase activity, beta-mannososyltransferase activities, and phosphomannosyltransferase activities, and further includes one or more nucleic acid molecules encoding one or more glycosylation enzyme activities that enable it to produce glycoproteins that have N-glycans that have predominantly at least a Man₅GlcNAc₂ oligosaccharide structure. Thus, these strains are capable of producing recombinant proteins that are not contaminated with detectable host cell antigens. These glycoengineered strains grow less robustly than wild-type strains such as GS115. However, these glycoengineered strains are capable of producing high quality glycoproteins that can be used as therapeutics in humans; however, in particular cases, such as shown here for producing rHuGCSF, the yield and quality of rHuGCSF were unsatisfactory. Thus, producing rHuGCSF of therapeutic quality and in high yield in Pichia pastoris presented a series of challenges: (1) reducing the peptidase activity that is “clipping” the N- and C-termini of the rHuGCSF, (2) reducing O-glycosylation to an extent sufficient to eliminate rHuGCSF molecules that contain mannobiose or larger O-glycans, and (3) increase the yield of rHuGCSF produced in the 2.0 strain.

The present invention has solved these identified problems to the extent that it provides a means for producing high quality rHuGCSF (e.g., essentially full length and intact) in high yield (i.e., yields of 50 mg/L or more). The present invention also provides rHuGCSF compositions in which the rHuGCSF molecules lack mannobiose or larger O-glycans and about 40 to 60% of the rHuGCSF molecules are O-glycosylated with a single mannose residue and in which the compositions lack detectable cross-reactivity with antibodies made against HCA.

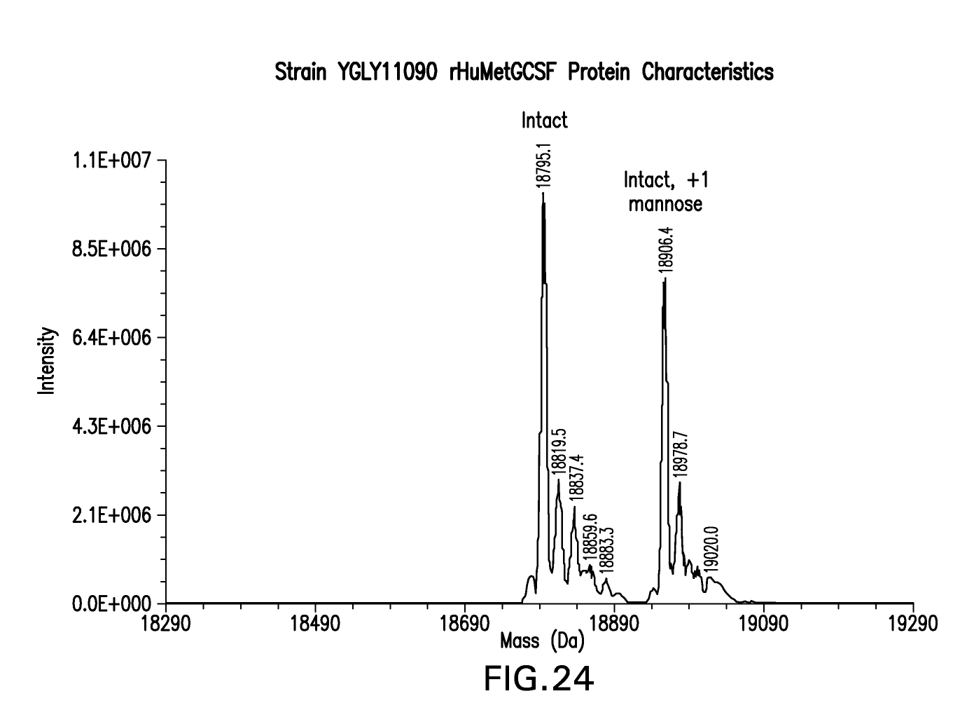

In resolving the first challenge, the applicants determined that N-terminal clipping (TP diaminopeptidase activity) can be abrogated by deleting or disrupting the STE13 and DAP2 genes in the Pichia pastoris production strain encoding the Ste13p and Dap2p proteases or by modifying the nucleic acid molecule encoding the rHuGCSF to further encode an N-terminal methionine residue. Identification and deletion of the STE13 or DAP2 genes in Pichia pastoris has been described in Published PCT Application No. WO2007148345 and in Pabha et al., Protein Express. Purif. 64: 155-161 (2009). FIG. 24 shows that deleting both the STE13 and DAP2 genes and/or producing the rHuGCSF with an N-terminal methionine residue abrogated N-terminal clipping. While producing the rHuGCSF with an N-terminal residue will substantially abrogate N-terminal clipping, there is still a risk that during production lysed cells in the production medium will release Ste13p and Dap2p into the production medium where they have the opportunity at least during the production time period to interact with secreted rHuGCSF and cleave off N-terminal residues. Therefore, in further aspects, in addition to producing the rHuGCSF with an N-terminal methionine, the method further includes deletions or disruptions of the STE13 and DAP2 genes.

To further abrogate protease digestion of rHuGCSF during production, production medium usually contains Pepstatin A and Chymostatin, protease inhibitors of endoproteases protease A (PrA) and protease B (PrB), respectively. Compositions of rHuGCSF produced from Pichia pastoris grown in medium that does not contain these inhibitors usually contain degraded molecules. As an alternative to use of these protease inhibitors, the pep4 and prb1 genes encoding PrA and PrB, respectively, can be deleted or disrupted. Recombinant glycoengineered Pichia pastoris that further include disruption of these two genes further improve the integrity of the rHuGCSF that is produced. An additional benefit to including these two deletions is that the production medium does not need to include Chymostatin and Pepstatin A, thus providing a reduction in production costs. A further still benefit is that the prb1 deletion or disruption causes a reduction in cellular growth rate, which allows for an extended induction period for producing the rHuGCSF, thus improving the yield of rHuGCSF.

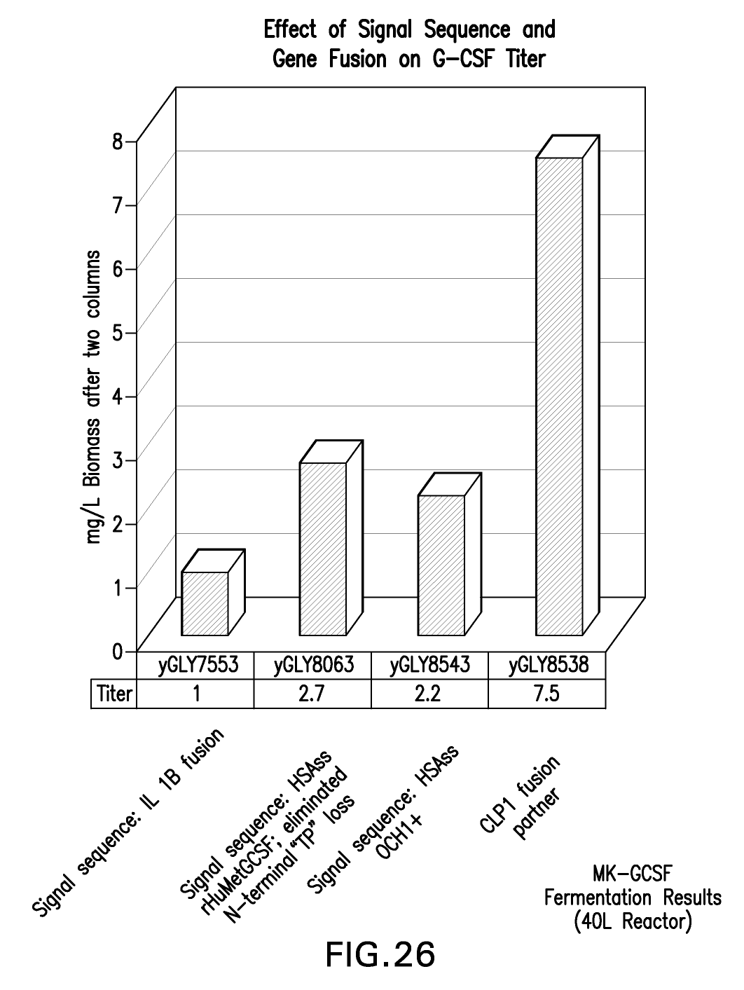

Initially, the rHuGCSF was expressed as a fusion protein in which the N-terminus of rHuGCSF was fused to a linker peptide containing a Kex2 cleavage site at the C-terminus and which in turn was fused at its N-terminus to the C-terminus of a fusion protein consisting of human IL1β fused to a Saccharomyces cerevisiae mating factor signal sequence. However, as shown in FIG. 26, the yield of rHuGCSF produced was only about 1 mg/L. Producing rHuGCSF fused to the human serum albumin signal peptide appeared to improve yield almost three-fold (FIG. 26). However, it was found that by expressing the rHuGCSF as a fusion protein wherein it was coupled to well expressed Pichia pastoris glycoprotein protein Clp1p (encoded by CLP1 gene: cellulase-like protein 1), the yield of rHuGCSF increased over seven-fold (FIG. 26).

Therefore, for producing rHuGCSF, the rHuGCSF is encoded as a fusion protein in which the N-terminus of the rHuGCSF is covalently linked by peptide bond to a linker peptide containing a Kex2p protease cleavage site which in turn is linked by peptide bond to the C-terminus of a glycoprotein that is well expressed in Pichia pastoris. While the methods herein have been exemplified using the well expressed Pichia pastoris Clp1p glycoprotein, other well-expressed Pichia pastoris glycoproteins are also expected to improve the yield of rHuGCSF similar to Clp1p. The Kex2 cleavage site in the linker is positioned so that the Kex2p cleaves the peptide bond between the linker and the rHuGCSF to produce a rHuGCSF free of the linker and Clp1p. Fusing the Clp1p to the rHuGCSF is believed to increase the yield of rHuGCSF by using the Clp1p to pull the rHuGCSF through the secretory pathway. The Kex2p cleaves the Kex2 site towards the end of the secretory pathway.

Proteins that are destined for the vacuole are sorted from proteins destined for the cell surface in the late Golgi compartment. The sorting process is similar to the mammalian lysosomal sorting system; however, unlike the mammalian lysosomal sorting system where the sorting signal is a carbohydrate moiety, in yeast the sorting signal is contained within the polypeptide chains themselves. The most thoroughly studied vacuolar protein in S. cerevisiae is carboxypeptidase Y (CPY encoded by PRC1), which has a sorting signal at the N-terminus of its prosegment that is QRPL (SEQ ID NO:32). This sorting signal sequence is recognized by the CPY sorting receptor Vps10p/Pep1p, which binds and directs the CPY to the vacuole. Human GCSF has a short amino acid sequence in its N-terminal region (QSFL, SEQ ID NO:33) that appears similar to the CPY sorting signal sequence QRPL (SEQ ID NO:32). Mutational analysis of the sorting signal sequence by Van Voosrt et al., J. Biol. Chem. 271: 841-846 (1996) suggests that the QSFL (SEQ ID NO:33) sequence found in human GCSF is a cryptic sorting signal that might be capable of directing a substantial amount of the rHuGCSF to the vacuole where it is degraded. Therefore, it was reasoned that the yield of rHuGCSF could be increased by deleting or disrupting the VPS10-1 gene.

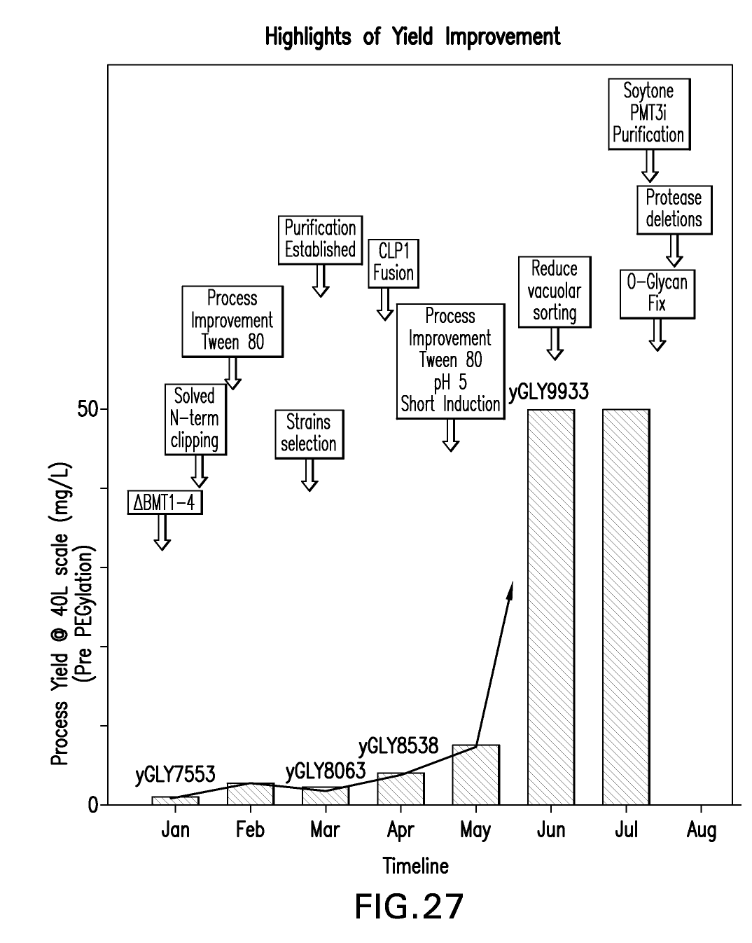

The VPS10-1 gene in Pichia pastoris was identified and the gene deleted in the above glycoengineered Pichia pastoris to produce a Pichia pastoris strain that lacked CPY sorting mediated by the Vps10-1p. Production of rHuGCSF in this strain resulted in a substantial increase in yield, from about 7.5 mg/L to about 50 mg/L (See FIG. 27). Therefore, the present invention further provides that the glycoengineered Pichia pastoris lack a functional CPY sorting receptor, e.g., Vps10-1p.

The above glycoengineered Pichia pastoris strains also overexpress a chimeric fungal α-1,2-mannosidase I comprising a signal sequence for directing extracellular secretion. Production or rHuGCSF in these strains results in rHuGCSF compositions in which ratio of no O-glycans to mannose and mannobiose O-glycans is about 38:18:44. It was found that engineering the strains to overexpress a second copy of the chimeric fungal α-1,2-mannosidase I resulted in rHuGCSF compositions in which about 40 to 60% of the rHuGCSF lack O-glycans and for those molecules that are O-glycosylated, the O-glycans contain a single mannose residue. Mannobiose O-glycans were not detected. The lack of mannobiose O-glycans reduces the risk of having cross-reactivity to antibodies against HCA.

In light of the above, the provided are Pichia pastoris host cells genetically engineered to produce rHuGCSF that is intact and wherein at least some of the rHuGCSF molecules have mannose O-glycans but not mannobiose or larger O-glycans. Further provided are compositions comprising the rHuGCSF wherein the compositions lack detectable cross-reactivity with host cell antigen and wherein the rHuGCSF is intact and wherein at least some of the rHuGCSF molecules have mannose O-glycans but not mannobiose or larger O-glycans. In particular aspects, the rHuGCSF includes an N-terminal methionine.

The Pichia pastoris host cells that are used to produce the rHuGCSF are genetically engineered to produce glycoproteins in general that have human-like or humanized N-glycans, to lack diaminopeptidase activity encoded by ste13 and dap2, and to lack carboxypeptidase Y (CPY) sorting. In further aspects, the host cells also lack one or both protease activities selected from Protease A (PrA, encoded by PEP4) and Protease B (PrB, encoded by PRB1). Therefore, in particular aspects, the host cells are provided that lack ste13p and dap2p activities; lack ste13p, dap2p, and PrA activities; lack ste13p, dap2p, and PrB activities; or lack ste13p, dap2p, PrA, and PrB activities. As used herein, lacking an activity can be achieved by deleting or disrupting the gene encoding the activity or using antisense or siRNA to inhibit expression of mRNA encoding the activity. Alternatively, one or more of the protease activities can be inhibited using an inhibitor of the activity. For example, Pepstatin A can be used to inhibit PrA activity and Chymostatin can be used to inhibit PrB activity. In general, the host cells are rendered lacking in CPY sorting by deleting or disrupting VPS10-1 gene encoding the CPY sorting receptor.

The host cells are also modified to overexpress a secreted chimeric fungal α-1,2-mannosidase I comprising a signal sequence for directing extracellular secretion of the chimeric mannosidase I fused to the N-terminus of at least the catalytic domain of an α-1,2-mannosidase. These host cells are capable of producing rHuGCSF compositions wherein about 40 to 60% of the rHuGCSF lack O-glycans and wherein for those molecules that are O-glycosylated, the O-glycans contain a single mannose residue and no detectable mannobiose O-glycans. In general, the host cells express two or more secreted chimeric mannosidase I enzymes encoded on the same or on different nucleic acid molecules and the secreted chimeric mannosidase Is can be the same or different. In particular aspects, the α-1,2-mannosidase I is a fungal α-1,2-mannosidase I. Examples of fungal α-1,2-mannosidase I include but are not limited to Trichoderma reesei α-1,2-mannosidase I, Saccharomyces sp. α-1,2-mannosidase I, Aspergillus sp. α-1,2-mannosidase I, Coccidiodes sp. α-1,2-mannosidase I, Coccidiodes posadasii α-1,2-mannosidase I, and Coccidiodes immitis α-1,2-mannosidase I. Any signal sequence that directs a protein for processing through the secretory pathway can be used. Examples of such signal sequences include but are not limited to Saccharomyces cerevisiae mating factor pre-signal peptide MRFPSIFTAVLFAASSALA (SEQ ID NO:25), Saccharomyces cerevisiae mating factor pre-pro signal peptide MRFPSIFTAVLFAASSALASLNCTLRDSQQKSLVMSGPYELKALVKR (SEQ ID NO:27), Alpha amylase signal peptide from Aspergillus niger α-amylase MVAWWSLFLY GLQVAAPALA (SEQ ID NO:23), and human serum albumin (HSA) signal peptide MKWVTFISLLFLFSSAYS (SEQ ID NO:29). Nucleic acid molecules encoding the secreted chimeric mannosidase I can be operably linked to a constitutive or inducible lower eukaryote-specific promoter. Examples of such promoters include but are not limited to the Saccharomyces cerevisiae TEF-1 promoter, Pichia pastoris GAPDH promoter, Pichia pastoris GUT1 promoter, PMA-1 promoter, Pichia pastoris PCK-1 promoter, and Pichia pastoris AOX-1 and AOX-2 promoters.

Modifying Pichia pastoris host cells to express glycoproteins in which the glycosylation pattern is human-like or humanized can be achieved by eliminating selected endogenous glycosylation enzymes and/or supplying exogenous enzymes as described by for example, Gerngross, U.S. Pat. No. 7,029,872 and Gerngross et al., U.S. Published Application No. 20040018590. For example, a host cell can be selected or engineered to be depleted in 1,6-mannosyl transferase activities (e.g., ΔOCH1), which would otherwise add mannose residues onto the N-glycan on a glycoprotein.

In one embodiment, the host cell further includes an α-1,2-mannosidase catalytic domain fused to a cellular targeting signal peptide not normally associated with the catalytic domain and selected to target the α1,2-mannosidase activity to the ER or Golgi apparatus of the host cell where it can operate optimally. These host cells produce glycoproteins comprising a Man₅GlcNAc₂ glycoform. For example, U.S. Pat. No. 7,029,872 and U.S. Published Patent Application Nos. 2004/0018590 and 2005/0170452 disclose lower eukaryote host cells capable of producing a glycoprotein comprising a Man₅GlcNAc₂ glycoform.

In a further embodiment, the immediately preceding host cell further includes a GlcNAc transferase I (GnT I) catalytic domain fused to a cellular targeting signal peptide not normally associated with the catalytic domain and selected to target GlcNAc transferase I activity to the ER or Golgi apparatus of the host cell where it can operate optimally. These host cells produce glycoproteins comprising a GlcNAcMan₅GlcNAc₂ glycoform. U.S. Pat. No. 7,029,872 and U.S. Published Patent Application Nos. 2004/0018590 and 2005/0170452 disclose lower eukaryote host cells capable of producing a glycoprotein comprising a GlcNAcMan₅GlcNAc₂ glycoform.

In a further embodiment, the immediately preceding host cell further includes a mannosidase II catalytic domain fused to a cellular targeting signal peptide not normally associated with the catalytic domain and selected to target mannosidase II activity to the ER or Golgi apparatus of the host cell where it can operate optimally. These host cells produce glycoproteins comprising a GlcNAcMan₃GlcNAc₂ glycoform. U.S. Pat. No. 7,029,872 and U.S. Published Patent Application No. 2004/0230042 discloses lower eukaryote host cells that express mannosidase II enzymes and are capable of producing glycoproteins having predominantly a GlcNAc₂Man₃GlcNAc₂ glycoform.

In a further embodiment, the immediately preceding host cell further includes GlcNAc transferase II (GnT II) catalytic domain fused to a cellular targeting signal peptide not normally associated with the catalytic domain and selected to target GlcNAc transferase II activity to the ER or Golgi apparatus of the host cell where it can operate optimally. These host cells produce glycoproteins comprising a GlcNAc₂Man₃GlcNAc₂ glycoform. U.S. Pat. No. 7,029,872 and U.S. Published Patent Application Nos. 2004/0018590 and 2005/0170452 disclose lower eukaryote host cells capable of producing glycoproteins comprising a GlcNAc₂Man₃GlcNAc₂ glycoform.

In a further embodiment, the immediately preceding host cell further includes a galactosyltransferase catalytic domain fused to a cellular targeting signal peptide not normally associated with the catalytic domain and selected to target galactosyltransferase activity to the ER or Golgi apparatus of the host cell where it can operate optimally. These host cells produce glycoproteins comprising a GalGlcNAc₂Man₃GlcNAc₂ or Gal₂GlcNAc₂Man₃GlcNAc₂ glycoform, or mixture thereof. U.S. Pat. No. 7,029,872 and U.S. Published Patent Application No. 2006/0040353 discloses lower eukaryote host cells capable of producing glycoproteins comprising a Gal₂GlcNAc₂Man₃GlcNAc₂ glycoform.

In a further embodiment, the immediately preceding host cell further includes a sialyltransferase catalytic domain fused to a cellular targeting signal peptide not normally associated with the catalytic domain and selected to target sialytransferase activity to the ER or Golgi apparatus of the host cell. These host cells produce glycoproteins comprising predominantly a NANA₂Gal₂GlcNAc₂Man₃GlcNAc₂ glycoform or NANAGal₂GlcNAc₂Man₃GlcNAc₂ glycoform or mixture thereof. It is useful that the host cell further include a means for providing CMP-sialic acid for transfer to the N-glycan. U.S. Published Patent Application No. 2005/0260729 discloses a method for genetically engineering lower eukaryotes to have a CMP-sialic acid synthesis pathway and U.S. Published Patent Application No. 2006/0286637 discloses a method for genetically engineering lower eukaryotes to produce sialylated glycoproteins.

Any one of the preceding host cells can further include one or more GlcNAc transferase selected from the group consisting of GnT III, GnT IV, GnT V, GnT VI, and GnT IX to produce glycoproteins having bisected (GnT III) and/or multiantennary (GnT IV, V, VI, and IX) N-glycan structures such as disclosed in U.S. Published Patent Application Nos. 2004/074458 and 2007/0037248.

In further embodiments, the host cell that produces glycoproteins that have predominantly GlcNAcMan₅GlcNAc₂ N-glycans further includes a galactosyltransferase, catalytic domain fused to a cellular targeting signal peptide not normally associated with the catalytic domain and selected to target Galactosyltransferase activity to the ER or Golgi apparatus of the host cell. These host cells produce glycoproteins comprising predominantly the GalGlcNAcMan₅GlcNAc₂ glycoform.

In a further embodiment, the immediately preceding host cell that produced glycoproteins that have predominantly the GalGlcNAcMan₅GlcNAc₂ N-glycans further includes a sialyltransferase catalytic domain fused to a cellular targeting signal peptide not normally associated with the catalytic domain and selected to target sialytransferase activity to the ER or Golgi apparatus of the host cell. These host cells produce glycoproteins comprising a NANAGalGlcNAcMan₅GlcNAc₂ glycoform.

Various of the preceding host cells further include one or more sugar transporters such as UDP-GlcNAc transporters (for example, Kluyveromyces lactis and Mus musculus UDP-GlcNAc transporters), UDP-galactose transporters (for example, Drosophila melanogaster UDP-galactose transporter), and CMP-sialic acid transporter (for example, human sialic acid transporter). Because Pichia pastoris lacks the above transporters, it is preferable that the Pichia pastoris be genetically engineered to include the above transporters.

To reduce or eliminate detectable cross reactivity to antibodies against host cell protein, the recombinant glycoengineered Pichia pastoris host cells are genetically engineered to eliminate glycoproteins having α-mannosidase-resistant N-glycans by deleting or disrupting one or more of the β-mannosyltransferase genes (e.g., BMT1, BMT2, BMT3, and BMT4) (See, U.S. Published Patent Application No. 2006/0211085) and glycoproteins having phosphomannose residues by deleting or disrupting one or both of the phosphomannosyl transferase genes PNO1 and MNN4B (See for example, U.S. Pat. Nos. 7,198,921 and 7,259,007), which in further aspects can also include deleting or disrupting the MNN4A gene. Disruption includes disrupting the open reading frame encoding the particular enzymes or disrupting expression of the open reading frame or abrogating translation of RNAs encoding one or more of the β-mannosyltransferases and/or phosphomannosyltransferases using interfering RNA, antisense RNA, or the like. The host cells can further include any one of the aforementioned host cells modified to produce particular N-glycan structures.

Regulatory sequences which may be used in the practice of the methods disclosed herein include signal sequences, promoters, and transcription terminator sequences. Examples of promoters include promoters from numerous species, including but not limited to alcohol-regulated promoter, tetracycline-regulated promoters, steroid-regulated promoters (e.g., glucocorticoid, estrogen, ecdysone, retinoid, thyroid), metal-regulated promoters, pathogen-regulated promoters, temperature-regulated promoters, and light-regulated promoters. Specific examples of regulatable promoter systems well known in the art include but are not limited to metal-inducible promoter systems (e.g., the yeast copper-metallothionein promoter), plant herbicide safner-activated promoter systems, plant heat-inducible promoter systems, plant and mammalian steroid-inducible promoter systems, Cym repressor-promoter system (Krackeler Scientific, Inc. Albany, N.Y.), RheoSwitch System (New England Biolabs, Beverly Mass.), benzoate-inducible promoter systems (See WO2004/043885), and retroviral-inducible promoter systems. Other specific regulatable promoter systems well-known in the art include the tetracycline-regulatable systems (See for example, Berens & Hillen, Eur J Biochem 270: 3109-3121 (2003)), RU 486-inducible systems, ecdysone-inducible systems, and kanamycin-regulatable system. Lower eukaryote-specific promoters include but are not limited to the Saccharomyces cerevisiae TEF-1 promoter, Pichia pastoris GAPDH promoter, Pichia pastoris GUT1 promoter, PMA-1 promoter, Pichia pastoris PCK-1 promoter, and Pichia pastoris AOX-1 and AOX-2 promoters.

Examples of transcription terminator sequences include transcription terminators from numerous species and proteins, including but not limited to the Saccharomyces cerevisiae cytochrome C terminator; and Pichia pastoris ALG3 and PMA1 terminators.

Yeast selectable markers include drug resistance markers and genetic functions which allow the yeast host cell to synthesize essential cellular nutrients, e.g. amino acids. Drug resistance markers which are commonly used in yeast include chloramphenicol, kanamycin, methotrexate, G418 (geneticin), Zeocin, and the like. Genetic functions which allow the yeast host cell to synthesize essential cellular nutrients are used with available yeast strains having auxotrophic mutations in the corresponding genomic function. Common yeast selectable markers provide genetic functions for synthesizing leucine (LEU2), tryptophan (TRP1 and TRP2), proline (PRO1), uracil (URA3, URA5, URA6), histidine (HIS3), lysine (LYS2), adenine (ADE1 or ADE2), and the like. Other yeast selectable markers include the ARR3 gene from S. cerevisiae, which confers arsenite resistance to yeast cells that are grown in the presence of arsenite (Bobrowicz et al., Yeast, 13:819-828 (1997); Wysocki et al., J. Biol. Chem. 272:30061-30066 (1997)).

A number of suitable integration sites include those enumerated in U.S. Published application No. 2007/0072262 and include homologs to loci known for Saccharomyces cerevisiae and other yeast or fungi. Methods for integrating vectors into yeast are well known, for example, See U.S. Pat. No. 7,479,389, PCT Published Application No. WO2007136865, and PCT/US2008/13719. Examples of insertion sites include, but are not limited to, Pichia ADE genes; Pichia TRP (including TRP1 through TRP2) genes; Pichia MCA genes; Pichia CYM genes; Pichia PEP genes; Pichia PRB genes; and Pichia LEU genes. The Pichia ADE1 and ARG4 genes have been described in Lin Cereghino et al., Gene 263:159-169 (2001) and U.S. Pat. No. 4,818,700, the HIS3 and TRP1 genes have been described in Cosano et al., Yeast 14:861-867 (1998), HIS4 has been described in GenBank Accession No. X56180.

It is well known that the properties of certain proteins can be modulated by attachment of polyethylene glycol (PEG) polymers, which increases the hydrodynamic volume of the protein and thereby slows its clearance by kidney filtration. (See, for example, Clark et al., J. Biol. Chem. 271: 21969-21977 (1996)). Therefore, it is envisioned that the core peptide residues can be PEGylated to provide enhanced therapeutic benefits such as, for example, increased efficacy by extending half-life in vivo. Thus, PEGylating the rHuGCSFs will improve the pharmacokinetics and pharmacodynamics of the rHuGCSFs.

Therefore, in further still embodiments, the rHuGCSFs are modified by PEGylation, cholesterylation, or palmitoylation. The modification can be to any amino acid residue in the rHuGCSF, however, in current envisioned embodiments, the modification is to the N-terminal amino acid of the rHuGCSF, either directly to the N-terminal amino acid or by way coupling to the thiol group of a cysteine residue added to the N-terminus or a linker added to the N-terminus such as Ttds.

As used herein the general term “polyethylene glycol chain” or “PEG chain”, refers to mixtures of condensation polymers of ethylene oxide and water, in a branched or straight chain, represented by the general formula H(OCH₂CH₂)ₙOH, wherein n is at least 9. Absent any further characterization, the term is intended to include polymers of ethylene glycol with an average total molecular weight selected from the range of 500 to 40,000 Daltons: “polyethylene glycol chain” or “PEG chain” is used in combination with a numeric suffix to indicate the approximate average molecular weight thereof. For example, PEG-5,000 refers to polyethylene glycol chain having a total molecular weight average of about 5,000.

As used herein the term “PEGylated” and like terms refers to a compound that has been modified from its native state by linking a polyethylene glycol chain to the compound. A “PEGylated rHuGCSF peptide” is a rHuGCSF that has a PEG chain covalently bound thereto.

Peptide PEGylation methods are well known in the literature and described in the following references, each of which is incorporated herein by reference: Lu et al., Int. J. Pept. Protein Res. 43: 127-38 (1994); Lu et al., Pept. Res. 6: 140-6 (1993); Felix et J. Pept. Protein Res. 46: 253-64 (1995); Gaertner et al., Bioconjug. Chem. 7: 38-44 (1996); Tsutsumi et al., Thromb. Haemost. 77: 168-73 (1997); Francis et al., Int. J. Hematol. 68: 1-18 (1998); Roberts et al., J. Pharm. Sci. 87: 1440-45 (1998); and Tan et al., Protein Expr. Purif. 12: 45-52 (1998). Polyethylene glycol or PEG is meant to encompass any of the forms of PEG that have been used to derivatize other proteins, including, but not limited to, mono-(C₁₋₁₀) alkoxy or aryloxy-polyethylene glycol. Suitable PEG moieties include, for example, 40 kDa methoxy poly(ethylene glycol) propionaldehyde (Dow, Midland, Mich.); 60 kDa methoxy poly(ethylene glycol) propionaldehyde (Dow, Midland, Mich.); 40 kDa methoxy poly(ethylene glycol) maleimido-propionamide (Dow, Midland, Mich.); 31 kDa alpha-methyl-w-(3-oxopropoxy), polyoxyethylene (NOF Corporation, Tokyo); mPEG₂-NHS-40k (Nektar); mPEG₂-MAL-40k (Nektar), SUNBRIGHT GL2-400MA ((PEG)₂40 kDa) (NOF Corporation, Tokyo), SUNBRIGHT ME-200MA (PEG20 kDa) (NOF Corporation, Tokyo). The PEG groups are generally attached to the rHuGCSFs via acylation or alkylation through a reactive group on the PEG moiety (for example, a maleimide, an aldehyde, amino, thiol, or ester group) to a reactive group on the rHuGCSF (for example, an aldehyde, amino, thiol, a maleimide, or ester group).

The PEG molecule(s) may be covalently attached to any Lys, Cys, or K(CO(CH₂)₂SH) residues at any position in the rHuGCSF. The rHuGCSFs described herein can be PEGylated directly to any amino acid at the N-terminus by way of the N-terminal amino group. A “linker arm” may be added to the rHuGCSF to facilitate PEGylation. PEGylation at the thiol side-chain of cysteine has been widely reported (See, e.g., Caliceti & Veronese, Adv. Drug Deliv. Rev. 55: 1261-77 (2003)). If there is no cysteine residue in the peptide, a cysteine residue can be introduced through substitution or by adding a cysteine to the N-terminal amino acid. Those rHuGCSFs, which have been PEGylated, have been PEGylated through the side chains of a cysteine residue added to the N-terminal amino acid.

In some aspects, the PEG molecule(s) may be covalently attached to an amide group in the C-terminus of the rHuGCSF. In general, there is at least one PEG molecule covalently attached to the rHuGCSF. In particular aspects, the PEG molecule is branched while in other aspects, the PEG molecule may be linear. In particular aspects, the PEG molecule is between 1 kDa and 100 kDa in molecular weight. In further aspects, the PEG molecule is selected from 10, 20, 30, 40, 50, 60, and 80 kDa. In further still aspects, it is selected from 20, 40, or 60 kDa. Where there are two PEG molecules covalently attached to the rHuGCSF of the present invention, each is 1 to 40 kDa and in particular aspects, they have molecular weights of 20 and 20 kDa, 10 and 30 kDa, 30 and 30 kDa, 20 and 40 kDa, or 40 and 40 kDa. In particular aspects, the rHuGCSFs contain mPEG-cysteine. The mPEG in mPEG-cysteine can have various molecular weights. The range of the molecular weight is preferably 5 kDa to 200 kDa, more preferably 5 kDa to 100 kDa, and further preferably 20 kDa to 60 kD. The mPEG can be linear or branched.

Currently, it is preferable that the rHuGCSFs are PEGylated through the side chains of a cysteine added to the N-terminal amino acid. Currently, the agonists preferably contain mPEG-cysteine. The mPEG in mPEG-cysteine can have various molecular weights. The range of the molecular weight is preferably 5 kDa to 200 kDa, more preferably 5 kDa to 100 kDa, and further preferably 20 kDa to 60 kDA. The mPEG can be linear or branched.

A useful strategy for the PEGylation of synthetic rHuGCSFs consists of combining, through forming a conjugate linkage in solution, a peptide, and a PEG moiety, each bearing a special functionality that is mutually reactive toward the other. The rHuGCSFs can be easily prepared with conventional solid phase synthesis. The rHuGCSF is “preactivated” with an appropriate functional group at a specific site. The precursors are purified and fully characterized prior to reacting with the PEG moiety. Conjugation of the peptide with PEG usually takes place in aqueous phase and can be easily monitored by reverse phase analytical HPLC. The PEGylated rHuGCSF can be easily purified by cation exchange chromatography or preparative HPLC and characterized by analytical HPLC, amino acid analysis and laser desorption mass spectrometry.

The rHuGCSF can comprise other non-sequence modifications, for example, glycosylation, lipidation, acetylation, phosphorylation, carboxylation, methylation, or any other manipulation or modification, such as conjugation with a labeling component. While, in particular aspects, the rHuGCSF herein utilize naturally-occurring amino acids or D isoforms of naturally occurring amino acids, substitutions with non-naturally occurring amino acids (for example., methionine sulfoxide, methionine methylsulfonium, norleucine, epsilon-aminocaproic acid, 4-aminobutanoic acid, tetrahydroisoquinoline-3-carboxylic acid, 8-aminocaprylic acid, 4 aminobutyric acid, Lys(N(epsilon)-trifluoroacetyl) or synthetic analogs, for example, o-aminoisobutyric acid, p or y-amino acids, and cyclic analogs. In further still aspects, the rHuGCSFs comprise a fusion protein that having a first moiety, which is a rHuGCSF, and a second moiety, which is a heterologous peptide.

### Pharmaceutical Compositions

The rHuGCSF disclosed herein may be used in a pharmaceutical composition when combined with a pharmaceutically acceptable carrier. Such compositions comprise a therapeutically-effective amount of the rHuGCSF and a pharmaceutically acceptable carrier. Such a composition may also be comprised of (in addition to rHuGCSF and a carrier) diluents, fillers, salts, buffers, stabilizers, solubilizers, and other materials well known in the art. Compositions comprising the rHuGCSF can be administered, if desired, in the form of salts provided the salts are pharmaceutically acceptable. Salts may be prepared using standard procedures known to those skilled in the art of synthetic organic chemistry.

The term “pharmaceutically acceptable salts” refers to salts prepared from pharmaceutically acceptable non-toxic bases or acids including inorganic or organic bases and inorganic or organic acids. Salts derived from inorganic bases include aluminum, ammonium, calcium, copper, ferric, ferrous, lithium, magnesium, manganic salts, manganous, potassium, sodium, zinc, and the like. Particularly preferred are the ammonium, calcium, magnesium, potassium, and sodium salts. Salts derived from pharmaceutically acceptable organic non-toxic bases include salts of primary, secondary, and tertiary amines, substituted amines including naturally occurring substituted amines, cyclic amines, and basic ion exchange resins, such as arginine, betaine, caffeine, choline, N,N′-dibenzylethylenediamine, diethylamine, 2-diethylaminoethanol, 2-dimethylaminoethanol, ethanolamine, ethylenediamine, N-ethyl-morpholine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylamine, trimethylamine, tripropylamine, tromethamine, and the like. The term “pharmaceutically acceptable salt” further includes all acceptable salts such as acetate, lactobionate, benzenesulfonate, laurate, benzoate, malate, bicarbonate, maleate, bisulfate, mandelate, bitartrate, mesylate, borate, methylbromide, bromide, methylnitrate, calcium edetate, methylsulfate, camsylate, mucate, carbonate, napsylate, chloride, nitrate, clavulanate, N-methylglucamine, citrate, ammonium salt, dihydrochloride, oleate, edetate, oxalate, edisylate, pamoate (embonate), estolate, palmitate, esylate, pantothenate, fumarate, phosphate/diphosphate, gluceptate, polygalacturonate, gluconate, salicylate, glutamate, stearate, glycollylarsanilate, sulfate, hexylresorcinate, subacetate, hydrabamine, succinate, hydrobromide, tannate, hydrochloride, tartrate, hydroxynaphthoate, teoclate, iodide, tosylate, isethionate, triethiodide, lactate, panoate, valerate, and the like which can be used as a dosage form for modifying the solubility or hydrolysis characteristics or can be used in sustained release or pro-drug formulations. It will be understood that, as used herein, references to the rHuGCSF disclosed herein are meant to also include the pharmaceutically acceptable salts.

As utilized herein, the term “pharmaceutically acceptable” means a non-toxic material that does not interfere with the effectiveness of the biological activity of the active ingredient(s), approved by a regulatory agency of the Federal or a state government or listed in the U.S. Pharmacopoeia or other generally recognized pharmacopoeia for use in animals and, more particularly, in humans. The term “carrier” refers to a diluent, adjuvant, excipient, or vehicle with which the therapeutic is administered and includes, but is not limited to such sterile liquids as water and oils. The characteristics of the carrier will depend on the route of administration. The rHuGCSF disclosed herein may be in multimers (for example, heterodimers or homodimers) or complexes with itself or other peptides. As a result, pharmaceutical compositions of the invention may comprise one or more rHuGCSF molecules disclosed herein in such multimeric or complexed form.

As used herein, the term “therapeutically effective amount” means the total amount of each active component of the pharmaceutical composition or method that is sufficient to show a meaningful patient benefit, i.e., treatment, healing, prevention or amelioration of the relevant medical condition, or an increase in rate of treatment, healing, prevention or amelioration of such conditions. When applied to an individual active ingredient, administered alone, the term refers to that ingredient alone. When applied to a combination, the term refers to combined amounts of the active ingredients that result in the therapeutic effect, whether administered in combination, serially, or simultaneously.

The following examples are intended to promote a further understanding of the present invention.

### Example 1

This Example illustrates the construction of a recombinant Pichia pastoris that can produce the rHuGCSF of the present invention.

Strains and Media. E. coli strain TOP10 was used for recombinant DNA work. All primers, sequences, and selected Pichia pastoris strains used are listed in Tables 1, 3, and Table of Sequences.

TABLE 1
List of Primer Sequences

| SEQ ID | Primer | |
|---|---|---|
| NO. | Name | Sequence |
| 1 | MAM281 | ctcgaggagtcctcttATGacaccattagga |
| | | cctgcttcctcc |
| 2 | MAM227 | Ctcgaggagtcctctt acaccattaggacctgcttc |
| 3 | MAM228 | gagctcggccggccttattatggttgagcc |
| 4 | MAM304 | aaaaaagaattccgaaaaatgagcaccctgacattgc |
| 5 | MAM305 | aaaaaaaggcctcttaaccaaagaacctccacctt |
| | | cgtccgtacgagcacagccggtgatagaagtg |

Protein expression was carried out with buffered glycerol-complex medium (BMGY) consisting of 1% yeast extract, 2% peptone, 100 mM potassium phosphate buffer, pH 6.0, 1.34% yeast nitrogen base, 4×10-5% biotin, and 1% glycerol as a growth medium; and buffered methanol-complex medium (BMMY) consisting of 1% methanol instead of glycerol in BMGY as an induction medium. YMD is 1% yeast extract, 2% peptone, 2% dextrose and 2% agar. Restriction and modification enzymes were from New England BioLabs (Beverly, Mass.). Oligonucleotides were obtained from Integrated DNA Technologies (Coralville, Iowa). Salts and buffering agents were from Sigma (St. Louis, Mo.).

Transformation of Yeast Strains. Yeast transformations with expression/integration vectors were as follows. Pichia pastoris strains were grown in 50 mL YMD media (yeast extract (1%), martone (2%), dextrose (2%)) overnight to an OD of between about 0.2 to 6. After incubation on ice for 30 minutes, cells were pelleted by centrifugation at 2500-3000 rpm for 5 minutes. Media was removed and the cells washed three times with ice cold sterile 1M sorbitol before re-suspension in 0.5 ml ice cold sterile 1M sorbitol. Ten μL linearized DNA (1-10 μg) and 100 μL cell suspension were combined in an electroporation cuvette and incubated for 5 minutes on ice. Electroporation was in a Bio-Rad GenePulser Xcell following the preset Pichia pastoris protocol (2 kV, 25 μF, 200Ω), immediately followed by the addition of 1 mL YMDS recovery media (YMD media plus 1 M sorbitol). The transformed cells were allowed to recover for four hours to overnight at room temperature (26° C.) before plating the cells on selective media.

Construction of a GCSF expression plasmidS. DNA (SEQ ID NO:7) encoding the mature Homo sapiens granulocyte-cytokine stimulatory factor protein (SEQ ID NO:8) was synthesized by DNA2.0 (Menlo Park, Calif.) and inserted into a pUC19 family plasmid to make plasmid pGLY4316. The precursor human GCSF, GenBank NP_{—}757373, has the amino acid sequence shown in SEQ ID NO:6.

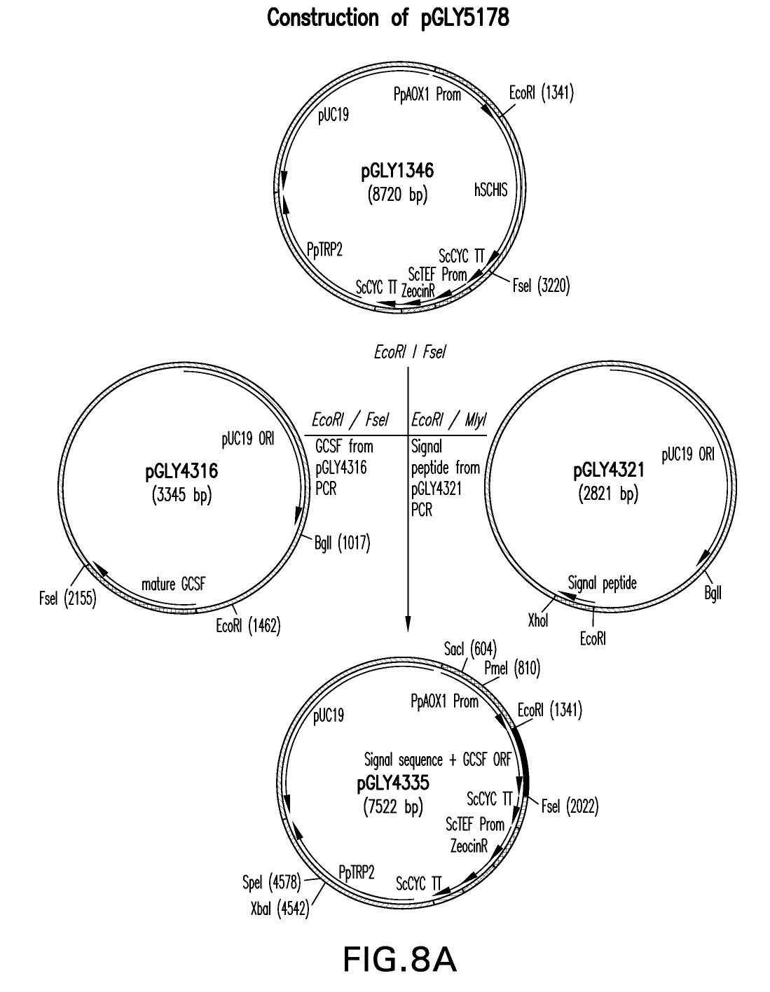

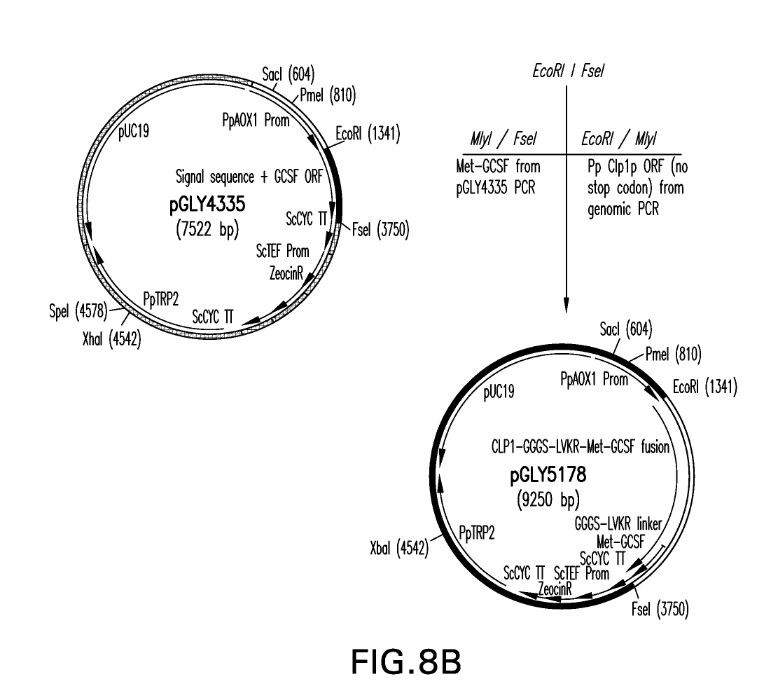

A subsequent plasmid was constructed that contained the DNA encoding the mature GCSF PCR amplified from pGLY4316 with PCR primers MAM227 (SEQ ID NO:2) and MAM228 (SEQ ID NO:3). PCR primer MAM227 introduced XhoI and MlyI sites at the 5′ end of DNA encoding the mature GCSF and an FseI site at the 3′ end of the DNA encoding the mature GCSF. A DNA fragment encoding a mating factor-IL1β signal peptide (Han et al., Biochem. Biophys. Res. Commun. 18; 337(2):557-62. (2005); Lee et al., Biotechnol Prog. 15(5):884-90 (1999)) that directs the GCSF to the secretory pathway was removed from plasmid pGLY4321 with EcoRI and MlyI digestion. The PCR amplified product was digested with FseI and MlyI and was triple-ligated with the signal peptide encoding fragment into plasmid pGLY1346 digested with EcoRI and FseI to make plasmid pGLY4335 in which the 5′ end of the open reading frame (ORE) encoding the mature GCSF is ligated in frame with the 3′ end of the ORF encoding the signal peptide and which produces a fusion protein in which the N-terminus of the mature GCSF is fused to the C-terminus of the signal peptide. Plasmid pGLY4335 is shown in FIG. 8A.

DNA encoding the mature GCSF was PCR amplified from plasmid pGLY4335 by PCR using PCR primers MAM281 (SEQ ID NO:1) and MAM228 (SEQ ID NO:3). The PCR amplified product (encodes GCSF without the signal peptide) was digested with the MlyI and FseI restriction enzymes. Primer MAM281 contains an ATG codon in frame with the GCSF ORF. Thus, the resulting digested amplified PCR product contains an in-frame addition of the ATG translation start codon to the 5′ end of the open reading frame (ORF) encoding the mature GCSF. The PCR amplified product encodes a recombinant human GCSF with an N-terminal Met (rHuMetGCSF). The amino acid sequence of rHuMetGCSF is shown in SEQ ID NO:14. Thus, the amplified PCR product encodes the mature GCSF with an N-terminal methionine residue, which is identical to the amino acid sequence of filgrastim.

The P. pastoris CLP1 gene was PCR amplified from Pichia pastoris strain NRRL-Y11430 chromosomal DNA using PCR primers MAM304 (SEQ ID NO:4) and MAM305 (SEQ ID NO:5) and the amplified PCR product (PpClp1) was digested with EcoRI and StuI. PCR primer MAM305 was designed to encode the peptide linker GGGSLVKR (SEQ ID NO:15; encoded by SEQ ID NO:16) in-frame between the ORE encoding the Clp1p protein and the ORE encoding the rHuMetGCSF. A three piece ligation reaction was performed with the EcoRI/StuI digested fragment encoding the P. pastoris CLP1, the MlyI/FseI digested fragment encoding the rHuMetGCSF, and plasmid pGLY1346 (digested with EcoRI and FseI) to generate plasmid pGLY5178 as shown in FIG. 8B. The Zeocin^{R} expression cassette comprises a nucleic acid molecule encoding the Sh ble ORF (SEQ ID NO:59) operably linked at the 5′ end to the S. cerevisiae TEF1 promoter (SEQ ID NO:58) and at the 3′ end to the S. cerevisiae CYC termination sequence (SEQ ID NO:57). The vector targets the TRP2 locus (SEQ ID NO:40) or the AOX1 promoter for integration. When the AOX1 promoter locus is selected, the plasmid is linearized at the PmeI site and the vector integrates into the locus by single-crossover homologous recombination with antibiotic selection. The insert DNA was sequenced to verify fidelity.

The complete ORF of pGLY5178 is transcriptionally regulated by the AOX1 (alcohol oxidase) promoter and encodes Clp1p-rHuMetGCSF fusion protein (SEQ ID NO:12 encoded by SEQ ID NO:11) comprising starting from the N-terminus, the complete P. pastoris Clp1p protein (SEQ ID NO:9) followed by the linker peptide GGGSLVKR (SEQ ID NO:15) and the ORF encoding rHuMetGCSF protein sequence (SEQ ID NO:14). Upon methanol induction of DNA transcription and translation of the DNA encoding the Clp1p-rHuMetGCSF fusion protein in Pichia pastoris, the Clp1p-rHuMetGCSF fusion protein enters the endoplasmic reticulum due to the Clp1p signal peptide. During transport through the Golgi apparatus, the fusion protein is further processed in the Golgi apparatus by the Kex2p protease, which cleaves after the arginine residue in the linker sequence. This produces two proteins: a Clp1 protein with linker at C-terminus (SEQ ID NO:13) and a rHuMetGCSF (SEQ ID NO:14), both which are subsequently found in the supernatant fraction (See U.S. Pub. Patent Application No. 2006/0252096).

Plasmids pGLY4335 and pGLY4354 were similar to pGLY5178 except that the Clp1p-rHuMetGCSF expression cassette was replaced with an expression cassette encoding rHGCSF fused to the S. cerevisiae mating factor pre-pro signal peptide (encoded by SEQ ID NO:26) or the HSA signal peptide (encoded by SEQ ID NO:28), respectively.

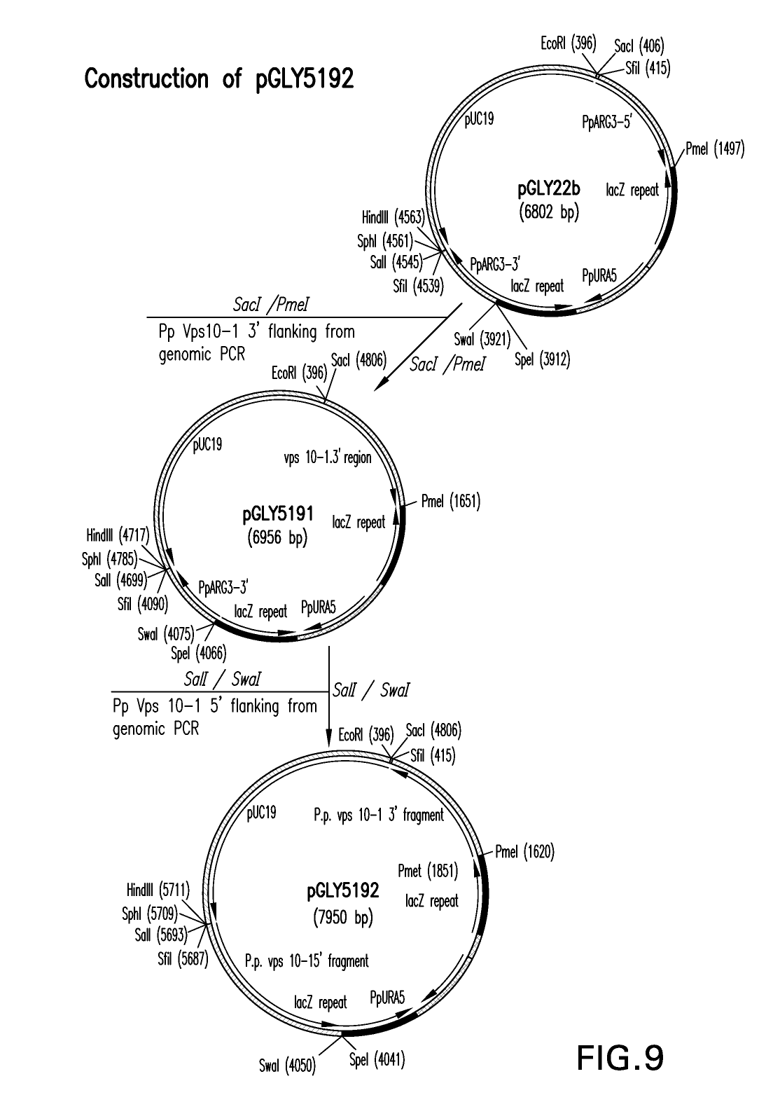

Generation of VPS10-1, PEP4, and PRIM deletion plasmids. The plasmid pGLY5192 was constructed to delete the ORF of the VPS10-1 gene (SEQ ID NO:17) and create a yeast strain deficient in vacuolar sorting receptor (Vps10-1p) activity. To generate the vps10-1 knock-out plasmid pGLY5192, the upstream 5′ flanking region of the VPS10-1 was first amplified using routine PCR conditions and Pichia pastoris strain NRRL-Y11430 genomic DNA as the template. The resulting PCR amplified product was cloned into plasmid pGLY22b digested with SacI and PmeI to generate plasmid pGLY5191. The downstream 3′ flanking region the VPS10-1 was amplified using routine PCR conditions and Pichia pastoris NRRL-Y11430 genomic DNA as the template. The resulting PCR amplified product was cloned into plasmid pGLY5191 digested with SalI and SwaI to generate plasmid pGLY5192. Both the upstream 5′ and the downstream 3′ cloned PCR amplified products of pGLY5192 were sequenced to verify fidelity. The construction of pGLY5192 is shown in FIG. 9.

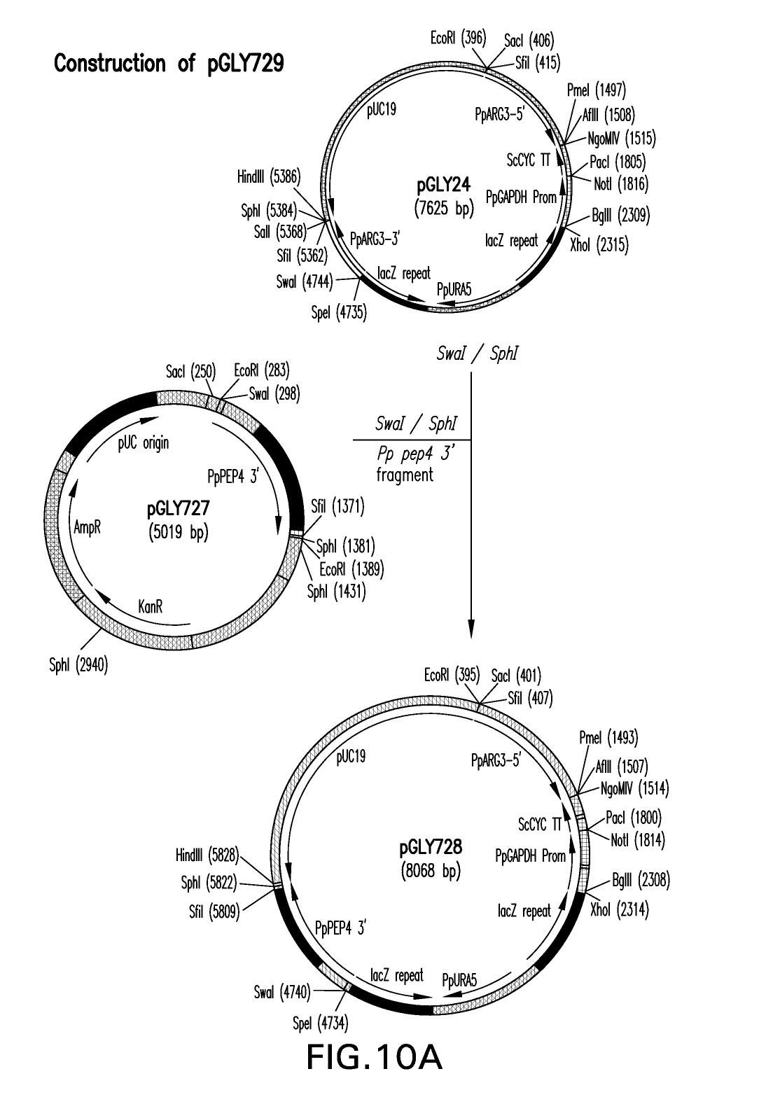

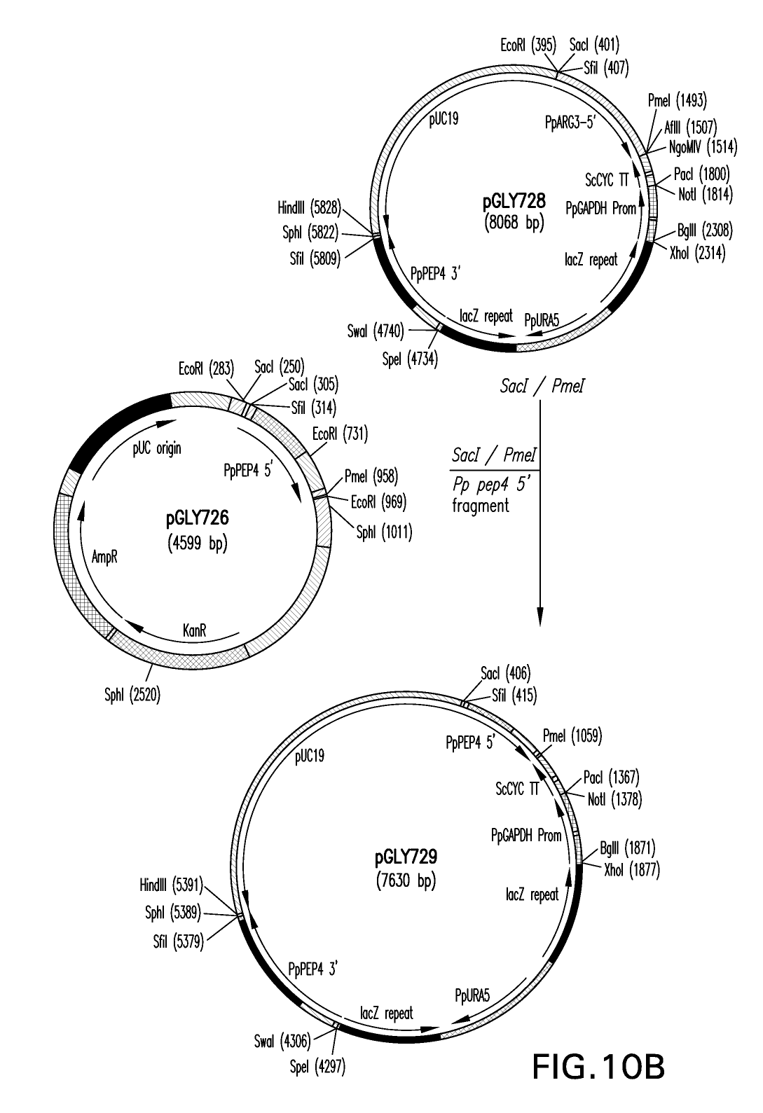

The plasmid pGLY729 was constructed to delete the open reading frame (ORF) of the PEP4 gene (SEQ ID NO:18) and create a yeast strain deficient in vacuolar endoproteinase Proteinase A (PrA) activity. To generate pGLY729, the downstream 3′ flanking region was first PCR amplified using routine PCR conditions and Pichia pastoris strain NRRL-Y11430 genomic DNA as the template. The resulting PCR amplified product was cloned into plasmid pCR2.1 (Invitrogen® Cat# K450040) to generate pGLY727. The PEP4 downstream 3′ flanking region was then isolated from plasmid pGLY727 using restriction enzymes SwaI and SphI and the DNA fragment cloned into plasmid pGLY24 digested with SwaI and SphI to generate plasmid pGLY728. The upstream 5′ flanking region was PCR amplified using routine PCR conditions and Pichia pastoris strain NRRL-Y11430 genomic DNA as the template. The resulting PCR amplified product was cloned into plasmid pCR2.1 to generate plasmid pGLY726. The PEP4 upstream 5′ flanking region was then isolated from plasmid pGLY726 using restriction enzymes SacI and PmeI and cloned into pGLY728 digested with SacI and PmeI to generate pGLY729. Both upstream 5′ and downstream 3′ fragments of pGLY729 were sequenced to verify fidelity. The construction of pGLY729 is shown in FIG. 10A-B.

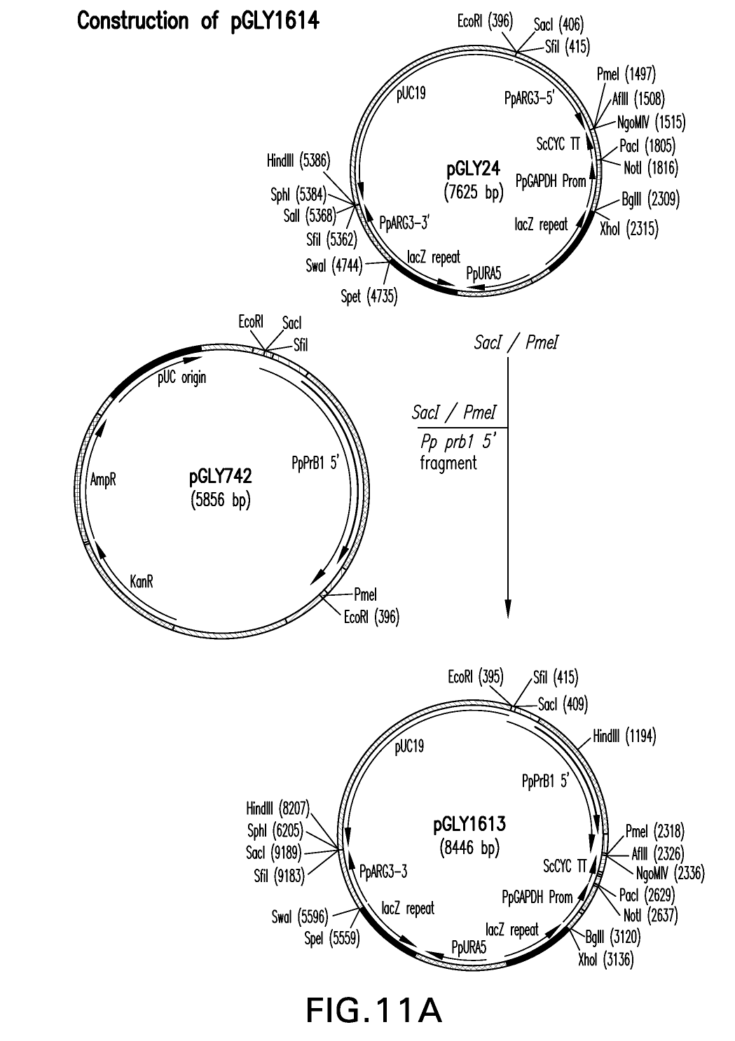

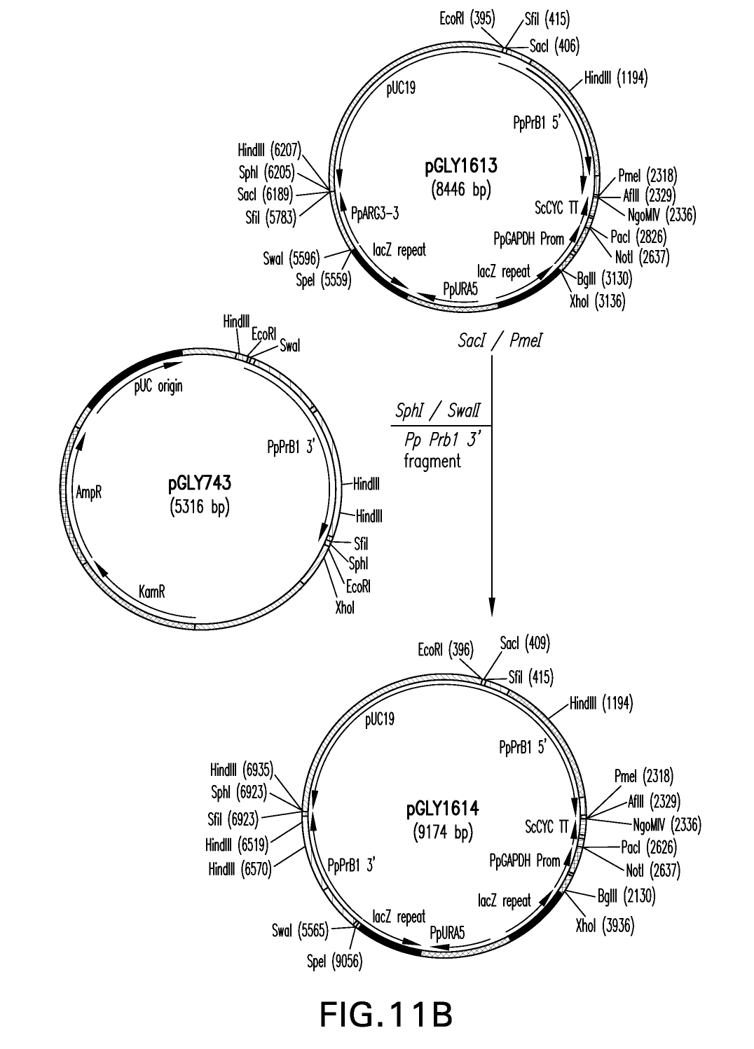

The plasmid pGLY1614 was constructed to delete the ORF of the PRB1 gene (SEQ ID NO:19) and create a yeast strain deficient in vacuolar endoproteinase Proteinase B (PrB) activity. To generate plasmid pGLY1614, the upstream 5′ flanking region was first amplified using routine PCR conditions and Pichia pastoris strain NRRL-Y11430 genomic DNA as the template. The resulting PCR amplified product was cloned into plasmid pCR2.1 to generate plasmid pGLY742. The PRB1 upstream 5′ flanking region was then isolated from plasmid pGLY742 using restriction enzymes SacI and PmeI and cloned into plasmid pGLY24 digested with SacI and PmeI to generate plasmid pGLY1613. The downstream 3′ flanking region was amplified using routine PCR conditions and Pichia pastoris strain NRRL-Y11430 genomic DNA as the template. The resulting PCR amplified product was cloned into plasmid pCR2.1 to generate plasmid pGLY743. The PRB1 downstream 3′ flanking region was then isolated from plasmid pGLY743 using restriction enzymes SphI and SwaI and cloned into plasmid pGLY1613 digested with SphI and SwaI to generate plasmid pGLY1614. Both the upstream 5′ and downstream 3′ fragments in pGLY1614 were sequenced to verify fidelity. The construction of pGLY1614 is shown in FIG. 11A-B.

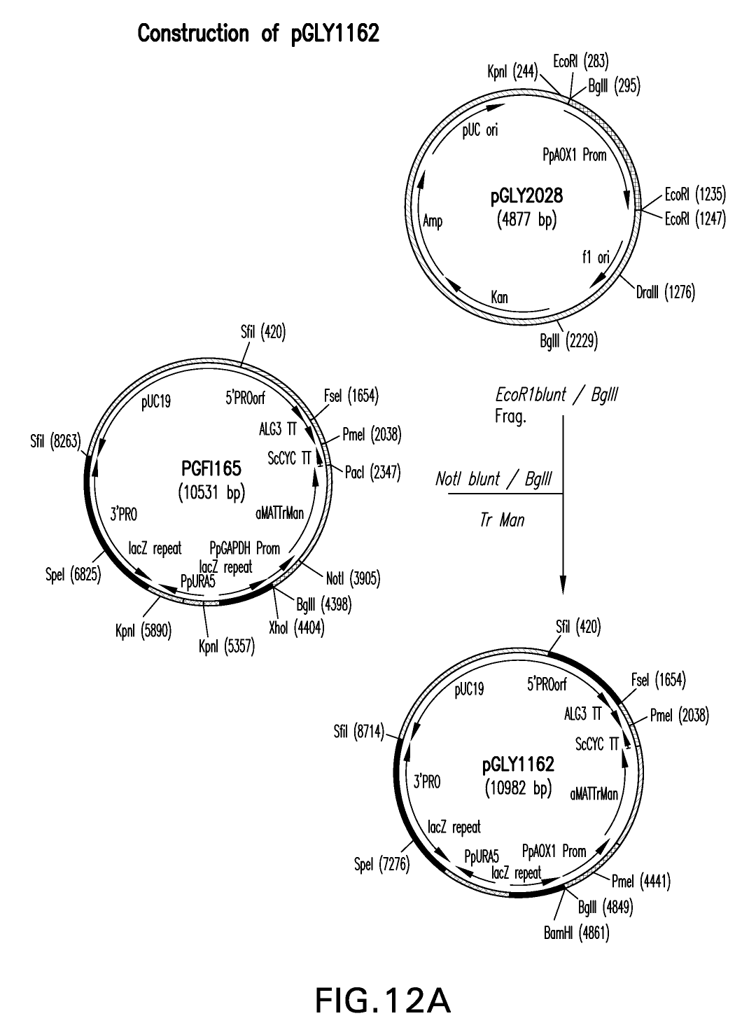

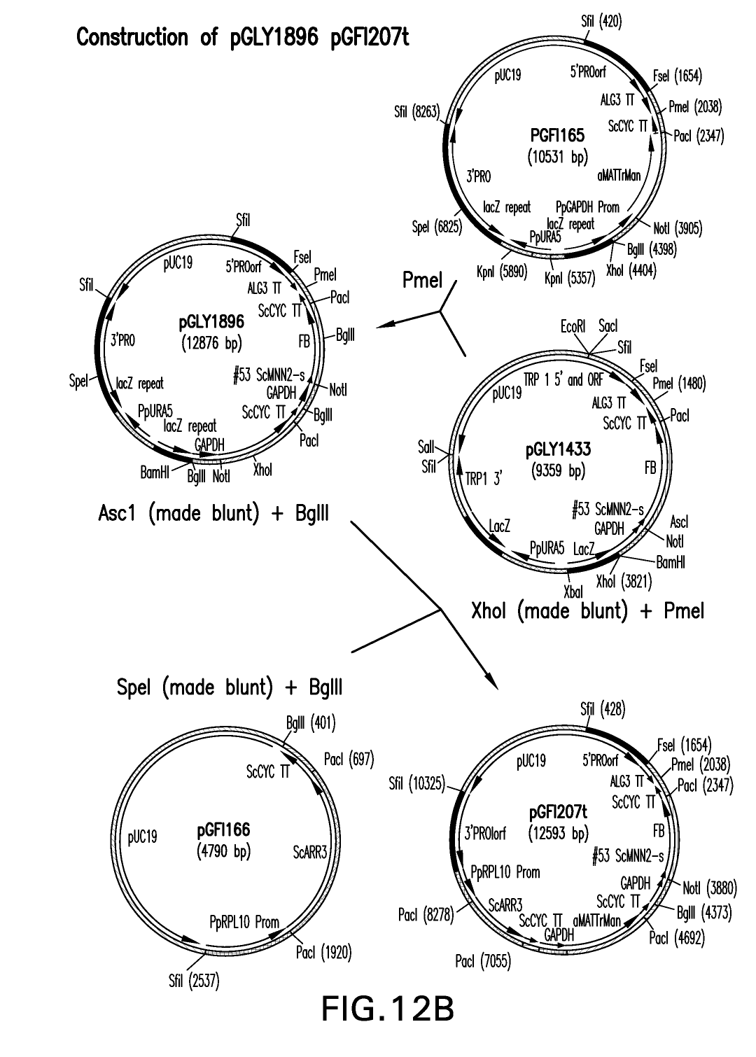

Generation of O-glycan modification plasmids. Construction of plasmids pGLY1162, pGLY1896, and pGFI204t was as follows. All Trichoderma reesei α-1,2-mannosidase expression plasmid vectors were derived from plasmids pGFI165, which encodes the T. reesei α-1,2-mannosidase catalytic domain (SEQ ID NO:34; Published International Application No. WO2007061631) fused to S. cerevisiae αMATpre signal peptide (SEQ ID NO:25) wherein expression is under the control of the Pichia pastoris GAPDH promoter (referred to as TrMDSI). Integration of the plasmid vector is targeted to the Pichia pastoris PRO1 locus and selection is achieved using the Pichia pastoris URA5 gene. A map of plasmid vector pGFI165 is shown in FIGS. 12A and 12B. Construction of these plasmids is also disclosed in PCT/US2009/33507).

Plasmid vector pGLY1896 is a KINKO vector that contains an expression cassette comprising a nucleic acid molecule (SEQ ID NO:63) encoding the mouse α-1,2-mannosidase catalytic domain (FB) fused to the S. cerevisiae MNN2 membrane insertion leader peptide (53; encoded by SEQ ID NO:64) (See Choi et al., Proc. Natl. Acad. Sci. USA 100: 5022 (2003)) inserted into plasmid vector pGFI165. This was accomplished by isolating the GAPDH promoter-ScMNN2-mouse MNSI expression cassette from pGLY1433 digested with XhoI (and the ends made blunt) and PmeI, and inserting the fragment into pGFI165 that digested with PmeI. The two expression cassettes are flanked on one side by a nucleic acid molecule comprising a nucleotide sequence from the 5′ region and complete open reading frame (ORF) of the PRO1 gene (SEQ ID NO:61) followed by a P. pastoris ALG3 termination sequence (SEQ ID NO:55) and on the other side by a nucleic acid molecule comprising a nucleotide sequence from the 3′ region of the PRO1 gene (SEQ ID NO:62). KINKO (Knock-In with little or No Knock-Out) integration vectors enable insertion of heterologous DNA into a targeted locus without disrupting expression of the gene at the targeted locus and have been described in U.S. Published Application No. 20090124000. A map of plasmid vector pGLY1896 is shown in FIG. 12B.

Plasmid vector pGLY1162 was made by replacing the GAPDH promoter in pGFI165 with the Pichia pastoris AOX1 (PpAOX1) promoter (SEQ ID NO:56). This was accomplished by isolating the PpAOX1 promoter as an EcoRI (made blunt)-BglII fragment from pGLY2028, and inserting into pGFI165 that was digested with Nod (ends made blunt) and BglII. Integration of the plasmid vector is to the Pichia pastoris PRO1 locus and selection is using the Pichia pastoris URA5 gene. A map of plasmid vector pGLY1162 is shown in FIG. 12A.

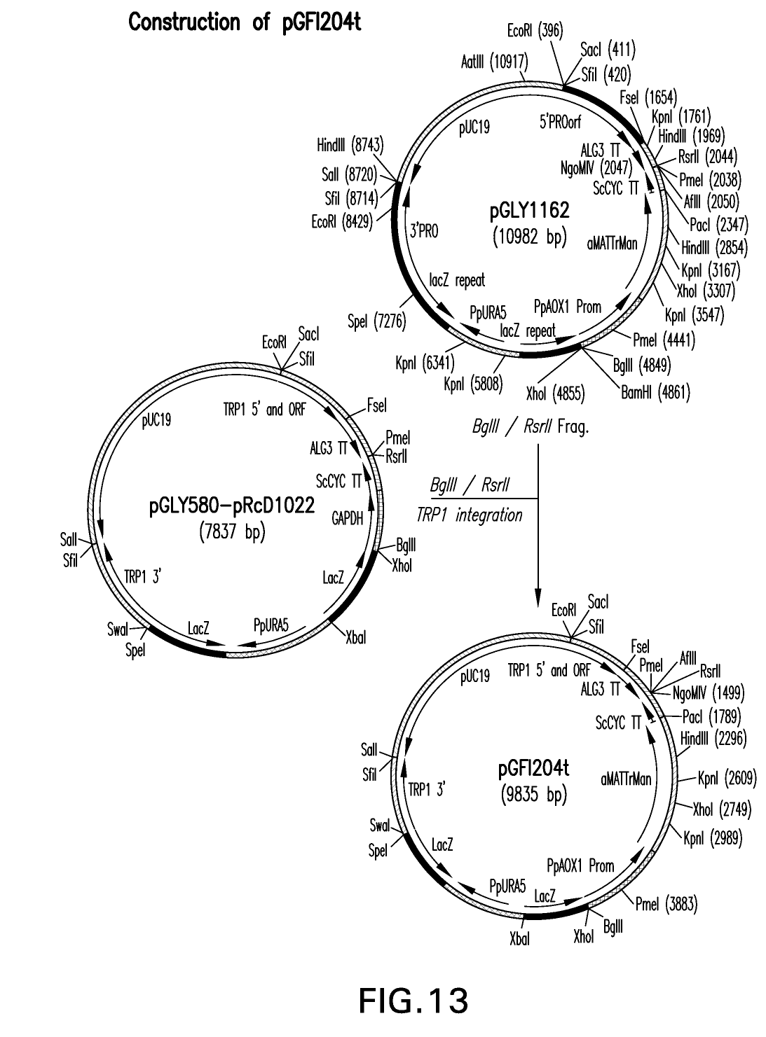

Plasmid vector pGFI204t was made by replacing the PRO1 integration locus in pGLY1162 with TRP1 integration locus from pGLY580. (See Cosano et al., Yeast 14:861-867 (1998) for the TRP1 locus.) This was accomplished by isolating the TRP1 integration locus as BglII-RsrII fragment from pGLY580, and inserting into pGLY1162 that was digested with BglII and RsrII. The two expression cassettes are flanked on one side by a nucleic acid molecule comprising a nucleotide sequence from the 5′ region and complete open reading frame (ORE) of the TRP1 gene (SEQ ID NO:68) followed by a P. pastoris ALG3 termination sequence and on the other side by a nucleic acid molecule comprising a nucleotide sequence from the 3′ region of the TRP1 gene (SEQ ID NO:69). Integration of the plasmid vector is to the Pichia pastoris TRP1 locus and selection is using the Pichia pastoris URA5 gene. Plasmid pGFI204t is a KINKO vector. A map of plasmid vector pGFI204t is shown in FIG. 13.

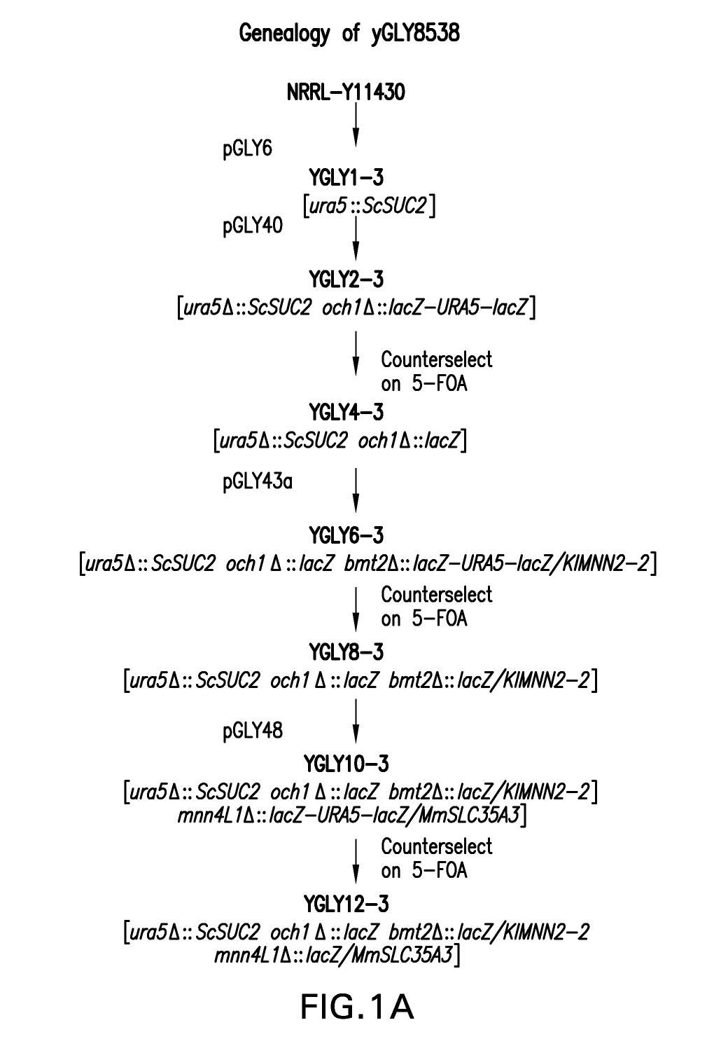

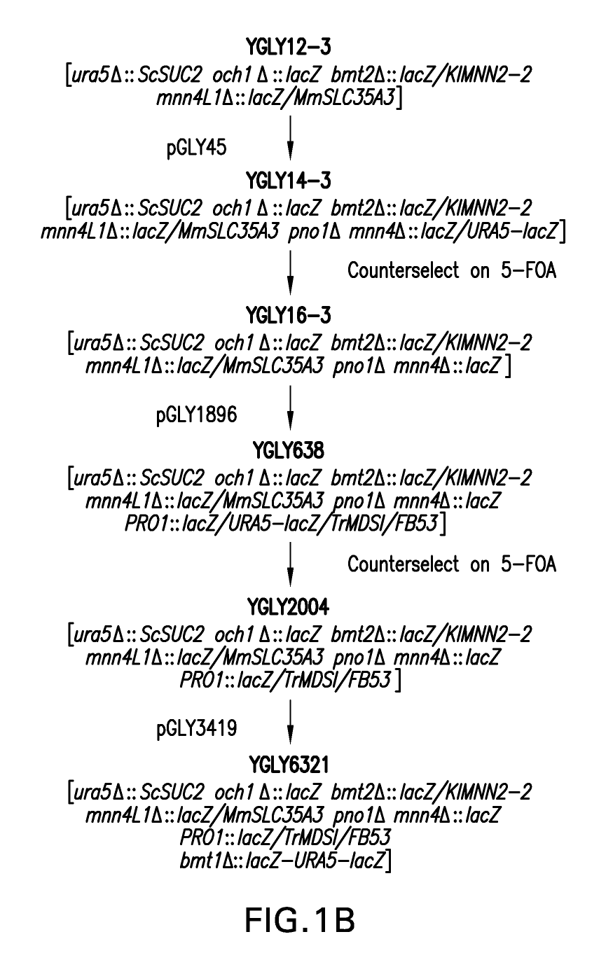

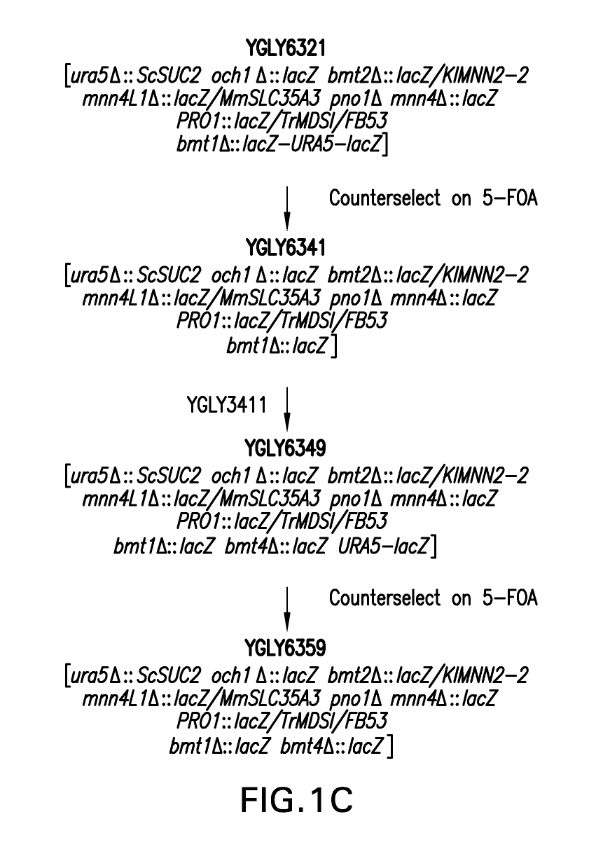

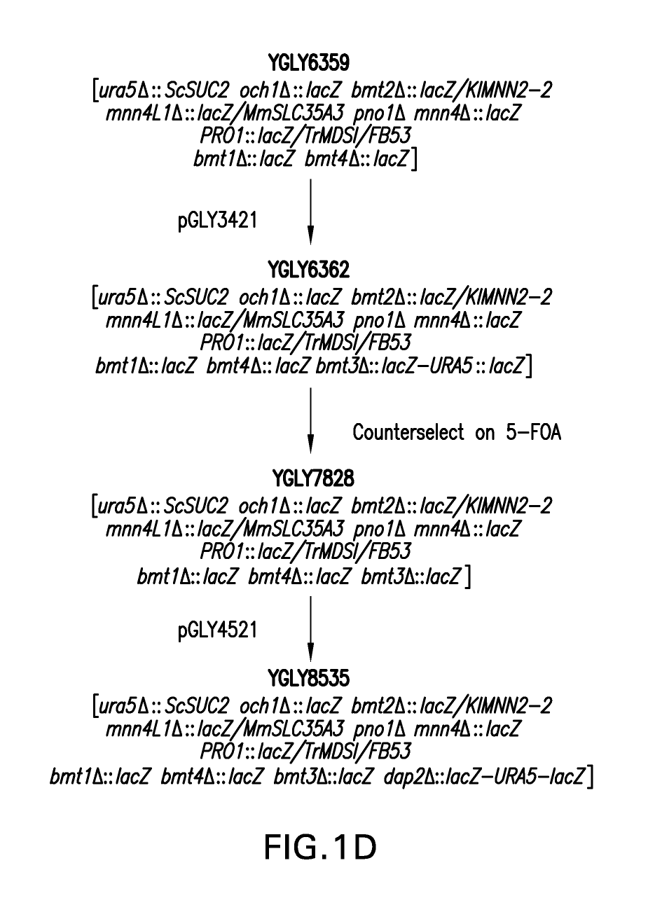

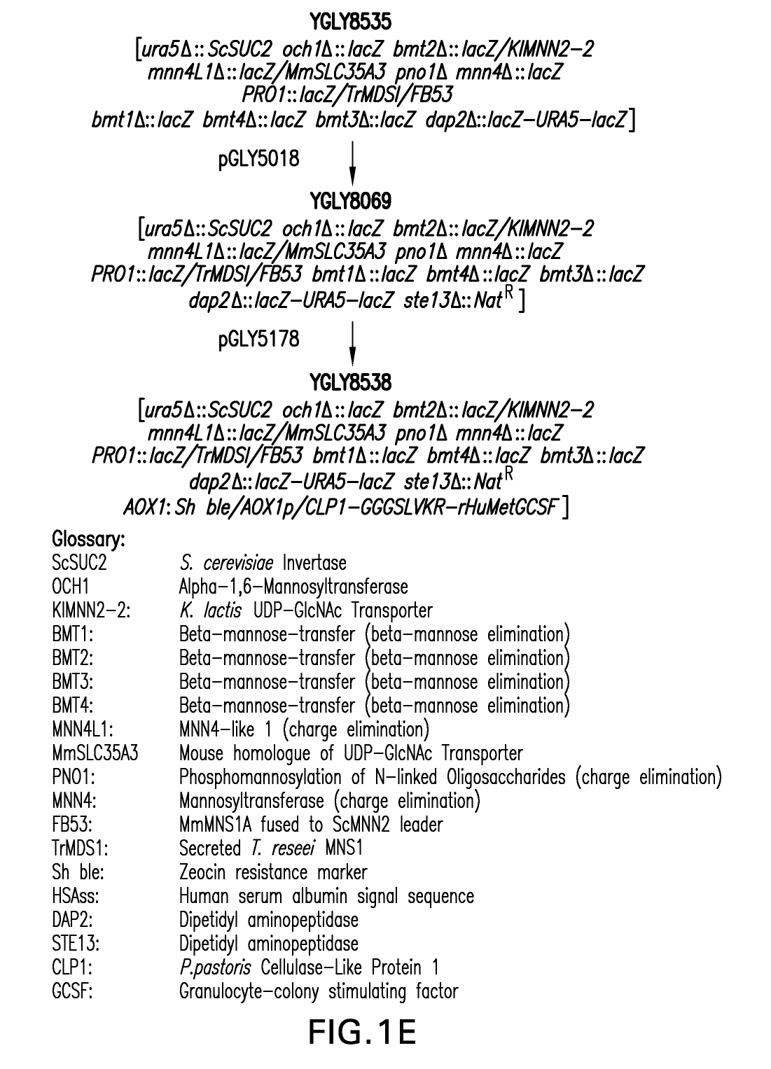

Construction of Genetically Engineered Pichia 2.0 strain YGLY8538 for producing rHuMetGCSF. Strain YGLY8538 was constructed from wild-type Pichia pastoris strain NRRL-Y 11430 as shown in FIG. 1A-1E and briefly described below using methods described earlier (See for example, U.S. Pat. No. 7,449,308; U.S. Pat. No. 7,479,389; U.S. Published Application No. 20090124000; U.S. Published Application No. 2008/0139470; Published PCT Application No. WO2009085135; Nett and Gerngross, Yeast 20:1279 (2003); Choi et al., Proc. Natl. Acad. Sci. USA 100:5022 (2003); Hamilton et al., Science 301:1244 (2003)). All plasmids were made in a pUC19 plasmid using standard molecular biology procedures. For nucleotide sequences that were optimized for expression in P. pastoris, the native nucleotide sequences were analyzed by the GENEOPTIMIZER software (GeneArt, Regensburg, Germany) and the results used to generate nucleotide sequences in which the codons were optimized for P. pastoris expression. Yeast strains were transformed by electroporation (using standard techniques as recommended by the manufacturer of the electroporator BioRad). Methods for integrating heterologous nucleic acid molecules into the genome of Pichia pastoris are well known in the art and have been described in numerous references, including but not limited to, U.S. Pat. No. 7,479,389, PCT Published Application No. WO2007/136865, and PCT/US2008/13719.

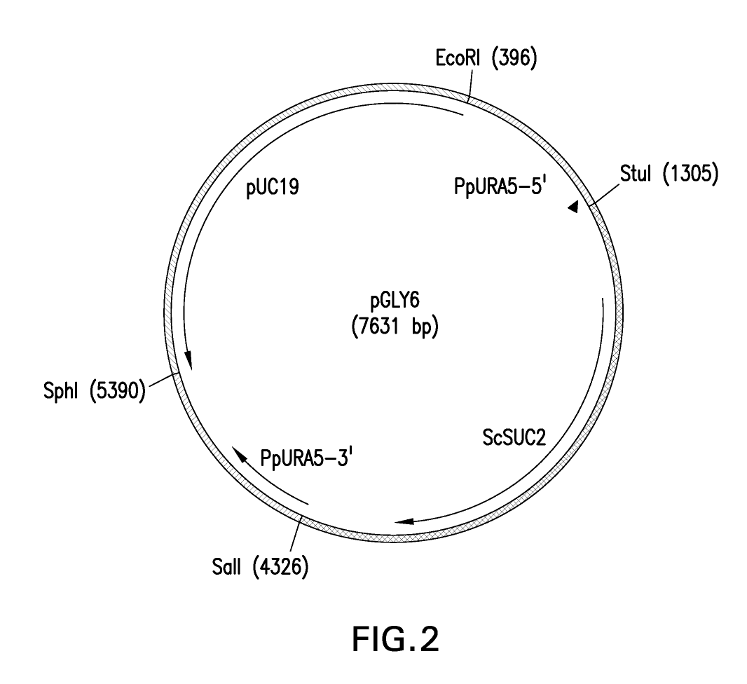

Plasmid pGLY6 (FIG. 2) is an integration vector that targets the URA5 locus contains a nucleic acid molecule comprising the S. cerevisiae invertase gene or transcription unit (ScSUC2; SEQ ID NO:65) flanked on one side by a nucleic acid molecule comprising a nucleotide sequence from the 5′ region of the P. pastoris URA5 gene (SEQ ID NO:35) and on the other side by a nucleic acid molecule comprising the a nucleotide sequence from the 3′ region of the P. pastoris URA5 gene (SEQ ID NO:36). Plasmid pGLY6 was linearized and the linearized plasmid transformed into wild-type strain NRRL-Y 11430 to produce a number of strains in which the ScSUC2 gene was inserted into the URA5 locus by double-crossover homologous recombination. Strain YGLY1-3 was selected from the strains produced and is auxotrophic for uracil.

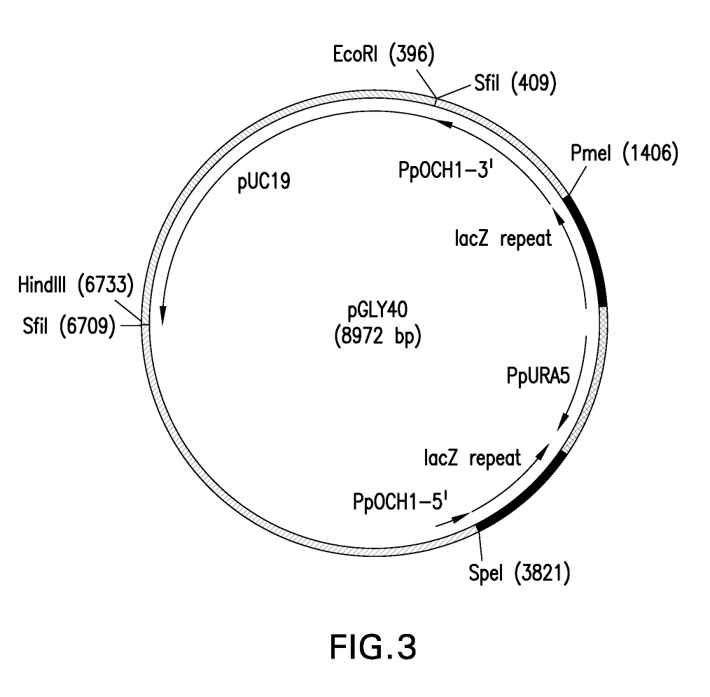

Plasmid pGLY40 (FIG. 3) is an integration vector that targets the OCH1 locus and contains a nucleic acid molecule comprising the P. pastoris URA5 gene or transcription unit (SEQ ID NO:37) flanked by nucleic acid molecules comprising lacZ repeats (SEQ ID NO:38) which in turn is flanked on one side by a nucleic acid molecule comprising a nucleotide sequence from the 5′ region of the OCH1 gene (SEQ ID NO:39) and on the other side by a nucleic acid molecule comprising a nucleotide sequence from the 3′ region of the OCH1 gene (SEQ ID NO:40). Plasmid pGLY40 was linearized with SfiI and the linearized plasmid transformed into strain YGLY1-3 to produce to produce a number of strains in which the URA5 gene flanked by the lacZ repeats has been inserted into the OCH1 locus by double-crossover homologous recombination. Strain YGLY2-3 was selected from the strains produced and is prototrophic for URA5. Strain YGLY2-3 was counterselected in the presence of 5-fluoroorotic acid (5-FOA) to produce a number of strains in which the URA5 gene has been lost and only the lacZ repeats remain in the OCH1 locus (See U.S. Pat. No. 7,514,253). This renders the strain auxotrophic for uracil. Strain YGLY4-3 was selected.

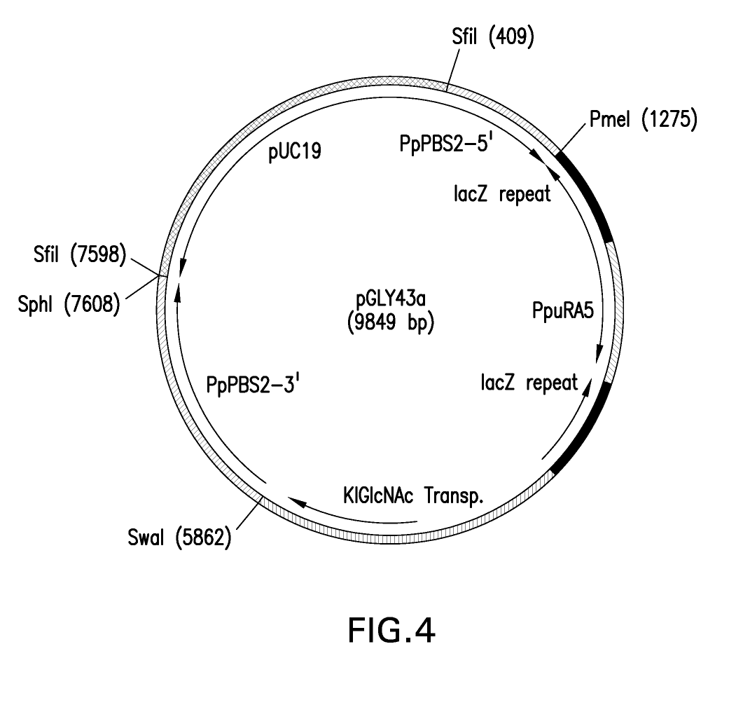

Plasmid pGLY43a (FIG. 4) is an integration vector that targets the BMT2 locus and contains a nucleic acid molecule comprising the K lactis UDP-N-acetylglucosamine (UDP-GlcNAc) transporter gene or transcription unit (KlMNN2-2, SEQ ID NO:66) adjacent to a nucleic acid molecule comprising the P. pastoris URA5 gene or transcription unit flanked by nucleic acid molecules comprising lacZ repeats. The adjacent genes are flanked on one side by a nucleic acid molecule comprising a nucleotide sequence from the 5′ region of the BMT2 gene (SEQ ID NO: 41) and on the other side by a nucleic acid molecule comprising a nucleotide sequence from the 3′ region of the BMT2 gene (SEQ ID NO:42). Plasmid pGLY43a was linearized with SfiI and the linearized plasmid transformed into strain YGLY4-3 to produce to produce a number of strains in which the KlMNN2-2 gene and URA5 gene flanked by the lacZ repeats has been inserted into the BMT2 locus by double-crossover homologous recombination. The BMT2 gene has been disclosed in Mille et al., J. Biol. Chem. 283: 9724-9736 (2008) and U.S. Pat. No. 7,465,557. Strain YGLY6-3 was selected from the strains produced and is prototrophic for uracil. Strain YGLY6-3 was counterselected in the presence of 5-FOA to produce strains in which the URA5 gene has been lost and only the lacZ repeats remain. This renders the strain auxotrophic for uracil. Strain YGLY8-3 was selected.

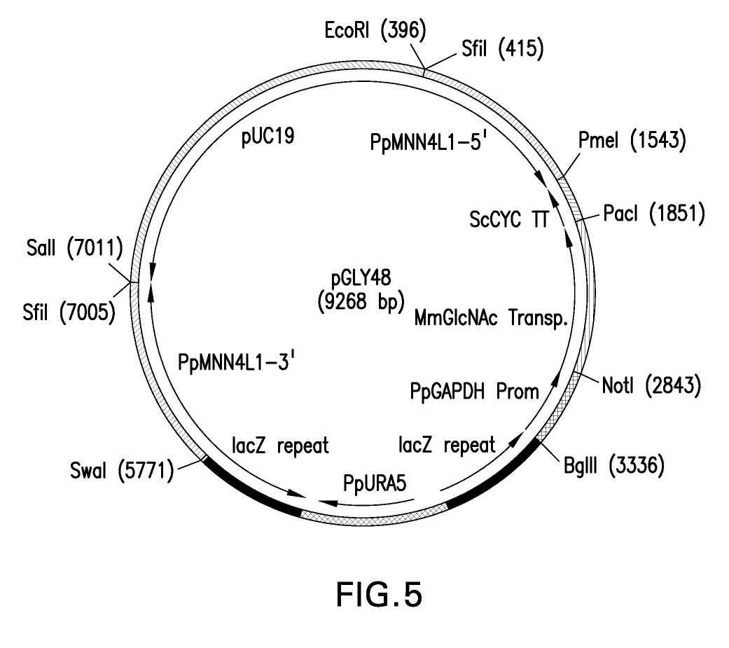

Plasmid pGLY48 (FIG. 5) is an integration vector that targets the MNN4L1 locus and contains an expression cassette comprising a nucleic acid molecule encoding the mouse homologue of the UDP-GlcNAc transporter (SEQ ID NO:67) open reading frame (ORF) operably linked at the 5′ end to a nucleic acid molecule comprising the P. pastoris GAPDH promoter (SEQ ID NO:54) and at the 3′ end to a nucleic acid molecule comprising the S. cerevisiae CYC termination sequences (SEQ ID NO:57) adjacent to a nucleic acid molecule comprising the P. pastoris URA5 gene flanked by lacZ repeats and in which the expression cassettes together are flanked on one side by a nucleic acid molecule comprising a nucleotide sequence from the 5′ region of the P. Pastoris MNN4L1 gene (SEQ ID NO:51) and on the other side by a nucleic acid molecule comprising a nucleotide sequence from the 3′ region of the MNN4L1 gene (SEQ ID NO:52). Plasmid pGLY48 was linearized with SfiI and the linearized plasmid transformed into strain YGLY8-3 to produce a number of strains in which the expression cassette encoding the mouse UDP-GlcNAc transporter and the URA5 gene have been inserted into the MNN4L1 locus by double-crossover homologous recombination. The MNN4L1 gene (also referred to as MNN4B) has been disclosed in U.S. Pat. No. 7,259,007. Strain YGLY10-3 was selected from the strains produced and then counterselected in the presence of 5-FOA to produce a number of strains in which the URA5 gene has been lost and only the lacZ repeats remain. Strain YGLY1Z-3 was selected.

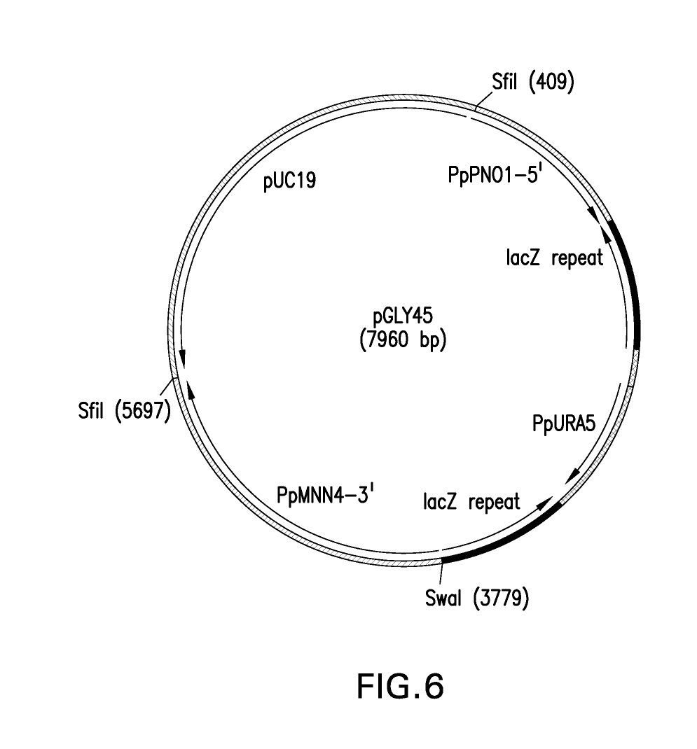

Plasmid pGLY45 (FIG. 6) is an integration vector that targets the PNO1/MNN4 loci contains a nucleic acid molecule comprising the P. pastoris URA5 gene or transcription unit flanked by nucleic acid molecules comprising lacZ repeats which in turn is flanked on one side by a nucleic acid molecule comprising a nucleotide sequence from the 5′ region of the PNO1 gene (SEQ ID NO: 49) and on the other side by a nucleic acid molecule comprising a nucleotide sequence from the 3′ region of the MNN4 gene (SEQ ID NO:50). Plasmid pGLY45 was linearized with SfiI and the linearized plasmid transformed into strain YGLY12-3 to produce to produce a number of strains in which the URA5 gene flanked by the lacZ repeats has been inserted into the PNO1/MNN4 loci by double-crossover homologous recombination. The PNO1 gene has been disclosed in U.S. Pat. No. 7,198,921 and the MNN4 gene (also referred to as MNN4B) has been disclosed in U.S. Pat. No. 7,259,007. Strain YGLY14-3 was selected from the strains produced and then counterselected in the presence of 5-FOA to produce a number of strains in which the URA5 gene has been lost and only the lacZ repeats remain. Strain YGLY16-3 was selected.

Strain YGLY16-3 was transfected with plasmid pGLY1896 described as above as encoding a secreted T. reesei mannosidase I and a mouse α-1,2-mannosdiase I targeted to the ER/Golgi to produce a number of strains of which strain YGLY638 was selected Strain YGLY2004 was constructed by counterselecting strain YGLY638 with 5-FOA to remove the URA5 gene leaving behind the lacZ repeats.

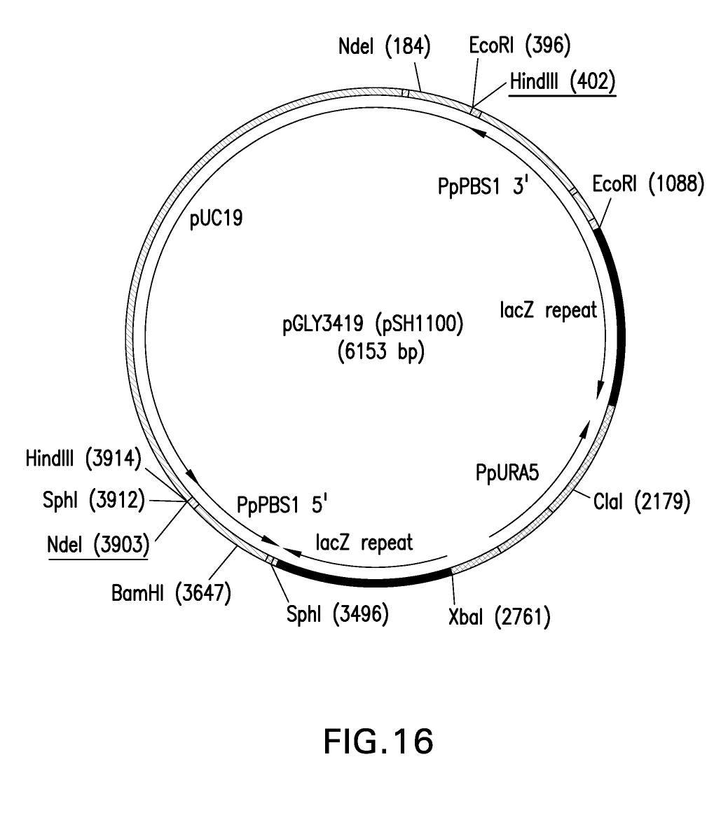

Plasmid pGLY3419 (FIG. 16) is an integration vector that contains the expression cassette comprising the P. pastoris URA5 gene flanked by lacZ repeats flanked on one side with the 5′ nucleotide sequence of the P. pastoris BMT1 gene (SEQ ID NO:43) and on the other side with the 3′ nucleotide sequence of the P. pastoris BMT1 gene (SEQ ID NO:44). Plasmid pGLY3419 was linearized and the linearized plasmid transformed into YGLY2004 to produce a number of strains in which the URA5 expression cassette has been inserted into the BMT1 locus by double-crossover homologous recombination. Strain YGLY6321 was selected from the strains produced. Strain YGLY6321 was then counterselected in the presence of 5-FOA as above to produce a number of strains now auxotrophic for uridine of which strain YGLY6341 was selected.

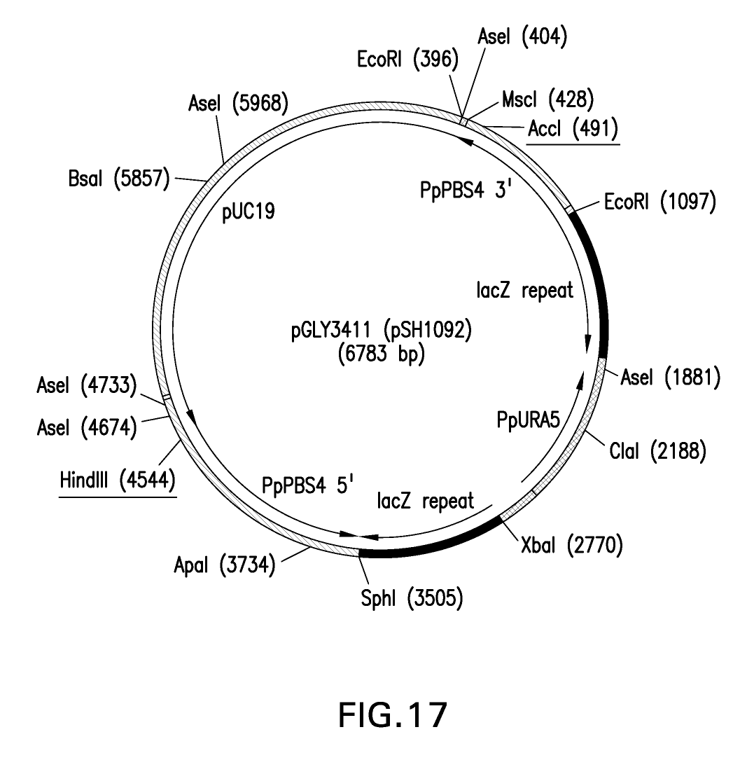

Plasmid pGLY3411 (FIG. 17) is an integration vector that contains the expression cassette comprising the P. pastoris URA5 gene flanked by lacZ repeats flanked on one side with the 5′ nucleotide sequence of the P. pastoris BMT4 gene (SEQ ID NO:47) and on the other side with the 3′ nucleotide sequence of the P. pastoris BMT4 gene (SEQ ID NO:48). Plasmid pGLY3411 was linearized and the linearized plasmid transformed into strain YGLY6341 to produce a number of strains in which the URA5 expression cassette has been inserted into the BMT4 locus by double-crossover homologous recombination. The strain YGLY6349 was selected from the strains produced. Strain YGLY6349 was then counterselected in the presence of 5-FOA as above to produce a number of strains now auxotrophic for uridine of which strain YGLY6359 was selected.

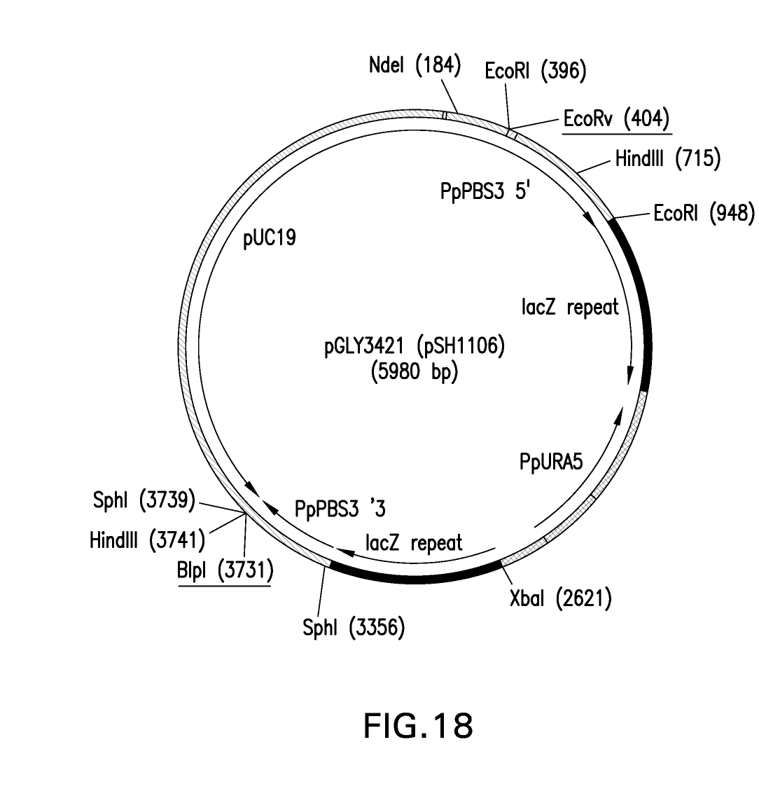

Plasmid pGLY3421 (FIG. 18) is an integration vector that contains the expression cassette comprising the P. pastoris URA5 gene flanked by lacZ repeats flanked on one side with the 5′ nucleotide sequence of the P. pastoris BMT3 gene (SEQ ID NO:45) and on the other side with the 3′ nucleotide sequence of the P. pastoris BMT3 gene (SEQ ID NO:46). Plasmid pGLY3421 was linearized and the linearized plasmid transformed into strain YGLY6359 to produce a number of strains in which the URA5 expression cassette has been inserted into the BMT3 locus by double-crossover homologous recombination. Strain YGLY6362 was selected from the strains produced. Strain YGLY6362 was then counterselected in the presence of 5-FOA as above to produce a number of strains now auxotrophic for uridine of which strain YGLY7828 was selected.

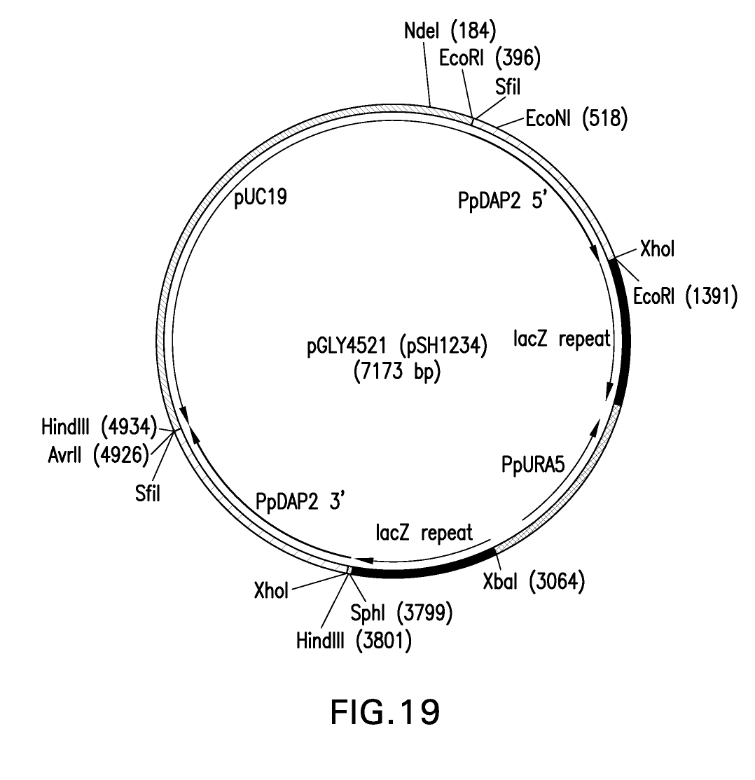

Plasmid pGLY4521 (FIG. 19) is an integration vector that contains the expression cassette comprising the P. pastoris URA5 gene flanked by lacZ repeats flanked on one side with the 5′ nucleotide sequence of the P. pastoris DAP2 gene and on the other side with the 3′ nucleotide sequence of the P. pastoris DAP2 gene. The DAP2 ORF is shown in SEQ ID NO:21. Plasmid pGLY4521 was linearized and the linearized plasmid transformed into strain YGLY7828 to produce a number of strains in which the URA5 expression cassette has been inserted into the DAP2 locus by double-crossover homologous recombination. Strain YGLY8535 was selected from the strains produced.

Plasmid pGLY5018 (FIG. 20) is an integration vector that contains an expression cassette comprising a nucleic acid molecule encoding the Nourseothricin resistance (NAT^{R}) ORF (originally from pAG25 from EROSCARF, Scientific Research and Development GmbH, Daimlerstrasse 13a, D-61352 Bad Homburg, Germany, See Goldstein et al., Yeast 15: 1541 (1999)) ORF (SEQ ID NO:60) operably linked to the P. pastoris TEF1 promoter and P. pastoris TEF1 termination sequences flanked one side with the 5′ nucleotide sequence of the P. pastoris STE13 gene and on the other side with the 3′ nucleotide sequence of the P. pastoris STE13 gene. The STE13 ORE is shown in SEQ ID NO:20. Plasmid pGLY5018 was linearized and the linearized plasmid transformed into strain YGLY8535 to produce a number of strains in which the NAT^{R} expression cassette has been inserted into the STE13 locus by double-crossover homologous recombination. The strain YGLY8069 was selected from the strains produced.

Strain YGLY8069 was transformed with plasmid pGLY5178 (FIG. 8B) to produce strain YGLY8538 encoding the rHuMetGCSF fused to the CLP1 protein and secreting rHuMetGCSF into the medium. Plasmid pGLY5178 was linearized with PmeI and used to transform strain YGLY8069 by roll-in single crossover homologous recombination. A number of strains were produced of which strain YGLY8538 was selected. The strain contains several copies of the expression cassette encoding the rHuMetGCSF integrated into the AOX1 locus (FIG. 1E). The strain secretes rHuMetGCSF into the medium. The genotype of strain YGLY8538 is ura5Δ::ScSUC2 och1Δ::lacZ bmt2Δ::lacZ/KlMNN2-2 mnn4L1Δ::lacZ/MmSLC35A3 pno1Δ mnn4Δ::lacZ PRO1::lacZ/TrMDSI/FB53 bmt1Δ::lacZ bmt4Δ::lacZ bmt3Δ::lacZ dap2Δ::lacZ-URA5-lacZ ste13Δ::NatR AOX1:Sh ble/AOX1p/CLP1-GGGSLVKR-MetGCSF.

### Example 2

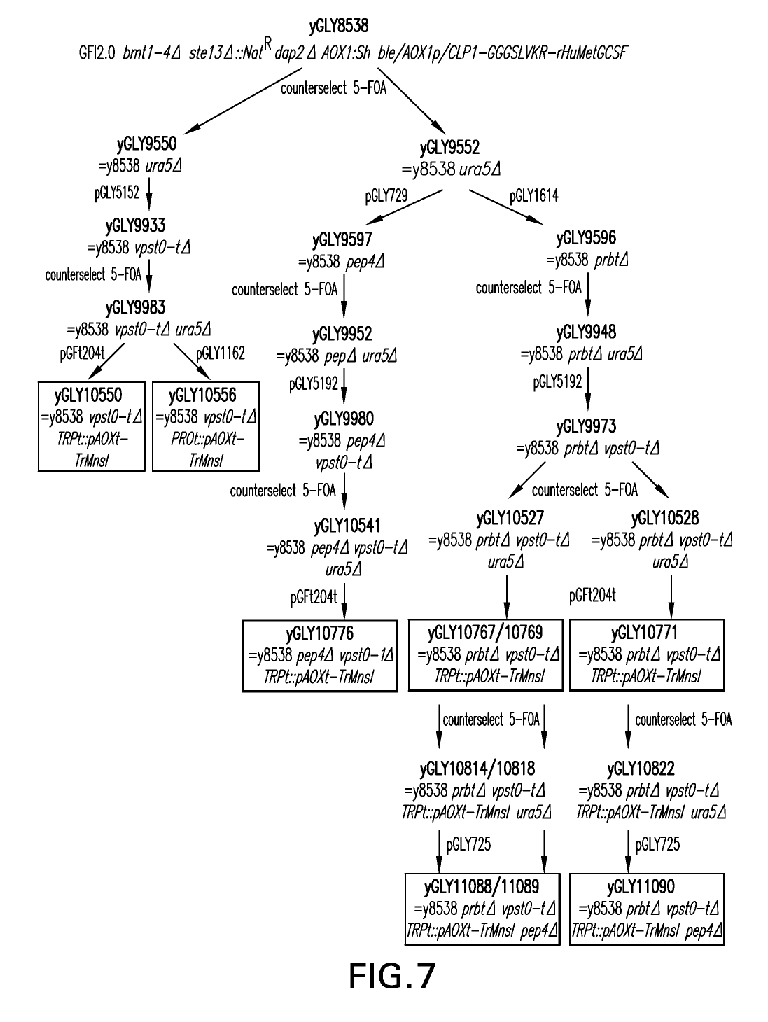

Construction of Optimized GCSF-expressing Pichia Cell Lines. Generation of optimized isogenic yeast strains from YGLY8538 were performed by homologous recombination as described previously (Nett et al., op. cit.). Parental ura5Δ strains were transformed with linearized plasmids containing approximately 500-1000 by flanking DNA upstream and downstream of the desired target gene insertion site. Transformants were selected on URA drop-out plates after gaining the lacZ-URA5-lacZ cassette and analyzed by PCR to verify the correct genetic profile. The following plasmids are used for optimization: pGLY5192 (VPS10-1 knock-out plasmid), pGLY729 (PEP4 knock-out plasmid), pGLY1614 (PRB1 knock-out plasmid), pGLY1162 (PRO1::pAOX1-TrMnsI), and pGFI204t (PRO1::pAOX1-TrMnsI) (See FIGS. 9-13). A flowchart of optimized strain expansion is shown in FIG. 7. Examples of optimized rHuGCSF-expression strains, of which any may be a suitable production cell lineage, and their associated genotypes, are listed in Table 2.

TABLE 2
List of rHuGCSF Strain Genotypes

| Strain | |
|---|---|
| Name | Genotype |
| YGLY10550 | ura5Δ::SCSUC2 och1Δ::lacZ bmt2Δ::lacZ/KlMNN2-2 |
| | mnn4L1Δ::lacZ/MmSLC35A3 pno1 Δmnn4Δ::lacZ |
| | PRO1::lacZ/TrMDSI/FB53 bmt1Δ::lacZ bmt4Δ::lacZ |
| | bmt3Δ::lacZ dap2Δ::lacZ ste13Δ::NatR AOX1::Sh ble/ |
| | AOX1p/CLP1-GGGSLVKR-rHuMetGCSF vps10-1Δ:: |
| | lacZ TRP1::lacZ-URA5-lacZ/AOXp/TrMDSI |
| YGLY10556 | ura5Δ::ScSUC2 och1Δ::lacZ bmt2Δ::lacZIKlMNN2-2 |
| | mnn4L1Δ::lacZ/MmSLC35A3 pno1Δmnn4Δ::lacZ |
| | bmt1Δ::lacZ bmt4Δ::lacZ bmt3Δ::lacZ dap2Δ::lacZ |
| | ste13Δ::NatR AOX1:Sh ble/AOX1p/CLP1-GGGSLVKR- |
| | rHuMetGCSF vps10-1Δ::lacZ PRO1::lacZ-URA5-lacZ/ |
| | AOXp/TrMDSI |
| YGLY10776 | ura5Δ::ScSUC2 och1Δ::lacZ bmt2Δ::lacZ/KlMNN2-2 |
| | mnn4L1Δ::lacZ/MnSLC35A3 pno1Δmnn4Δ::lacZ |
| | PRO1::lacZ/TrMDSI/FB53 bmt1Δ::lacZ bmt4Δ::lacZ |
| | bmt3Δ::lacZ dap2Δ::lacZ ste13Δ::NatR AOX1:Sh ble/ |
| | AOX1p/CLP1-GGGSLVKR-rHuMetGCSF pep4Δ::lacZ |
| | vps10-1Δ::lacZ TRP1::lacZ-URA5-lacZ/AOXp/TrMDSI |
| YGLY10767 | ura5Δ::ScSUC2 och1Δ::lacZ bmt2Δ::lacZ/KlMNN2-2 |
| | mnn4L1Δ::lacZ/MmSLC35A3 pno1Δmnn4Δ::lacZ |
| | PRO1::lacZ/TrMDSI/FB53 bmt1Δ::lacZ bmt4Δ::lacZ |
| | bmt3Δ::lacZ dap2Δ::lacZ ste13Δ::NatR AOX1:Sh ble/ |
| | AOX1p/CLP1-GGGSLVKR-rHuMetGCSF prb1Δ::lacZ |
| | vps10-1Δ::lacZ TRP1::lacZ-URA5-lacZ/AOXp/TrMDSI |
| YGLY10769 | ura5Δ::ScSUC2 och1Δ::lacZ bmt2Δ::lacZ/KlMNN2-2 |
| | mnn4L1Δ::lacZ/MmSLC35A3 pno1Δmnn4Δ::lacZ |
| | PRO1::lacZ/TrMDSI/FB53 bmt1Δ::lacZ bmt4Δ::lacZ |
| | bmt3Δ::lacZdap2Δ::lacZ ste13Δ::NatR AOX1:Sh ble/ |
| | AOX1p/CLP1-GGGSLVKR-rHuMetGCSF prb1Δ::lacZ |
| | vps10-1Δ::lacZ TRP1::lacZ-URA5-lacZ/AOXp/TrMDSI |
| YGLY10771 | ura5Δ::ScSUC2 och1Δ::lacZ bmt2Δ::lacZ/KlMNN2-2 |
| | mnn4L1Δ::lacZ/MmSLC35A3 pno1Δmnn4Δ::lacZ |
| | PRO1::lacZ/TrMDSI/FB53 bmt1Δ::lacZ bmt4Δ::lacZ |
| | bmt3Δ::lacZ dap2Δ::lacZ ste13Δ::NatR AOX1:Sh ble/ |
| | AOX1p/CLP1-GGGSLVKR-rHuMetGCSF prb1Δ::lacZ |
| | vps10-1Δ::lacZ TRP1::lacZ-URA5-lacZ/AOXp/TrMDSI |
| YGLY11088 | ura5Δ::ScSUC2 och1Δ::lacZ bmt2Δ::lacZ/KlMNN2-2 |
| | mnn4L1Δ::lacZ/MmSLC35A3 pno1Δmnn4Δ::lacZ |
| | PRO1::lacZ/TrMDSI/FB53 bmt1Δ::lacZ bmt4Δ::lacZ |
| | bmt3Δ::lacZ dap2Δ::lacZ ste13Δ::NatR AOX1:Sh ble/ |
| | AOX1p/CLP1-GGGSLVKR-rHuMetGCSF prb1Δ::lacZ |
| | vps10-1Δ::lacZ TRP1::lacZ/AOXp/TrMDSIpepΔ::lacZ- |
| | URA5-lacZ |
| yGLY11089 | ura5Δ::ScSUC2 och1Δ::lacZ bmt2Δ::lacZ/KlMNN2-2 |
| | mnn4L1Δ::lacZ/MmSLC35A3 pno1Δmnn4Δ::lacZ |
| | PRO1::lacZ/TrMDSI/FB53 bmt1Δ::lacZ bmt4Δ::lacZ |
| | bmt3Δ::lacZ dap2Δ::lacZ ste13Δ::NatR AOX1:Sh ble/ |
| | AOX1p/CLP1-GGGSLVKR-rHuMetGCSF prb1Δ::lacZ |
| | vps10-1Δ::lacZ TRP1::lacZ/AOXp/TrMDSI pepΔ::lacZ- |
| | URA5-lacZ |
| yGLY11090 | ura5Δ::ScSUC2 och1Δ::lacZ bmt2Δ::lacZ/KlMNN2-2 |
| | mnn4L1Δ::lacZ/MmSLC35A3 pno1Δmnn4Δ::lacZ |
| | PRO1::lacZ/TrMDSI/FB53 bmt1Δ::lacZ bmt4Δ::lacZ |
| | bmt3Δ::lacZ dap2Δ::lacZ ste13Δ::NatR AOX1:Sh ble/ |
| | AOX1p/CLP1-GGGSLYKR-rHuMetGCSF prb1Δ::lacZ |
| | vps10-1Δ::lacZ TRP1::lacZ/AOXp/TrMDSI pepΔ::lacZ- |
| | URA5-lacZ |

### Example 3

Glycoengineered Pichia pastoris has proven to be an excellent recombinant protein production platform. Here, glycoengineered. Pichia is used to produce recombinant human granulocyte-colony stimulating factor. This example illustrates the development of a Pichia pastoris strain capable of producing high quality rHuGCSF in high yield and with no detectable cross-reactivity with antibodies to host cell antigen and with limited O-glycosylation.

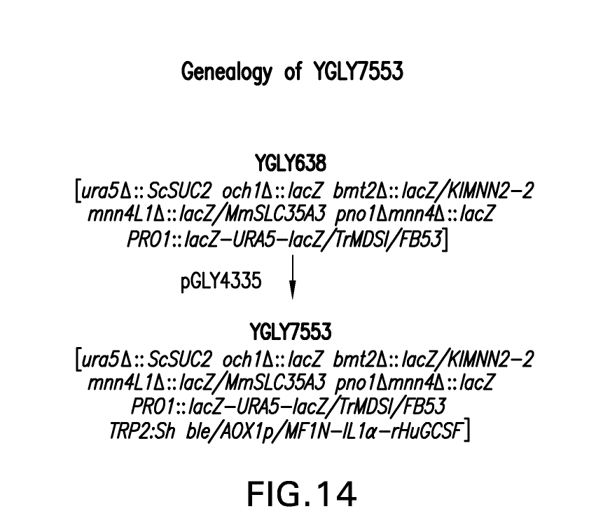

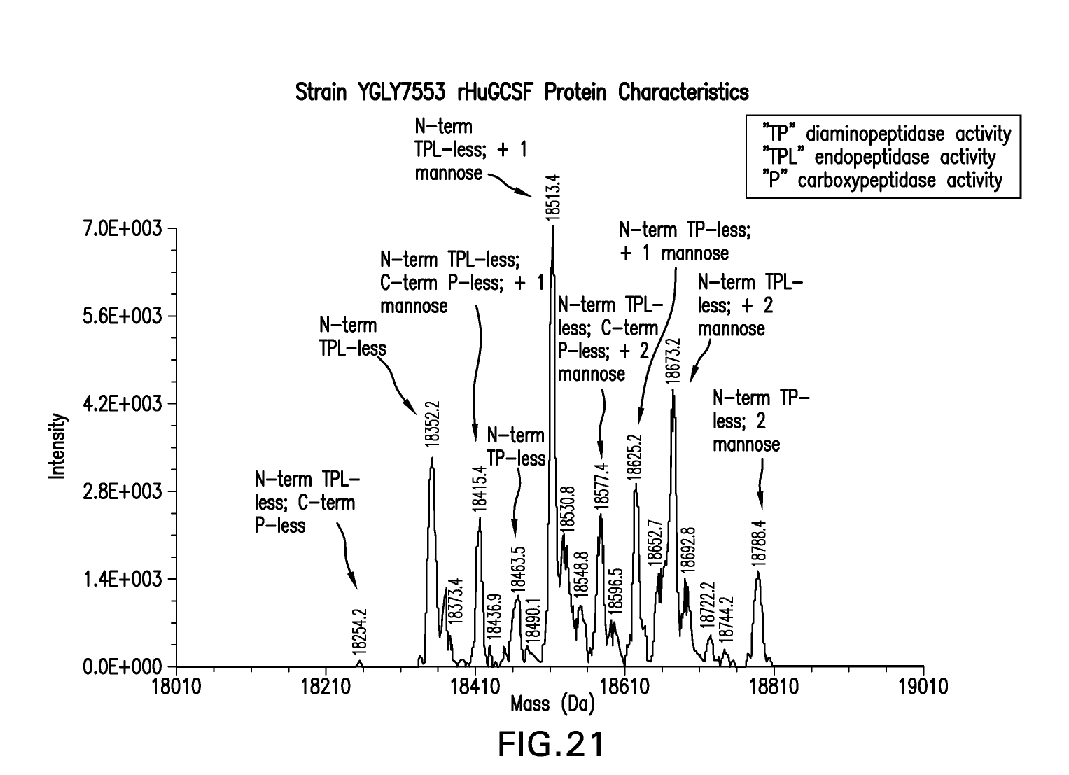

Initial Quality of rHuGCSF expressed in Glycoengineered Pichia pastoris. The first series of experiments resulted in the strain YGLY7553 (FIG. 14). The strain YGLY7553 expresses GCSF using the MFIL-1β prepro signal peptide. Following import to the ER, the mating factor signal peptide is cleaved off the polypeptide and the remaining pro-peptide is cleaved away from rHuGCSF by the Kex2 protease. The secreted rHuGCSF protein does not contain an N-terminal methionine. Following fermentation of this strain in a 40 L bioreactor, the purified protein was subjected to intact electrospray mass spectroscopy to monitor protein characteristics. As seen in FIG. 21, the rHuGCSF derived from YGLY7553 is subjected to aminopeptidase activity (N-term TP-less), endoprotease activity (TPL-less), and carboxypeptidase activity (C-term P-less). The protein also has varying degrees of O-glycosylation, whereby there is protein with no O-mannose, a single O-mannose (mannose), and two O-mannose (mannobiose) glycans (FIG. 21). Subsequent peptide mapping revealed the O-mannose is attached only to Thr133 and may have a chain length of one or two mannose sugars (data not shown). Furthermore, the titer of rHuGCSF from strain YGLY7553 was low (Table 3). In all, this data indicates rHuGCSF secreted from YGLY7553 is of insufficient quality and yield for therapeutic use.

Removal of Diaminopeptidase Activity. We next sought to improve the rHuGCSF protein by eliminating N-terminal TP (Threonine and proline) cleavage. A series of experiments resulted in two independent solutions. Published data in Saccharomyces cerevisiae identified genes responsible for diaminopeptidase activity (e.g., STE13 and DAP2) (Julius et al., Cell 32: 839-52 (1983); Suarez Rendueles & Wolf, 3. Bacteriol. 169: 4041-8 (1987)). The genes encoding dipeptidyl aminopeptidases were genetically deleted from the glycoengineered Pichia strains using standard methods for deleting genes and the like from yeast genomes. The DNA sequences encoding Ste13p and Dap2 in Pichia pastoris are shown in SEQ ID NOs: 20 and 21, respectively.

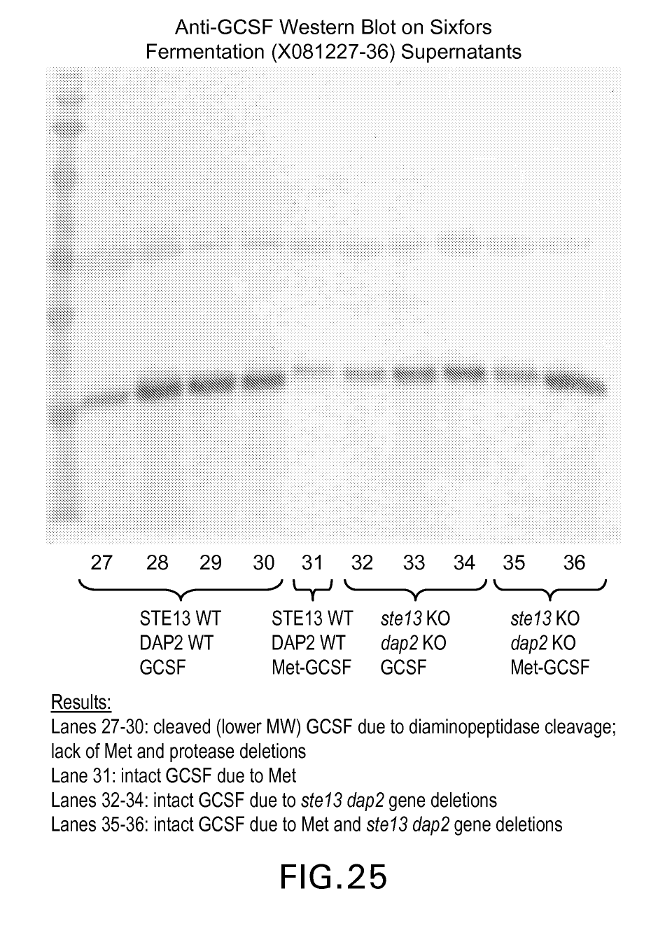

When rHuGCSF is expressed in a cell line with both ste13Δ and dap2A gene deletions, the amino terminal TP residues are not removed. Following a Sixfors fermentation, rHuGCSF expressed from wild-type or mutant STE13 and DAP2 strains were tested for TP cleavage by Western Blot analysis (FIG. 25). When the TP is present on rHuGCSF, the protein migrates as a slightly larger size on SDS-PAGE and verified by N-terminal sequencing (data not shown). For strains with wild-type diaminopeptidase activities (lanes 27-30), rHuGCSF is smaller compared to protein generated in the double mutant background (lanes 32-34). As an alternative means of protecting the N-terminus, an N-terminal methionine was added to rHuGCSF to produce rHuMetGCSF. When rHuMetOCSF is expressed in cells containing diaminopeptidase activity (lane 31), the protein migrates slower to indicate the N-terminus is not degraded by STE13 and DAP2 (verified by N-terminal sequencing but not shown here). Since both solutions of diaminopeptidase cleavage did not result in expression defects for rHuGCSF, all subsequent strains listed here contained the ste13Δ dap2Δ double mutation and N-terminal Methionine (lanes 35-36).

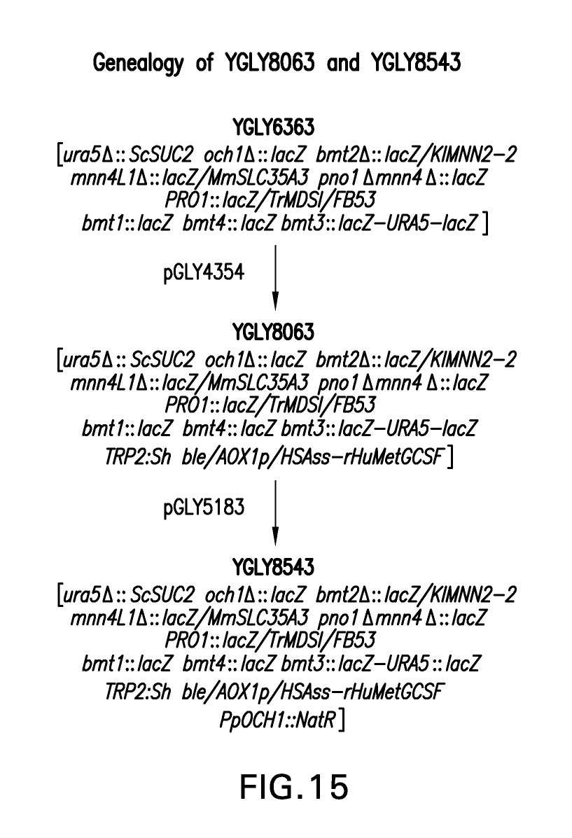

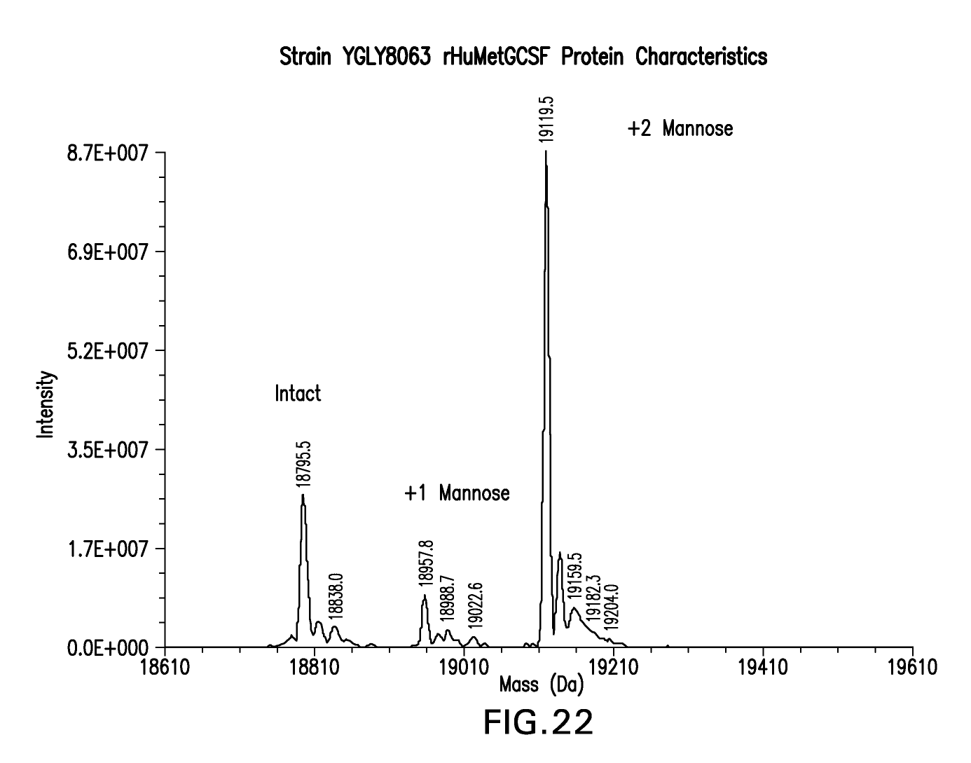

Strain YGLY8063 was constructed in which the rHUGCSF has an N-terminal methionine residue and the leader peptide is the human serum albumin signal peptide (See FIG. 15). Purified rHuMetGCSF from YGLY8063 fermentation was analyzed by electrospray mass spectroscopy to reveal the N-terminus is fully protected from diaminopeptidase cleavage (FIG. 22).

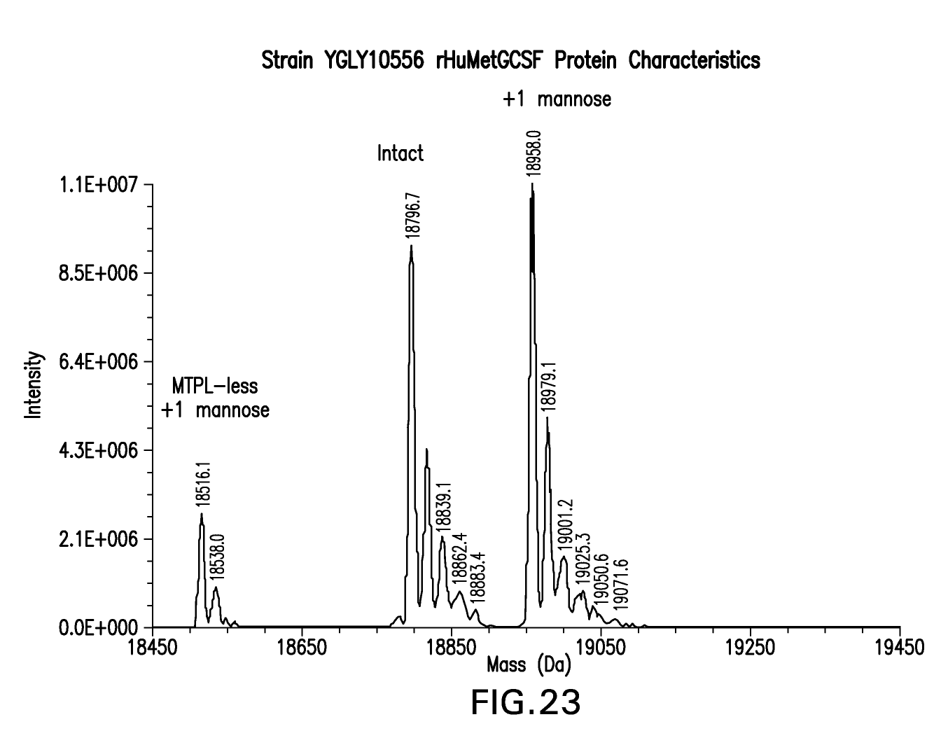

Elimination of Mannobiose O-glycosylation. Following elimination of diaminopeptidase activity, rHuMetGCSF still contained a high percentage of a single O-glycan site with two mannose residues linked by an α-1,2 linkage (FIG. 22). To reduce the mannobiose O-glycan to a single O-mannose, we engineered the strain to secrete α1,2-mannosidase activity to the culture supernatant. YGLY10556 is a strain that was engineered to express an expression cassette encoding the T. reesei mannosidase I catalytic domain fused to the αMATpre signal peptide and operably linked to the AOX1 promoter (AOXp-TrMDSI). When rHuMetGCSF is analyzed from a fermentation of YGLY10556 (FIG. 7 and Table 3), the amount of rHuMetGCSF with mannobiose was dramatically reduced to baseline levels (FIG. 23). However, we did observe an appreciable amount of endoproteolytic activity (MetThrProLeu-less (MTPL-less)) in material from YGLY10556 (FIG. 14).

Elimination of Residual Proteolysis on rHuMetGCSF. To reduce the “MTPL-less” species and C-terminal “P-less” species (as seen in FIG. 21), we were unsure as to the identity of specific proteases that generated these activities. Therefore, we targeted genes whose deletion would reduce or eliminate a large set of putative endoproteases or carboxypeptidases.

It is well published that proteinase A (PrA, encoded by PEP4 gene) and proteinase B (PrB, encoded by PRB1 gene) have key functions in S. cerevisiae and P. pastoris protein degradation, as these proteins not only act upon protein substrates directly but also activate other proteases in a proteolytic cascade (Van Den Hazel et al., Yeast. 12(1):1-16 (1996)). Furthermore, many studies have shown these proteases are key proteases that contribute to recombinant protein degradation in yeast (Jahic et al., Biotechnol Prog. 22(6):1465-73. (2006)). Therefore, we hypothesized a double mutant of pep4Δ prb1Δ may prevent the MTPL-less cleavage product. PEP4 and PRB1 are encoded by SEQ ID NO:18 and SEQ ID NO:19, respectively.

In an effort to increase titer (see below), we also targeted a gene deletion in the Pp VPS10-1 gene (SEQ ID NO:17) that encodes the vacuolar sorting receptor. In S. cerevisiae, the Vps10 receptor functions to deliver vacuolar proteases from the late Golgi network, including carboxypeptidase B, a putative carboxypeptidase acting on rHuMetGCSF. We hypothesized that eliminating this receptor in a rHuMetGCSF strain would lead to secretion of the inactive precursor (pro-carboxypeptidase), eliminating its function on rHuMetGCSF. A series of mutational experiments identified a strain, YGLY11090, with gene deletions of ste13Δ dap2Δ pep4Δ prb1Δ vps10-1Δ, which expresses rHuMetGCSF with background levels of aminopeptidase, endoprotease, and carboxypeptidase activities (FIG. 24). Since this strain also expresses AOXp-TrMDSI, the final purified rHuMetGCSF contains only two species: intact protein with no O-glycosylation and intact protein with a single O-mannose at Thr134. The intact species without O-glycosylation has characteristics that appear similar to NEUPOGEN, which contains an N-terminal Methionine and is produced in E. coli.

Yield Improvement of rHuGCSF. The expression of rHuGCSF at high titers is of similar importance as achieving minimal proteolytic degradation. As seen in Table 3, our initial titers from strain YGLY7553 were quite low at 1 μg/L. To improve our recovery yield of rHuGCSF, we performed many experiments that focused on strain, fermentation, and purification improvements. For example, as shown in. FIG. 15, strain YGLY8063 was transformed with pGLY5183, which inserted the OCH1 gene back into the strain to render the strain OCH1. Many of these improvements were achieved simultaneously, whereby yield improvements were a combination of two or more new factors, as seen in FIGS. 26 and 27 and in Table 3.

TABLE 3
Yield Improvement of rHuGCSF in P. pastoris

| | Process Yield | |
|---|---|---|
| Improvement | (μg/L) | Description |
| Strain YGLY7553 | 1.0 | Initial rHuGCSF strain |
| Strain YGLY8063 | 2.7 | HSAss-rHuMetGCSF |
| Strain YGLY8543 | 2.2 | HSAss-rHuMetGCSF (OCH1+) |
| Strain YGLY8538 | 3.7 | CLP1-rHuMetGCSF fusion |
| Strain YGLY8538 | 7.5 | YGLY8538 process improvements |
| Strain YGLY9933 | 50.0 | VPS10-1 deletion with process |
| | | improvements |

Process improvements- Tween 80, pH 5.0, short induction

Initial improvements were achieved by improving the import or folding of the polypeptide in the endoplasmic reticulum through modifications of the signal peptide or generating gene fusions. Upon DNA transcription in methanol-containing media, the translated polypeptide enters the endoplasmic reticulum by the signal peptide. The polypeptide is further processed in the Golgi apparatus by the Kex2 protease after the arginine residue in the linker sequence, releasing the two proteins of fusion partner and rHuGCSF to the supernatant fraction (See U.S. Published Application No. 2006/0252069). DNA and amino acid sequences of above genes and proteins are listed in the Table of Sequences. Improvements of rHuGCSF yield were obtained with the HSAss and CLP1 prepro fusion partner (Table 3).

With the development of strains yGLY8063 and GLY8538, fermentation and purification processes also improved the yield of rHuMetGCSF. Fermentation experiments demonstrated a high methanol feed rate during induction improved yield significantly. Also, data from literature suggested addition of Tween 80 aided in the recovery of rHuGCSF (Bae et al., Appl. Microbiol. Biotechnol. 52: 338-44 (1999)). Experiments on our glycoengineered strains revealed Tween 80 addition improved rHuMetGCSF yield (Table 3).

A major improvement in rHuMetGCSF yield occurred by deleting the VPS10-1 gene (Table 3). In Saccharomyces cerevisiae, the Vps10p (also known as Pep1 or Vpt1) receptor (and possibly three additional homologs) is responsible for binding pro-carboxypeptidase Y (pro-Cpy, also known as Prc1) via a “QRPL-like” sorting signal and localizing the protein to the vacuole (Marcusson et al., Cell 77: 579-86 (1994); Valls et al., Cell 48: 887-97 (1987)). Most studies focus on the sorting of Cpy in S. cerevisiae to examine binding interactions. These studies identified two regions of the Vps10p luminal receptor domain, each with distinct ligand binding affinities (Jorgensen et al. Eur. J. Biochem. 260: 461-9 (1999); Cereghino et al., Mol. Biol. Cell 6: 1089-102 (1995); Cooper. & Stevens, J. Cell Biol 133: 529-41 (1996)). Mutagenesis of the Cpy “QRPL” peptide near the amino terminus revealed multiple substitutions are capable of interacting with Vps10 (van Voorst et al., J. Biol. Chem. 271: 841-846 (1996)). The S. cerevisiae Vps10p receptor was also shown to interact with recombinant proteins, such as E. coli β-lactamase, in an unknown mechanism not involving a “QRPL-like” sorting domain (Holkeri & Makarow, FEBS Lett. 429: 162-166 (1998)).

In our efforts to express recombinant human granulocyte-colony stimulating factor (G-CSF) in glycoengineered P. pastoris, we identified a sequence (“QSFL”) near the amino termini with characteristics of a Vps10p sorting sequence (van Voorst et al., J. Biol. Chem. 271: 841-6 (1996)). Each of the four amino acid positions in the putative Vps10p binding domain of rHuGCSF were compared to previous mutagenesis results for Cpy vacuolar targeting to reveal no less than 85% activity of Cpy targeting (van Voorst et al., J. Biol. Chem. 271: 841-846 (1996); Tamada, et al., Proc. Natl. Acad. Sci. USA 103: 3135-3140 (2006)). Furthermore, the “QSFL” peptide maps to a surfaced-exposed region of the protein capable of interacting with Vps10p (Tamada et al., Proc. Natl. Acad. Sci. USA 103: 3135-3140 (2006); Hill et al., Proc. Natl. Acad. Sci. USA 90: 5167-5171 (1993)). Based on the likelihood of Vps10p receptor binding and surface exposure, we hypothesized mutations in the P. pastoris VPS10 homologs would improve secretory yields of rHuGCSF by eliminating aberrant sorting of recombinant protein to the vacuole. The expression strain YGLY8538 was counterselected using 5-Fluoroorotic acid (5-FOA) and transformed with pGLY5192 to generate the vps10-1Δ mutant strain YGLY9933 (See FIG. 7). Strain YGLY9933 was fermented and revealed the rHuMetGCSF titer to be dramatically higher compared to YGLY8538 (Table 3). Further optimizations in fermentation, including extending induction times and increased Tween 80 concentration, boosted the yield even further. In total, these improvement strategies improved the yield over 200-fold to generate a complete process that allows for rHuMetGCSF to be produced at high enough yield and of high quality to be used as a human protein therapeutic.

### General Methods

Bioreactor Screening. Bioreactor Screenings (SIXFORS) for rHuGCSF expression were done in 0.5 L vessels (Sixfors multi-fermentation system, ATR Biotech, Laurel, Md.) under the following conditions: pH at 6.5, 24° C., 0.3 SLPM, and an initial stirrer speed of 550 rpm with an initial working volume of 350 mL (330 mL BMGY medium and 20 mL inoculum). IRIS multi-fermentor software (ATR Biotech, Laurel, Md.) was used to linearly increase the stirrer speed from 550 rpm to 1200 rpm over 10 hours, one hour after inoculation. Seed cultures (200 mL of BMGY in a 1 L baffled flask) were inoculated directly from agar plates. The seed flasks were incubated for 72 hours at 24° C. to reach optical densities (OD₆₀₀) between 95 and 100. The fermentors were inoculated with 200 mL stationary phase flask cultures that were concentrated to 20 mL by centrifugation. The batch phase ended on completion of the initial charge glycerol (18-24 h) fermentation and were followed by a second batch phase that was initiated by the addition of 17 mL of glycerol feed solution (50% [w/w] glycerol, 5 mg/L Biotin, 12.5 mL/L PTM1 salts (65 g/L FeSO4.7H2O, 20 g/L ZnCl₂, 9 g/L H2SO4, 6 g/L CuSO4.5H2O, 5 g/L H2SO4, 3 g/L MnSO4.7H2O, 500 mg/L CoCl2.6H2O, 200 mg/L NaMoO4.2H2O, 200 mg/L biotin, 80 mg/L NaI, 20 mg/L H3BO4)). Upon completion of the second batch phase, as signaled by a spike in dissolved oxygen, the induction phase was initiated by feeding a methanol feed solution (100% MeOH 5 mg/L biotin, 12.5 mL/L PTM1) at 0.6 g/h for 32-40 hours. The cultivation is harvested by centrifugation.

Platform Fermentation Process: Bioreactor cultivations were done in 3 L and 15 L glass bioreactors (Applikon, Foster City, Calif.) and a 40 L stainless steel, steam in place bioreactor (Applikon, Foster City, Calif.). Seed cultures were prepared by inoculating BMGY media directly with frozen stock vials at a 1% volumetric ratio. Seed flasks were incubated at 24° C. for 48 hours to obtain an optical density (OD₆₀₀) of 20±5 to ensure that cells are growing exponentially upon transfer. The cultivation medium contained 40 g glycerol, 18.2 g sorbitol, 2.3 g K₂HPO₄, 11.9 g KH₂PO₄, 10 g yeast extract (BD, Franklin Lakes, N.J.), 20 g peptone (BD, Franklin Lakes, N.J.), 4×10⁻³ g biotin and 13.4 g Yeast Nitrogen Base (BD, Franklin Lakes, N.J.) per liter. The bioreactor was inoculated with a 10% volumetric ratio of seed to initial media. Cultivations were done in fed-batch mode under the following conditions: temperature set at 24±0.5° C., pH controlled at to 6.5±0.1 with NH₄OH, dissolved oxygen was maintained at 1.7±0.1 mg/L by cascading agitation rate on the addition of O₂. The airflow rate was maintained at 0.7 vvm. After depletion of the initial charge glycerol (40 g/L), a 50% (w/w) glycerol solution (containing 12.5 ml/L of PTM2 salts and 12.5 ml/L of 25XBiotin) was fed exponentially at a rate of 0.08 h⁻¹ starting at 5.33 g/L/hr (50% of the maximum growth rate) for eight hours. Induction was initiated after a 30 minute starvation phase when methanol (containing 12.5 ml/L of PTM2 salts and 12.5 ml/L of 25XBiotin) was fed exponentially to maintain a specific growth rate of 0.01 h⁻¹ starting at 2 g/L/hr.

Improved Fermentation Processes: Process development on various rHuGCSF expression strains included optimization of fermentation cultivation for improved product yield and properties.

For YGLY7553, the platform fermentation process was used to generate rHuGCSF.

For YGLY8063, an excess methanol experiment was performed using a methanol sensor (Raven methanol sensor) and identified the maximum growth rate. Qp vs. mu study was performed at different growth rates (methanol feed rates) and identified that high methanol feed rate (6.33 g/L/hr) was beneficial in improving the titer. Tween80 was also evaluated and found to be attractive as addition of 0.68 g/L Tween 80 into the methanol boosted the titer. The glycerol batch and fed-batch phase for the high methanol feed rate experiment was identical to that of platform process.

For YGLY8538, rHuMetGCSF was generated using high methanol feed rate (ramped the methanol feed rate from 2.33 g/L/hr to 6.33 g/L/hr in a 6 hr period and maintained at 6.33 g/L/hr for the entire course of induction) and by adding 0.68 g/L of Tween 80 into the methanol. Fermentation pH was reduced to 5.0 as a process improvement for this and the following strains.

For YGLY9933, the high methanol feed rate, 0.68 g/L Tween 80, and fermentation pH 5.0 was utilized.

Finally, YGLY11090 was cultivated using the high methanol feed rate and 0.68 g/L Tween 80 in Methanol. Fermentation pH was 5.0.

GCSF Titer Determination. Cleared supernatant fractions were assayed for rHuGCSF titer with a standard ELISA protocol. Briefly, polyclonal anti-GCSF antibodies (R&D Systems®, Cat#MAB214) was coated onto a 96 well high binding plate (Corning®, Cat#3922), blocked, and washed. A rHuGCSF protein standard (R&D Systems®, Cat. #214-CS) and serial dilutions of cell-free supernatant fluid were applied to the above plate and incubated for 1 hour. Following a washing step, monoclonal anti-GCSF antibodies (R&D Systems®, Cat#AB-2,4-NA) was added to the plate and incubated for one hour. After washing, an alkaline phosphatase-conjugated goat anti-mouse IgG Fc (Thermo Scientific®, Cat#31325) was added and incubated for one hour. The plate was washed and the fluorescent detection reagent 4-MUPS was added and incubated in the absence of light. Fluorescent intensities were measured on a TECAN fluorometer with 340 nm excitation and 465 nm emission properties.

Intact Electrospray Protocol. Protein quality of rHuGCSF was determined using intact mass spectroscopy to monitor proteolytic cleavage and O-glycosylation. Intact analysis was performed on the Waters Acquity HPLC and Thermo LTQ mass spectrometer. Twenty micrograms of purified sample was injected onto an Acquity BEH C8 1.7 um (2.1×100 mm) column at 50° C. The elution gradient is described in Table 4, whereby Buffer A was 0.1% Formic Acid in HPLC water and Buffer B was 0.1% Formic Acid in 90% Acetonitrile.

TABLE 4

| | Flow | | | |
|---|---|---|---|---|
| Time | (ml/min) | % A | % B | Curve |
| Initial | 0.5 | 80 | 20 | Initial |
| 5 | 0.5 | 80 | 20 | 1 |
| 15 | 0.5 | 20 | 80 | 6 |
| 20 | 0.5 | 20 | 80 | 1 |
| 25 | 0.5 | 95 | 5 | 1 |

Following LC elution, sample is sprayed into the Thermo LTQ mass spectrometer where the molecules are ionized. During ionization the protein acquires multiple charges. Mass deconvolution, using XCalibur Promass software, converts the multiply charged mass spectrum into a singly charged parent spectrum and calculates the molecular weight of the protein. rHuGCSF protein species with characteristic masses of intact molecule and/or multiple proteolytic cleaved species, each with varying degrees of O-glycan modification are identified based on theoretical versus measured mass calculations.

### Example 4

The rHuGCSF was modified to include a polyethylene glycol (PEG) polymer at the N-terminus. Provided is a representative procedure which has been used to PEGylate rHuMetGCSF from strain YGLY8538 with 20 kDa PEG.

The PEGylation reaction used mPEG-propionaldehyde (mPEG-PA) obtained from NOF Corporation (SUNBRIGHT ME 200AL; 20 kDa PEG; Cas No. 125061-88-3; α-methyl-ω-(3-oxopropoxy)polyoxyethylene); SM Sodium cyanoborohydride solution in 1M NaOH (Sigma Cat #296945); rHuGCSF purified from engineered Pichia pastoris (Conc. 1 mg/mL); and Sodium acetate, anhydrous (LT. Baker Cat #3473-05).

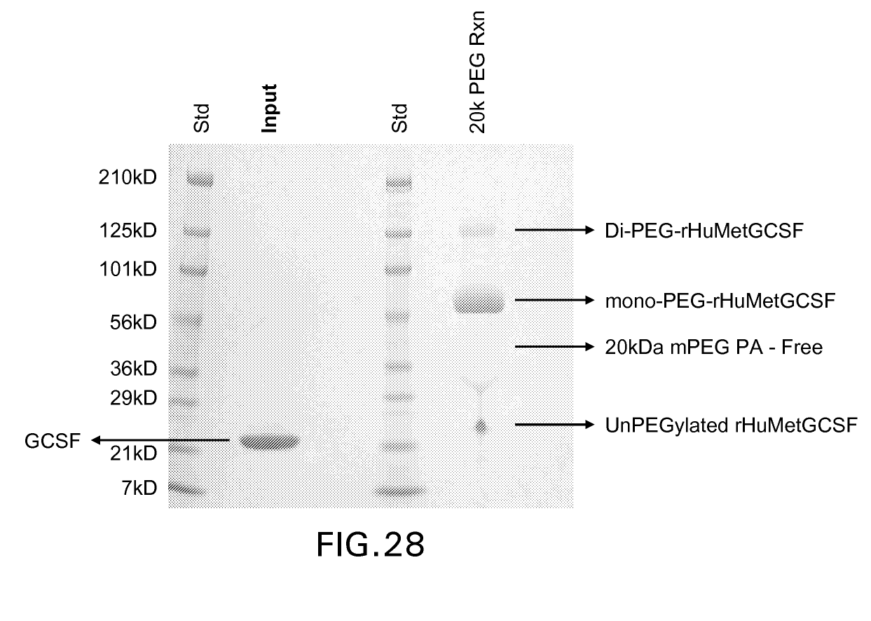

N-terminal Specific reaction was as follows. The rHuMetGCSF (1 mg/mL) was buffer-exchanged into 100 mM Sodium acetate pH 5.0. Then, 20 mM Sodium cyanoborohydride was added. Next, a mPEG-Propionaldehyde was added at a 1:10 ratio of Protein to mPEG-PA (e.g., 1 mg of rHuMetGCSF and 10 mg of mPEG-PA) and the reaction mixture stirred until the mPEG-PA was dissolved. The reaction was incubated at 4° C. for 12 hours. Afterwards, the reaction was stopped with the addition of 10 mM TRIS pH 6.0. The efficiency of formation of PEGylated rHuMetGCSF was determined by taking an aliquot of the reaction mixture and analyzing it by reverse-phase HPLC, SEC, and SDS-PAGE Gel electrophoresis. FIG. 28 shows an SDS polyacrylamide gel stained with Coomassie blue showing the amount of mono-PEGylated rHuMetGCSF that was formed.

### Example 5

This example provides a representative method for isolating and purifying mono PEGylated rHuMetGCSF from di-PEGylated and unPEGylated material.

GE Tricorn 10/300 or equivalent columns were packed with SP SEPHAROSE High Performance resin (GE health care Cat. 417-1087-01). A packed SP SEPHAROSE HP column was attached to an AKTA Explorer 100 or equivalent. The columns were washed with dH₂O and equilibrated with three column volumes (CV) of 20 mM Sodium acetate pH 4.0. The Post PEGylation reaction 1:10 mixture from Example 4 was diluted with distilled water and the pH adjusted to 4.0 with dilute HCl. The final concentration of PEGylated rHuMetGCSF (PEG-rHuMetGCSF) was about 2.0 mg total protein per mL. The pH-adjusted reaction mixture was loaded onto the pre-equilibrated SP SEPHAROSE HP column using AKTA Explorer program.

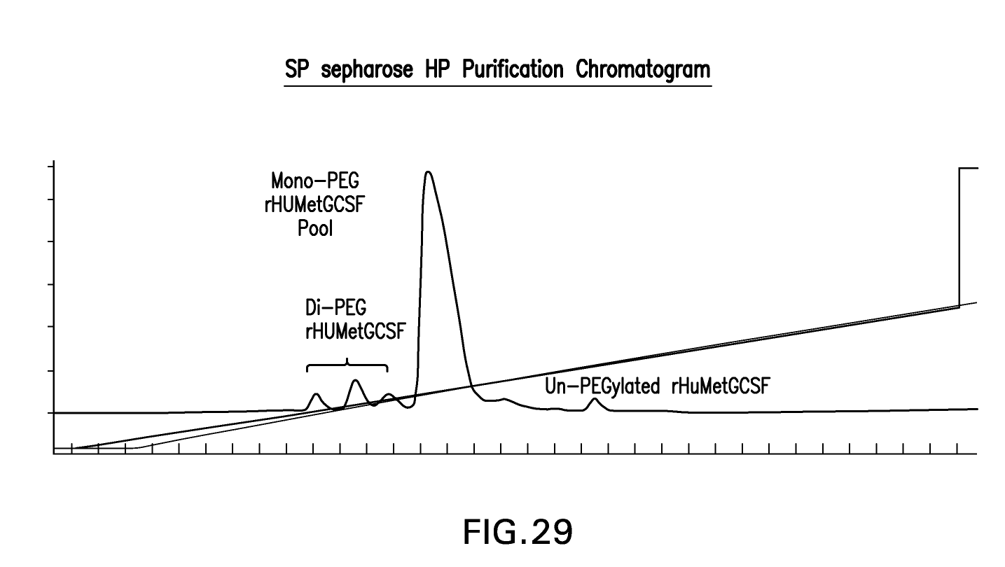

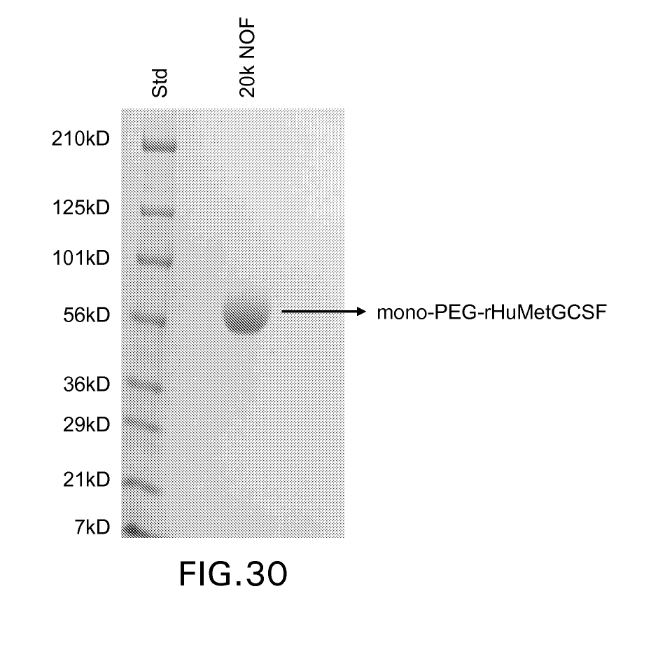

The loaded column was washed with two CV of 20 mM sodium acetate pH 4.0 to remove unbound material. The column was then washed with 8CV of 20 mM sodium acetate pH 4.0, 10 mM CHAPS, and 5 mM EDTA to remove endotoxin. The column was then washed with eight CV of 20 mM sodium acetate pH 4.0 to remove the CHAPS and EDTA. To elute the mono-PEG-rHuMetGCSF, a linear gradient of 15 CV from 0 to 500 mM NaCl in 20 mM sodium acetate pH 4.0 was performed and 5.0 mL fractions were collected. FIG. 29 shows a chromatogram of the column chromatography. The first three small peaks in the chromatogram refer to di-PEG-rHuMetGCSF. The fourth single huge peak for mono-PEG-rHuMetGCSF. An aliquot of the fourth peak was electrophoresed on and SDS-PAGE Gel. FIG. 30 shows an SDS polyacrylamide gel stained with Coomassie blue showing that the fourth peak contained mono-PEGylated rHuMetGCSF.

Based on the SDS-PAGE gel and chromatogram, the fractions containing the mono-PEG rHuMetGCSF were pooled and filtered through a 0.2 μm filter. The filtrate containing the mono-PEG rHuMetGCSF was stored at 4° C. To prepare the mono-PEG rHuMetGCSF formulation, the buffer-exchanged filtrate containing the mono-PEG rHuMetGCSF was buffer-exchanged into a solution of 10 mM Sodium acetate pH 4.0, 5% sorbitol, and 0.004% polysorbate 20. The mono-PEG rHuMetGCSF formulation can be stored at 4° C.

The source of the reagents used were as follows: sodium chloride (J.T. Baker Cat. #3624-07 Cas.No. 7647-14-5); sodium acetate, anhydrous (J.T. Baker Cat #3473-05 Cas No. 127-09-3); CHAPS (J.T. Baker Cat. #4145-02 Cas No. 75621-03-3); EDTA, disodium salt, dihydrate crystal (J.T. Baker Cat. #8993-01 Cas No. 6381-92-6); sorbitol (J.T. Baker Cat #V045-07 Cas No. 50-70-4); polysorbate 20, N.F. (J.T. Baker Cat #4116-04 Cas No. 9005-64-5).

Table of Sequences

| | | |
|---|---|---|
| SEQ ID | | |
| NO: | Description | Sequence |

| | | |
|---|---|---|
| 1 | Primer MAM281 | CTCGAGGAGTCCTCTTATGACACCATTAGGA |
| | | CCTGCTTCCTCC |
| 2 | Primer MAM227 | CTCGAGGAGTCCTCTT |
| | | ACACCATTAGGACCTGCTTC |
| 3 | Primer MAM228 | GAGCTCGGCCGGCCTTATTATGGTTGAGCC |
| 4 | Primer MAM304 | AAAAAAGAATTCCGAAAAATGAGCACCCTGA |
| | | CATTGC |
| 5 | Primer MAM305 | AAAAAAAGGCCTCTTAACCAAAGAACCTCCACC |
| | | TTCGTCCGTACGAGCACAGCCGGTGATAGAA |
| | | GTG |
| | | GGTTTCATGTCCTCCGGAAATCACTTCTATCA |
| | | CCGGCTGTGCTCGTACGGACGAAGGTGGAGG |
| | | TTCTTTGGTTAAGAGGATG |
| 6 | GCSF, GenBank | magpatqspmklmalqlllwhsalwtvqeaTPLGPASSLPQSF |
| | NP_757373, | LLKCLEQVRKIQGDGAALQEKLCATYKLCHPEE |
| | precursor molecule | LVLLGHSLGIPWAPLSSCPSQALQLAGCLSQLHS |
| | | GLFLYQGLLQALEGISPELGPTLDTLQLDVADFA |
| | | TTIWQQMEELGMAPALQPTQGAMPAFASAFQR |
| | | RAGGVLVASHLQSFLEVSYRVLRHLAQP |
| 7 | DNA encoding | ACACCATTAGGACCTGCTTCCTCCTTGCCCCA |
| | mature GCSF | ATCATTCCTTCTGAAGTGTTTGGAACAAGTGC |
| | synthesized from | GAAAGATACAAGGTGATGGAGCTGCCCTTCA |
| | DNA2.0 | AGAAAAACTATGTGCAACCTACAAGCTGTGTC |
| | | ATCCTGAGGAATTGGTACTGCTGGGACATTCA |
| | | TTAGGTATTCCATGGGCCCCATTGTCTTCTTGT |
| | | CCAAGTCAAGCTTTACAACTAGCCGGTTGTTT |
| | | GTCACAGTTACATTCTGGTTTGTTCCTATACCA |
| | | AGGATTACTGCAAGCACTGGAAGGAATTTCA |
| | | CCTGAATTGGGTCCTACATTAGATACTTTACA |
| | | ATTGGATGTTGCTGATTTCGCTACTACTATTTG |
| | | GCAACAAATGGAAGAGCTAGGTATGGCTCCA |
| | | GCACTTCAACCTACGCAAGGAGCAATGCCAG |
| | | CTTTTGCCTCTGCCTTTCAGCGTCGAGCTGGC |
| | | GGGGTGTTAGTTGCATCTCACTTACAGTCTTT |
| | | CCTGGAAGTTAGTTACCGTGTCCTAAGACATT |
| | | TGGCTCAACCATAATAAGGCCGGCC |
| 8 | Mature GCSF | TPLGPASSLPQSFLLKCLEQVRKIQGDGAALQEK |
| | | LCATYKLCHPEELVLLGHSLGIPWAPLSSCPSQA |
| | | LQLAGCLSQLHSGLFLYQGLLQALEGISPELGPT |
| | | LDTLQLDVADFATTIWQQMEELGMAPALQPTQ |
| | | GAMPAFASAFQRRAGGVLVASHLQSFLEVSYR |
| | | VLRHLAQP |
| 9 | P. pastoris CLP1 | ATGAGCACCCTGACATTGCTGGCTGTGCTGTT |
| | | GTCGCTTCAAAATTCAGCTCTTGCTGCTCAAG |
| | | CTGAAACTGCATCCCTATATCACCAATGTGGT |
| | | GGTGCAAACTGGGAGGGAGCAACCCAGTGTA |
| | | TTTCTGGTGCCTACTGTCAATCGCAGAACCCA |
| | | TACTACTATCAATGTGTTGCTACTTCTTGGGGT |
| | | TACTACACTAACACCTCAATCTCTTCGACGGC |
| | | CACCCTTCCTTCTTCTTCTACTACTGTCTCTCC |
| | | AACCAGCAGTGTGGTGCCCACTGGCTTGGTGT |
| | | CCCCATTGTATGGGCAATGTGGGGGACAGAA |
| | | TTGGAATGGAGCCACATCTTGTGCTCAGGGAA |
| | | GCTACTGCAAGTATATGAACAATTATTACTTC |
| | | CAATGTGTTCCTGAAGCTGATGGAAACCCTGC |
| | | AGAAATTAGCACTTTTTCCGAGAATGGAGAG |
| | | ATTATCGTTACTGCAATCGAAGCTCCTACATG |
| | | GGCTCAATGTGGTGGTCATGGCTACTACGGCC |
| | | CAACTAAATGTCAAGTGGGAACATCATGCCGT |
| | | GAATTAAACGCTTGGTATTATCAGTGTATCCC |
| | | AGACGATCACACCGATGCCTCTACTACCACTT |
| | | TGGATCCTACTTCCAGTTTTGTGAGTACGACA |
| | | TCATTATCGACTCTTCCAGCTTCTTCAGAAAC |
| | | GACAATTGTAACTCCTACCTCAATTGCTGCTG |
| | | AGCAAGTACCTCTTTGGGGACAATGTGGAGG |
| | | AATTGGTTACACTGGCTCTACGATTTGTGAGC |
| | | AGGGATCGTGTGTTTACTTGAACGATTGGTAC |
| | | TATCAGTGTCTAATAAGTGATCAAGGTACAGC |
| | | ATCAACTGCCAGTGCAACGACTAGTATAACTT |
| | | CCTTCAATGTTTCATCGTCGTCAGAAACGACG |
| | | GTAATAGCCCCTACCTCAATTTCTACTGAGGA |
| | | TGTCCCACTTTGGGGCCAATGTGGAGGAATTG |
| | | GATATACCGGTTCGACCACTTGTAGCCAGGGA |
| | | TCATGCATTTACTTAAATGACTGGTATTTTCA |
| | | ATGTTTACCAGAGGAGGAAACGACTTCATCA |
| | | ACTTCGTCATCTTCCTCATCTTCCTCATCTTCC |
| | | ACATCTTCCGCATCTTCCACATCTTCCACATC |
| | | ATCCACATCCTCCACATCCTCCACATCTTCCTC |
| | | AACAAGTAGCTCATCCATTCCGACTTCTACAA |
| | | GCTCATCGGGAGACTTTGAGACAATCCCCAAC |
| | | GGTTTCTCGGGAACTGGAAGAACCACGAGAT |
| | | ATTGGGATTGTTGTAAGCCAAGCTGCTCATGG |
| | | CCTGGGAAATCCAACAGCGTAACAGGACCAG |
| | | TGAGATCTTGTGGTGTCTCTGGCAACGTCCTG |
| | | GACGCCAACGCCCAAAGTGGATGTATTGGTG |
| | | GTGAAGCTTTCACTTGTGATGAGCAACAACCT |
| | | TGGTCCATCAACGACGACCTAGCCTATGGTTT |
| | | TGCCGCAGCAAGCCTAGCTGGTGGATCTGAG |
| | | GATTCCTCTTGCTGCACCTGTATGAAGCTGAC |
| | | ATTCACCTCATCTTCCATTGCTGGAAAGACAA |
| | | TGATCGTTCAACTGACCAATACTGGAGCTGAT |
| | | CTTGGATCGAATCACTTTGACATTGCTCTTCCT |
| | | GGTGGAGGGCTTGGAATCTTCACCGAAGGAT |
| | | GCTCTAGTCAATTTGGAAGCGGTTACCAATGG |
| | | GGTAACCAGTATGGTGGTATCTCTTCGCTTGC |
| | | TGAGTGTGATGGCCTACCATCAGAACTGCAGC |
| | | CAGGCTGTCAGTTTAGATTTGGCTGGTTTGAG |
| | | AACGCTGATAACCCTTCAGTGGAGTTTGAACA |
| | | GGTTTCATGTCCTCCGGAAATCACTTCTATCA |
| | | CCGGCTGTGCTCGTACGGACGAATAA |
| 10 | Clp1p | MSTLTLLAVLLSLQNSALAAQAETASLYHQCGG |
| | | ANWEGATQCISGAYCQSQNPYYYQCVATSWG |
| | | YYTNTSISSTATLPSSSTTVSPTSSVVPTGLVSPL |
| | | YGQCGGQNWNGATSCAQGSYCKYMNNYYFQC |
| | | VPEADGNPAEISTFSENGEIIVTAIEAPTWAQCGG |
| | | HGYYGPTKCQVGTSCRELNAWYYQCIPDDHTD |
| | | ASTTTLDPTSSFVSTTSLSTLPASSETTIVTPTSIA |
| | | AEQVPLWGQCGGIGYTGSTICEQGSCVYLNDW |
| | | YYQCLISDQGTASTASATTSITSFNVSSSSETTVI |
| | | APTSISTEDVPLWGQCGGIGYTGSTTCSQGSCIY |
| | | LNDWYFQCLPEEETTSSTSSSSSSSSSSTSSASSTS |
| | | STSSTSSTSSTSSSTSSSSIPTSTSSSGDFETIPNGFS |
| | | GTGRTTRYWDCCKPSCSWPGKSNSVTGPVRSC |
| | | GVSGNVLDANAQSGCIGGEAFTCDEQQPWSIND |
| | | DLAYGFAAASLAGGSEDSSCCTCMKLTFTSSSIA |
| | | GKTMIVQLTNTGADLGSNHFDIALPGGGLGIFTE |
| | | GCSSQFGSGYQWGNQYGGISSLAECDGLPSELQ |
| | | PGCQFRFGWFENADNPSVEFEQVSCPPEITSITG |
| | | CARTDE |
| 11 | CLP1- | ATGAGCACCCTGACATTGCTGGCTGTGCTGTT |
| | rHuMetGCSF gene | GTCGCTTCAAAATTCAGCTCTTGCTGCTCAAG |
| | fusion | CTGAAACTGCATCCCTATATCACCAATGTGGT |
| | | GGTGCAAACTGGGAGGGAGCAACCCAGTGTA |
| | | TTTCTGGTGCCTACTGTCAATCGCAGAACCCA |
| | | TACTACTATCAATGTGTTGCTACTTCTTGGGGT |
| | | TACTACACTAACACCTCAATCTCTTCGACGGC |
| | | CACCCTTCCTTCTTCTTCTACTACTGTCTCTCC |
| | | AACCAGCAGTGTGGTGCCCACTGGCTTGGTGT |
| | | CCCCATTGTATGGGCAATGTGGGGGACAGAA |
| | | TTGGAATGGAGCCACATCTTGTGCTCAGGGAA |
| | | GCTACTGCAAGTATATGAACAATTATTACTTC |
| | | CAATGTGTTCCTGAAGCTGATGGAAACCCTGC |
| | | AGAAATTAGCACTTTTTCCGAGAATGGAGAG |
| | | ATTATCGTTACTGCAATCGAAGCTCCTACATG |
| | | GGCTCAATGTGGTGGTCATGGCTACTACGGCC |
| | | CAACTAAATGTCAAGTGGGAACATCATGCCGT |
| | | GAATTAAACGCTTGGTATTATCAGTGTATCCC |
| | | AGACGATCACACCGATGCCTCTACTACCACTT |
| | | TGGATCCTACTTCCAGTTTTGTGAGTACGACA |
| | | TCATTATCGACTCTTCCAGCTTCTTCAGAAAC |
| | | GACAATTGTAACTCCTACCTCAATTGCTGCTG |
| | | AGCAAGTACCTCTTTGGGGACAATGTGGAGG |
| | | AATTGGTTACACTGGCTCTACGATTTGTGAGC |
| | | AGGGATCGTGTGTTTACTTGAACGATTGGTAC |
| | | TATCAGTGTCTAATAAGTGATCAAGGTACAGC |
| | | ATCAACTGCCAGTGCAACGACTAGTATAACTT |
| | | CCTTCAATGTTTCATCGTCGTCAGAAACGACG |
| | | GTAATAGCCCCTACCTCAATTTCTACTGAGGA |
| | | TGTCCCACTTTGGGGCCAATGTGGAGGAATTG |
| | | GATATACCGGTTCGACCACTTGTAGCCAGGGA |
| | | TCATGCATTTACTTAAATGACTGGTATTTTCA |
| | | ATGTTTACCAGAGGAGGAAACGACTTCATCA |
| | | ACTTCGTCATCTTCCTCATCTTCCTCATCTTCC |
| | | ACATCTTCCGCATCTTCCACATCTTCCACATC |
| | | ATCCACATCCTCCACATCCTCCACATCTTCCTC |
| | | AACAAGTAGCTCATCCATTCCGACTTCTACAA |
| | | GCTCATCGGGAGACTTTGAGACAATCCCCAAC |
| | | GGTTTCTCGGGAACTGGAAGAACCACGAGAT |
| | | ATTGGGATTGTTGTAAGCCAAGCTGCTCATGG |
| | | CCTGGGAAATCCAACAGCGTAACAGGACCAG |
| | | TGAGATCTTGTGGTGTCTCTGGCAACGTCCTG |
| | | GACGCCAACGCCCAAAGTGGATGTATTGGTG |
| | | GTGAAGCTTTCACTTGTGATGAGCAACAACCT |
| | | TGGTCCATCAACGACGACCTAGCCTATGGTTT |
| | | TGCCGCAGCAAGCCTAGCTGGTGGATCTGAG |
| | | GATTCCTCTTGCTGCACCTGTATGAAGCTGAC |
| | | ATTCACCTCATCTTCCATTGCTGGAAAGACAA |
| | | TGATCGTTCAACTGACCAATACTGGAGCTGAT |
| | | CTTGGATCGAATCACTTTGACATTGCTCTTCCT |
| | | GGTGGAGGGCTTGGAATCTTCACCGAAGGAT |
| | | GCTCTAGTCAATTTGGAAGCGGTTACCAATGG |
| | | GGTAACCAGTATGGTGGTATCTCTTCGCTTGC |
| | | TGAGTGTGATGGCCTACCATCAGAACTGCAGC |
| | | CAGGCTGTCAGTTTAGATTTGGCTGGTTTGAG |
| | | AACGCTGATAACCCTTCAGTGGAGTTTGAACA |
| | | GGTTTCATGTCCTCCGGAAATCACTTCTATCA |
| | | CCGGCTGTGCTCGTACGGACGAAGGTGGAGG |
| | | TTCTTTGGTTAAGAGGATGacaccattaggacctgcttcct |
| | | ccttgccccaatcattccttctgaagtgtttggaacaagtgcgaaagatacaa |
| | | ggtgatggagctgcccttcaagaaaaactatgtgcaacctacaagctgtgtc |
| | | atcctgaggaattggtactgctgggacattcattaggtattccatgggccccat |
| | | tgtcttcttgtccaagtcaagctttacaactagccggttgtttgtcacagttacat |
| | | tctggtttgttcctataccaaggattactgcaagcactggaaggaatttcacct |
| | | gaattgggtcctacattagatactttacaattggatgttgctgatttcgctactac |
| | | tatttggcaacaaatggaagagctaggtatggctccagcacttcaacctacg |
| | | caaggagcaatgccagcttttgcctctgcctttcagcgtcgagctggcgggg |
| | | tgttagttgcatctcacttacagtctttcctggaagttagttaccgtgtcctaaga |
| | | catttggctcaaccaTAATAA |
| 12 | Clp1p- | MSTLTLLAVLLSLQNSALAAQAETASLYHQCGG |
| | rHuMetGCSF | ANWEGATQCISGAYCQSQNPYYYQCVATSWG |
| | fusion protein | YYTNTSISSTATLPSSSTTVSPTSSVVPTGLVSPL |
| | | YGQCGGQNWNGATSCAQGSYCKYMNNYYFQC |
| | | VPEADGNPAEISTFSENGEIIVTAIEAPTWAQCGG |
| | | HGYYGPTKCQVGTSCRELNAWYYQCIPDDHTD |
| | | ASTTTLDPTSSFVSTTSLSTLPASSETTIVTPTSIA |
| | | AEQVPLWGQCGGIGYTGSTICEQGSCVYLNDW |
| | | YYQCLISDQGTASTASATTSITSFNVSSSSETTVI |
| | | APTSISTEDVPLWGQCGGIGYTGSTTCSQGSCIY |
| | | LNDWYFQCLPEEETTSSTSSSSSSSSSSTSSASSTS |
| | | STSSTSSTSSTSSSTSSSSIPTSTSSSGDFETIPNGFS |
| | | GTGRTTRYWDCCKPSCSWPGKSNSVTGPVRSC |
| | | GVSGNVLDANAQSGCIGGEAFTCDEQQPWSIND |
| | | DLAYGFAAASLAGGSEDSSCCTCMKLTFTSSSIA |
| | | GKTMIVQLTNTGADLGSNHFDIALPGGGLGIFTE |
| | | GCSSQFGSGYQWGNQYGGISSLAECDGLPSELQ |
| | | PGCQFRFGWFENADNPSVEFEQVSCPPEITSITG |
| | | CARTDEgggslvkrMTPLGPASSLPQSFLLKCLEQV |
| | | RKIQGDGAALQEKLCATYKLCHPEELVLLGHSL |
| | | GIPWAPLSSCPSQALQLAGCLSQLHSGLFLYQGL |
| | | LQALEGISPELGPTLDTLQLDVADFATTIWQQME |
| | | ELGMAPALQPTQGAMPAFASAFQRRAGGVLVA |
| | | SHLQSFLEVSYRVLRHLAQP |
| 13 | Secreted Clp1p | AQAETASLYHQCGGANWEGATQCISGAYCQSQ |
| | fusion protein | NPYYYQCVATSWGYYTNTSISSTATLPSSSTTVS |
| | | PTSSVVPTGLVSPLYGQCGGQNWNGATSCAQG |
| | | SYCKYMNNYYFQCVPEADGNPAEISTFSENGEII |
| | | VTAIEAPTWAQCGGHGYYGPTKCQVGTSCREL |
| | | NAWYYQCIPDDHTDASTTTLDPTSSFVSTTSLST |
| | | LPASSETTIVTPTSIAAEQVPLWGQCGGIGYTGST |
| | | ICEQGSCVYLNDWYYQCLISDQGTASTASATTSI |
| | | TSFNVSSSSETTVIAPTSISTEDVPLWGQCGGIGY |
| | | TGSTTCSQGSCIYLNDWYFQCLPEEETTSSTSSSS |
| | | SSSSSSTSSASSTSSTSSTSSTSSTSSSTSSSSIPTST |
| | | SSSGDFETIPNGFSGTGRTTRYWDCCKPSCSWP |
| | | GKSNSVTGPVRSCGVSGNVLDANAQSGCIGGEA |
| | | FTCDEQQPWSINDDLAYGFAAASLAGGSEDSSC |
| | | CTCMKLTFTSSSIAGKTMIVQLTNTGADLGSNHF |
| | | DIALPGGGLGIFTEGCSSQFGSGYQWGNQYGGIS |
| | | SLAECDGLPSELQPGCQFRFGWFENADNPSVEF |
| | | EQVSCPPEITSITGCARTDEGGGSLVKR |
| 14 | Secreted | MTPLGPASSLPQSFLLKCLEQVRKIQGDGAALQE |
| | rHuMetGCSF | KLCATYKLCHPEELVLLGHSLGIPWAPLSSCPSQ |
| | protein | ALQLAGCLSQLHSGLFLYQGLLQALEGISPELGP |
| | | TLDTLQLDVADFATTIWQQMEELGMAPALQPT |
| | | QGAMPAFASAFQRRAGGVLVASHLQSFLEVSY |
| | | RVLRHLAQP |
| 15 | Kex2 linker | GGGSLVKR |
| 16 | Kex2 linker | GGTGGAGGTTCTTTGGTTAAGAGG |
| 17 | VPS10-1 region | aaactaagtgggccagattatataaatatggatcaacatgaagccttgaaag |
| | (including upstream | atttcaaggacaggcttaggaattacgaaaaagtttacgagactattgacgac |
| | knock-out | caggaggaagaggagaacgaacggtacaatattcagtatctgaagataatc |
| | fragment, promoter, | aacgcaggaaagaagatagtcagttataacataaatgggtatttatcgtccca |
| | open reading frame, | caccgttttttatctcctgaatttcaatcttgcagaacgtcaaatatggttgacga |
| | and downstream | cgaatggagagacagagtataaccttcaaaataggattggaggtgattccaa |
| | knock-out | attaagcaatgagggatggaaatttgccaaagcattgcccaagtttatagcac |
| | fragment) | agaaaagaaaagagtttcaacttagacagttgaccaaacactatatcgagac |
| | | tcaaacgcccattgaagacgtaccgttggaggagcacaccaagccagtcaa |
| | | atattctgatctgcatttccatgtttggtcatcggctttaaagagatctactcaat |
| | | caacaacattttttccatcggaaaattactctctgaagcaattcagaacgttga |
| | | atgatctctgttgcggatcactggatggtttgactgaacaagagttcaaaagta |
| | | aatacaaagaagaataccagaattctcagactgataaactgagtttcagtttcc |
| | | ctggtatcggtggggagtcttatttggacgtgatcaaccgtttgagaccacta |
| | | atagttgaactagaaaggttgccagaacatgtcctggtcattacccaccgggt |
| | | catagtaaggattttactaggatatttcatgaatttggatagaaatctgttgaca |
| | | gatttggaaattttgcatgggtatgtttattgtattgagccgaaaccttatggttt |
| | | agacttaaagatctggcagtatgatgaggcggacaacgagtttaatgaagtt |
| | | gataagctggaattcatgaaaagaagaagaaaatcgatcaacgtcaacacg |
| | | acagatttcagaatgcagttaaacaaagagttgcaacaggacgctctcaata |
| | | atagtcctggtaataatagtccgggcgtatcatctctatcttcatactcgtcgtc |
| | | ctcttccctttccgctgacgggagcgagggagaaacattaataccacaagta |
| | | tcccaggcggagagctacaactttgaatttaactctctttcatcatcagtttcat |
| | | cgttgaaaaggacgacatcttcttcccaacatttgagctccaatcctagttgtct |
| | | gagcatgcataatgcctcattggacgagaatgacgacgaacatttaatagac |
| | | ccggcttctacagacgacaagctaaacatggtattacaggacaaaacgcta |
| | | attaaaagctcaaaagtttactacttgacgaggccgaaggctagacaatcc |
| | | acagttaattttgatactgtactttataacgagtaacatacatatcttatgtaatca |
| | | tctatgtcacgtcacgtgcgcgcgacattattccgagaacttgcgccctgcta |
| | | gctccactgtcagagtgataacttccccaaaataggatccaactgtttccaatt |
| | | gcttttggaaatgtggattgaaagaaacctcatagcgtctatattactattttca |
| | | acttcagcttatgcggcattcaaacccaggatagttaaaaaggaatttgatga |
| | | ccttttgaatccaatatactttaacgattcatcgacagtactaggtctagtagat |
| | | cagacgctgttaatttccaacgatgatggaaaatcatggactaacttgcagga |
| | | ggttattacacctggggaaattgatccgctgacaattgtaaacattgaattcaa |
| | | tccatccgcatctaaggcttttgtattcactgctagtaagcactaccttactttag |
| | | acaaaggatccacctggaaagaatttcaaattcctcttgaaaaatatggtaac |
| | | agaatagcctacgacgttgagtttaattttgttaacgaagaacatgcaatcata |
| | | agaacaaggtcttgcaaacgtcgttttgattgtaaggatgagtatttttattcgtt |
| | | agatgacttgcaaagcgttgacaagatcaccatttctgacgaaattgtcaattg |
| | | ccagttttcacaatcttccactagctcagattcccgcaaaaacgatgccatca |
| | | cttgcgtaacgcgtaaactggattccaaccgacacttcttggagtcgaacgtt |
| | | ctgacaaccttgaactttttcaaggatgttactagcttgcccgccagtgatcca |
| | | ttaactaagatgcttatcaaggatatacgtgttgttcaaaattacattgtattgttt |
| | | gtcagttcggatagatacaacaaatattcacccactcttcttttcatttccaaag |
| | | atggaaatacgtttaaggaagccagtttaccagattctgaaggtacatcaccg |
| | | tcggtgcactttttgaaaagtcctaatcccaatttgataagagcaattcggcta |
| | | gggaaaaagaactcactagatggtggtggcttttattcagaagttctacaatct |
| | | gactctacagggttacactttcacgttcttctggaccacttagaagcaaatttg |
| | | ctttcgtactatcaaatagagaacttagcgaaccttgaaggaatctggattgcc |
| | | aaccaaatcgacacttccagcaagtttggctcaaaatccgttataacatttgat |
| | | gcaggtttaacgtggtctcctgtgacagtagatgaagacgaagataaaagttt |
| | | gcacatcattgcgtttgctggtgaaaatagcctttatgagtccaagtttccggtt |
| | | tcgactccaggaattgccttgaggatagggcttattggcgatagtagtgatgc |
| | | acttgatattggcagctataggacatttttaaccagagatgcagggctaacat |
| | | ggtctcaagtttttgataatgtctctgtttgcggctttggaaactatggaaacatc |
| | | atattatgctgttcgtatgatccactacttcgatctgagcctttgaaatttcgttat |
| | | tctttggatcaaggtcttaactgggaaagtattgatttaggcttcaacggagtc |
| | | gctgttggcgttttgaacaatatagacaatagcagtcctcaattccttgtgatga |
| | | cgattgccacggatggtaagtcttcaaaggctcagcatttcttgtattcagttg |
| | | atttttctgatgcgtatgagaagaaaatatgtgatgttacaaaagacgaattatt |
| | | tgaagaatggacgggaagaatagatccggtgacgaagctgcctatttgtgtt |
| | | aacggtcacaaggaaaaattcagaagacggaaggctgacgctgaatgcttc |
| | | tctggtgaactttttcaagacctaactccaattgaagagccatgtgattgtgatc |
| | | cggatattgattacgaatgttcgcttggatttgagttcgatgcagagtctaacc |
| | | gatgtgagccaaatttgtcaatcctgtccagtcactattgtgttgggaaaaactt |
| | | aaagagaaaagtgaaagtagatagaaagtcgaaagttgcaggcacaaaat |
| | | gtaaaaaggatgtcaaacttaaggataattctttcactttagactgttccaaaac |
| | | atctgaaccagatctcagcgagcaaagaattgttagtaccaccataagctttg |
| | | aaggttctccagtacaatacatttatttgaaacaggggaccaacacaaccctt |
| | | cttgacgaaacagtcattttaagaacatcactacgaactgtgtacgtgtctcat |
| | | aacgggggaacaacttttgatagagttagtatcgaagatgatgtgtcatttatt |
| | | gacatctatacaaaccattactttccagataatgtttatttgatcactgatacaga |
| | | tgagctgtacgtttcggataatagagctatactttccagaaagttgacatgcct |
| | | tcaagagctggtttggagcttggagttcgagctctaacctttcataagagtga |
| | | ccctaacaagtttatttggttcggtgagaaagattgtaactctatttttgacaga |
| | | agttgtcaaacacaagcttatattacggaagacaacggcttatctttcaagcct |
| | | cttttggaaaatgttagatcatgttactttgttggaacaacttttgattccaagct |
| | | gtatgattttgacccgaacttaatcttttgcgagcagagagttccaaatcaacg |
| | | tttcttgaaacttgtagccagtaaggactatttctatgatgacaaagaagagct |
| | | gtatcctaagattattggaattgctactaccatgagctttgttatcgtagcgact |
| | | atcaacgaagacaatagatcattgaaggcgtttataaccgcggatgggtcta |
| | | cttttgcggagcaattgtttcctgcagatctggattttggaagagaagtagcgt |
| | | acacagttattgacaattgggaatcaaaaacacccaatttctttttccatttgac |
| | | aacttctgaagataaagatttggaatttggagctttactgaaatcaaactacaat |
| | | ggaacaacctatacgcttgctgccaacaatgtcaatagaaacgatagaggtt |
| | | acgttgactatgaaatcgttctaaacttaaacggcattgctctcatcaatacagt |
| | | tattaactcgaaggaacttgaatccgagcagtcccttgaaactgctaaaaaac |
| | | tgaaaactcaaataacgtacaacgacgggtctgaatgggtgtatctgaaacc |
| | | gccaaccattgattcagaaaagaacaagttttcgtgcgtcaaagataagttga |
| | | gcttggaaaaatgctcattgaacctcaagggtgccactgatcggccagaca |
| | | gcagagactccatttcttctggttctgctgttggtctactttttggagtaggtaac |
| | | gttggggaatacctgaaccaagattcatcaggtctagcattgtatttttcgaag |
| | | gatgcgggcatctcttggaaggagattgccaaaggagattatatgtgggaat |
| | | ttggagatcaaggaacaatcctcgtaattgttgagttcaagaagaaggttgac |
| | | actttgaaatactcattggatgaaggagaaacgtggttcgactacaagtttgc |
| | | aaatgaaaaaacatatgttttggacctagcaactgtgccttcagatacttcacg |
| | | gaagttcatcatcctcgccaacagaggcgaggagggagatcatgaaactgt |
| | | tgttcacacaatagacttcagtaaggttcaccagcgtcaatgtttattgaattta |
| | | caagatagtaacgctggtgatgatttcgaatattggagtccgaagaacccaa |
| | | gcgctgttgacgggtgtatgctagggcatgaagagtcttacctaaaaaggatt |
| | | gcatcccactcggattgttttattgggaacgcacccctatcagagaaatacaa |
| | | agtgattaagaactgcgcttgcacaaggagagattacgaatgtgattacaatt |
| | | ttgctcttgccaatgatggaacttgtaaattggtggaaggagagtctcctttgg |
| | | attactctgaagtttgtagaagggatccaacttccattgaatattttttgcctact |
| | | gggtacagaaaggtgggattgagtacttgtgaaggcggactagaactggat |
| | | aattggaatcccgttccatgtccaggaaaaaccagagaattcaatagaaaat |
| | | acggcaccggcgccaccggatacaagattgtggtcatagtagcagtgccttt |
| | | attggttctcttgagcgccacttggttcctatatgagaaaggaataaaaagga |
| | | atggaggttttgccagatttggagttattcgattaggcgaagatgacgacgat |
| | | gacttgcaaatgattgaggagaataatactgacaaagtagtcaatgttgtagt |
| | | gaaaggcctcattcatgcattcagagcagtttttgtgagctatttatttttccgca |
| | | aacgtgcggccaagatgtttggtggatcgtccttttcacacagacacatattg |
| | | cctcaagatgaggatgctcaagcctttttagccagcgacttggagtcagaga |
| | | gtggagagcttttccgatatgcaagcgacgatgacgatgcccgagagattg |
| | | acagcgtgatcgagggaggaattgatgtcgaagacgacgacgaggagaat |
| | | atcaattttgattcccggtagatagctcacccacggtcacacacacaaacaca |
| | | catacacattaacacacagagttattagttaacagagaaaactctaacaaagt |
| | | atttattttcgttacgtaatccgacttttctttttaccgttttctattgctcctctcattt |
| | | gcccctaaaagttgctcctcattactaaaatcaccacaccatgctcgaatatg |
| | | atgttactaaatgcaaattgtagtcgtgcctcttgtggtaatactatagggaata |
| | | tctctcgattactcgattctggttaattttttctttttttataggggaagtttttttttct |
| | | tcccctttctctccagtttatttatttactaagaaaatccaacagataccaaccac |
| | | ccaaaaagatcctaaacagcctgtttttgaggagtttttcagcagctaagcttc |
| | | atcagttttttaatacttaatttattgcccttcactttgtttcttgtggcttttaaggct |
| | | ctccggaacagcggtttcaaaatcaaatctcagttatttgtttgctccgctttgt |
| | | cagttcaaagatcatggtttccgaaaacaagaatcaatcttcgattttgatgga |
| | | caactccaagaagctctctccgaagcccattttgaataacaagaatgaaccg |
| | | tttggcatcggcgtcgatggacttcaacatcctcaaccgactttatgccgcac |
| | | agaatcggaactcttgttcaacttgagccaagtcaataaatcccaaataacttt |
| | | ggacggtgcagttactccacctgctgatggtaatgggaatgaagcaaaaag |
| | | agcaaatctcatctcttttgatgttccatcgtctcaagtgaaacatagagggtct |
| | | attagtgcaaggccctcggcagtgaatgtgtcccaaattaccggggccatt |
| | | ctcaatccggatcttctagaaatccctacgatcaaacacagtcacctccacct |
| | | agcacttacgcctccaggcagaactccacccatggaaataatatcgatagct |
| | | tgcaatatttggcaacaagagatcttagtgctttaaggctggaaagagatgctt |
| | | ccgcacgagaagctacctcttctgcagtgtccactcctgttcagttcgatgtac |
| | | ccaaacaacatcatctccttcatttagaacaagacccgacaaggcccatccc |
| | | tattgccgacaaaaag |
| 18 | PEP4 region | atttgagtcacctgctttagggctggaagatatttggttactagattttagtacaa |
| | (including upstream | actcttgctttgtcaatgacattaaaataggcaagaatcgcaaaactcaaatat |
| | knock-out | ttcatggagatgagatatgcttgttcaaagatgcccagaaaaaagagcaact |
| | fragment, promoter, | cgtttatagggttcatattgatgatggaacaggccttttccagggaggtgaaa |
| | open reading frame, | gaacccaagccaattctgatgacattctggatattgatgaggttgatgaaaag |
| | and downstream | ttaagagaactattgacaagagcctcaaggaaacggcatatcacccctgcat |
| | knock-out | tggaaactcctgataaacgtgtaaaaagagcttatttgaacagtattactgata |
| | fragment) | actcttgatggaccttaaagatgtataatagtagacagaattcataatggtgag |
| | | attaggtaatcgtccggaataggaatagtggtttggggcgattaatcgcacct |
| | | gccttatatggtaagtaccttgaccgataaggtggcaactatttagaacaaag |
| | | caagccacctttctttatctgtaactctgtcgaagcaagcatctttactagagaa |
| | | catctaaaccattttacattctagagttccatttctcaattactgataatcaattta |
| | | aagatgatatttgacggtactacgatgtcaattgccattggtttgctctctactct |
| | | aggtattggtgctgaagccaaagttcattctgctaagatacacaagcatccag |
| | | tctcagaaactttaaaagaggccaattttgggcagtatgtctctgctctggaac |
| | | ataaatatgtttctctgttcaacgaacaaaatgctttgtccaagtcgaattttatg |
| | | tctcagcaagatggttttgccgttgaagcttcgcatgatgctccacttacaaac |
| | | tatcttaacgctcagtattttactgaggtatcattaggtacccctccacaatcgtt |
| | | caaggtgattcttgacacaggatcctccaatttatgggttcctagcaaagattg |
| | | tggatcattagcttgcttcttgcatgctaagtatgaccatgatgagtcttctactt |
| | | ataagaagaatggtagtagctttgaaattaggtatggatccggttccatggaa |
| | | gggtatgtttctcaggatgtgttgcaaattggggatttgaccattcccaaagtt |
| | | gattttgctgaggccacatcggagccggggttggccttcgcttttggcaaattt |
| | | gacggaattttggggcttgcttatgattcaatatcagtaaataagattgttcctc |
| | | caatttacaaggctttggaattagatctccttgacgaaccaaaatttgccttcta |
| | | cttgggggatacggacaaagatgaatccgatggcggtttggccacatttggt |
| | | ggtgtggacaaatctaagtatgaaggaaagatcacctggttgcctgtcagaa |
| | | gaaaggcttactgggaggtctcttttgatggtgtaggtttgggatccgaatatg |
| | | ctgaatgcaaaaaactggtgcagccatcgacactggaacctcattgattgct |
| | | ttgcccagtggcctagctgaaattctcaatgcagaaattggtgctaccaagg |
| | | gttggtctggtcaatacgctgtggactgtgacactagagactctttgccagac |
| | | ttaactttaaccttcgccggttacaactttaccattactccatatgactatactttg |
| | | gaggtttctgggtcatgtattagtgctttcacccccatggactttcctgaaccaa |
| | | taggtcctttggcaatcattggtgactcgttcttgagaaaatattactcagtttat |
| | | gacctaggcaaagatgcagtaggtttagccaagtctatttaggcaagaataa |
| | | aagttgctcagctgaacttatttggttacttatcaggtagtgaagatgtagaga |
| | | atatatgtttaggtatttttttttagtttttctcctataactcatcttcagtacgtgatt |
| | | gcttgtcagctaccttgacaggggcgcataagtgatatcgtgtactgctcaat |
| | | caagatttgcctgctccattgataagggtataagagacccacctgctcctcttt |
| | | aaaattctctcttaactgttgtgaaaatcatcttcgaagcaaattcgagtttaaat |
| | | ctatgcggttggtaactaaaggtatgtcatggtggtatatagtttttcattttacct |
| | | tttactaatcagttttacagaagaggaacgtctttctcaagatcgaaataggac |
| | | taaatactggagacgatggggtccttatttgggtgaaaggcagtgggctaca |
| | | gtaagggaagactattccgatgatggagatgcttggtctgcttttccttttgag |
| | | caatctcatttgagaacttatcgctggggagaggatggactagctggagtctc |
| | | agacaatcatcaactaatttgtttctcaatggcactgtggaatgagaatgatga |
| | | tattttgaaggagcgattatttggggtcactggagaggctgcaaatcatggag |
| | | aggatgttaaggagctttattattatcttgataatacaccttctcactcttatatga |
| | | aatacctttacaaatatccacaatcgaaatttccttacgaagaattgatttcaga |
| | | gaaccgtaaacgttccagattagaaagagagtacgagattactgactctgaa |
| | | gtactgaaggataacagatattttgatgtgatctttgaaatggcaaaggacgat |
| | | gaagatgagaatgaactttactttagaattaccgcttacaaccgaggtcccac |
| | | ccctgcccctttacatgtcgctccacaggtaacctttagaaatacctggtcctg |
| | | gggtatagatgaggaaaaggatcacgacaaacctatagcttgcaaggaata |
| | | ccaagacaacaactattctattcggttagatagtt |
| 19 | PRB1 region | actaaacgtgaatgaagatgcgaggaagggtgtggcagaatgaaggaaga |
| | (including upstream | attggtggcaatactgacctggctaaaacctattcaaactgggctaaatacag |
| | knock-out | gattcatgagtttcctgatctcaatatttttcagtcctccttgcccttgcaacgtttt |
| | fragment, promoter, | cttattcaatgcccaaactctcccatcgacgtcgcctcgaaactttctgaaaat |
| | open reading frame, | catgaccgtctgtttaatctcccgagactcttatctctatgaacattcactcgtt |
| | and downstream | agcttccctaaatgagtcaattagaaatcttttttaaaaagattcattctacgatt |
| | knock-out | cggcttcccgaaaaagaggcaagtgaattgctcaagaaacaattgactatga |
| | fragment) | acccaaaatctcctcatctcccaaaacttcaagtggatctacagaatcaatctg |
| | | aacaaaccataagcaaattcgtgcaagatcaacagttctttggtggcgactg |
| | | ggctcggttcgaaagccttattgtcagctatttaaaatttgttagaaactttgac |
| | | ccctggtcgatattgaaatccattgatctaatgattaacgttgttgacgagttgg |
| | | caagttctctcaacaaacaacagcattacaagtacctgtttgggactcttgttg |
| | | attatgtcattcttttgcatcctcttgtcaaattggttgataaaaaattgctaattat |
| | | caaaaagaggaacagctattatccaaggcttacgcagatgtctaccattttgc |
| | | agaaagctttcaacaatattagaaatcaaagagatccaaccggccagatatc |
| | | aagggaccaacaactggtcttattcttgcttggtataaagacttgctacatcta |
| | | ctttaacatcaatcatctcttgagatgcaatgatatcttctccaacatgaacgtg |
| | | ttgaacttggacgccaaaattatccctaagtcccagctaattcagtatagatttt |
| | | tgttgggaaagtttaacttcatacagaataacttcatgactgcatttgttcaattg |
| | | aactggtgtttgaacaacgcctacatcaataataccaatcatcggacgaaaa |
| | | atatggaattaatactaaaatatcttatcccctccagtcttatagttggtaagata |
| | | ccaaatttgaacatcctgaaccagctgctgtcatctcaagaggcacaccctct |
| | | gattgagctttatcgaccactgatttcaaccctcaaaaagggtaatgttttcga |
| | | attccacaaatacctgtttgataatgagtcatactttttaaagatgaacgttctcc |
| | | tgccgctacttcaacggttgcgtattttgctgttcagaaatctggtccgaaagc |
| | | tggcccttatagagccaccagtcaacaactctctgagattttcatccatcaaaa |
| | | cagcccttttcgtttccatttcacccaatcaaaacgcatactttcagaacaatta |
| | | ttcatacctgattgttaccaacgagtcccagatagacgactcctttgtggagaa |
| | | cctcatgatcagtctaatcgatcaaaacctaattaagggtaaactcgtcaacg |
| | | ataaccaccgaataattgtctccaaggccgatacattcccggagatccctac |
| | | gatttattcgactaagtttgccgtagactcgtcattcgattggctggaccaata |
| | | gacgtcctttttttttttttttttatcgtgtctgccgtttaatgtcacgcctcatgtttc |
| | | aagttacgataacttatcatgcagatactaaatagtcacatgacgaatgacga |
| | | ttttttgcgggttgctcagaggaatatgcctctgataagcgaggtaaatgtcga |
| | | gcataagccacttactgtataaatacccctttatcgccactttatcttttctccttg |
| | | tccgttatctacaacaccccagtaaaacattacaaacactctagtgttgttttac |
| | | tgtcccttttaactctcttcaaacaaatctccatattatttaaactatgcaattgcg |
| | | tcattccgttggattggctatcttatctgccatagcagtccaaggattgctaatt |
| | | cctaacattgagtcattacccagccagtttggtgctaatggtgacagtgaaca |
| | | aggtgtattagcccaccatggtaaacatcctaaagttgatatggctcaccatg |
| | | gaaagcatcctaaaatcgctaaggattccaagggacaccctaagctttgccc |
| | | tgaagctttgaagaagatgaaagaaggccacccttcggctccagtcattact |
| | | acccattccgcttctaaaaacttaatcccttactcttatattatagtcttcaagaa |
| | | gggtgtcacttcagaggatatcgacttccaccgtgaccttatctccactcttca |
| | | tgaagagtctgtgagcaaattaagagagtcagatccaaatcactcatttttcgt |
| | | ttctaatgagaatggcgaaacaggttacaccggtgacttctccgttggtgactt |
| | | gctcaagggttacaccggatacttcacggatgacactttagagcttatcagta |
| | | agcatccagcagttgctttcattgaaagggattcgagagtatttgccaccgatt |
| | | ttgaaactcaaaacggtgctccttggggtttggccagagtctctcacagaaa |
| | | gcctctttccctaggcagcttcaacaagtacttatatgatggagctggtggtga |
| | | aggtgttacttcctatgttatcgatacaggtatccacgtcactcacaaagaattc |
| | | cagggtagagcatcttggggtaagaccattccagctggagacgttgatgac |
| | | gatggaaacggtcacggaactcactgtgctggtaccattgcttctgaaagct |
| | | acggtgttgccaagaaggctaatgttgttgccatcaaggtcttgagatctaat |
| | | ggttctggttcgatgtcagatgttctgaagggtgttgagtatgccacccaatcc |
| | | cacttggatgctgttaaaaagggcaacaagaaatttaagggctctaccgcta |
| | | acatgtcactgggtggtggtaaatctcctgctttggaccttgcagtcaatgctg |
| | | ctgttaagaatggtattcactttgccgttgcagcaggtaacgaaaaccaagat |
| | | gcttgtaacacctcgccagcagctgctgagaatgccatcaccgtcggtgcat |
| | | caaccttatcagacgctagagcttacttttctaactacggtaaatgtgttgacat |
| | | tttcgctccaggtttaaacattctttctacctacactggttcggatgacgcaact |
| | | gctaccttgtctggtacttcaatggcctctcctcacattgctggtctgttgactta |
| | | cttcctatcattgcagcctgctgctggatctctgtactctaacggaggatctga |
| | | gggtgtcacacctgctcaattgaaaaagaacctcctcaagtatgcatctgtcg |
| | | gagtattagaggatgttccagaagacactccaaacctcttggtttacaatggt |
| | | ggtggacaaaacctttcttctttctggggaaaggagacagaagacaatgttg |
| | | cttcctccgacgatactggtgagtttcactcttttgtgaacaagcttgaatcagc |
| | | tgttgaaaacttggcccaagagtttgcacattcagtgaaggagctggcttctg |
| | | aacttatttagattggagaaaaggaatacacaaggagttaaaaaaagtgtggt |
| | | agaaagtgcatttgtcataattttccatatgttgctgtcactgtaatcttttatatttt |
| | | gttttgttttatgtagtatttcaaaaggttcttatcatcttactggcataaacttgat |
| | | gtacgcagagatagcaaccgttgcttaggtaagcatagtaaaaatggctggt |
| | | tttctgtcttattttaaggccactgttgggacaaaacacaataactagattttatc |
| | | ggattgaacagtgtaaaggcttcactggcttatatcttgtatgagtacgataca |
| | | ttatccagttccatcaaggcctgtggaaatattacagccaggacatgaacctg |
| | | aaagggagtttagtgggatcactgtagataataggaacagacttaatgaaga |
| | | aaagtattatcagacgaaaatagacgaagcgttgaaaaggggcacagaaa |
| | | gacgttacgttgatgatcatagcagaggtcatgagtctccaagttcagatttg |
| | | gaggacactccggatcaattcttggaatttcacattcatgataacggagatag |
| | | gaagatttcaaggccagacactgcttcgtcattgattagtgaaaacgacatgg |
| | | actacgatgatttgtttgttgacagaaagcaaccaaaacatgctacttctcatgt |
| | | aaagcagtttattaggaagaatgtgttccaaaagaagactcatctaccaaaca |
| | | ttggggctagagaactggaattacagaaacggcttgctttattagagggccc |
| | | aatagatgacgatgagattattagtgctatgcccatggtagcgtgtccctctga |
| | | ctataacgatcaacctgctgattcaaattcaagtaaagcgttacagagttcaac |
| | | cgcctctaatccctccagttcattgcctaaaaaagaagaggaggcaattaaa |
| | | gctgtacgggaagatgagcaggatactgcaccagacggagatgcctatgg |
| | | cattggaagcttggtggcagacgctgcttttaagtttctcaactacattttgcctt |
| | | cggattctagctccaaccccagttcgacagctatctccacagtagataaggc |
| | | attgccgccagctccaacatttatgtcgtcaggtccctgtttagatggtgctag |
| | | acccagttcaacttctccctgtacgagaaccacgccgctttattcgtacatgg |
| | | ctccaaaagattcaagcagaaatcaaacggtaattttgaaagctttcaaacgc |
| | | ccattttcaaagaaatcaagttcaagcgtctctcctaagcgggaaaatcacac |
| | | tgaattaattcctagtactggccccttgtgg |
| 20 | Pichia pastoris | ATGACATCTCGGACAGCTGAGAACCCGTTCGA |
| | STE13 ORF | TATAGAGCTTCAAGAGAATCTAAGTCCACGTT |
| | | CTTCCAATTCGTCCATATTGGAAAACATTAAT |
| | | GAGTATGCTAGAAGACATCGCAATGATTCGCT |
| | | TTCCCAAGAATGTGATAATGAAGATGAGAAC |
| | | GAAAATCTCAATTATACTGATAACTTGGCCAA |
| | | GTTTTCAAAGTCTGGAGTATCAAGAAAGAGCT |
| | | GTATGCTAATATTTGGTATTTGCTTTGTTATCT |
| | | GGCTGTTTCTCTTTGCCTTGTATGCGAGGGAC |
| | | AATCGATTTTCCAATTTGAACGAGTACGTTCC |
| | | AGATTCAAACAGCCACGGAACTGCTTCTGCCA |
| | | CCACGTCTATCGTTGAACCAAAACAGACTGAA |
| | | TTACCTGAAAGCAAAGATTCTAACACTGATTA |
| | | TCAAAAAGGAGCTAAATTGAGCCTTAGCGGC |
| | | TGGAGATCAGGTCTGTACAATGTCTATCCAAA |
| | | ACTGATCTCTCGTGGTGAAGATGACATATACT |
| | | ATGAACACAGTTTTCATCGTATAGATGAAAAG |
| | | AGGATTACAGACTCTCAACACGGTCGAACTGT |
| | | ATTTAACTATGAGAAAATTGAAGTAAATGGA |
| | | ATCACGTATACAGTGTCATTTGTCACCATTTCT |
| | | CCTTACGATTCTGCCAAATTCTTAGTCGCATG |
| | | CGACTATGAAAAACACTGGAGACATTCTACGT |
| | | TTGCAAAATATTTCATATATGATAAGGAAAGC |
| | | GACCAAGAGGATAGCTTTGTACCTGTCTACGA |
| | | TGACAAGGCATTGAGCTTCGTTGAATGGTCGC |
| | | CCTCAGGTGATCATGTAGTATTCGTTTTTGAA |
| | | AACAATGTATACCTCAAACAACTCTCAACTTT |
| | | AGAGGTTAAGCAGGTAACTTTTGATGGTGATG |
| | | AGAGTATTTACAATGGTAAGCCTGACTGGATC |
| | | TATGAAGAGGAAGTTTTAAGTAGCGACAGAG |
| | | CCATATGGTGGAATGACGATGGATCGTACTTT |
| | | ACGTTCTTGAGACTTGATGACAGCAATGTCCC |
| | | AACCTTCAACTTGCAGCATTTTTTTGAAGAAA |
| | | CAGGCTCTGTGTCGAAATATCCGGTCATTGAT |
| | | CGATTGAAATATCCAAAACCAGGATTTGACA |
| | | ACCCCCTGGTTTCTTTGTTTAGTTACAACGTTG |
| | | CCAAGCAAAAGTTAGAAAAGCTAAATATTGG |
| | | AGCAGCAGTTTCTTTGGGAGAAGACTTCGTGC |
| | | TTTACAGTTTAAAATGGATAGACAATTCTTTT |
| | | TTCTTGTCGAAGTTCACAGACCGCACTTCGAA |
| | | AAAAATGGAAGTTACTCTAGTGGACATTGAA |
| | | GCCAATTCTGCTTCGGTGGTGAGAAAACATGA |
| | | TGCAACTGAGTATAACGGCTGGTTCACTGGAG |
| | | AATTTTCTGTTTATCCTGTCGTTGGAGATACCA |
| | | TTGGTTACATTGATGTAATCTATTATGAGGAC |
| | | TACGATCACTTGGCTTATTATCCAGACTGCAC |
| | | ATCCGATAAGTATATTGTGCTTACAGATGGTT |
| | | CATGGAATGTTGTTGGACCTGGAGTTTTAGAA |
| | | GTGCTTGAAGATAGAGTCTACTTTATCGGCAC |
| | | CAAAGAATCATCAATGGAACATCACTTGTATT |
| | | ATACATCATTAACGGGACCCAAGGTTAAGGCT |
| | | GTTATGGATATCAAAGAACCTGGGTACTTTGA |
| | | TGTAAACATTAAGGGAAAATATGCTTTACTAT |
| | | CTTACAGAGGCCCCAAACTCCCATACCAGAA |
| | | ATTTATTGATCTTTCTGACCCTAGTACAACAA |
| | | GTCTTGATGACATTTTATCGTCTAATAGAGGA |
| | | ATTGTCGAGGTTAGTTTAGCAACTCACAGCGT |
| | | TCCTGTTTCTACCTATACTAATGTAACACTTGA |
| | | GGACGGCGTCACACTGAACATGATTGAAGTG |
| | | TTGCCTGCCAATTTTAATCCTAGCAAGAAGTA |
| | | CCCACTGTTGGTCAACATTTATGGTGGACCGG |
| | | GCTCCCAGAAGTTAGATGTGCAGTTCAACATT |
| | | GGGTTTGAGCATATTATTTCTTCGTCACTGGA |
| | | TGCAATAGTGCTTTACATAGATCCGAGAGGTA |
| | | CTGGAGGTAAAAGCTGGGCTTTTAAATCTTAC |
| | | GCTACAGAGAAAATAGGCTACTGGGAACCAC |
| | | GAGACATCACTGCAGTAGTTTCCAAGTGGATT |
| | | TCAGATCACTCATTTGTGAATCCTGACAAAAC |
| | | TGCGATATGGGGGTGGTCTTACGGTGGGTTCA |
| | | CTACGCTTAAGACATTGGAATATGATTCTGGA |
| | | GAGGTTTTCAAATATGGTATGGCTGTTGCTCC |
| | | AGTAACTAATTGGCTTTTGTATGACTCCATCT |
| | | ACACTGAAAGATACATGAACCTTCCAAAGGA |
| | | CAATGTTGAAGGCTACAGTGAACACAGCGTC |
| | | ATTAAGAAGGTTTCCAATTTTAAGAATGTAAA |
| | | CCGATTCTTGGTTTGTCACGGGACTACTGATG |
| | | ATAACGTGCATTTTCAGAACACACTAACCTTA |
| | | CTGGACCAGTTCAATATTAATGGTGTTGTGAA |
| | | TTACGATCTTCAGGTGTATCCCGACAGTGAAC |
| | | ATAGCATTGCCCATCACAACGCAAATAAAGT |
| | | GATCTACGAGAGGTTATTCAAGTGGTTAGAGC |
| | | GGGCATTTAACGATAGATTTTTGTAA |
| 21 | Pichia pastoris | ATGTATCCCGAACACAAGTATCGGGAGTATCA |
| | DAP2 ORF | ACGGAGGGTGCCCTTATGGCAGTACTCCCTGT |
| | | TGGTGATTGTACTGCTATACGGGTCTCATTTG |
| | | CTTATCAGCACCATCAACTTGATACACTATAA |
| | | CCACAAAAATTATCATGCACACCCAGTCAATA |
| | | GTGGTATCGTTCTTAATGAGTTTGCTGATGAC |
| | | GATTCATTCTCTTTGAATGGCACTCTGAACTT |
| | | GGAGAACTGGAGAAATGGTACCTTTTCCCCTA |
| | | AATTTCATTCCATTCAGTGGACCGAAATAGGT |
| | | CAGGAAGATGACCAGGGATATTACATTCTCTC |
| | | TTCCAATTCCTCTTACATAGTAAAGTCTTTATC |
| | | CGACCCAGACTTTGAATCTGTTCTATTCAACG |
| | | AGTCTACAATCACTTACAACGGTGAAGAACAT |
| | | CATGTGGAAGACGTCATAGTGTCCAATAATCT |
| | | TCAATATGCATTGGTAGTTACGGATAAGAGAC |
| | | ATAATTGGCGCCATTCTTTTTTTGCGAATTACT |
| | | GGCTGTATAAAGTCAACAATCCTGAACAGGTT |
| | | CAGCCTTTGTTTGATACAGATCTATCGTTGAA |
| | | TGGTCTTATTAGCCTTGTCCATTGGTCTCCGGA |
| | | TTCTTCCCAAGTTGCATTTGTGTTGGAAAATA |
| | | ACATATATTTGAAGCATCTTAACAACTTTTCT |
| | | GATTCAAGGATTGATCAACTAACTTATGATGG |
| | | AGGCGAAAACATATTTTATGGCAAACCAGATT |
| | | GGGTTTATGAAGAAGAAGTGTTTGAAAGCAA |
| | | CTCTGCTATGTGGTGGTCTCCAAATGGAAAGT |
| | | TTTTATCAATATTGCGAACTAATGACACCCAA |
| | | GTGCCTGTCTATCCTATTCCATATTTTGTTCAG |
| | | TCTGATGCTGAAACAGCTATCGATGAATACCC |
| | | TCTTCTGAAACACATAAAATACCCAAAGGCA |
| | | GGATTTCCCAATCCAGTTGTTGATGTGATTGT |
| | | ATACGATGTTCAACGCCAGCACATATCTAGGT |
| | | TACCTGCTGGTGATCCTTTCTACAACGATGAG |
| | | AACATTACCAATGAGGACAGACTTATCACTGA |
| | | GATCATCTGGGTTGGTGATTCACGGTTCCTGA |
| | | CCAAGATTACGAACAGGGAAAGTGACTTGTT |
| | | AGCATTTTATCTGGTAGACGCTGAGGCTAACA |
| | | ATAGTAAGCTGGTAAGATTCCAAGATGCTAA |
| | | GAGCACCAAGTCTTGGTTTGAAATTGAACACA |
| | | ACACATTGTATATTCCTAAGGATACTTCAGTG |
| | | GGAAGGGCACAAGATGGCTACATCGACACCA |
| | | TAGATGTTAACGGCTACAACCATTTAGCCTAT |
| | | TTCTCACCACCAGACAACCCAGACCCCAAGGT |
| | | CATTCTTACGCGTGGTGATTGGGAAGTCGTTG |
| | | ACAGTCCATCTGCATTTGACTTCAAAAGAAAT |
| | | TTGGTTTACTTTACAGCAACCAAGAAATCCTC |
| | | AATAGAAAGACATGTTTATTGTGTTGGGATAG |
| | | ACGGGAAACAATTCAACAATGTAACTGATGTT |
| | | TCATCAGATGGATACTACAGTACAAGCTTTTC |
| | | CCCTGGAGCAAGATATGTATTGCTATCACACC |
| | | AAGGTCCCCGTGTACCTTATCAAAAGATGATA |
| | | GATCTTGTCAAAGGCACCGAAGAAATAATCG |
| | | AATCTAACGAAGATTTGAAAGACTCCGTTGCT |
| | | TTATTTGATTTACCTGATGTCAAGTACGGCGA |
| | | AATCGAGCTTGAAAAAGGTGTCAAGTCAAAC |
| | | TACGTTGAGATCAGGCCTAAGAACTTCGATGA |
| | | AAGCAAAAAGTATCCGGTTTTATTTTTTGTGT |
| | | ATGGGGGGCCAGGTTCCCAATTGGTAACAAA |
| | | GACATTTTCTAAGAGTTTCCAGCATGTTGTAT |
| | | CCTCTGAGCTTGACGTCATTGTTGTCACGGTG |
| | | GATGGAAGAGGGACTGGATTTAAAGGTAGAA |
| | | AATATAGATCCATAGTGCGGGACAACTTGGGT |
| | | CATTATGAATCCCTGGACCAAATCACGGCAGG |
| | | AAAAATTTGGGCAGCAAAGCCTTACGTTGATG |
| | | AGAATAGACTGGCCATTTGGGGTTGGTCTTAT |
| | | GGAGGTTACATGACGCTAAAGGTTTTAGAAC |
| | | AGGATAAAGGTGAAACATTCAAATATGGAAT |
| | | GTCTGTTGCCCCTGTGACGAATTGGAAATTCT |
| | | ATGATTCTATCTACACAGAAAGATACATGCAC |
| | | ACTCCTCAGGACAATCCAAACTATTATAATTC |
| | | GTCAATCCATGAGATTGATAATTTGAAGGGAG |
| | | TGAAGAGGTTCTTGCTAATGCACGGAACTGGT |
| | | GACGACAATGTTCACTTCCAAAATACACTCAA |
| | | AGTTCTAGATTTATTTGATTTACATGGTCTTGA |
| | | AAACTATGATATCCACGTGTTCCCTGATAGTG |
| | | ATCACAGTATTAGATATCACAACGGTAATGTT |
| | | ATAGTGTATGATAAGCTATTCCATTGGATTAG |
| | | GCGTGCATTCAAGGCTGGCAAA |
| 22 | Alpha amylase | ATGGTTGCTT GGTGGTCCTT GTTCTTGTAC |
| | signal peptide (from | GGATTGCAAG TTGCTGCTCC AGCTTTGGCT |
| | Aspergillus niger α- | |
| | amylase) DNA | |
| 23 | Alpha amylase | MVAWWSLFLY GLQVAAPALA |
| | signal peptide (from | |
| | Aspergillus niger α- | |
| | amylase) | |
| 24 | Saccharomyces | ATG AGA TTC CCA TCC ATC TTC ACT GCT |
| | cerevisiae mating | GTT TTG TTC GCT GCT TCT TCT GCT TTG GCT |
| | factor pre-signal | |
| | peptide DNA | |
| 25 | Saccharomyces | MRFPSIFTAVLFAASSALA |
| | cerevisiae mating | |
| | factor pre-signal | |
| | peptide | |
| 26 | Saccharomyces | ATGCGATTTCCTTCCATTTTTACTGCTGTTTTG |
| | cerevisiae mating | TTTGCCGCCTCCTCAGCTTTGGCCTCACTGAA |
| | factor pre-pro signal | CTGTACACTGCGTGATTCACAGCAGAAAAGTC |
| | peptide (MFIL-1β | TGGTCATGTCCGGACCATACGAACTTAAAGCC |
| | prepro) DNA | TTAGTTAAAAGA |
| 27 | Saccharomyces | MRFPSIFTAVLFAASSALASLNCTLRDSQQKSLV |
| | cerevisiae mating | MSGPYELKALVKR |
| | factor pre-pro signal | |
| | peptide (MFIL-1β | |
| | prepro) | |
| 28 | HSA signal peptide | ATGAAGTGGGTTACCTTTATCTCTTTGTTGTTT |
| | DNA | CTTTTCTCTTCTGCTTACTCT |
| 29 | HSA signal peptide | MKWVTFISLLFLFSSAYS |
| 30 | Pichia pastoris | atggctatattcgccgtttctgtcatttgcgttttgtacggaccctcacaacaatt |
| | OCH1 | atcatctccaaaaatagactatgatccattgacgctccgatcacttgatttgaa |
| | | gactttggaagctccttcacagttgagtccaggcaccgtagaagataatcttc |
| | | gaagacaattggagtttcattttccttaccgcagttacgaaccttttccccaaca |
| | | tatttggcaaacgtggaaagtttctccctctgatagttcctttccgaaaaacttc |
| | | aaagacttaggtgaaagttggctgcaaaggtccccaaattatgatcattttgtg |
| | | atacccgatgatgcagcatgggaacttattcaccatgaatacgaacgtgtac |
| | | cagaagtcttggaagctttccacctgctaccagagcccattctaaaggccga |
| | | ttttttcaggtatttgattctttttgcccgtggaggactgtatgctgacatggaca |
| | | ctatgttattaaaaccaatagaatcgtggctgactttcaatgaaactattggtgg |
| | | agtaaaaaacaatgctgggttggtcattggtattgaggctgatcctgatagac |
| | | ctgattggcacgactggtatgctagaaggatacaattttgccaatgggcaatt |
| | | cagtccaaacgaggacacccagcactgcgtgaactgattgtaagagttgtca |
| | | gcacgactttacggaaagagaaaagcggttacttgaacatggtggaaggaa |
| | | aggatcgtggaagtgatgtgatggactggacgggtccaggaatatttacaga |
| | | cactctatttgattatatgactaatgtcaatacaacaggccactcaggccaag |
| | | gaattggagctggctcagcgtattacaatgccttatcgttggaagaacgtgat |
| | | gccctctctgcccgcccgaacggagagatgttaaaagagaaagtcccaggt |
| | | aaatatgcacagcaggttgttttatgggaacaatttaccaacctgcgctcccc |
| | | caaattaatcgacgatattcttattcttccgatcaccagcttcagtccagggatt |
| | | ggccacagtggagctggagatttgaaccatcaccttgcatatattaggcatac |
| | | atttgaaggaagttggaaggac |
| 31 | Och1p | MAIFAVSVICVLYGPSQQLSSPKIDYDPLTLRSLD |
| | | LKTLEAPSQLSPGTVEDNLRRQLEFHFPYRSYEP |
| | | FPQHIWQTWKVSPSDSSFPKNFKDLGESWLQRS |
| | | PNYDHFVIPDDAAWELIHHEYERVPEVLEAFHL |
| | | LPEPILKADFFRYLILFARGGLYADMDTMLLKPI |
| | | ESWLTFNETIGGVKNNAGLVIGIEADPDRPDWH |
| | | DWYARRIQFCQWAIQSKRGHPALRELIVRVVST |
| | | TLRKEKSGYLNMVEGKDRGSDVMDWTGPGIFT |
| | | DTLFDYMTNVNTTGHSGQGIGAGSAYYNALSLE |
| | | ERDALSARPNGEMLKEKVPGKYAQQVVLWEQF |
| | | TNLRSPKLIDDILILPITSFSPGIGHSGAGDLNHHL |
| | | AYIRHTFEGSWKD |
| 32 | CPY sorting signal | QRPL |
| 33 | Cryptic CPY | QSFL |
| | sorting signal in | |
| | GCSF | |
| 34 | Tricoderma reesei | CGCGCCGGATCTCCCAACCCTACGAGGGCGG |
| | α-1,2-mannosidase | CAGCAGTCAAGGCCGCATTCCAGACGTCGTG |
| | catalytic domain | GAACGCTTACCACCATTTTGCCTTTCCCCATG |
| | | ACGACCTCCACCCGGTCAGCAACAGCTTTGAT |
| | | GATGAGAGAAACGGCTGGGGCTCGTCGGCAA |
| | | TCGATGGCTTGGACACGGCTATCCTCATGGGG |
| | | GATGCCGACATTGTGAACACGATCCTTCAGTA |
| | | TGTACCGCAGATCAACTTCACCACGACTGCGG |
| | | TTGCCAACCAAGGCATCTCCGTGTTCGAGACC |
| | | AACATTCGGTACCTCGGTGGCCTGCTTTCTGC |
| | | CTATGACCTGTTGCGAGGTCCTTTCAGCTCCT |
| | | TGGCGACAAACCAGACCCTGGTAAACAGCCT |
| | | TCTGAGGCAGGCTCAAACACTGGCCAACGGC |
| | | CTCAAGGTTGCGTTCACCACTCCCAGCGGTGT |
| | | CCCGGACCCTACCGTCTTCTTCAACCCTACTG |
| | | TCCGGAGAAGTGGTGCATCTAGCAACAACGT |
| | | CGCTGAAATTGGAAGCCTGGTGCTCGAGTGG |
| | | ACACGGTTGAGCGACCTGACGGGAAACCCGC |
| | | AGTATGCCCAGCTTGCGCAGAAGGGCGAGTC |
| | | GTATCTCCTGAATCCAAAGGGAAGCCCGGAG |
| | | GCATGGCCTGGCCTGATTGGAACGTTTGTCAG |
| | | CACGAGCAACGGTACCTTTCAGGATAGCAGC |
| | | GGCAGCTGGTCCGGCCTCATGGACAGCTTCTA |
| | | CGAGTACCTGATCAAGATGTACCTGTACGACC |
| | | CGGTTGCGTTTGCACACTACAAGGATCGCTGG |
| | | GTCCTTGCTGCCGACTCGACCATTGCGCATCT |
| | | CGCCTCTCACCCGTCGACGCGCAAGGACTTGA |
| | | CCTTTTTGTCTTCGTACAACGGACAGTCTACG |
| | | TCGCCAAACTCAGGACATTTGGCCAGTTTTGC |
| | | CGGTGGCAACTTCATCTTGGGAGGCATTCTCC |
| | | TGAACGAGCAAAAGTACATTGACTTTGGAATC |
| | | AAGCTTGCCAGCTCGTACTTTGCCACGTACAA |
| | | CCAGACGGCTTCTGGAATCGGCCCCGAAGGC |
| | | TTCGCGTGGGTGGACAGCGTGACGGGCGCCG |
| | | GCGGCTCGCCGCCCTCGTCCCAGTCCGGGTTC |
| | | TACTCGTCGGCAGGATTCTGGGTGACGGCACC |
| | | GTATTACATCCTGCGGCCGGAGACGCTGGAG |
| | | AGCTTGTACTACGCATACCGCGTCACGGGCGA |
| | | CTCCAAGTGGCAGGACCTGGCGTGGGAAGCG |
| | | TTCAGTGCCATTGAGGACGCATGCCGCGCCGG |
| | | CAGCGCGTACTCGTCCATCAACGACGTGACGC |
| | | AGGCCAACGGCGGGGGTGCCTCTGACGATAT |
| | | GGAGAGCTTCTGGTTTGCCGAGGCGCTCAAGT |
| | | ATGCGTACCTGATCTTTGCGGAGGAGTCGGAT |
| | | GTGCAGGTGCAGGCCAACGGCGGGAACAAAT |
| | | TTGTCTTTAACACGGAGGCGCACCCCTTTAGC |
| | | ATCCGTTCATCATCACGACGGGGCGGCCACCT |
| | | TGCTTAA |
| 35 | Sequence of the 5′- | ATCGGCCTTTGTTGATGCAAGTTTTACGTGGA |
| | Region used for | TCATGGACTAAGGAGTTTTATTTGGACCAAGT |
| | knock out of | TCATCGTCCTAGACATTACGGAAAGGGTTCTG |
| | PpURA5: | CTCCTCTTTTTGGAAACTTTTTGGAACCTCTGA |
| | | GTATGACAGCTTGGTGGATTGTACCCATGGTA |
| | | TGGCTTCCTGTGAATTTCTATTTTTTCTACATT |
| | | GGATTCACCAATCAAAACAAATTAGTCGCCAT |
| | | GGCTTTTTGGCTTTTGGGTCTATTTGTTTGGAC |
| | | CTTCTTGGAATATGCTTTGCATAGATTTTTGTT |
| | | CCACTTGGACTACTATCTTCCAGAGAATCAAA |
| | | TTGCATTTACCATTCATTTCTTATTGCATGGGA |
| | | TACACCACTATTTACCAATGGATAAATACAGA |
| | | TTGGTGATGCCACCTACACTTTTCATTGTACTT |
| | | TGCTACCCAATCAAGACGCTCGTCTTTTCTGT |
| | | TCTACCATATTACATGGCTTGTTCTGGATTTGC |
| | | AGGTGGATTCCTGGGCTATATCATGTATGATG |
| | | TCACTCATTACGTTCTGCATCACTCCAAGCTG |
| | | CCTCGTTATTTCCAAGAGTTGAAGAAATATCA |
| | | TTTGGAACATCACTACAAGAATTACGAGTTAG |
| | | GCTTTGGTGTCACTTCCAAATTCTGGGACAAA |
| | | GTCTTTGGGACTTATCTGGGTCCAGACGATGT |
| | | GTATCAAAAGACAAATTAGAGTATTTATAAA |
| | | GTTATGTAAGCAAATAGGGGCTAATAGGGAA |
| | | AGAAAAATTTTGGTTCTTTATCAGAGCTGGCT |
| | | CGCGCGCAGTGTTTTTCGTGCTCCTTTGTAATA |
| | | GTCATTTTTGACTACTGTTCAGATTGAAATCA |
| | | CATTGAAGATGTCACTCGAGGGGTACCAAAA |
| | | AAGGTTTTTGGATGCTGCAGTGGCTTCGC |
| 36 | Sequence of the 3′- | GGTCTTTTCAACAAAGCTCCATTAGTGAGTCA |
| | Region used for | GCTGGCTGAATCTTATGCACAGGCCATCATTA |
| | knock out of | ACAGCAACCTGGAGATAGACGTTGTATTTGGA |
| | PpURA5: | CCAGCTTATAAAGGTATTCCTTTGGCTGCTAT |
| | | TACCGTGTTGAAGTTGTACGAGCTCGGCGGCA |
| | | AAAAATACGAAAATGTCGGATATGCGTTCAA |
| | | TAGAAAAGAAAAGAAAGACCACGGAGAAGG |
| | | TGGAAGCATCGTTGGAGAAAGTCTAAAGAAT |
| | | AAAAGAGTACTGATTATCGATGATGTGATGAC |
| | | TGCAGGTACTGCTATCAACGAAGCATTTGCTA |
| | | TAATTGGAGCTGAAGGTGGGAGAGTTGAAGG |
| | | TAGTATTATTGCCCTAGATAGAATGGAGACTA |
| | | CAGGAGATGACTCAAATACCAGTGCTACCCA |
| | | GGCTGTTAGTCAGAGATATGGTACCCCTGTCT |
| | | TGAGTATAGTGACATTGGACCATATTGTGGCC |
| | | CATTTGGGCGAAACTTTCACAGCAGACGAGA |
| | | AATCTCAAATGGAAACGTATAGAAAAAAGTA |
| | | TTTGCCCAAATAAGTATGAATCTGCTTCGAAT |
| | | GAATGAATTAATCCAATTATCTTCTCACCATT |
| | | ATTTTCTTCTGTTTCGGAGCTTTGGGCACGGC |
| | | GGCGGGTGGTGCGGGCTCAGGTTCCCTTTCAT |
| | | AAACAGATTTAGTACTTGGATGCTTAATAGTG |
| | | AATGGCGAATGCAAAGGAACAATTTCGTTCAT |
| | | CTTTAACCCTTTCACTCGGGGTACACGTTCTG |
| | | GAATGTACCCGCCCTGTTGCAACTCAGGTGGA |
| | | CCGGGCAATTCTTGAACTTTCTGTAACGTTGT |
| | | TGGATGTTCAACCAGAAATTGTCCTACCAACT |
| | | GTATTAGTTTCCTTTTGGTCTTATATTGTTCAT |
| | | CGAGATACTTCCCACTCTCCTTGATAGCCACT |
| | | CTCACTCTTCCTGGATTACCAAAATCTTGAGG |
| | | ATGAGTCTTTTCAGGCTCCAGGATGCAAGGTA |
| | | TATCCAAGTACCTGCAAGCATCTAATATTGTC |
| | | TTTGCCAGGGGGTTCTCCACACCATACTCCTT |
| | | TTGGCGCATGC |
| 37 | Sequence of the | TCTAGAGGGACTTATCTGGGTCCAGACGATGT |
| | PpURA5 | GTATCAAAAGACAAATTAGAGTATTTATAAA |
| | auxotrophic marker: | GTTATGTAAGCAAATAGGGGCTAATAGGGAA |
| | | AGAAAAATTTTGGTTCTTTATCAGAGCTGGCT |
| | | CGCGCGCAGTGTTTTTCGTGCTCCTTTGTAATA |
| | | GTCATTTTTGACTACTGTTCAGATTGAAATCA |
| | | CATTGAAGATGTCACTGGAGGGGTACCAAAA |
| | | AAGGTTTTTGGATGCTGCAGTGGCTTCGCAGG |
| | | CCTTGAAGTTTGGAACTTTCACCTTGAAAAGT |
| | | GGAAGACAGTCTCCATACTTCTTTAACATGGG |
| | | TCTTTTCAACAAAGCTCCATTAGTGAGTCAGC |
| | | TGGCTGAATCTTATGCTCAGGCCATCATTAAC |
| | | AGCAACCTGGAGATAGACGTTGTATTTGGACC |
| | | AGCTTATAAAGGTATTCCTTTGGCTGCTATTA |
| | | CCGTGTTGAAGTTGTACGAGCTGGGCGGCAA |
| | | AAAATACGAAAATGTCGGATATGCGTTCAAT |
| | | AGAAAAGAAAAGAAAGACCACGGAGAAGGT |
| | | GGAAGCATCGTTGGAGAAAGTCTAAAGAATA |
| | | AAAGAGTACTGATTATCGATGATGTGATGACT |
| | | GCAGGTACTGCTATCAACGAAGCATTTGCTAT |
| | | AATTGGAGCTGAAGGTGGGAGAGTTGAAGGT |
| | | TGTATTATTGCCCTAGATAGAATGGAGACTAC |
| | | AGGAGATGACTCAAATACCAGTGCTACCCAG |
| | | GCTGTTAGTCAGAGATATGGTACCCCTGTCTT |
| | | GAGTATAGTGACATTGGACCATATTGTGGCCC |
| | | ATTTGGGCGAAACTTTCACAGCAGACGAGAA |
| | | ATCTCAAATGGAAACGTATAGAAAAAAGTAT |
| | | TTGCCCAAATAAGTATGAATCTGCTTCGAATG |
| | | AATGAATTAATCCAATTATCTTCTCACCATTA |
| | | TTTTCTTCTGTTTCGGAGCTTTGGGCACGGCG |
| | | GCGGATCC |
| 38 | Sequence of the | CCTGCACTGGATGGTGGCGCTGGATGGTAAGC |
| | part of the Ec lacZ | CGCTGGCAAGCGGTGAAGTGCCTCTGGATGTC |
| | gene that was used | GCTCCACAAGGTAAACAGTTGATTGAACTGCC |
| | to construct the | TGAACTACCGCAGCCGGAGAGCGCCGGGCAA |
| | PpURA5 blaster | CTCTGGCTCACAGTACGCGTAGTGCAACCGAA |
| | (recyclable | CGCGACCGCATGGTCAGAAGCCGGGCACATC |
| | auxotrophic | AGCGCCTGGCAGCAGTGGCGTCTGGCGGAAA |
| | marker) | ACCTCAGTGTGACGCTCCCCGCCGCGTCCCAC |
| | | GCCATCCCGCATCTGACCACCAGCGAAATGG |
| | | ATTTTTGCATCGAGCTGGGTAATAAGCGTTGG |
| | | CAATTTAACCGCCAGTCAGGCTTTCTTTCACA |
| | | GATGTGGATTGGCGATAAAAAACAACTGCTG |
| | | ACGCCGCTGCGCGATCAGTTCACCCGTGCACC |
| | | GCTGGATAACGACATTGGCGTAAGTGAAGCG |
| | | ACCCGCATTGACCCTAACGCCTGGGTCGAACG |
| | | CTGGAAGGCGGCGGGCCATTACCAGGCCGAA |
| | | GCAGCGTTGTTGCAGTGCACGGCAGATACACT |
| | | TGCTGATGCGGTGCTGATTACGACCGCTCACG |
| | | CGTGGCAGCATCAGGGGAAAACCTTATTTATC |
| | | AGCCGGAAAACCTACCGGATTGATGGTAGTG |
| | | GTCAAATGGCGATTACCGTTGATGTTGAAGTG |
| | | GCGAGCGATACACCGCATCCGGCGCGGATTG |
| | | GCCTGAACTGCCAG |
| 39 | Sequence of the 5′- | AAAACCTTTTTTCCTATTCAAACACAAGGCAT |
| | Region used for | TGCTTCAACACGTGTGCGTATCCTTAACACAG |
| | knock out of | ATACTCCATACTTCTAATAATGTGATAGACGA |
| | PpOCH1: | ATACAAAGATGTTCACTCTGTGTTGTGTCTAC |
| | | AAGCATTTCTTATTCTGATTGGGGATATTCTA |
| | | GTTACAGCACTAAACAACTGGCGATACAAAC |
| | | TTAAATTAAATAATCCGAATCTAGAAAATGAA |
| | | CTTTTGGATGGTCCGCCTGTTGGTTGGATAAA |
| | | TCAATACCGATTAAATGGATTCTATTCCAATG |
| | | AGAGAGTAATCCAAGACACTCTGATGTCAAT |
| | | AATCATTTGCTTGCAACAACAAACCCGTCATC |
| | | TAATCAAAGGGTTTGATGAGGCTTACCTTCAA |
| | | TTGCAGATAAACTCATTGCTGTCCACTGCTGT |
| | | ATTATGTGAGAATATGGGTGATGAATCTGGTC |
| | | TTCTCCACTCAGCTAACATGGCTGTTTGGGCA |
| | | AAGGTGGTACAATTATACGGAGATCAGGCAA |
| | | TAGTGAAATTGTTGAATATGGCTACTGGACGA |
| | | TGCTTCAAGGATGTACGTCTAGTAGGAGCCGT |
| | | GGGAAGATTGCTGGCAGAACCAGTTGGCACG |
| | | TCGCAACAATCCCCAAGAAATGAAATAAGTG |
| | | AAAACGTAACGTCAAAGACAGCAATGGAGTC |
| | | AATATTGATAACACCACTGGCAGAGCGGTTCG |
| | | TACGTCGTTTTGGAGCCGATATGAGGCTCAGC |
| | | GTGCTAACAGCACGATTGACAAGAAGACTCT |
| | | CGAGTGACAGTAGGTTGAGTAAAGTATTCGCT |
| | | TAGATTCCCAACCTTCGTTTTATTCTTTCGTAG |
| | | ACAAAGAAGCTGCATGCGAACATAGGGACAA |
| | | CTTTTATAAATCCAATTGTCAAACCAACGTAA |
| | | AACCCTCTGGCACCATTTTCAACATATATTTG |
| | | TGAAGCAGTACGCAATATCGATAAATACTCAC |
| | | CGTTGTTTGTAACAGCCCCAACTTGCATACGC |
| | | CTTCTAATGACCTCAAATGGATAAGCCGCAGC |
| | | TTGTGCTAACATACCAGCAGCACCGCCCGCGG |
| | | TCAGCTGCGCCCACACATATAAAGGCAATCTA |
| | | CGATCATGGGAGGAATTAGTTTTGACCGTCAG |
| | | GTCTTCAAGAGTTTTGAACTCTTCTTCTTGAAC |
| | | TGTGTAACCTTTTAAATGACGGGATCTAAATA |
| | | CGTCATGGATGAGATCATGTGTGTAAAAACTG |
| | | ACTCCAGCATATGGAATCATTCCAAAGATTGT |
| | | AGGAGCGAACCCACGATAAAAGTTTCCCAAC |
| | | CTTGCCAAAGTGTCTAATGCTGTGACTTGAAA |
| | | TCTGGGTTCCTCGTTGAAGACCCTGCGTACTA |
| | | TGCCCAAAAACTTTCCTCCACGAGCCCTATTA |
| | | ACTTCTCTATGAGTTTCAAATGCCAAACGGAC |
| | | ACGGATTAGGTCCAATGGGTAAGTGAAAAAC |
| | | ACAGAGCAAACCCCAGCTAATGAGCCGGCCA |
| | | GTAACCGTCTTGGAGCTGTTTCATAAGAGTCA |
| | | TTAGGGATCAATAACGTTCTAATCTGTTCATA |
| | | ACATACAAATTTTATGGCTGCATAGGGAAAA |
| | | ATTCTCAACAGGGTAGCCGAATGACCCTGATA |
| | | TAGACCTGCGACACCATCATACCCATAGATCT |
| | | GCCTGACAGCCTTAAAGAGCCCGCTAAAAGA |
| | | CCCGGAAAACCGAGAGAACTCTGGATTAGCA |
| | | GTCTGAAAAAGAATCTTCACTCTGTCTAGTGG |
| | | AGCAATTAATGTCTTAGCGGCACTTCCTGCTA |
| | | CTCCGCCAGCTACTCCTGAATAGATCACATAC |
| | | TGCAAAGACTGCTTGTCGATGACCTTGGGGTT |
| | | ATTTAGCTTCAAGGGCAATTTTTGGGACATTT |
| | | TGGACACAGGAGACTCAGAAACAGACACAGA |
| | | GCGTTCTGAGTCCTGGTGCTCCTGACGTAGGC |
| | | CTAGAACAGGAATTATTGGCTTTATTTGTTTG |
| | | TCCATTTCATAGGCTTGGGGTAATAGATAGAT |
| | | GACAGAGAAATAGAGAAGACCTAATATTTTTT |
| | | GTTCATGGCAAATCGCGGGTTCGCGGTCGGGT |
| | | CACACACGGAGAAGTAATGAGAAGAGCTGGT |
| | | AATCTGGGGTAAAAGGGTTCAAAAGAAGGTC |
| | | GCCTGGTAGGGATGCAATACAAGGTTGTCTTG |
| | | GAGTTTACATTGACCAGATGATTTGGCTTTTT |
| | | CTCTGTTCAATTCACATTTTTCAGCGAGAATC |
| | | GGATTGACGGAGAAATGGCGGGGTGTGGGGT |
| | | GGATAGATGGCAGAAATGCTCGCAATCACCG |
| | | CGAAAGAAAGACTTTATGGAATAGAACTACT |
| | | GGGTGGTGTAAGGATTACATAGCTAGTCCAAT |
| | | GGAGTCCGTTGGAAAGGTAAGAAGAAGCTAA |
| | | AACCGGCTAAGTAACTAGGGAAGAATGATCA |
| | | GACTTTGATTTGATGAGGTCTGAAAATACTCT |
| | | GCTGCTTTTTCAGTTGCTTTTTCCCTGCAACCT |
| | | ATCATTTTCCTTTTCATAAGCCTGCCTTTTCTG |
| | | TTTTCACTTATATGAGTTCCGCCGAGACTTCC |
| | | CCAAATTCTCTCCTGGAACATTCTCTATCGCT |
| | | CTCCTTCCAAGTTGCGCCCCCTGGCACTGCCT |
| | | AGTAATATTACCACGCGACTTATATTCAGTTC |
| | | CACAATTTCCAGTGTTCGTAGCAAATATCATC |
| | | AGCCATGGCGAAGGCAGATGGCAGTTTGCTCT |
| | | ACTATAATCCTCACAATCCACCCAGAAGGTAT |
| | | TACTTCTACATGGCTATATTCGCCGTTTCTGTC |
| | | ATTTGCGTTTTGTACGGACCCTCACAACAATT |
| | | ATCATCTCCAAAAATAGACTATGATCCATTGA |
| | | CGCTCCGATCACTTGATTTGAAGACTTTGGAA |
| | | GCTCCTTCACAGTTGAGTCCAGGCACCGTAGA |
| | | AGATAATCTTCG |
| 40 | Sequence of the 3′- | AAAGCTAGAGTAAAATAGATATAGCGAGATT |
| | Region used for | AGAGAATGAATACCTTCTTCTAAGCGATCGTC |
| | knock out of | CGTCATCATAGAATATCATGGACTGTATAGTT |
| | PpOCH1: | TTTTTTTTGTACATATAATGATTAAACGGTCAT |
| | | CCAACATCTCGTTGACAGATCTCTCAGTACGC |
| | | GAAATCCCTGACTATCAAAGCAAGAACCGAT |
| | | GAAGAAAAAAACAACAGTAACCCAAACACCA |
| | | CAACAAACACTTTATCTTCTCCCCCCCAACAC |
| | | CAATCATCAAAGAGATGTCGGAACCAAACAC |
| | | CAAGAAGCAAAAACTAACCCCATATAAAAAC |
| | | ATCCTGGTAGATAATGCTGGTAACCCGCTCTC |
| | | CTTCCATATTCTGGGCTACTTCACGAAGTCTG |
| | | ACCGGTCTCAGTTGATCAACATGATCCTCGAA |
| | | ATGGGTGGCAAGATCGTTCCAGACCTGCCTCC |
| | | TCTGGTAGATGGAGTGTTGTTTTTGACAGGGG |
| | | ATTACAAGTCTATTGATGAAGATACCCTAAAG |
| | | CAACTGGGGGACGTTCCAATATACAGAGACT |
| | | CCTTCATCTACCAGTGTTTTGTGCACAAGACA |
| | | TCTCTTCCCATTGACACTTTCCGAATTGACAA |
| | | GAACGTCGACTTGGCTCAAGATTTGATCAATA |
| | | GGGCCCTTCAAGAGTCTGTGGATCATGTCACT |
| | | TCTGCCAGCACAGCTGCAGCTGCTGCTGTTGT |
| | | TGTCGCTACCAACGGCCTGTCTTCTAAACCAG |
| | | ACGCTCGTACTAGCAAAATACAGTTCACTCCC |
| | | GAAGAAGATCGTTTTATTCTTGACTTTGTTAG |
| | | GAGAAATCCTAAACGAAGAAACACACATCAA |
| | | CTGTACACTGAGCTCGCTCAGCACATGAAAAA |
| | | CCATACGAATCATTCTATCCGCCACAGATTTC |
| | | GTCGTAATCTTTCCGCTCAACTTGATTGGGTTT |
| | | ATGATATCGATCCATTGACCAACCAACCTCGA |
| | | AAAGATGAAAACGGGAACTACATCAAGGTAC |
| | | AAGGCCTTCCA |
| 41 | Sequence of the 5′- | GGCCGAGCGGGCCTAGATTTTCACTACAAATT |
| | Region used for | TCAAAACTACGCGGATTTATTGTCTCAGAGAG |
| | knock out of | CAATTTGGCATTTCTGAGCGTAGCAGGAGGCT |
| | PpBMT2: | TCATAAGATTGTATAGGACCGTACCAACAAAT |
| | | TGCCGAGGCACAACACGGTATGCTGTGCACTT |
| | | ATGTGGCTACTTCCCTACAACGGAATGAAACC |
| | | TTCCTCTTTCCGCTTAAACGAGAAAGTGTGTC |
| | | GCAATTGAATGCAGGTGCCTGTGCGCCTTGGT |
| | | GTATTGTTTTTGAGGGCCCAATTTATCAGGCG |
| | | CCTTTTTTCTTGGTTGTTTTCCCTTAGCCTCAA |
| | | GCAAGGTTGGTCTATTTCATCTCCGCTTCTATA |
| | | CCGTGCCTGATACTGTTGGATGAGAACACGAC |
| | | TCAACTTCCTGCTGCTCTGTATTGCCAGTGTTT |
| | | TGTCTGTGATTTGGATCGGAGTCCTCCTTACTT |
| | | GGAATGATAATAATCTTGGCGGAATCTCCCTA |
| | | AACGGAGGCAAGGATTCTGCCTATGATGATCT |
| | | GCTATCATTGGGAAGCTTCAACGACATGGAG |
| | | GTCGACTCCTATGTCACCAACATCTACGACAA |
| | | TGCTCCAGTGCTAGGATGTACGGATTTGTCTT |
| | | ATCATGGATTGTTGAAAGTCACCCCAAAGCAT |
| | | GACTTAGCTTGCGATTTGGAGTTCATAAGAGC |
| | | TCAGATTTTGGACATTGACGTTTACTCCGCCA |
| | | TAAAAGACTTAGAAGATAAAGCCTTGACTGT |
| | | AAAACAAAAGGTTGAAAAACACTGGTTTACG |
| | | TTTTATGGTAGTTCAGTCTTTCTGCCCGAACAC |
| | | GATGTGCATTACCTGGTTAGACGAGTCATCTT |
| | | TTCGGCTGAAGGAAAGGCGAACTCTCCAGTA |
| | | ACATC |
| 42 | Sequence of the 3′- | CCATATGATGGGTGTTTGCTCACTCGTATGGA |
| | Region used for | TCAAAATTCCATGGTTTCTTCTGTACAACTTGT |
| | knock out of | ACACTTATTTGGACTTTTCTAACGGTTTTTCTG |
| | PpBMT2: | GTGATTTGAGAAGTCCTTATTTTGGTGTTCGC |
| | | AGCTTATCCGTGATTGAACCATCAGAAATACT |
| | | GCAGCTCGTTATCTAGTTTCAGAATGTGTTGT |
| | | AGAATACAATCAATTCTGAGTCTAGTTTGGGT |
| | | GGGTCTTGGCGACGGGACCGTTATATGCATCT |
| | | ATGCAGTGTTAAGGTACATAGAATGAAAATG |
| | | TAGGGGTTAATCGAAAGCATCGTTAATTTCAG |
| | | TAGAACGTAGTTCTATTCCCTACCCAAATAAT |
| | | TTGCCAAGAATGCTTCGTATCCACATACGCAG |
| | | TGGACGTAGCAAATTTCACTTTGGACTGTGAC |
| | | CTCAAGTCGTTATCTTCTACTTGGACATTGAT |
| | | GGTCATTACGTAATCCACAAAGAATTGGATAG |
| | | CCTCTCGTTTTATCTAGTGCACAGCCTAATAG |
| | | CACTTAAGTAAGAGCAATGGACAAATTTGCAT |
| | | AGACATTGAGCTAGATACGTAACTCAGATCTT |
| | | GTTCACTCATGGTGTACTCGAAGTACTGCTGG |
| | | AACCGTTACCTCTTATCATTTCGCTACTGGCTC |
| | | GTGAAACTACTGGATGAAAAAAAAAAAAGAG |
| | | CTGAAAGCGAGATCATCCCATTTTGTCATCAT |
| | | ACAAATTCACGCTTGCAGTTTTGCTTCGTTAA |
| | | CAAGACAAGATGTCTTTATCAAAGACCCGTTT |
| | | TTTCTTCTTGAAGAATACTTCCCTGTTGAGCAC |
| | | ATGCAAACCATATTTATCTCAGATTTCACTCA |
| | | ACTTGGGTGCTTCCAAGAGAAGTAAAATTCTT |
| | | CCCACTGCATCAACTTCCAAGAAACCCGTAGA |
| | | CCAGTTTCTCTTCAGCCAAAAGAAGTTGCTCG |
| | | CCGATCACCGCGGTAACAGAGGAGTCAGAAG |
| | | GTTTCACACCCTTCCATCCCGATTTCAAAGTC |
| | | AAAGTGCTGCGTTGAACCAAGGTTTTCAGGTT |
| | | GCCAAAGCCCAGTCTGCAAAAACTAGTTCCA |
| | | AATGGCCTATTAATTCCCATAAAAGTGTTGGC |
| | | TACGTATGTATCGGTACCTCCATTCTGGTATTT |
| | | GCTATTGTTGTCGTTGGTGGGTTGACTAGACT |
| | | GACCGAATCCGGTCTTTCCATAACGGAGTGGA |
| | | AACCTATCACTGGTTCGGTTCCCCCACTGACT |
| | | GAGGAAGACTGGAAGTTGGAATTTGAAAAAT |
| | | ACAAACAAAGCCCTGAGTTTCAGGAACTAAA |
| | | TTCTCACATAACATTGGAAGAGTTCAAGTTTA |
| | | TATTTTCCATGGAATGGGGACATAGATTGTTG |
| | | GGAAGGGTCATCGGCCTGTCGTTTGTTCTTCC |
| | | CACGTTTTACTTCATTGCCCGTCGAAAGTGTT |
| | | CCAAAGATGTTGCATTGAAACTGCTTGCAATA |
| | | TGCTCTATGATAGGATTCCAAGGTTTCATCGG |
| | | CTGGTGGATGGTGTATTCCGGATTGGACAAAC |
| | | AGCAATTGGCTGAACGTAACTCCAAACCAACT |
| | | GTGTCTCCATATCGCTTAACTACCCATCTTGG |
| | | AACTGCATTTGTTATTTACTGTTACATGATTTA |
| | | CACAGGGCTTCAAGTTTTGAAGAACTATAAGA |
| | | TCATGAAACAGCCTGAAGCGTATGTTCAAATT |
| | | TTCAAGCAAATTGCGTCTCCAAAATTGAAAAC |
| | | TTTCAAGAGACTCTCTTCAGTTCTATTAGGCCT |
| | | GGTG |
| 43 | Sequence of the 5′- | CATATGGTGAGAGCCGTTCTGCACAACTAGAT |
| | Region used for | GTTTTCGAGCTTCGCATTGTTTCCTGCAGCTCG |
| | knock out of | ACTATTGAATTAAGATTTCCGGATATCTCCAA |
| | BMT1 | TCTCACAAAAACTTATGTTGACCACGTGCTTT |
| | | CCTGAGGCGAGGTGTTTTATATGCAAGCTGCC |
| | | AAAAATGGAAAACGAATGGCCATTTTTCGCCC |
| | | AGGCAAATTATTCGATTACTGCTGTCATAAAG |
| | | ACAGTGTTGCAAGGCTCACATTTTTTTTTAGG |
| | | ATCCGAGATAAAGTGAATACAGGACAGCTTA |
| | | TCTCTATATCTTGTACCATTCGTGAATCTTAAG |
| | | AGTTCGGTTAGGGGGACTCTAGTTGAGGGTTG |
| | | GCACTCACGTATGGCTGGGCGCAGAAATAAA |
| | | ATTCAGGCGCAGCAGCACTTATCGATG |
| 44 | Sequence of the 3′- | GAATTCACAGTTATAAATAAAAACAAAAACT |
| | Region used for | CAAAAAGTTTGGGCTCCACAAAATAACTTAAT |
| | knock out of BMT1 | TTAAATTTTTGTCTAATAAATGAATGTAATTC |
| | | CAAGATTATGTGATGCAAGCACAGTATGCTTC |
| | | AGCCCTATGCAGCTACTAATGTCAATCTCGCC |
| | | TGCGAGCGGGCCTAGATTTTCACTACAAATTT |
| | | CAAAACTACGCGGATTTATTGTCTCAGAGAGC |
| | | AATTTGGCATTTCTGAGCGTAGCAGGAGGCTT |
| | | CATAAGATTGTATAGGACCGTACCAACAAATT |
| | | GCCGAGGCACAACACGGTATGCTGTGCACTTA |
| | | TGTGGCTACTTCCCTACAACGGAATGAAACCT |
| | | TCCTCTTTCCGCTTAAACGAGAAAGTGTGTCG |
| | | CAATTGAATGCAGGTGCCTGTGCGCCTTGGTG |
| | | TATTGTTTTTGAGGGCCCAATTTATCAGGCGC |
| | | CTTTTTTCTTGGTTGTTTTCCCTTAGCCTCAAG |
| | | CAAGGTTGGTCTATTTCATCTCCGCTTCTATAC |
| | | CGTGCCTGATACTGTTGGATGAGAACACGACT |
| | | CAACTTCCTGCTGCTCTGTATTGCCAGTGTTTT |
| | | GTCTGTGATTTGGATCGGAGTCCTCCTTACTT |
| | | GGAATGATAATAATCTTGGCGGAATCTCCCTA |
| | | AACGGAGGCAAGGATTCTGCCTATGATGATCT |
| | | GCTATCATTGGGAAGCTT |
| 45 | Sequence of the 5′- | GATATCTCCCTGGGGACAATATGTGTTGCAAC |
| | Region used for | TGTTCGTTGTTGGTGCCCCAGTCCCCCAACCG |
| | knock out of BMT3 | GTACTAATCGGTCTATGTTCCCGTAACTCATA |
| | | TTCGGTTAGAACTAGAACAATAAGTGCATCAT |
| | | TGTTCAACATTGTGGTTCAATTGTCGAACATT |
| | | GCTGGTGCTTATATCTACAGGGAAGACGATAA |
| | | GCCTTTGTACAAGAGAGGTAACAGACAGTTA |
| | | ATTGGTATTTCTTTGGGAGTCGTTGCCCTCTAC |
| | | GTTGTCTCCAAGACATACTACATTCTGAGAAA |
| | | CAGATGGAAGACTCAAAAATGGGAGAAGCTT |
| | | AGTGAAGAAGAGAAAGTTGCCTACTTGGACA |
| | | GAGCTGAGAAGGAGAACCTGGGTTCTAAGAG |
| | | GCTGGACTTTTTGTTCGAGAGTTAAACTGCAT |
| | | AATTTTTTCTAAGTAAATTTCATAGTTATGAA |
| | | ATTTCTGCAGCTTAGTGTTTACTGCATCGTTTA |
| | | CTGCATCACCCTGTAAATAATGTGAGCTTTTT |
| | | TCCTTCCATTGCTTGGTATCTTCCTTGCTGCTG |
| | | TTT |
| 46 | Sequence of the 3′- | ACAAAACAGTCATGTACAGAACTAACGCCTTT |
| | Region used for | AAGATGCAGACCACTGAAAAGAATTGGGTCC |
| | knock out of BMT3 | CATTTTTCTTGAAAGACGACCAGGAATCTGTC |
| | | CATTTTGTTTACTCGTTCAATCCTCTGAGAGTA |
| | | CTCAACTGCAGTCTTGATAACGGTGCATGTGA |
| | | TGTTCTATTTGAGTTACCACATGATTTTGGCAT |
| | | GTCTTCCGAGCTACGTGGTGCCACTCCTATGC |
| | | TCAATCTTCCTCAGGCAATCCCGATGGCAGAC |
| | | GACAAAGAAATTTGGGTTTCATTCCCAAGAAC |
| | | GAGAATATCAGATTGCGGGTGTTCTGAAACA |
| | | ATGTACAGGCCAATGTTAATGCTTTTTGTTAG |
| | | AGAAGGAACAAACTTTTTTGCTGAGC |
| 47 | Sequence of the 5′- | AAGCTTGTTCACCGTTGGGACTTTTCCGTGGA |
| | Region used for | CAATGTTGACTACTCCAGGAGGGATTCCAGCT |
| | knock out of BMT4 | TTCTCTACTAGCTCAGCAATAATCAATGCAGC |
| | | CCCAGGCGCCCGTTCTGATGGCTTGATGACCG |
| | | TTGTATTGCCTGTCACTATAGCCAGGGGTAGG |
| | | GTCCATAAAGGAATCATAGCAGGGAAATTAA |
| | | AAGGGCATATTGATGCAATCACTCCCAATGGC |
| | | TCTCTTGCCATTGAAGTCTCCATATCAGCACT |
| | | AACTTCCAAGAAGGACCCCTTCAAGTCTGACG |
| | | TGATAGAGCACGCTTGCTCTGCCACCTGTAGT |
| | | CCTCTCAAAACGTCACCTTGTGCATCAGCAAA |
| | | GACTTTACCTTGCTCCAATACTATGACGGAGG |
| | | CAATTCTGTCAAAATTCTCTCTCAGCAATTCA |
| | | ACCAACTTGAAAGCAAATTGCTGTCTCTTGAT |
| | | GATGGAGACTTTTTTCCAAGATTGAAATGCAA |
| | | TGTGGGACGACTCAATTGCTTCTTCCAGCTCC |
| | | TCTTCGGTTGATTGAGGAACTTTTGAAACCAC |
| | | AAAATTGGTCGTTGGGTCATGTACATCAAACC |
| | | ATTCTGTAGATTTAGATTCGACGAAAGCGTTG |
| | | TTGATGAAGGAAAAGGTTGGATACGGTTTGTC |
| | | GGTCTCTTTGGTATGGCCGGTGGGGTATGCAA |
| | | TTGCAGTAGAAGATAATTGGACAGCCATTGTT |
| | | GAAGGTAGAGAAAAGGTCAGGGAACTTGGGG |
| | | GTTATTTATACCATTTTACCCCACAAATAACA |
| | | ACTGAAAAGTACCCATTCCATAGTGAGAGGT |
| | | AACCGACGGAAAAAGACGGGCCCATGTTCTG |
| | | GGACCAATAGAACTGTGTAATCCATTGGGACT |
| | | AATCAACAGACGATTGGCAATATAATGAAAT |
| | | AGTTCGTTGAAAAGCCACGTCAGCTGTCTTTT |
| | | CATTAACTTTGGTCGGACACAACATTTTCTAC |
| | | TGTTGTATCTGTCCTACTTTGCTTATCATCTGC |
| | | CACAGGGCAAGTGGATTTCCTTCTCGCGCGGC |
| | | TGGGTGAAAACGGTTAACGTGAA |
| 48 | Sequence of the 3′- | GCCTTGGGGGACTTCAAGTCTTTGCTAGAAAC |
| | Region used for | TAGATGAGGTCAGGCCCTCTTATGGTTGTGTC |
| | knock out of BMT4 | CCAATTGGGCAATTTCACTCACCTAAAAAGCA |
| | | TGACAATTATTTAGCGAAATAGGTAGTATATT |
| | | TTCCCTCATCTCCCAAGCAGTTTCGTTTTTGCA |
| | | TCCATATCTCTCAAATGAGCAGCTACGACTCA |
| | | TTAGAACCAGAGTCAAGTAGGGGTGAGCTCA |
| | | GTCATCAGCCTTCGTTTCTAAAACGATTGAGT |
| | | TCTTTTGTTGCTACAGGAAGCGCCCTAGGGAA |
| | | CTTTCGCACTTTGGAAATAGATTTTGATGACC |
| | | AAGAGCGGGAGTTGATATTAGAGAGGCTGTC |
| | | CAAAGTACATGGGATCAGGCCGGCCAAATTG |
| | | ATTGGTGTGACTAAACCATTGTGTACTTGGAC |
| | | ACTCTATTACAAAAGCGAAGATGATTTGAAGT |
| | | ATTACAAGTCCCGAAGTGTTAGAGGATTCTAT |
| | | CGAGCCCAGAATGAAATCATCAACCGTTATCA |
| | | GCAGATTGATAAACTCTTGGAAAGCGGTATCC |
| | | CATTTTCATTATTGAAGAACTACGATAATGAA |
| | | GATGTGAGAGACGGCGACCCTCTGAACGTAG |
| | | ACGAAGAAACAAATCTACTTTTGGGGTACAAT |
| | | AGAGAAAGTGAATCAAGGGAGGTATTTGTGG |
| | | CCATAATACTCAACTCTATCATTAATG |
| 49 | Sequence of the 5′- | TCATTCTATATGTTCAAGAAAAGGGTAGTGAA |
| | Region used for | AGGAAAGAAAAGGCATATAGGCGAGGGAGA |
| | knock out of | GTTAGCTAGCATACAAGATAATGAAGGATCA |
| | PpPNO1 and | ATAGCGGTAGTTAAAGTGCACAAGAAAAGAG |
| | PpMNN4: | CACCTGTTGAGGCTGATGATAAAGCTCCAATT |
| | | ACATTGCCACAGAGAAACACAGTAACAGAAA |
| | | TAGGAGGGGATGCACCACGAGAAGAGCATTC |
| | | AGTGAACAACTTTGCCAAATTCATAACCCCAA |
| | | GCGCTAATAAGCCAATGTCAAAGTCGGCTACT |
| | | AACATTAATAGTACAACAACTATCGATTTTCA |
| | | ACCAGATGTTTGCAAGGACTACAAACAGACA |
| | | GGTTACTGCGGATATGGTGACACTTGTAAGTT |
| | | TTTGCACCTGAGGGATGATTTCAAACAGGGAT |
| | | GGAAATTAGATAGGGAGTGGGAAAATGTCCA |
| | | AAAGAAGAAGCATAATACTCTCAAAGGGGTT |
| | | AAGGAGATCCAAATGTTTAATGAAGATGAGC |
| | | TCAAAGATATCCCGTTTAAATGCATTATATGC |
| | | AAAGGAGATTACAAATCACCCGTGAAAACTT |
| | | CTTGCAATCATTATTTTTGCGAACAATGTTTCC |
| | | TGCAACGGTCAAGAAGAAAACCAAATTGTAT |
| | | TATATGTGGCAGAGACACTTTAGGAGTTGCTT |
| | | TACCAGCAAAGAAGTTGTCCCAATTTCTGGCT |
| | | AAGATACATAATAATGAAAGTAATAAAGTTT |
| | | AGTAATTGCATTGCGTTGACTATTGATTGCAT |
| | | TGATGTCGTGTGATACTTTCACCGAAAAAAAA |
| | | CACGAAGCGCAATAGGAGCGGTTGCATATTA |
| | | GTCCCCAAAGCTATTTAATTGTGCCTGAAACT |
| | | GTTTTTTAAGCTCATCAAGCATAATTGTATGC |
| | | ATTGCGACGTAACCAACGTTTAGGCGCAGTTT |
| | | AATCATAGCCCACTGCTAAGCC |
| 50 | Sequence of the 3′- | CGGAGGAATGCAAATAATAATCTCCTTAATTA |
| | Region used for | CCCACTGATAAGCTCAAGAGACGCGGTTTGA |
| | knock out of | AAACGATATAATGAATCATTTGGATTTTATAA |
| | PpPNO1 and | TAAACCCTGACAGTTTTTCCACTGTATTGTTTT |
| | PpMNN4: | AACACTCATTGGAAGCTGTATTGATTCTAAGA |
| | | AGCTAGAAATCAATACGGCCATACAAAAGAT |
| | | GACATTGAATAAGCACCGGCTTTTTTGATTAG |
| | | CATATACCTTAAAGCATGCATTCATGGCTACA |
| | | TAGTTGTTAAAGGGCTTCTTCCATTATCAGTA |
| | | TAATGAATTACATAATCATGCACTTATATTTG |
| | | CCCATCTCTGTTCTCTCACTCTTGCCTGGGTAT |
| | | ATTCTATGAAATTGCGTATAGCGTGTCTCCAG |
| | | TTGAACCCCAAGCTTGGCGAGTTTGAAGAGA |
| | | ATGCTAACCTTGCGTATTCCTTGCTTCAGGAA |
| | | ACATTCAAGGAGAAACAGGTCAAGAAGCCAA |
| | | ACATTTTGATCCTTCCCGAGTTAGCATTGACT |
| | | GGCTACAATTTTCAAAGCCAGCAGCGGATAG |
| | | AGCCTTTTTTGGAGGAAACAACCAAGGGAGC |
| | | TAGTACCCAATGGGCTCAAAAAGTATCCAAG |
| | | ACGTGGGATTGCTTTACTTTAATAGGATACCC |
| | | AGAAAAAAGTTTAGAGAGCCCTCCCCGTATTT |
| | | ACAACAGTGCGGTACTTGTATCGCCTCAGGGA |
| | | AAAGTAATGAACAACTACAGAAAGTCCTTCTT |
| | | GTATGAAGCTGATGAACATTGGGGATGTTCGG |
| | | AATCTTCTGATGGGTTTCAAACAGTAGATTTA |
| | | TTAATTGAAGGAAAGACTGTAAAGACATCATT |
| | | TGGAATTTGCATGGATTTGAATCCTTATAAAT |
| | | TTGAAGCTCCATTCACAGACTTCGAGTTCAGT |
| | | GGCCATTGCTTGAAAACCGGTACAAGACTCAT |
| | | TTTGTGCCCAATGGCCTGGTTGTCCCCTCTATC |
| | | GCCTTCCATTAAAAAGGATCTTAGTGATATAG |
| | | AGAAAAGCAGACTTCAAAAGTTCTACCTTGA |
| | | AAAAATAGATACCCCGGAATTTGACGTTAATT |
| | | ACGAATTGAAAAAAGATGAAGTATTGCCCAC |
| | | CCGTATGAATGAAACGTTGGAAACAATTGACT |
| | | TTGAGCCTTCAAAACCGGACTACTCTAATATA |
| | | AATTATTGGATACTAAGGTTTTTTCCCTTTCTG |
| | | ACTCATGTCTATAAACGAGATGTGCTCAAAGA |
| | | GAATGCAGTTGCAGTCTTATGCAACCGAGTTG |
| | | GCATTGAGAGTGATGTCTTGTACGGAGGATCA |
| | | ACCACGATTCTAAACTTCAATGGTAAGTTAGC |
| | | ATCGACACAAGAGGAGCTGGAGTTGTACGGG |
| | | CAGACTAATAGTCTCAACCCCAGTGTGGAAGT |
| | | ATTGGGGGCCCTTGGCATGGGTCAACAGGGA |
| | | ATTCTAGTACGAGACATTGAATTAACATAATA |
| | | TACAATATACAATAAACACAAATAAAGAATA |
| | | CAAGCCTGACAAAAATTCACAAATTATTGCCT |
| | | AGACTTGTCGTTATCAGCAGCGACCTTTTTCC |
| | | AATGCTCAATTTCACGATATGCCTTTTCTAGCT |
| | | CTGCTTTAAGCTTCTCATTGGAATTGGCTAAC |
| | | TCGTTGACTGCTTGGTCAGTGATGAGTTTCTC |
| | | CAAGGTCCATTTCTCGATGTTGTTGTTTTCGTT |
| | | TTCCTTTAATCTCTTGATATAATCAACAGCCTT |
| | | CTTTAATATCTGAGCCTTGTTCGAGTCCCCTGT |
| | | TGGCAACAGAGCGGCCAGTTCCTTTATTCCGT |
| | | GGTTTATATTTTCTCTTCTACGCCTTTCTACTT |
| | | CTTTGTGATTCTCTTTACGCATCTTATGCCATT |
| | | CTTCAGAACCAGTGGCTGGCTTAACCGAATAG |
| | | CCAGAGCCTGAAGAAGCCGCACTAGAAGAAG |
| | | CAGTGGCATTGTTGACTATGG |
| 51 | Sequence of the 5′- | GATCTGGCCATTGTGAAACTTGACACTAAAGA |
| | Region used for | CAAAACTCTTAGAGTTTCCAATCACTTAGGAG |
| | knock out of | ACGATGTTTCCTACAACGAGTACGATCCCTCA |
| | PpMNN4L1: | TTGATCATGAGCAATTTGTATGTGAAAAAAGT |
| | | CATCGACCTTGACACCTTGGATAAAAGGGCTG |
| | | GAGGAGGTGGAACCACCTGTGCAGGCGGTCT |
| | | GAAAGTGTTCAAGTACGGATCTACTACCAAAT |
| | | ATACATCTGGTAACCTGAACGGCGTCAGGTTA |
| | | GTATACTGGAACGAAGGAAAGTTGCAAAGCT |
| | | CCAAATTTGTGGTTCGATCCTCTAATTACTCTC |
| | | AAAAGCTTGGAGGAAACAGCAACGCCGAATC |
| | | AATTGACAACAATGGTGTGGGTTTTGCCTCAG |
| | | CTGGAGACTCAGGCGCATGGATTCTTTCCAAG |
| | | CTACAAGATGTTAGGGAGTACCAGTCATTCAC |
| | | TGAAAAGCTAGGTGAAGCTACGATGAGCATT |
| | | TTCGATTTCCACGGTCTTAAACAGGAGACTTC |
| | | TACTACAGGGCTTGGGGTAGTTGGTATGATTC |
| | | ATTCTTACGACGGTGAGTTCAAACAGTTTGGT |
| | | TTGTTCACTCCAATGACATCTATTCTACAAAG |
| | | ACTTCAACGAGTGACCAATGTAGAATGGTGTG |
| | | TAGCGGGTTGCGAAGATGGGGATGTGGACAC |
| | | TGAAGGAGAACACGAATTGAGTGATTTGGAA |
| | | CAACTGCATATGCATAGTGATTCCGACTAGTC |
| | | AGGCAAGAGAGAGCCCTCAAATTTACCTCTCT |
| | | GCCCCTCCTCACTCCTTTTGGTACGCATAATT |
| | | GCAGTATAAAGAACTTGCTGCCAGCCAGTAAT |
| | | CTTATTTCATACGCAGTTCTATATAGCACATA |
| | | ATCTTGCTTGTATGTATGAAATTTACCGCGTTT |
| | | TAGTTGAAATTGTTTATGTTGTGTGCCTTGCAT |
| | | GAAATCTCTCGTTAGCCCTATCCTTACATTTA |
| | | ACTGGTCTCAAAACCTCTACCAATTCCATTGC |
| | | TGTACAACAATATGAGGCGGCATTACTGTAGG |
| | | GTTGGAAAAAAATTGTCATTCCAGCTAGAGAT |
| | | CACACGACTTCATCACGCTTATTGCTCCTCAT |
| | | TGCTAAATCATTTACTCTTGACTTCGACCCAG |
| | | AAAAGTTCGCC |
| 52 | Sequence of the 3′- | GCATGTCAAACTTGAACACAACGACTAGATA |
| | Region used for | GTTGTTTTTTCTATATAAAACGAAACGTTATC |
| | knock out of | ATCTTTAATAATCATTGAGGTTTACCCTTATA |
| | PpMNN4L1: | GTTCCGTATTTTCGTTTCCAAACTTAGTAATCT |
| | | TTTGGAAATATCATCAAAGCTGGTGCCAATCT |
| | | TCTTGTTTGAAGTTTCAAACTGCTCCACCAAG |
| | | CTACTTAGAGACTGTTCTAGGTCTGAAGCAAC |
| | | TTCGAACACAGAGACAGCTGCCGCCGATTGTT |
| | | CTTTTTTGTGTTTTTCTTCTGGAAGAGGGGCAT |
| | | CATCTTGTATGTCCAATGCCCGTATCCTTTCTG |
| | | AGTTGTCCGACACATTGTCCTTCGAAGAGTTT |
| | | CCTGACATTGGGCTTCTTCTATCCGTGTATTAA |
| | | TTTTGGGTTAAGTTCCTCGTTTGCATAGCAGT |
| | | GGATACCTCGATTTTTTTGGCTCCTATTTACCT |
| | | GACATAATATTCTACTATAATCCAACTTGGAC |
| | | GCGTCATCTATGATAACTAGGCTCTCCTTTGTT |
| | | CAAAGGGGACGTCTTCATAATCCACTGGCACG |
| | | AAGTAAGTCTGCAACGAGGCGGCTTTTGCAAC |
| | | AGAACGATAGTGTCGTTTCGTACTTGGACTAT |
| | | GCTAAACAAAAGGATCTGTCAAACATTTCAAC |
| | | CGTGTTTCAAGGCACTCTTTACGAATTATCGA |
| | | CCAAGACCTTCCTAGACGAACATTTCAACATA |
| | | TCCAGGCTACTGCTTCAAGGTGGTGCAAATGA |
| | | TAAAGGTATAGATATTAGATGTGTTTGGGACC |
| | | TAAAACAGTTCTTGCCTGAAGATTCCCTTGAG |
| | | CAACAGGCTTCAATAGCCAAGTTAGAGAAGC |
| | | AGTACCAAATCGGTAACAAAAGGGGGAAGCA |
| | | TATAAAACCTTTACTATTGCGACAAAATCCAT |
| | | CCTTGAAAGTAAAGCTGTTTGTTCAATGTAAA |
| | | GCATACGAAACGAAGGAGGTAGATCCTAAGA |
| | | TGGTTAGAGAACTTAACGGGACATACTCCAGC |
| | | TGCATCCCATATTACGATCGCTGGAAGACTTT |
| | | TTTCATGTACGTATCGCCCACCAACCTTTCAA |
| | | AGCAAGCTAGGTATGATTTTGACAGTTCTCAC |
| | | AATCCATTGGTTTTCATGCAACTTGAAAAAAC |
| | | CCAACTCAAACTTCATGGGGATCCATACAATG |
| | | TAAATCATTACGAGAGGGCGAGGTTGAAAAG |
| | | TTTCCATTGCAATCACGTCGCATCATGGCTAC |
| | | TGAAAGGCCTTAAC |
| 53 | Sequence of the | TAATGGCCAAACGGTTTCTCAATTACTATATA |
| | PpTRP2 gene | CTACTAACCATTTACCTGTAGCGTATTTCTTTT |
| | integration locus: | CCCTCTTCGCGAAAGCTCAAGGGCATCTTCTT |
| | | GACTCATGAAAAATATCTGGATTTCTTCTGAC |
| | | AGATCATCACCCTTGAGCCCAACTCTCTAGCC |
| | | TATGAGTGTAAGTGATAGTCATCTTGCAACAG |
| | | ATTATTTTGGAACGCAACTAACAAAGCAGATA |
| | | CACCCTTCAGCAGAATCCTTTCTGGATATTGT |
| | | GAAGAATGATCGCCAAAGTCACAGTCCTGAG |
| | | ACAGTTCCTAATCTTTACCCCATTTACAAGTT |
| | | CATCCAATCAGACTTCTTAACGCCTCATCTGG |
| | | CTTATATCAAGCTTACCAACAGTTCAGAAACT |
| | | CCCAGTCCAAGTTTCTTGCTTGAAAGTGCGAA |
| | | GAATGGTGACACCGTTGACAGGTACACCTTTA |
| | | TGGGACATTCCCCCAGAAAAATAATCAAGAC |
| | | TGGGCCTTTAGAGGGTGCTGAAGTTGACCCCT |
| | | TGGTGCTTCTGGAAAAAGAACTGAAGGGCAC |
| | | CAGACAAGCGCAACTTCCTGGTATTCCTCGTC |
| | | TAAGTGGTGGTGCCATAGGATACATCTCGTAC |
| | | GATTGTATTAAGTACTTTGAACCAAAAACTGA |
| | | AAGAAAACTGAAAGATGTTTTGCAACTTCCGG |
| | | AAGCAGCTTTGATGTTGTTCGACACGATCGTG |
| | | GCTTTTGACAATGTTTATCAAAGATTCCAGGT |
| | | AATTGGAAACGTTTCTCTATCCGTTGATGACT |
| | | CGGACGAAGCTATTCTTGAGAAATATTATAAG |
| | | ACAAGAGAAGAAGTGGAAAAGATCAGTAAAG |
| | | TGGTATTTGACAATAAAACTGTTCCCTACTAT |
| | | GAACAGAAAGATATTATTCAAGGCCAAACGT |
| | | TCACCTCTAATATTGGTCAGGAAGGGTATGAA |
| | | AACCATGTTCGCAAGCTGAAAGAACATATTCT |
| | | GAAAGGAGACATCTTCCAAGCTGTTCCCTCTC |
| | | AAAGGGTAGCCAGGCCGACCTCATTGCACCC |
| | | TTTCAACATCTATCGTCATTTGAGAACTGTCA |
| | | ATCCTTCTCCATACATGTTCTATATTGACTATC |
| | | TAGACTTCCAAGTTGTTGGTGCTTCACCTGAA |
| | | TTACTAGTTAAATCCGACAACAACAACAAAAT |
| | | CATCACACATCCTATTGCTGGAACTCTTCCCA |
| | | GAGGTAAAACTATCGAAGAGGACGACAATTA |
| | | TGCTAAGCAATTGAAGTCGTCTTTGAAAGACA |
| | | GGGCCGAGCACGTCATGCTGGTAGATTTGGCC |
| | | AGAAATGATATTAACCGTGTGTGTGAGCCCAC |
| | | CAGTACCACGGTTGATCGTTTATTGACTGTGG |
| | | AGAGATTTTCTCATGTGATGCATCTTGTGTCA |
| | | GAAGTCAGTGGAACATTGAGACCAAACAAGA |
| | | CTCGCTTCGATGCTTTCAGATCCATTTTCCCAG |
| | | CAGGAACCGTCTCCGGTGCTCCGAAGGTAAG |
| | | AGCAATGCAACTCATAGGAGAATTGGAAGGA |
| | | GAAAAGAGAGGTGTTTATGCGGGGGCCGTAG |
| | | GACACTGGTCGTACGATGGAAAATCGATGGA |
| | | CACATGTATTGCCTTAAGAACAATGGTCGTCA |
| | | AGGACGGTGTCGCTTACCTTCAAGCCGGAGGT |
| | | GGAATTGTCTACGATTCTGACCCCTATGACGA |
| | | GTACATCGAAACCATGAACAAAATGAGATCC |
| | | AACAATAACACCATCTTGGAGGCTGAGAAAA |
| | | TCTGGACCGATAGGTTGGCCAGAGACGAGAA |
| | | TCAAAGTGAATCCGAAGAAAACGATCAATGA |
| | | ACGGAGGACGTAAGTAGGAATTTATGGTTTG |
| | | GCCAT |
| 54 | Sequence of the | TTTTTGTAGAAATGTCTTGGTGTCCTCGTCCAA |
| | PpGAPDH | TCAGGTAGCCATCTCTGAAATATCTGGCTCCG |
| | promoter: | TTGCAACTCCGAACGACCTGCTGGCAACGTAA |
| | | AATTCTCCGGGGTAAAACTTAAATGTGGAGTA |
| | | ATGGAACCAGAAACGTCTCTTCCCTTCTCTCT |
| | | CCTTCCACCGCCCGTTACCGTCCCTAGGAAAT |
| | | TTTACTCTGCTGGAGAGCTTCTTCTACGGCCC |
| | | CCTTGCAGCAATGCTCTTCCCAGCATTACGTT |
| | | GCGGGTAAAACGGAGGTCGTGTACCCGACCT |
| | | AGCAGCCCAGGGATGGAAAAGTCCCGGCCGT |
| | | CGCTGGCAATAATAGCGGGCGGACGCATGTC |
| | | ATGAGATTATTGGAAACCACCAGAATCGAAT |
| | | ATAAAAGGCGAACACCTTTCCCAATTTTGGTT |
| | | TCTCCTGACCCAAAGACTTTAAATTTAATTTA |
| | | TTTGTCCCTATTTCAATCAATTGAACAACTATC |
| | | AAAACACA |
| 55 | Sequence of the | ATTTACAATTAGTAATATTAAGGTGGTAAAAA |
| | PpALG3 | CATTCGTAGAATTGAAATGAATTAATATAGTA |
| | terminator: | TGACAATGGTTCATGTCTATAAATCTCCGGCT |
| | | TCGGTACCTTCTCCCCAATTGAATACATTGTC |
| | | AAAATGAATGGTTGAACTATTAGGTTCGCCAG |
| | | TTTCGTTATTAAGAAAACTGTTAAAATCAAAT |
| | | TCCATATCATCGGTTCCAGTGGGAGGACCAGT |
| | | TCCATCGCCAAAATCCTGTAAGAATCCATTGT |
| | | CAGAACCTGTAAAGTCAGTTTGAGATGAAATT |
| | | TTTCCGGTCTTTGTTGACTTGGAAGCTTCGTTA |
| | | AGGTTAGGTGAAACAGTTTGATCAACCAGCG |
| | | GCTCCCGTTTTCGTCGCTTAGTAG |
| 56 | Sequence of the | AACATCCAAAGACGAAAGGTTGAATGAAACC |
| | PpAOX1 promoter | TTTTTGCCATCCGACATCCACAGGTCCATTCT |
| | and integration | CACACATAAGTGCCAAACGCAACAGGAGGGG |
| | locus: | ATACACTAGCAGCAGACCGTTGCAAACGCAG |
| | | GACCTCCACTCCTCTTCTCCTCAACACCCACTT |
| | | TTGCCATCGAAAAACCAGCCCAGTTATTGGGC |
| | | TTGATTGGAGCTCGCTCATTCCAATTCCTTCTA |
| | | TTAGGCTACTAACACCATGACTTTATTAGCCT |
| | | GTCTATCCTGGCCCCCCTGGCGAGGTTCATGT |
| | | TTGTTTATTTCCGAATGCAACAAGCTCCGCAT |
| | | TACACCCGAACATCACTCCAGATGAGGGCTTT |
| | | CTGAGTGTGGGGTCAAATAGTTTCATGTTCCC |
| | | CAAATGGCCCAAAACTGACAGTTTAAACGCT |
| | | GTCTTGGAACCTAATATGACAAAAGCGTGATC |
| | | TCATCCAAGATGAACTAAGTTTGGTTCGTTGA |
| | | AATGCTAACGGCCAGTTGGTCAAAAAGAAAC |
| | | TTCCAAAAGTCGGCATACCGTTTGTCTTGTTT |
| | | GGTATTGATTGACGAATGCTCAAAAATAATCT |
| | | CATTAATGCTTAGCGCAGTCTCTCTATCGCTT |
| | | CTGAACCCCGGTGCACCTGTGCCGAAACGCA |
| | | AATGGGGAAACACCCGCTTTTTGGATGATTAT |
| | | GCATTGTCTCCACATTGTATGCTTCCAAGATT |
| | | CTGGTGGGAATACTGCTGATAGCCTAACGTTC |
| | | ATGATCAAAATTTAACTGTTCTAACCCCTACT |
| | | TGACAGCAATATATAAACAGAAGGAAGCTGC |
| | | CCTGTCTTAAACCTTTTTTTTTATCATCATTAT |
| | | TAGCTTACTTTCATAATTGCGACTGGTTCCAA |
| | | TTGACAAGCTTTTGATTTTAACGACTTTTAAC |
| | | GACAACTTGAGAAGATCAAAAAACAACTAAT |
| | | TATTCGAAACG |
| 57 | Sequence of the | ACAGGCCCCTTTTCCTTTGTCGATATCATGTA |
| | ScCYC1 | ATTAGTTATGTCACGCTTACATTCACGCCCTC |
| | terminator: | CTCCCACATCCGCTCTAACCGAAAAGGAAGG |
| | | AGTTAGACAACCTGAAGTCTAGGTCCCTATTT |
| | | ATTTTTTTTAATAGTTATGTTAGTATTAAGAAC |
| | | GTTATTTATATTTCAAATTTTTCTTTTTTTTCTG |
| | | TACAAACGCGTGTACGCATGTAACATTATACT |
| | | GAAAACCTTGCTTGAGAAGGTTTTGGGACGCT |
| | | CGAAGGCTTTAATTTGCAAGCTGCCGGCTCTT |
| | | AAG |
| 58 | Sequence of the | GATCCCCCACACACCATAGCTTCAAAATGTTT |
| | ScTEF1 promoter: | CTACTCCTTTTTTACTCTTCCAGATTTTCTCGG |
| | | ACTCCGCGCATCGCCGTACCACTTCAAAACAC |
| | | CCAAGCACAGCATACTAAATTTCCCCTCTTTC |
| | | TTCCTCTAGGGTGTCGTTAATTACCCGTACTA |
| | | AAGGTTTGGAAAAGAAAAAAGAGACCGCCTC |
| | | GTTTCTTTTTCTTCGTCGAAAAAGGCAATAAA |
| | | AATTTTTATCACGTTFCTTTTTCTTGAAAATTT |
| | | TTTTTTTTGATTTTTTTCTCTTTCGATGACCTCC |
| | | CATTGATATTTAAGTTAATAAACGGTCTTCAA |
| | | TTTCTCAAGTTTCAGTTTCATTTTTCTTGTTCT |
| | | ATTACAACTTTTTTTACTTCTTGCTCATTAGAA |
| | | AGAAAGCATAGCAATCTAATCTAAGTTTTAAT |
| | | TACAAA |
| 59 | Sequence of the Shble | ATGGCCAAGTTGACCAGTGCCGTTCCGGTGCT |
| | ORF (Zeocin | CACCGCGCGCGACGTCGCCGGAGCGGTCGAG |
| | resistance marker): | TTCTGGACCGACCGGCTCGGGTTCTCCCGGGA |
| | | CTTCGTGGAGGACGACTTCGCCGGTGTGGTCC |
| | | GGGACGACGTGACCCTGTTCATCAGCGCGGTC |
| | | CAGGACCAGGTGGTGCCGGACAACACCCTGG |
| | | CCTGGGTGTGGGTGCGCGGCCTGGACGAGCT |
| | | GTACGCCGAGTGGTCGGAGGTCGTGTCCACG |
| | | AACTTCCGGGACGCCTCCGGGCCGGCCATGA |
| | | CCGAGATCGGCGAGCAGCCGTGGGGGCGGGA |
| | | GTTCGCCCTGCGCGACCCGGCCGGCAACTGCG |
| | | TGCACTTCGTGGCCGAGGAGCAGGACTGA |
| 60 | NATR ORF | ATGGGTACCACTCTTGACGACACGGCTTACCG |
| | | GTACCGCACCAGTGTCCCGGGGGACGCCGAG |
| | | GCCATCGAGGCACTGGATGGGTCCTTCACCAC |
| | | CGACACCGTCTTCCGCGTCACCGCCACCGGGG |
| | | ACGGCTTCACCCTGCGGGAGGTGCCGGTGGA |
| | | CCCGCCCCTGACCAAGGTGTTCCCCGACGACG |
| | | AATCGGACGACGAATCGGACGACGGGGAGGA |
| | | CGGCGACCCGGACTCCCGGACGTTCGTCGCGT |
| | | ACGGGGACGACGGCGACCTGGCGGGCTTCGT |
| | | GGTCGTCTCGTACTCCGGCTGGAACCGCCGGC |
| | | TGACCGTCGAGGACATCGAGGTCGCCCCGGA |
| | | GCACCGGGGGCACGGGGTCGGGCGCGCGTTG |
| | | ATGGGGCTCGCGACGGAGTTCGCCCGCGAGC |
| | | GGGGCGCCGGGCACCTCTGGCTGGAGGTCAC |
| | | CAACGTCAACGCACCGGCGATCCACGCGTAC |
| | | CGGCGGATGGGGTTCACCCTCTGCGGCCTGGA |
| | | CACCGCCCTGTACGACGGCACCGCCTCGGAC |
| | | GGCGAGCAGGCGCTCTACATGAGCATGCCCT |
| | | GCCCCTAATCAGTACTG |
| 61 | Sequence of the 5′- | GAAGGGCCATCGAATTGTCATCGTCTCCTCAG |
| | region that was | GTGCCATCGCTGTGGGCATGAAGAGAGTCAA |
| | used to knock into | CATGAAGCGGAAACCAAAAAAGTTACAGCAA |
| | the PpPRO1 locus: | GTGCAGGCATTGGCTGCTATAGGACAAGGCC |
| | | GTTTGATAGGACTTTGGGACGACCTTTTCCGT |
| | | CAGTTGAATCAGCCTATTGCGCAGATTTTACT |
| | | GACTAGAACGGATTTGGTCGATTACACCCAGT |
| | | TTAAGAACGCTGAAAATACATTGGAACAGCTT |
| | | ATTAAAATGGGTATTATTCCTATTGTCAATGA |
| | | GAATGACACCCTATCCATTCAAGAAATCAAAT |
| | | TTGGTGACAATGACACCTTATCCGCCATAACA |
| | | GCTGGTATGTGTCATGCAGACTACCTGTTTTT |
| | | GGTGACTGATGTGGACTGTCTTTACACGGATA |
| | | ACCCTCGTACGAATCCGGACGCTGAGCCAATC |
| | | GTGTTAGTTAGAAATATGAGGAATCTAAACGT |
| | | CAATACCGAAAGTGGAGGTTCCGCCGTAGGA |
| | | ACAGGAGGAATGACAACTAAATTGATCGCAG |
| | | CTGATTTGGGTGTATCTGCAGGTGTTACAACG |
| | | ATTATTTGCAAAAGTGAACATCCCGAGCAGAT |
| | | TTTGGACATTGTAGAGTACAGTATCCGTGCTG |
| | | ATAGAGTCGAAAATGAGGCTAAATATCTGGT |
| | | CATCAACGAAGAGGAAACTGTGGAACAATTT |
| | | CAAGAGATCAATCGGTCAGAACTGAGGGAGT |
| | | TGAACAAGCTGGACATTCCTTTGCATACACGT |
| | | TTCGTTGGCCACAGTTTTAATGCTGTTAATAA |
| | | CAAAGAGTTTTGGTTACTCCATGGACTAAAGG |
| | | CCAACGGAGCCATTATCATTGATCCAGGTTGT |
| | | TATAAGGCTATCACTAGAAAAAACAAAGCTG |
| | | GTATTCTTCCAGCTGGAATTATTTCCGTAGAG |
| | | GGTAATTTCCATGAATACGAGTGTGTTGATGT |
| | | TAAGGTAGGACTAAGAGATCCAGATGACCCA |
| | | CATTCACTAGACCCCAATGAAGAACTTTACGT |
| | | CGTTGGCCGTGCCCGTTGTAATTACCCCAGCA |
| | | ATCAAATCAACAAAATTAAGGGTCTACAAAG |
| | | CTCGCAGATCGAGCAGGTTCTAGGTTACGCTG |
| | | ACGGTGAGTATGTTGTTCACAGGGACAACTTG |
| | | GCTTTCCCAGTATTTGCCGATCCAGAACTGTT |
| | | GGATGTTGTTGAGAGTACCCTGTCTGAACAGG |
| | | AGAGAGAATCCAAACCAAATAAATAG |
| 62 | Sequence of the 3′- | AATTTCACATATGCTGCTTGATTATGTAATTAT |
| | region that was | ACCTTGCGTTCGATGGCATCGATTTCCTCTTCT |
| | used to knock into | GTCAATCGCGCATCGCATTAAAAGTATACTTT |
| | the PpPRO1 locus: | TTTTTTTTTCCTATAGTACTATTCGCCTTATTA |
| | | TAAACTTTGCTAGTATGAGTTCTACCCCCAAG |
| | | AAAGAGCCTGATTTGACTCCTAAGAAGAGTC |
| | | AGCCTCCAAAGAATAGTCTCGGTGGGGGTAA |
| | | AGGCTTTAGTGAGGAGGGTTTCTCCCAAGGGG |
| | | ACTTCAGCGCTAAGCATATACTAAATCGTCGC |
| | | CCTAACACCGAAGGCTCTTCTGTGGCTTCGAA |
| | | CGTCATCAGTTCGTCATCATTGCAAAGGTTAC |
| | | CATCCTCTGGATCTGGAAGCGTTGCTGTGGGA |
| | | AGTGTGTTGGGATCTTCGCCATTAACTCTTTCT |
| | | GGAGGGTTCCACGGGCTTGATCCAACCAAGA |
| | | ATAAAATAGACGTTCCAAAGTCGAAACAGTC |
| | | AAGGAGACAAAGTGTTCTTTCTGACATGATTT |
| | | CCACTTCTCATGCAGCTAGAAATGATCACTCA |
| | | GAGCAGCAGTTACAAACTGGACAACAATCAG |
| | | AACAAAAAGAAGAAGATGGTAGTCGATCTTC |
| | | TTTTTCTGTTTCTTCCCCCGCAAGAGATATCCG |
| | | GCACCCAGATGTACTGAAAACTGTCGAGAAA |
| | | CATCTTGCCAATGACAGCGAGATCGACTCATC |
| | | TTTACAACTTCAAGGTGGAGATGTCACTAGAG |
| | | GCATTTATCAATGGGTAACTGGAGAAAGTAGT |
| | | CAAAAAGATAACCCGCCTTTGAAACGAGCAA |
| | | ATAGTTTTAATGATTTTTCTTCTGTGCATGGTG |
| | | ACGAGGTAGGCAAGGCAGATGCTGACCACGA |
| | | TCGTGAAAGCGTATTCGACGAGGATGATATCT |
| | | CCATTGATGATATCAAAGTTCCGGGAGGGATG |
| | | CGTCGAAGTTTTTTATTACAAAAGCATAGAGA |
| | | CCAACAACTTTCTGGACTGAATAAAACGGCTC |
| | | ACCAACCAAAACAACTTACTAAACCTAATTTC |
| | | TTCACGAACAACTTTATAGAGTTTTTGGCATT |
| | | GTATGGGCATTTTGCAGGTGAAGATTTGGAGG |
| | | AAGACGAAGATGAAGATTTAGACAGTGGTTC |
| | | CGAATCAGTCGCAGTCAGTGATAGTGAGGGA |
| | | GAATTCAGTGAGGCTGACAACAATTTGTTGTA |
| | | TGATGAAGAGTCTCTCCTATTAGCACCTAGTA |
| | | CCTCCAACTATGCGAGATCAAGAATAGGAAG |
| | | TATTCGTACTCCTACTTATGGATCTTTCAGTTC |
| | | AAATGTTGGTTCTTCGTCTATTCATCAGCAGTT |
| | | AATGAAAAGTCAAATCCCGAAGCTGAAGAAA |
| | | CGTGGACAGCACAAGCATAAAACACAATCAA |
| | | AAATACGCTCGAAGAAGCAAACTACCACCGT |
| | | AAAAGCAGTGTTGCTGCTATTAAA |
| 63 | DNA encodes Mm | GAGCCCGCTGACGCCACCATCCGTGAGAAGA |
| | ManI catalytic | GGGCAAAGATCAAAGAGATGATGACCCATGC |
| | doman (FB) | TTGGAATAATTATAAACGCTATGCGTGGGGCT |
| | | TGAACGAACTGAAACCTATATCAAAAGAAGG |
| | | CCATTCAAGCAGTTTGTTTGGCAACATCAAAG |
| | | GAGCTACAATAGTAGATGCCCTGGATACCCTT |
| | | TTCATTATGGGCATGAAGACTGAATTTCAAGA |
| | | AGCTAAATCGTGGATTAAAAAATATTTAGATT |
| | | TTAATGTGAATGCTGAAGTTTCTGTTTTTGAA |
| | | GTCAACATACGCTTCGTCGGTGGACTGCTGTC |
| | | AGCCTACTATTTGTCCGGAGAGGAGATATTTC |
| | | GAAAGAAAGCAGTGGAACTTGGGGTAAAATT |
| | | GCTACCTGCATTTCATACTCCCTCTGGAATAC |
| | | CTTGGGCATTGCTGAATATGAAAAGTGGGATC |
| | | GGGCGGAACTGGCCCTGGGCCTCTGGAGGCA |
| | | GCAGTATCCTGGCCGAATTTGGAACTCTGCAT |
| | | TTAGAGTTTATGCACTTGTCCCACTTATCAGG |
| | | AGACCCAGTCTTTGCCGAAAAGGTTATGAAA |
| | | ATTCGAACAGTGTTGAACAAACTGGACAAAC |
| | | CAGAAGGCCTTTATCCTAACTATCTGAACCCC |
| | | AGTAGTGGACAGTGGGGTCAACATCATGTGTC |
| | | GGTTGGAGGACTTGGAGACAGCTTTTATGAAT |
| | | ATTTGCTTAAGGCGTGGTTAATGTCTGACAAG |
| | | ACAGATCTCGAAGCCAAGAAGATGTATTTTGA |
| | | TGCTGTTCAGGCCATCGAGACTCACTTGATCC |
| | | GCAAGTCAAGTGGGGGACTAACGTACATCGC |
| | | AGAGTGGAAGGGGGGCCTCCTGGAACACAAG |
| | | ATGGGCCACCTGACGTGCTTTGCAGGAGGCAT |
| | | GTTTGCACTTGGGGCAGATGGAGCTCCGGAA |
| | | GCCCGGGCCCAACACTACCTTGAACTCGGAG |
| | | CTGAAATTGCCCGCACTTGTCATGAATCTTAT |
| | | AATCGTACATATGTGAAGTTGGGACCGGAAG |
| | | CGTTTCGATTTGATGGCGGTGTGGAAGCTATT |
| | | GCCACGAGGCAAAATGAAAAGTATTACATCT |
| | | TACGGCCCGAGGTCATCGAGACATACATGTAC |
| | | ATGTGGCGACTGACTCACGACCCCAAGTACA |
| | | GGACCTGGGCCTGGGAAGCCGTGGAGGCTCT |
| | | AGAAAGTCACTGCAGAGTGAACGGAGGCTAC |
| | | TCAGGCTTACGGGATGTTTACATTGCCCGTGA |
| | | GAGTTATGACGATGTCCAGCAAAGTTTCTTCC |
| | | TGGCAGAGACACTGAAGTATTTGTACTTGATA |
| | | TTTTCCGATGATGACCTTCTTCCACTAGAACA |
| | | CTGGATCTTCAACACCGAGGCTCATCCTTTCC |
| | | CTATACTCCGTGAACAGAAGAAGGAAATTGA |
| | | TGGCAAAGAGAAATGA |
| 64 | DNA encodes | ATGCTGCTTACCAAAAGGTTTTCAAAGCTGTT |
| | Mnn2 leader (53) | CAAGCTGACGTTCATAGTTTTGATATTGTGCG |
| | | GGCTGTTCGTCATTACAAACAAATACATGGAT |
| | | GAGAACACGTCG |
| 65 | S. cerevisiae | AGGCCTCGCAACAACCTATAATTGAGTTAAGT |
| | invertase gene | GCCTTTCCAAGCTAAAAAGTTTGAGGTTATAG |
| | (ScSUC2) | GGGCTTAGCATCCACACGTCACAATCTCGGGT |
| | | ATCGAGTATAGTATGTAGAATTACGGCAGGA |
| | | GGTTTCCCAATGAACAAAGGACAGGGGCACG |
| | | GTGAGCTGTCGAAGGTATCCATTTTATCATGT |
| | | TTCGTTTGTACAAGCACGACATACTAAGACAT |
| | | TTACCGTATGGGAGTTGTTGTCCTAGCGTAGT |
| | | TCTCGCTCCCCCAGCAAAGCTCAAAAAAGTAC |
| | | GTCATTTAGAATAGTTTGTGAGCAAATTACCA |
| | | GTCGGTATGCTACGTTAGAAAGGCCCACAGTA |
| | | TTCTTCTACCAAAGGCGTGCCTTTGTTGAACT |
| | | CGATCCATTATGAGGGCTTCCATTATTCCCCG |
| | | CATTTTTATTACTCTGAACAGGAATAAAAAGA |
| | | AAAAACCCAGTTTAGGAAATTATCCGGGGGC |
| | | GAAGAAATACGCGTAGCGTTAATCGACCCCA |
| | | CGTCCAGGGTTTTTCCATGGAGGTTTCTGGAA |
| | | AAACTGACGAGGAATGTGATTATAAATCCCTT |
| | | TATGTGATGTCTAAGACTTTTAAGGTACGCCC |
| | | GATGTTTGCCTATTACCATCATAGAGACGTTT |
| | | CTTTTCGAGGAATGCTTAAACGACTTTGTTTG |
| | | ACAAAAATGTTGCCTAAGGGCTCTATAGTAAA |
| | | CCATTTGGAAGAAAGATTTGACGACTTTTTTT |
| | | TTTTGGATTTCGATCCTATAATCCTTCCTCCTG |
| | | AAAAGAAACATATAAATAGATATGTATTATTC |
| | | TTCAAAACATTCTCTTGTTCTTGTGCTTTTTTT |
| | | TTACCATATATCTTACTTTTTTTTTTCTCTCAG |
| | | AGAAACAAGCAAAACAAAAAGCTTTTCTTTTC |
| | | ACTAACGTATATGATGCTTTTGCAAGCTTTCC |
| | | TTTTCCTTTTGGCTGGTTTTGCAGCCAAAATAT |
| | | CTGCATCAATGACAAACGAAACTAGCGATAG |
| | | ACCTTTGGTCCACTTCACACCCAACAAGGGCT |
| | | GGATGAATGACCCAAATGGGTTGTGGTACGA |
| | | TGAAAAAGATGCCAAATGGCATCTGTACTTTC |
| | | AATACAACCCAAATGACACCGTATGGGGTAC |
| | | GCCATTGTTTTGGGGCCATGCTACTTCCGATG |
| | | ATTTGACTAATTGGGAAGATCAACCCATTGCT |
| | | ATCGCTCCCAAGCGTAACGATTCAGGTGCTTT |
| | | CTCTGGCTCCATGGTGGTTGATTACAACAACA |
| | | CGAGTGGGTTTTTCAATGATACTATTGATCCA |
| | | AGACAAAGATGCGTTGCGATTTGGACTTATAA |
| | | CACTCCTGAAAGTGAAGAGCAATACATTAGCT |
| | | ATTCTCTTGATGGTGGTTACACTTTTACTGAAT |
| | | ACCAAAAGAACCCTGTTTTAGCTGCCAACTCC |
| | | ACTCAATTCAGAGATCCAAAGGTGTTCTGGTA |
| | | TGAACCTTCTCAAAAATGGATTATGACGGCTG |
| | | CCAAATCACAAGACTACAAAATTGAAATTTAC |
| | | TCCTCTGATGACTTGAAGTCCTGGAAGCTAGA |
| | | ATCTGCATTTGCCAATGAAGGTTTCTTAGGCT |
| | | ACCAATACGAATGTCCAGGTTTGATTGAAGTC |
| | | CCAACTGAGCAAGATCCTTCCAAATCTTATTG |
| | | GGTCATGTTTATTTCTATCAACCCAGGTGCAC |
| | | CTGCTGGCGGTTCCTTCAACCAATATTTTGTTG |
| | | GATCCTTCAATGGTACTCATTTTGAAGCGTTT |
| | | GACAATCAATCTAGAGTGGTAGATTTTGGTAA |
| | | GGACTACTATGCCTTGCAAACTTTCTTCAACA |
| | | CTGACCCAACCTACGGTTCAGCATTAGGTATT |
| | | GCCTGGGCTTCAAACTGGGAGTACAGTGCCTT |
| | | TGTCCCAACTAACCCATGGAGATCATCCATGT |
| | | CTTTGGTCCGCAAGTTTTCTTTGAACACTGAA |
| | | TATCAAGCTAATCCAGAGACTGAATTGATCAA |
| | | TTTGAAAGCCGAACCAATATTGAACATTAGTA |
| | | ATGCTGGTCCCTGGTCTCGTTTTGCTACTAAC |
| | | ACAACTCTAACTAAGGCCAATTCTTACAATGT |
| | | CGATTTGAGCAACTCGACTGGTACCCTAGAGT |
| | | TTGAGTTGGTTTACGCTGTTAACACCACACAA |
| | | ACCATATCCAAATCCGTCTTTGCCGACTTATC |
| | | ACTTTGGTTCAAGGGTTTAGAAGATCCTGAAG |
| | | AATATTTGAGAATGGGTTTTGAAGTCAGTGCT |
| | | TCTTCCTTCTTTTTGGACCGTGGTAACTCTAAG |
| | | GTCAAGTTTGTCAAGGAGAACCCATATTTCAC |
| | | AAACAGAATGTCTGTCAACAACCAACCATTCA |
| | | AGTCTGAGAACGACCTAAGTTACTATAAAGTG |
| | | TACGGCCTACTGGATCAAAACATCTTGGAATT |
| | | GTACTTCAACGATGGAGATGTGGTTTCTACAA |
| | | ATACCTACTTCATGACCACCGGTAACGCTCTA |
| | | GGATCTGTGAACATGACCACTGGTGTCGATAA |
| | | TTTGTTCTACATTGACAAGTTCCAAGTAAGGG |
| | | AAGTAAAATAGAGGTTATAAAACTTATTGTCT |
| | | TTTTTATTTTTTTCAAAAGCCATTCTAAAGGGC |
| | | TTTAGCTAACGAGTGACGAATGTAAAACTTTA |
| | | TGATTTCAAAGAATACCTCCAAACCATTGAAA |
| | | ATGTATTTTTATTTTTATTTTCTCCCGACCCCA |
| | | GTTACCTGGAATTTGTTCTTTATGTACTTTATA |
| | | TAAGTATAATTCTCTTAAAAATTTTTACTACTT |
| | | TGCAATAGACATCATTTTTTCACGTAATAAAC |
| | | CCACAATCGTAATGTAGTTGCCTTACACTACT |
| | | AGGATGGACCTTTTTGCCTTTATCTGTTTTGTT |
| | | ACTGACACAATGAAACCGGGTAAAGTATTAG |
| | | TTATGTGAAAATTTAAAAGCATTAAGTAGAAG |
| | | TATACCATATTGTAAAAAAAAAAAGCGTTGTC |
| | | TTCTACGTAAAAGTGTTCTCAAAAAGAAGTAG |
| | | TGAGGGAAATGGATACCAAGCTATCTGTAAC |
| | | AGGAGCTAAAAAATCTCAGGGAAAAGCTTCT |
| | | GGTTTGGGAAACGGTCGAC |
| 66 | K. lactis UDP- | AAACGTAACGCCTGGCACTCTATTTTCTCAAA |
| | GlcNAc transporter | CTTCTGGGACGGAAGAGCTAAATATTGTGTTG |
| | gene (KIMNN2-2) | CTTGAACAAACCCAAAAAAACAAAAAAATGA |
| | | ACAAACTAAAACTACACCTAAATAAACCGTG |
| | | TGTAAAACGTAGTACCATATTACTAGAAAAG |
| | | ATCACAAGTGTATCACACATGTGCATCTCATA |
| | | TTACATCTTTTATCCAATCCATTCTCTCTATCC |
| | | CGTCTGTTCCTGTCAGATTCTTTTTCCATAAAA |
| | | AGAAGAAGACCCCGAATCTCACCGGTACAAT |
| | | GCAAAACTGCTGAAAAAAAAAGAAAGTTCAC |
| | | TGGATACGGGAACAGTGCCAGTAGGCTTCAC |
| | | CACATGGACAAAACAATTGACGATAAAATAA |
| | | GCAGGTGAGCTTCTTTTTCAAGTCACGATCCC |
| | | TTTATGTCTCAGAAACAATATATACAAGCTAA |
| | | ACCCTTTTGAACCAGTTCTCTCTTCATAGTTAT |
| | | GTTCACATAAATTGCGGGAACAAGACTCCGCT |
| | | GGCTGTCAGGTACACGTTGTAACGTTTTCGTC |
| | | CGCCCAATTATTAGCACAACATTGGCAAAAA |
| | | GAAAAACTGCTCGTTTTCTCTACAGGTAAATT |
| | | ACAATTTTTTTCAGTAATTTTCGCTGAAAAATT |
| | | TAAAGGGCAGGAAAAAAAGACGATCTCGACT |
| | | TTGCATAGATGCAAGAACTGTGGTCAAAACTT |
| | | GAAATAGTAATTTTGCTGTGCGTGAACTAATA |
| | | AATATATATATATATATATATATATATTTGTGT |
| | | ATTTTGTATATGTAATTGTGCACGTCTTGGCTA |
| | | TTGGATATAAGATTTTCGCGGGTTGATGACAT |
| | | AGAGCGTGTACTACTGTAATAGTTGTATATTC |
| | | AAAAGCTGCTGCGTGGAGAAAGACTAAAATA |
| | | GATAAAAAGCACACATTTTGACTTCGGTACCG |
| | | TCAACTTAGTGGGACAGTCTTTTATATTTGGT |
| | | GTAAGCTCATTTCTGGTACTATTCGAAACAGA |
| | | ACAGTGTTTTCTGTATTACCGTCCAATCGTTTG |
| | | TCATGAGTTTTGTATTGATTTTGTCGTTAGTGT |
| | | TCGGAGGATGTTGTTCCAATGTGATTAGTTTC |
| | | GAGCACATGGTGCAAGGCAGCAATATAAATT |
| | | TGGGAAATATTGTTACATTCACTCAATTCGTG |
| | | TCTGTGACGCTAATTCAGTTGCCCAATGCTTT |
| | | GGACTTCTCTCACTTTCCGTTTAGGTTGCGAC |
| | | CTAGACACATTCCTCTTAAGATCCATATGTTA |
| | | GCTGTGTTTTTGTTCTTTACCAGTTCAGTCGCC |
| | | AATAACAGTGTGTTTAAATTTGACATTTCCGT |
| | | TCCGATTCATATTATCATTAGATTTTCAGGTAC |
| | | CACTTTGACGATGATAATAGGTTGGGCTGTTT |
| | | GTAATAAGAGGTACTCCAAACTTCAGGTGCA |
| | | ATCTGCCATCATTATGACGCTTGGTGCGATTG |
| | | TCGCATCATTATACCGTGACAAAGAATTTTCA |
| | | ATGGACAGTTTAAAGTTGAATACGGATTCAGT |
| | | GGGTATGACCCAAAAATCTATGTTTGGTATCT |
| | | TTGTTGTGCTAGTGGCCACTGCCTTGATGTCA |
| | | TTGTTGTCGTTGCTCAACGAATGGACGTATAA |
| | | CAAGTACGGGAAACATTGGAAAGAAACTTTG |
| | | TTCTATTCGCATTTCTTGGCTCTACCGTTGTTT |
| | | ATGTTGGGGTACACAAGGCTCAGAGACGAAT |
| | | TCAGAGACCTCTTAATTTCCTCAGACTCAATG |
| | | GATATTCCTATTGTTAAATTACCAATTGCTAC |
| | | GAAACTTTTCATGCTAATAGCAAATAACGTGA |
| | | CCCAGTTCATTTGTATCAAAGGTGTTAACATG |
| | | CTAGCTAGTAACACGGATGCTTTGACACTTTC |
| | | TGTCGTGCTTCTAGTGCGTAAATTTGTTAGTCT |
| | | TTTACTCAGTGTCTACATCTACAAGAACGTCC |
| | | TATCCGTGACTGCATACCTAGGGACCATCACC |
| | | GTGTTCCTGGGAGCTGGTTTGTATTCATATGG |
| | | TTCGGTCAAAACTGCACTGCCTCGCTGAAACA |
| | | ATCCACGTCTGTATGATACTCGTTTCAGAATT |
| | | TTTTTGATTTTCTGCCGGATATGGTTTCTCATC |
| | | TTTACAATCGCATTCTTAATTATACCAGAACG |
| | | TAATTCAATGATCCCAGTGACTCGTAACTCTT |
| | | ATATGTCAATTTAAGC |
| 67 | DNA encodes | ATGTCTGCCAACCTAAAATATCTTTCCTTGGG |
| | MmSLC35A3 | AATTTTGGTGTTTCAGACTACCAGTCTGGTTCT |
| | UDP-GlcNAc | AACGATGCGGTATTCTAGGACTTTAAAAGAG |
| | transporter | GAGGGGCCTCGTTATCTGTCTTCTACAGCAGT |
| | | GGTTGTGGCTGAATTTTTGAAGATAATGGCCT |
| | | GCATCTTTTTAGTCTACAAAGACAGTAAGTGT |
| | | AGTGTGAGAGCACTGAATAGAGTACTGCATG |
| | | ATGAAATTCTTAATAAGCCCATGGAAACCCTG |
| | | AAGCTCGCTATCCCGTCAGGGATATATACTCT |
| | | TCAGAACAACTTACTCTATGTGGCACTGTCAA |
| | | ACCTAGATGCAGCCACTTACCAGGTTACATAT |
| | | CAGTTGAAAATACTTACAACAGCATTATTTTC |
| | | TGTGTCTATGCTTGGTAAAAAATTAGGTGTGT |
| | | ACCAGTGGCTCTCCCTAGTAATTCTGATGGCA |
| | | GGAGTTGCTTTTGTACAGTGGCCTTCAGATTC |
| | | TCAAGAGCTGAACTCTAAGGACCTTTCAACAG |
| | | GCTCACAGTTTGTAGGCCTCATGGCAGTTCTC |
| | | ACAGCCTGTTTTTCAAGTGGCTTTGCTGGAGT |
| | | TTATTTTGAGAAAATCTTAAAAGAAACAAAAC |
| | | AGTCAGTATGGATAAGGAACATTCAACTTGGT |
| | | TTCTTTGGAAGTATATTTGGATTAATGGGTGT |
| | | ATACGTTTATGATGGAGAATTGGTCTCAAAGA |
| | | ATGGATTTTTTCAGGGATATAATCAACTGACG |
| | | TGGATAGTTGTTGCTCTGCAGGCACTTGGAGG |
| | | CCTTGTAATAGCTGCTGTCATCAAATATGCAG |
| | | ATAACATTTTAAAAGGATTTGCGACCTCCTTA |
| | | TCCATAATATTGTCAACAATAATATCTTATTTT |
| | | TGGTTGCAAGATTTTGTGCCAACCAGTGTCTT |
| | | TTTCCTTGGAGCCATCCTTGTAATAGCAGCTA |
| | | CTTTCTTGTATGGTTACGATCCCAAACCTGCA |
| | | GGAAATCCCACTAAAGCATAG |
| 68 | Sequence of the 5′- | GGCCTTGGAGGCCGCGGAAACGGCAGTAAAC |
| | region that was | AATGGAGCTTCATTAGTGGGTGTTATTATGGT |
| | used to knock into | CCCTGGCCGGGAACGAACGGTGAAACAAGAG |
| | the PpTRP1 locus: | GTTGCGAGGGAAATTTCGCAGATGGTGCGGG |
| | | AAAAGAGAATTTCAAAGGGCTCAAAATACTT |
| | | GGATTCCAGACAACTGAGGAAAGAGTGGGAC |
| | | GACTGTCCTCTGGAAGACTGGTTTGAGTACAA |
| | | CGTGAAAGAAATAAACAGCAGTGGTCCATTTT |
| | | TAGTTGGAGTTTTTCGTAATCAAAGTATAGAT |
| | | GAAATCCAGCAAGCTATCCACACTCATGGTTT |
| | | GGATTTCGTCCAACTACATGGGTCTGAGGATT |
| | | TTGATTCGTATATACGCAATATCCCAGTTCCT |
| | | GTGATTACCAGATACACAGATAATGCCGTCGA |
| | | TGGTCTTACCGGAGAAGACCTCGCTATAAATA |
| | | GGGCCCTGGTGCTACTGGACAGCGAGCAAGG |
| | | AGGTGAAGGAAAAACCATCGATTGGGCTCGT |
| | | GCACAAAAATTTGGAGAACGTAGAGGAAAAT |
| | | ATTTACTAGCCGGAGGTTTGACACCTGATAAT |
| | | GTTGCTCATGCTCGATCTCATACTGGCTGTATT |
| | | GGTGTTGACGTCTCTGGTGGGGTAGAAACAA |
| | | ATGCCTCAAAAGATATGGACAAGATCACACA |
| | | ATTTATCAGAAACGCTACATAA |
| 69 | Sequence of the 3′- | AAGTCAATTAAATACACGCTTGAAAGGACATT |
| | region that was | ACATAGCTTTCGATTTAAGCAGAACCAGAAAT |
| | used to knock into | GTAGAACCACTTGTCAATAGATTGGTCAATCT |
| | the PpTRP1 locus: | TAGCAGGAGCGGCTGGGCTAGCAGTTGGAAC |
| | | AGCAGAGGTTGCTGAAGGTGAGAAGGATGGA |
| | | GTGGATTGCAAAGTGGTGTTGGTTAAGTCAAT |
| | | CTCACCAGGGCTGGTTTTGCCAAAAATCAACT |
| | | TCTCCCAGGCTTCACGGCATTCTTGAATGACC |
| | | TCTTCTGCATACTTCTTGTTCTTGCATTCACCA |
| | | GAGAAAGCAAACTGGTTCTCAGGTTTTCCATC |
| | | AGGGATCTTGTAAATTCTGAACCATTCGTTGG |
| | | TAGCTCTCAACAAGCCCGGCATGTGCTTTTCA |
| | | ACATCCTCGATGTCATTGAGCTTAGGAGCCAA |
| | | TGGGTCGTTGATGTCGATGACGATGACCTTCC |
| | | AGTCAGTCTCTCCCTCATCCAACAAAGCCATA |
| | | ACACCGAGGACCTTGACTTGCTTGACCTGTCC |
| | | AGTGTAACCTACGGCTTCACCAATTTCGCAAA |
| | | CGTCCAATGGATCATTGTCACCCTTGGCCTTG |
| | | GTCTCTGGATGAGTGACGTTAGGGTCTTCCCA |
| | | TGTCTGAGGGAAGGCACCGTAGTTGTGAATGT |
| | | ATCCGTGGTGAGGGAAACAGTTACGAACGAA |
| | | ACGAAGTTTTCCCTTCTTTGTGTCCTGAAGAA |
| | | TTGGGTTCAGTTTCTCCTCCTTGGAAATCTCCA |
| | | ACTTGGCGTTGGTCCAACGGGGGACTTCAACA |
| | | ACCATGTTGAGAACCTTCTTGGATTCGTCAGC |
| | | ATAAAGTGGGATGTCGTGGAAAGGAGATACG |
| | | ACTTGGCCGTCTTGGCC |

While the present invention is described herein with reference to illustrated embodiments, it should be understood that the invention is not limited hereto. Those having ordinary skill in the art and access to the teachings herein will recognize additional modifications and embodiments within the scope thereof. Therefore, the present invention is limited only by the claims attached herein.

1. A composition comprising recombinant human granulocyte-colony stimulating factor (rHuGCSF) in a pharmaceutically acceptable carrier wherein about at least 18% of the rHuGCSF molecules in the composition have a mannose O-glycan.

2. The composition of claim 1, wherein about 40 to 50% of the rHuGCSF molecules in the composition have a mannose O-glycan.

3. The composition of claim 1, wherein the rHuGCSF molecules in the composition do not contain detectable mannobiose or larger O-glycans.

4. The composition of claim 1, wherein the rHuGCSF comprises at least one covalently attached hydrophilic polymer.

5. (canceled)

6. A Pichia pastoris host cell that produces a recombinant human granulocyte-colony stimulating factor (rHuGCSF) in which about 40 to 50% of the rHuGCSF obtained from the host cell have mannose O-glycans comprising:
(a) a nucleic acid molecule encoding the rHuGCSF; and
(b) one or more nucleic acid molecules, each encoding at least one secreted chimeric α-1,2-mannosidase I comprising at least the catalytic domain of an α-1,2-mannosidase I and a heterologous N-terminal signal sequence for directing extracellular secretion of the secreted chimeric α-1,2-mannosidase I, wherein when there is more than one secreted chimeric α-1,2-mannosidase I, the secreted chimeric α-1,2-mannosidase I can be the same or different.

7. The Pichia pastoris host cell of claim 6, wherein the α-1,2-mannosidase I is a fungal α-1,2-mannosidase I.

8. (canceled)

9. The Pichia pastoris host cell of claim 6, wherein the host cell further includes a deletion or disruption of its VPS10-1 gene.

10. The Pichia pastoris host cell of claim 6, wherein the host cell includes a deletion or disruption of its STE13 and/or DAP2 genes.

11. The Pichia pastoris host cell of claim 6, wherein the nucleic acid molecule in (a) encodes a rHuGCSF fusion protein having the structure A-B-C wherein A is a carrier protein having an N-terminal signal sequence for directing extracellular secretion of the fusion protein, B is a linker peptide that includes a protease cleavage site immediately preceding C, and C is the rHuGCSF.

12. (canceled)

13. The Pichia pastoris host cell of claim 11, wherein A is a Pichia pastoris cellulase-like protein 1 (Clp1p), the protease cleavage site in B is a Kex 2p cleavage site, and C is rHuGCSF with an N-terminal methionine residue.

14. A nucleic acid molecule encoding a fusion protein having the structure A-B-C wherein A is a carrier protein having an N-terminal signal sequence for directing extracellular secretion of the fusion protein, B is a linker peptide that includes a protease cleavage site immediately preceding C, and C is a rHuGCSF.

15. The nucleic acid molecule of claim 14, wherein A is human serum albumin, Pichia pastoris cellulase-like protein 1 (Clp1p), Aspergillus niger glucoamylase, or anti-CD20 light chain.

16. The nucleic acid molecule of claim 15, wherein A is a Pichia pastoris cellulase-like protein 1 (Clp1p), the protease cleavage site in B is a Kex 2p cleavage site, and C is rHuGCSF with an N-terminal methionine residue.

17. A method for making a composition of recombinant human granulocyte-colony stimulating factor (rHuGCSF) in which about 40 to 50% of the rHuGCSF in the composition have mannose O-glycans in Pichia pastoris comprising:
(a) providing a recombinant Pichia pastoris host cell that includes
(i) a nucleic acid molecule encoding the rHuGCSF; and
(ii) one or more nucleic acid molecules, each encoding at least one secreted chimeric α-1,2-mannosidase I comprising at least the catalytic domain of an α-1,2-mannosidase I and a heterologous N-terminal signal sequence for directing extracellular secretion of the secreted chimeric α-1,2-mannosidase I, wherein when there is more than one secreted chimeric α-1,2-mannosidase I, the secreted chimeric α-1,2-mannosidase I can be the same or different;
(b) growing the host cell in a medium under conditions that induce expression of the nucleic acid molecule encoding the rHuGCSF to produce the rHuGCSF, which secreted into the medium; and
(c) recovering the rHuGCSF from the medium to produce the composition of recombinant human granulocyte-colony stimulating factor (rHuGCSF) in which about 40 to 50% of the rHuGCSF in the composition have mannose O-glycans.

18. The method of claim 17, wherein the α-1,2-mannosidase I is a fungal α-1,2-mannosidase I.

19. (canceled)

20. The method of claim 17, wherein the host cell further includes a deletion or disruption of its VPS10-1 gene.

21. The method of claim 17, wherein the host cell includes a deletion or disruption of its STE13 and/or DAP2 genes.

22. The method of claim 17, wherein the nucleic acid molecule in (a) encodes a rHuGCSF fusion protein having the structure A-B-C wherein A is a carrier protein having an N-terminal signal sequence for directing extracellular secretion of the fusion protein, B is a linker peptide that includes a protease cleavage site immediately preceding C, and C is the rHuGCSF.

23. (canceled)

24. (canceled)

25. The method of claim 17, wherein further included is step wherein the rHuGCSF is conjugated to at least one hydrophilic polymer.
